(19) United States
(12) Patent Application Publication
(10) Pub. No.: US 2020/0115382 A1
(43) Pub. Date: Apr. 16, 2020

(54) CURABLE COMPOSITION, CURED FILM, OPTICAL FILTER, SOLID IMAGE PICKUP ELEMENT, IMAGE DISPLAY DEVICE, INFRARED SENSOR, DISPERSING AUXILIARY AGENT, DISPERSION, AND METHOD OF MANUFACTURING DISPERSION

(71) Applicant: FUJIFILM Corporation, Tokyo (JP)

(72) Inventors: Takuya TSURUTA, Haibara-gun (JP); Hiroaki TSUYAMA, Haibara-gun (JP); Kyohei ARAYAMA, Haibara-gun (JP); Suguru SAMEJIMA, Haibara-gun (JP); Tokihiko MATSUMURA, Haibara-gun (JP); Tetsushi MIYATA, Haibara-gun (JP); Kazutaka TAKAHASHI, Haibara-gun (JP)

(73) Assignee: FUJIFILM Corporation, Tokyo (JP)

(21) Appl. No.: 16/709,490

(22) Filed: Dec. 10, 2019

Related U.S. Application Data
(63) Continuation of application No. PCT/JP2018/021432, filed on Jun. 5, 2018.

(30) Foreign Application Priority Data
Jun. 12, 2017 (JP) 2017-115165
Jan. 18, 2018 (JP) 2018-006353
May 23, 2018 (JP) 2018-098421

(51) Int. Cl. C07D 487/04; C07F 5/02; G02B 5/22; C08L 101/00; C09B 67/00

(52) U.S. Cl. CPC C07D 487/04; C07F 5/027; C08L 2203/16; C08L 101/00; C09B 68/4673; G02B 5/22

(57) ABSTRACT

Provided are a curable composition, a cured film, an optical filter, a solid image pickup element, an image display device, an infrared sensor, a dispersing auxiliary agent, a dispersion, and a method of manufacturing a dispersion. With the curable composition, a cured film having excellent moisture resistance in which the formation of an aggregate derived from a compound having a colorant skeleton is suppressed can be formed. This curable composition includes: a compound A having a structure in which at least one functional group selected from an acid group having a pKa of 3 or lower and a Clog P value of −1.1 or higher, an anionic group obtained by dissociating one or more hydrogen atoms from the acid group, or a salt of the acid group is bonded to a π-conjugated structure of a colorant skeleton and having a maximum absorption wavelength in a wavelength range of 650 to 1200 nm; a curable compound; and a solvent.

### CROSS-REFERENCE TO RELATED APPLICATIONS

This application is a Continuation of PCT International Application No. PCT/JP2018/021432 filed on Jun. 5, 2018, which claims priority under 35 U.S.C § 119(a) to Japanese Patent Application No. 2017-115165 filed on Jun. 12, 2017, Japanese Patent Application No. 2018-006353 filed on Jan. 18, 2018, and Japanese Patent Application No. 2018-098421 filed on May 23, 2018. Each of the above application(s) is hereby expressly incorporated by reference, in its entirety, into the present application.

### BACKGROUND OF THE INVENTION

### 1. Field of the Invention

The present invention relates to a curable composition, a cured film, an optical filter, a solid image pickup element, an image display device, an infrared sensor, a dispersing auxiliary agent, a dispersion, and a method of manufacturing a dispersion

### 2. Description of the Related Art

Using a curable composition including a colorant, a curable compound, and a solvent, a cured film such as a color filter or a near infrared cut filter has been manufactured.

For example, JP2015-030742A describes that a color filter is manufactured using a coloring composition including a colorant polymer as a colorant, the colorant polymer including: a polymer anion that includes a repeating unit having an anion structure obtained by dissociating an organic acid having a lower pKa than the pKa of sulfuric acid; and a cation having a colorant structure.

In addition, JP2016-102191A describes that a color filter is manufactured using a coloring composition including a colorant compound, a curable compound, and a solvent, the colorant compound including: a colorant structure having a cation site; and a predetermined anion site, in which the anion site and the cation site are bonded to each other through a covalent bond and are present in the same molecule.

In addition, JP2015-071743A describes that a color filter is manufactured using a coloring composition including: a colorant in which 1 to 13 monovalent substituents including a repeating unit derived from a vinyl compound and 2 to 14 colorant structures are each independently bonded to a (m+n)-valent linking group; and a curable compound. Examples of the colorant structure include a colorant structure including a cation site and an anion site in one molecule.

### SUMMARY OF THE INVENTION

In a cured film that is formed using a curable composition including a colorant, a curable compound, and a solvent, an aggregate derived from a compound having a colorant skeleton may be formed. In addition, in general, a compound having an absorption in a near infrared range has a wide π-conjugated system. This compound is likely to aggregate particularly during film formation. In a case where an aggregate derived from a compound having a colorant skeleton is formed in the film, a variation in spectral characteristics may occur, and the smoothness of a film surface may deteriorate.

In addition, recently, further improvement of moisture resistance of a cured film has been desired, and the development of a cured film in which spectral characteristics and the like are not likely to vary even after being exposed to a high humidity environment has been desired.

Accordingly, an object of the present invention is to provide a curable composition with which a cured film having excellent moisture resistance in which the formation of an aggregate derived from a compound having a colorant skeleton is suppressed can be formed. In addition, another object of the present invention is to provide a cured film having excellent moisture resistance in which the formation of an aggregate derived from a compound having a colorant skeleton is suppressed, and an optical filter, a solid image pickup element, an image display device, and an infrared sensor that include the above-described cured film. In addition, still another object of the present invention is to provide a dispersing auxiliary agent, a dispersion, and a method of manufacturing a dispersion.

The present inventors conducted a thorough investigation under the above-described circumstances and found that with a curable composition including a compound A described below, a curable compound, and a solvent, a cured film having excellent moisture resistance in which the formation of an aggregate derived from a compound having a colorant skeleton is suppressed can be formed, thereby completing the present invention. The present invention provides the following.

<1> A curable composition comprising:

a compound A having a structure in which at least one functional group selected from an acid group having a pKa of 3 or lower and a Clog P value of −1.1 or higher, an anionic group obtained by dissociating one or more hydrogen atoms from the acid group, or a salt of the acid group is bonded to a π-conjugated structure of a colorant skeleton and having a maximum absorption wavelength in a wavelength range of 650 to 1200 nm;

a curable compound; and

a solvent.

<2> The curable composition according to <1>,

in which the functional group has at least one structure selected from an acid structure selected from an imide acid structure, a methide acid structure, a boronic acid structure, a carboxylic acid structure, or a sulfonic acid structure, an anion obtained by dissociating one or more hydrogen atoms from the acid structure, or a salt of the acid structure.

<3> The curable composition according to <1> or <2>,

in which the functional group includes a partial structure represented by the following Formula (1),

X¹—Y¹—Z¹ (1),

X¹ and Z¹ each independently represent —SO₂—, —CO—, —B(OH)—, or —P(═O)(OH)—,

Y¹ represents —NH—, —N—, or -NM¹-, and

M¹ represents an atom or an atomic group forming a salt.

<4> The curable composition according to <3>,

in which at least one of X¹ or Z¹ represents —SO₂—.

<5> The curable composition according to any one of <1> to <3>,

in which the functional group is a group represented by the following Formula (10),

-L¹⁰-R⁹—X₁₀—Y¹⁰—Z¹⁰—R¹⁰ (10),

in Formula (10), L¹⁰ represents a single bond or a divalent linking group,

X¹⁰ and Z¹⁰ each independently represent —SO₂—, —CO—, —B(OH)—, or —P(═O)(OH)—,

Y¹⁰ represents —NH—, —N—, or -NM¹-,

M¹ represents an atom or an atomic group forming a salt,

R⁹ represents a single bond or a hydrocarbon group having 1 or more carbon atoms which may include a substituent, and

R¹⁰ represents a halogen atom, a hydroxyl group, or a hydrocarbon group having 1 or more carbon atoms which may include a substituent.

<6> The curable composition according to <5>,

in which X¹⁰ represents —CO— and Z¹⁰ represents —SO₂—.

<7> The curable composition according to <5> or <6>,

in which R¹⁰ represents a hydrocarbon group having 1 or more carbon atoms which includes a fluorine atom.

<8> The curable composition according to <1> or <2>,

in which the functional group is a group represented by the following Formula (20) or the following Formula (30),

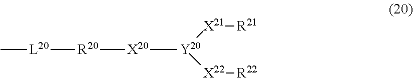

in Formula (20), L²⁰ represents a single bond or a divalent linking group,

X²⁰ to X²² each independently represent —SO₂—, —CO—, —B(OH)—, or —P(═O)(OH)—,

Y²⁰ represents —CH<, —C⁻<, or -CM²<,

M² represents an atom or an atomic group forming a salt,

R²⁰ represents a single bond or a hydrocarbon group having 1 or more carbon atoms which may include a substituent, and

R²¹ and R²² each independently represent a halogen atom, a hydroxyl group, or a hydrocarbon group having 1 or more carbon atoms which may include a substituent, and

-L³⁰-R³⁰—Y³⁰ (30),

in Formula (30), L³⁰ represents a single bond or a divalent linking group,

R³⁰ represents a hydrocarbon group having 1 or more carbon atoms which may include a substituent,

Y³⁰ represents —COOH, —COO⁻, —COOM³, —SO₃H, —SO₃⁻, —SO₃M³, or —B⁻(Rb1)(Rb2)(Rb3),

M³ represents an atom or an atomic group forming a salt, and

Rb1 to Rb3 each independently represent a halogen atom or a hydrocarbon group having 1 or more carbon atoms which may include a substituent.

<9> The curable composition according to any one of <1> to <8>,

in which the colorant skeleton is at least one selected from a pyrrolopyrrole colorant skeleton, a diimonium colorant skeleton, a phthalocyanine colorant skeleton, a naphthalocyanine colorant skeleton, a polymethine colorant skeleton, a pyrromethene colorant skeleton, or a perylene colorant skeleton.

<10> The curable composition according to any one of <1> to <8>,

in which the colorant skeleton is a pyrrolopyrrole colorant skeleton.

<11> The curable composition according to any one of <1> to <10>,

in which the compound A is a compound represented by Formula (A1),

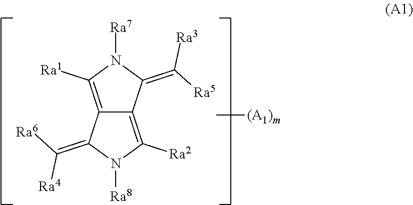

in Formula (A1), Ra¹ and Ra² each independently represent an alkyl group, an aryl group, or a heteroaryl group,

Ra³, Ra⁴, Ra⁵, and Ra⁶ each independently represent a cyano group, an acyl group, an alkoxycarbonyl group, an alkylsulfinyl group, an arylsulfinyl group, or a heteroaryl group,

Ra⁷ and Ra⁸ each independently represent a hydrogen atom, an alkyl group, an aryl group, a heteroaryl group, —BRa⁹Ra¹⁰, or a metal atom,

Ra⁷ may form a covalent bond or a coordinate bond with Ra¹, Ra³, or Ra⁵,

Ra⁸ may form a covalent bond or a coordinate bond with Ra², Ra⁴, or Ra⁶,

Ra⁹ and Ra¹⁰ each independently represent a hydrogen atom, a halogen atom, an alkyl group, an alkenyl group, an aryl group, a heteroaryl group, an alkoxy group, an aryloxy group, or a heteroaryloxy group,

Ra⁹ and Ra¹⁰ may be bonded to each other to form a ring,

A₁ represents at least one functional group selected from an acid group having a pKa of 3 or lower and a Clog P value of −1.1 or higher, an anionic group obtained by dissociating one or more hydrogen atoms from the acid group, or a salt of the acid group,

m represents an integer of 1 to 10, and

in a case where m represents 2 or more, a plurality of A₁'s may be the same as or different from each other.

<12> The curable composition according to any one of <1> to <10>,

in which the compound A is a compound represented by Formula (A2),

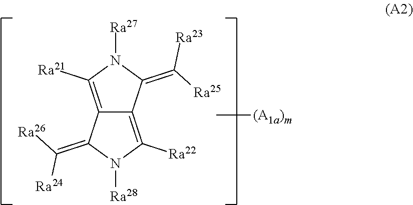

in Formula (A2), Ra²¹ and Ra²² each independently represent an alkyl group, an aryl group, or a heteroaryl group,

Ra²³, Ra²⁴, Ra²⁵, and Ra²⁶ each independently represent a cyano group, an acyl group, an alkoxycarbonyl group, an alkylsulfinyl group, an arylsulfinyl group, or a heteroaryl group,

Ra²⁷ and Ra²⁸ each independently represent —BRa²⁹Ra³⁰,

Ra²⁹ and Ra³⁰ each independently represent a hydrogen atom, a halogen atom, an alkyl group, an alkenyl group, an aryl group, a heteroaryl group, an alkoxy group, an aryloxy group, or a heteroaryloxy group,

Ra²⁹ and Ra³⁰ may be bonded to each other to form a ring,

A₁ₐ represents at least one functional group selected from an acid group having a pKa of 3 or lower and a Clog P value of −1.1 or higher, an anionic group obtained by dissociating one or more hydrogen atoms from the acid group, or a salt of the acid group,

m represents an integer of 1 to 10, and

in a case where m represents 2 or more, a plurality of A₁ₐ's may be the same as or different from each other.

<13> The curable composition according to any one of <1> to <12>, further comprising:

a colorant other than the compound A.

<14> A cured film which is formed using the curable composition according to any one of <1> to <13>.

<15> An optical filter comprising:

the cured film according to <14>.

<16> The optical filter according to <15>,

in which the optical filter is a near infrared cut filter or an infrared transmitting filter.

<17> A solid image pickup element comprising:

the cured film according to <14>.

<18> An image display device comprising:

the cured film according to <14>.

<19> An infrared sensor comprising:

the cured film according to <14>.

<20> A dispersing auxiliary agent comprising:

a compound having a structure in which at least one functional group selected from an acid group having a pKa of 3 or lower and a Clog P value of −1.1 or higher, an anionic group obtained by dissociating one or more hydrogen atoms from the acid group, or a salt of the acid group is bonded to a π-conjugated structure of a colorant skeleton.

<21> The dispersing auxiliary agent according to <20>,

in which the colorant skeleton is at least one selected from a pyrrolopyrrole colorant skeleton, a diimonium colorant skeleton, a phthalocyanine colorant skeleton, a naphthalocyanine colorant skeleton, a polymethine colorant skeleton, a xanthene colorant skeleton, a pyrromethene colorant skeleton, a quinacridone colorant skeleton, an azo colorant skeleton, a diketo pyrrolo pyrrole colorant skeleton, an anthraquinone colorant skeleton, a benzimidazolone colorant skeleton, a triazine colorant skeleton, an isophthalic acid colorant skeleton, an isoindoline colorant skeleton, a quinoline colorant skeleton, a benzothiazole colorant skeleton, a quinoxaline colorant skeleton, or a benzoxazole colorant skeleton.

<22> A dispersion comprising:

a pigment;

the dispersing auxiliary agent according to claim 20 or 21;

a dispersant; and

a solvent.

<23> A method of manufacturing a dispersion comprising:

a step of dispersing a pigment in the presence of the dispersing auxiliary agent according to <20> or <21>, a dispersant, and a solvent.

<24> A compound which is represented by Formula (A2),

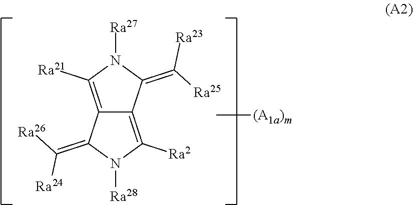

in Formula (A2), Ra²¹ and Ra²² each independently represent an alkyl group, an aryl group, or a heteroaryl group,

Ra²³, Ra²⁴, Ra²⁵, and Ra²⁶ each independently represent a cyano group, an acyl group, an alkoxycarbonyl group, an alkylsulfinyl group, an arylsulfinyl group, or a heteroaryl group,

Ra²⁷ and Ra²⁸ each independently represent —BRa²⁹Ra³⁰,

Ra²⁹ and Ra³⁰ each independently represent a hydrogen atom, a halogen atom, an alkyl group, an alkenyl group, an aryl group, a heteroaryl group, an alkoxy group, an aryloxy group, or a heteroaryloxy group,

Ra²⁹ and Ra³⁰ may be bonded to each other to form a ring,

A₁ₐ represents at least one functional group selected from an acid group having a pKa of 3 or lower and a Clog P value of −1.1 or higher, an anionic group obtained by dissociating one or more hydrogen atoms from the acid group, or a salt of the acid group,

m represents an integer of 1 to 10, and

in a case where m represents 2 or more, a plurality of A₁ₐ's may be the same as or different from each other.

<25> The compound according to <24>,

in which A₁ₐ includes a partial structure represented by the following Formula (1),

X¹—Y¹—Z¹ (1),

X¹ and Z¹ each independently represent —SO₂—, —CO—, —B(OH)—, or —P(═O)(OH)—,

Y¹ represents —NH—, —N—, or -NM¹-, and

M¹ represents an atom or an atomic group forming a salt.

<26> The compound according to claim 24,

in which A₁ₐ represents a group represented by the following Formula (10),

-L¹⁰-R⁹—X¹⁰—Y¹⁰—Z¹⁰—R¹⁰ (10),

in Formula (10), L¹⁰ represents a single bond or a divalent linking group,

X¹⁰ and Z¹⁰ each independently represent —SO₂—, —CO—, —B(OH)—, or —P(═O)(OH)—,

Y¹⁰ represents —NH—, —N—, or -NM¹-,

M¹ represents an atom or an atomic group forming a salt,

R⁹ represents a single bond or a hydrocarbon group having 1 or more carbon atoms which may include a substituent, and

R¹⁰ represents a halogen atom, a hydroxyl group, or a hydrocarbon group having 1 or more carbon atoms which may include a substituent.

<27> The compound according to <26>,

in which X¹⁰ represents —CO— and Z¹⁰ represents —SO₂—.

<28> The compound according to <26> or <27>,

in which R¹⁰ represents a hydrocarbon group having 1 or more carbon atoms which includes a fluorine atom.

<29> The compound according to <24>,

in which A₁ₐ represents a group represented by the following Formula (20) or the following Formula (30),

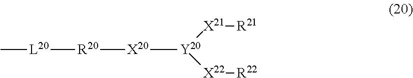

in Formula (20), L²⁰ represents a single bond or a divalent linking group,

X²⁰ to X²² each independently represent —SO₂—, —CO—, —B(OH)—, or —P(═O)(OH)—,

Y²⁰ represents —CH<, —C⁻<, or -CM²<,

M² represents an atom or an atomic group forming a salt,

R²⁰ represents a single bond or a hydrocarbon group having 1 or more carbon atoms which may include a substituent, and

R²¹ and R²² each independently represent a halogen atom, a hydroxyl group, or a hydrocarbon group having 1 or more carbon atoms which may include a substituent, and

-L³⁰-R³⁰—Y³⁰ (30),

in Formula (30), L³⁰ represents a single bond or a divalent linking group,

R³⁰ represents a hydrocarbon group having 1 or more carbon atoms which may include a substituent,

Y³⁰ represents —COOH, —COO⁻, —COOM³, —SO₃H, —SO₃⁻, —SO₃M³, or —B⁻(Rb1)(Rb2)(Rb3),

M³ represents an atom or an atomic group forming a salt, and

Rb1 to Rb3 each independently represent a halogen atom or a hydrocarbon group having 1 or more carbon atoms which may include a substituent.

According to the present invention, it is possible to provide a curable composition with which a cured film having excellent moisture resistance in which the formation of an aggregate derived from a compound having a colorant skeleton is suppressed can be formed. In addition, according to the present invention, it is possible to provide a cured film having excellent moisture resistance in which the formation of an aggregate derived from a compound having a colorant skeleton is suppressed, and an optical filter, a solid image pickup element, an image display device, and an infrared sensor that include the above-described cured film. In addition, according to the present invention, it is possible to provide a dispersing auxiliary agent having excellent dispersibility, a dispersion, and a method of manufacturing a dispersion.

### BRIEF DESCRIPTION OF THE DRAWINGS

FIG. 1 is a schematic diagram showing an embodiment of an infrared sensor.

### DESCRIPTION OF THE PREFERRED EMBODIMENTS

Hereinafter, the details of the present invention will be described.

In this specification, numerical ranges represented by “to” include numerical values before and after “to” as lower limit values and upper limit values.

In this specification, unless specified as a substituted group or as an unsubstituted group, a group (atomic group) denotes not only a group (atomic group) having no substituent but also a group (atomic group) having a substituent. For example, “alkyl group” denotes not only an alkyl group having no substituent (unsubstituted alkyl group) but also an alkyl group having a substituent (substituted alkyl group).

In this specification, unless specified otherwise, “exposure” denotes not only exposure using light but also drawing using a corpuscular beam such as an electron beam or an ion beam. Examples of the light used for exposure include an actinic ray or radiation, for example, a bright light spectrum of a mercury lamp, a far ultraviolet ray represented by excimer laser, an extreme ultraviolet ray (EUV ray), an X-ray, or an electron beam.

In this specification, “(meth)acrylate” denotes either or both of acrylate and methacrylate, “(meth)acryl” denotes either or both of acryl and methacryl, and “(meth)acryloyl” denotes either or both of acryloyl and methacryloyl.

In this specification, a weight-average molecular weight and a number-average molecular weight are defined as values in terms of polystyrene obtained by gel permeation chromatography (GPC).

In this specification, in a chemical formula, Me represents a methyl group, Et represents an ethyl group, Bu represents a butyl group, and Ph represents a phenyl group.

In this specification, near infrared light denotes light (electromagnetic wave) having a wavelength in a range of 700 to 2500 nm.

In this specification, a total solid content denotes the total mass of all the components of the composition excluding a solvent.

In this specification, the term “step” denotes not only an individual step but also a step which is not clearly distinguishable from another step as long as an effect expected from the step can be achieved.

<Curable Composition>

A curable composition according to an embodiment of the present invention comprises:

a compound A having a structure in which at least one functional group selected from an acid group having a pKa of 3 or lower and a Clog P value of −1.1 or higher, an anionic group obtained by dissociating one or more hydrogen atoms from the acid group, or a salt of the acid group is bonded to a π-conjugated structure of a colorant skeleton and having a maximum absorption wavelength in a wavelength range of 650 to 1200 nm;

a curable compound; and

a solvent.

Hereinafter, “at least one functional group selected from an acid group having a pKa of 3 or lower and a Clog P value of −1.1 or higher, an anionic group obtained by dissociating one or more hydrogen atoms from the acid group, or a salt of the acid group” will also be referred to as “functional group A”.

According to the present invention, by using the curable composition that includes the compound A having the functional group A, a cured film having excellent moisture resistance in which the formation of an aggregate derived from a compound having a colorant skeleton is suppressed can be formed. The mechanism in which the effect can be obtained is not clear but is presumed to be as follows. The compound A has an acid group having a pKa of 3 or lower or a group (an anionic group or a salt) derived from the acid group, and thus it is presumed that aggregation of the compound A in the film can be suppressed. In addition, the compound A has the acid group or the group derived from the acid group, and thus it is presumed that, in a case where a colorant (other colorant) other than the compound A is further included, aggregation of the compound A and the other colorant or aggregation of the other colorant can be suppressed. Therefore, it is presumed that the formation of an aggregate derived from a compound having a colorant skeleton in the film can be effectively suppressed. In addition, in general, a group having a low pKa has high hydrophilicity such that moisture resistance of the obtained cured film tends to deteriorate. The functional group A in the compound A is an acid group having a pKa of 3 or lower and a Clog P value of −1.1 or higher and a group derived from the acid group. By the compound A including this functional group, penetration of water into the cured film can be effectively suppressed, and thus it is presumed that excellent moisture resistance can be obtained. Therefore, according to the present invention, it is presumed that a cured film having excellent moisture resistance in which the formation of an aggregate derived from a compound having a colorant skeleton is suppressed can be formed.

In addition, the functional group A in the compound A is an acid group having a pKa of 3 or less or a group (an anionic group or a salt) derived from the acid group. Therefore, it is presumed that an interaction with a basic component functions more strongly. Therefore, for example, in a case where a compound that includes a resin (for example, a basic dispersant and/or an amphoteric dispersant) having a basic group is used as the curable compound, an interaction between the above-described functional group A in the compound A and the basic group in the basic dispersant or the amphoteric dispersant functions more strongly, and the dispersibility of the compound A in the composition can be further improved. Further, aggregation of the compound A or aggregation a colorant other than the compound A in the film can be more effectively suppressed, and a cured film in which the formation of an aggregate derived from a compound having a colorant skeleton is further suppressed can also be formed. In addition, the developability of the compound A itself for an alkali developer can be improved, and a curable composition having excellent developability can also be obtained.

Hereinafter, each of the components of the curable composition according to the embodiment of the present invention will be described.

<<Compound A>>

The curable composition according to the embodiment of the present invention comprises a compound A (hereinafter, also simply referred to as “compound A”) having a structure in which at least one functional group (functional group A) selected from an acid group having a pKa of 3 or lower and a Clog P value of −1.1 or higher, an anionic group obtained by dissociating one or more hydrogen atoms from the acid group, or a salt of the acid group is bonded to a π-conjugated structure of a colorant skeleton and having a maximum absorption wavelength in a wavelength range of 650 to 1200 nm.

In the present invention, the pKa and the Clog P value of the functional group A are values calculated by substituting a direct bond to the π-conjugated structure of the colorant skeleton with a methyl group. In addition, the pKa is a value in water and is obtained by predictive calculation using ACD/Labs Ver. 8.08 (manufactured by Advanced Chemistry Development Inc.). In addition, the Clog P value is a calculated value of Log P that is a common logarithm of a partition coefficient P of I-octanol/water, and is obtained by predictive calculation using ChemiBioDraw Ultra Ver. 13.02.3021 (manufactured by Cambridge Soft Corporation).

In addition, in a case where a terminal bonded to the functional group A in the z-conjugated structure of the colorant skeleton is —C(═O)—, —S(═O)₂—, or —P(═O)—, this group is included in the functional group A. That is, in the case of a compound 1 or 2 having the following structure, a group surrounded by a circle corresponds to the functional group A. The functional group A (CF₃—SO₂—NHCO—C₄H₈—O—) in the compound 1 having the following structure is an acid group having a Clog P value of 1.09 and a pKa of −1.43. In addition, the functional group A (SO₃H—C₃F₆—SO₂—) in the compound 2 having the following structure is an acid group having a Clog P value of 1.44 and a pKa of −3.38.

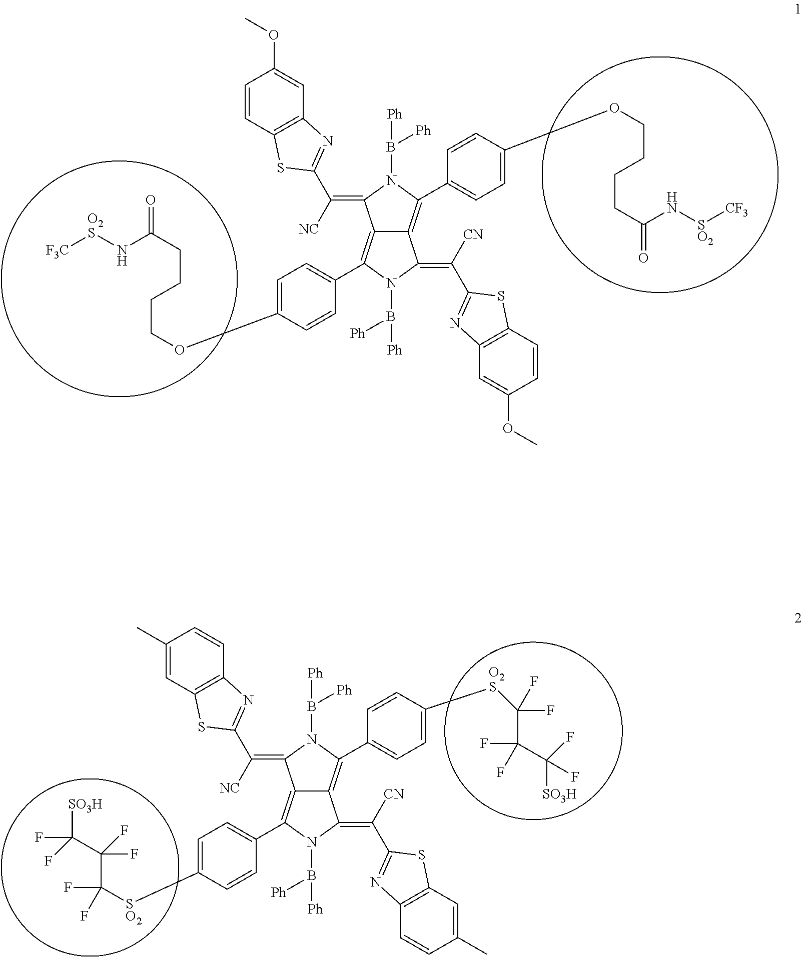

The compound A used in the curable composition according to the embodiment of the present invention has a maximum absorption wavelength in a wavelength range of 650 to 1200 nm. By using this compound, near infrared shielding properties of the obtained cured film can be improved. It is preferable that the compound A is a compound having a maximum absorption wavelength in a wavelength range of 700 to 1000 nm.

The compound A has a colorant skeleton having a π-conjugated structure. The number of atoms constituting the π-conjugated structure other than hydrogen is preferably 14 or more, more preferably 20 or more, still more preferably 25 or more, and still more preferably 30 or more. For example, the upper limit is preferably 80 or less and more preferably 50 or less. The number of monocyclic or fused aromatic rings in the π-conjugated structure included in the colorant skeleton is preferably 2 or more, more preferably 3 or more, still more preferably 4 or more, and still more preferably 5 or more. The upper limit is preferably 100 or less, more preferably 50 or less, and still more preferably 30 or less. Examples of the aromatic ring include a benzene ring, a naphthalene ring, a pentalene ring, an indene ring, an azulene ring, a heptalene ring, an indacene ring, a perylene ring, a pentacene ring, a quaterrylene ring, an acenaphthene ring, a phenanthrene ring, an anthracene ring, a naphthacene ring, a chrysene ring, a triphenylene ring, a fluorene ring, a pyridine ring, a quinoline ring, an isoquinoline ring, an imidazole ring, a benzimidazole ring, a pyrazole ring, a thiazole ring, a benzothiazole ring, a triazole ring, a benzotriazole ring, an oxazole ring, a benzoxazole ring, an imidazoline ring, a pyrazine ring, a quinoxaline ring, a pyrimidine ring, a quinazoline ring, a pyridazine ring, a triazine ring, a pyrrole ring, an indole ring, an isoindole ring, a carbazole ring, and a fused ring including the above-described ring.

It is preferable that the colorant skeleton in the compound A is a colorant skeleton derived from a colorant compound having an absorption in a near infrared range. Preferable specific examples of the colorant skeleton include at least one selected from a pyrrolopyrrole colorant skeleton, a diimonium colorant skeleton, a phthalocyanine colorant skeleton, a naphthalocyanine colorant skeleton, a polymethine colorant skeleton, a pyrromethene colorant skeleton, or a perylene colorant skeleton. In addition, examples of the polymethine colorant skeleton depending on the kind of an atomic group to be bonded include a cyanine colorant skeleton, a merocyanine colorant skeleton, a squarylium colorant skeleton, a croconium colorant skeleton, and an oxonol colorant skeleton. Among these, a cyanine colorant skeleton, a squarylium colorant skeleton, or an oxonol colorant skeleton is preferable, and a cyanine colorant skeleton or a squarylium colorant skeleton is more preferable. The colorant skeleton in the compound A is more preferably at least one selected from a pyrrolopyrrole colorant skeleton, a phthalocyanine colorant skeleton, a naphthalocyanine colorant skeleton, or a polymethine colorant skeleton, still more preferably a pyrrolopyrrole colorant skeleton or a polymethine colorant skeleton, and still more preferably a pyrrolopyrrole colorant skeleton.

The compound A has at least one functional group (functional group A) selected from an acid group having a pKa of 3 or lower and a Clog P value of −1.1 or higher, an anionic group obtained by dissociating one or more hydrogen atoms from the acid group, or a salt of the acid group. Here, in a case where the functional group A is the anionic group or the salt described above, the pKa of the functional group A itself may be 3 or lower and higher than 3. In addition, the Clog P value may be −1.1 or higher and lower than −1.1. That is, in a case where the functional group A is the anionic group or the salt described above, each of the pKa and the Clog P value of the acid group derived from the anionic group or the salt may be in the above-described range.

The pKa of the acid group in the functional group A is 3 or lower, preferably 2 or lower, more preferably 0 or lower, and still more preferably −1 or lower. In a case where the pKa of the acid group is 3 or lower, a cured film in which the formation of an aggregate derived from a compound having a colorant structure is suppressed can be formed. Further, for example, the dispersibility of the compound having a colorant structure in the composition can be improved.

The Clog P value of the acid group in the functional group A is −1.1 or higher, preferably −1 or higher, more preferably 0 or higher, and still more preferably 1 or higher. In a case where the Clog P value of the acid group is −1.1 or higher, a cured film having excellent moisture resistance can be formed.

It is preferable that the functional group A is a group having at least one structure selected from an acid structure selected from an imide acid structure, a methide acid structure, a boronic acid structure, a carboxylic acid structure, or a sulfonic acid structure, an anion obtained by dissociating one or more hydrogen atoms from the acid structure, or a salt of the acid structure, and from the viewpoint of easily adjusting the pKa or the Clog P value during synthesis of the compound or obtaining raw materials, it is more preferable that the functional group A is a group having at least one structure selected from an imide acid structure, an imide anion structure, or a salt of the imide acid structure. For example, in a case where the functional group A is a group having at least one structure selected from an imide acid structure, an imide anion structure, or a salt of the imide acid structure, the pKa or the Clog P value can be easily adjusted by changing a substituent bonded to the imido group.

In addition, it is preferable that the functional group A is a group having a partial structure represented by the following Formula (1).

X¹—Y¹—Z¹ (1)

In Formula (1), X¹ and Z¹ each independently represent —SO₂—, —CO—, —B(OH)—, or —P(═O)(OH)—, and it is preferable that at least one of X¹ or Z¹ represents —SO₂—. It is preferable one of X¹ or Z¹ represents —SO₂— and another one of X¹ or Z¹ represents —SO₂— or —CO—, and it is more preferable one of X¹ or Z¹ represents —SO₂— and another one of X¹ or Z¹ represents —CO—.

In Formula (1), Y¹ represents —NH—, —N⁻—, or —NM¹-, and M¹ represents an atom or an atomic group forming a salt. Examples of the atom or the atomic group M¹ forming a salt include an alkali metal ion (for example, Li⁺, Na⁺, or K⁺), an ammonium cation, a pyridine cation, an imidazole cation, and a sulfonium cation.

The functional group A in the compound A is preferably at least one group selected from a group represented by the following Formula (10), a group represented by the following Formula (20), or a group represented by the following Formula (30) and more preferably a group represented by the following Formula (10).

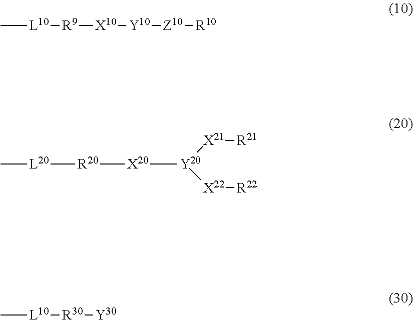

In Formula (10), L¹⁰ represents a single bond or a divalent linking group, X¹⁰ and Z¹⁰ each independently represent —SO₂—, —CO—, —B(OH)—, or —P(═O)(OH)—, Y¹⁰ represents —NH—, —N—, or -NM¹-, M¹ represents an atom or an atomic group forming a salt, R⁹ represents a single bond or a hydrocarbon group having 1 or more carbon atoms which may include a substituent, and R⁰ represents a halogen atom, a hydroxyl group, or a hydrocarbon group having 1 or more carbon atoms which may include a substituent.

In Formula (20), L²⁰ represents a single bond or a divalent linking group, X²⁰ to X²² each independently represent —SO₂—, —CO—, —B(OH)—, or —P(═O)(OH)—, Y²⁰ represents —CH<, —C⁻<, or -CM²<, M² represents an atom or an atomic group forming a salt, R²⁰ represents a single bond or a hydrocarbon group having 1 or more carbon atoms which may include a substituent, and R²¹ and R²² each independently represent a halogen atom, a hydroxyl group, or a hydrocarbon group having 1 or more carbon atoms which may include a substituent.

In Formula (30), L³⁰ represents a single bond or a divalent linking group, R³⁰ represents a hydrocarbon group having 1 or more carbon atoms which may include a substituent, Y³⁰ represents —COOH, —COO⁻, —COOM³, —SO₃H, —SO₃⁻, —SO₃M³, or —B⁻(Rb1)(Rb2)(Rb3), M³ represents an atom or an atomic group forming a salt, and Rb1 to Rb3 each independently represent a halogen atom or a hydrocarbon group having 1 or more carbon atoms which may include a substituent.

In Formula (10), L¹⁰ represents a single bond or a divalent linking group. Examples of the divalent linking group represented by L¹⁰ include —O—, —S—, —CO—, —COO—, —OCO—, —SO₂—, —NH—, —NHCO—, an alkylene group, an arylene group, a heterocyclic group, and a combination thereof. Examples of the heterocyclic group include a nitrogen-containing heterocyclic group. Specific examples of the nitrogen-containing heterocyclic group include a piperazine ring group, a pyrrolidine ring group, a pyrrole ring group, a piperidine ring group, a pyridine ring group, an imidazole ring group, a pyrazole ring group, an oxazole ring group, a thiazole ring group, a pyrazine ring group, a morpholine ring group, a thiazine ring group, an indole ring group, an isoindole ring group, a benzimidazole ring group, a purine ring group, a quinoline ring group, an isoquinoline ring group, a quinoxaline ring group, a cinnoline ring group, a carbazole ring group, and groups represented by the following Formulae (L-1) to (L-7).

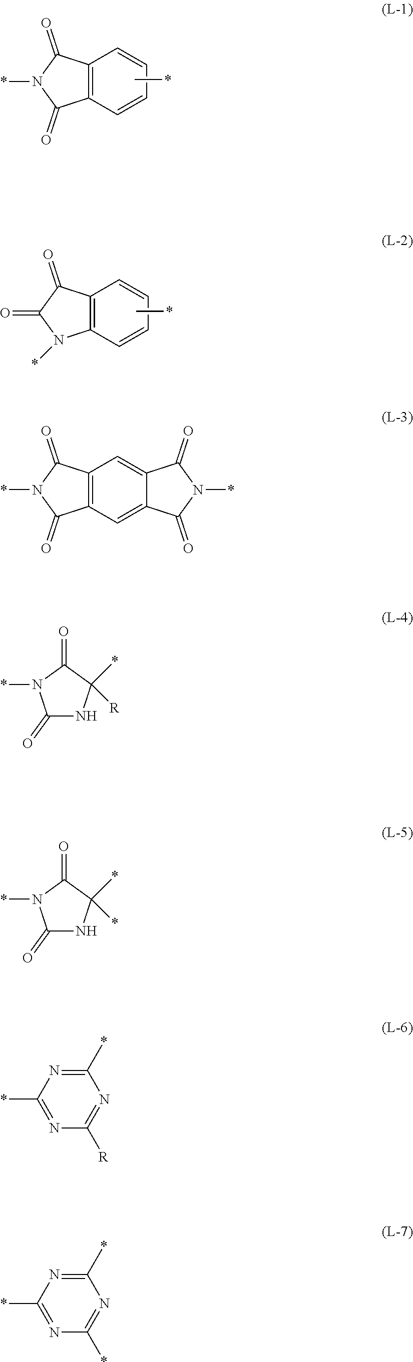

In the formula, * represents a direct bond. R represents a hydrogen atom or a substituent. Examples of the substituent include a substituent T described below.

Examples of a preferable aspect of the divalent linking group represented by L¹⁰ are as follows.

(a) an aspect where the divalent linking group represented by L¹⁰ is *—SO₂—

(b) an aspect where the divalent linking group represented by L¹⁰ is *—O—

(c) an aspect where the divalent linking group represented by L¹⁰ is *—COO—

(d) an aspect where the divalent linking group represented by L¹⁰ is *—OCO—

(e) an aspect where the divalent linking group represented by L¹⁰ is *—CONH-L^{10a}-O—

(f) an aspect where the divalent linking group represented by L¹⁰ is *—CONH-L^{10a}-COO—

(g) an aspect where the divalent linking group represented by L¹⁰ is *—CONH-L^{10a}-OCO—

(h) an aspect where the divalent linking group represented by L¹⁰ is *—NHCO-L^{10a}-O—

(i) an aspect where the divalent linking group represented by L¹⁰ is *—NHCO-L^{10a}-COO−

(j) an aspect where the divalent linking group represented by L¹⁰ is *—NHCO-L^{10a}-OCO—

(k) an aspect where the divalent linking group represented by L¹⁰ is *—CO-L^{10a}-O—

(l) an aspect where the divalent linking group represented by L¹⁰ is *—CO-L^{10a}-COO—

(n) an aspect where the divalent linking group represented by L¹⁰ is *—CO-L^{10a}-OCO—

(n) an aspect where the divalent linking group represented by L¹⁰ is *—CO-L^{10a}-O—

(o) an aspect where the divalent linking group represented by L¹⁰ is *—COO-L^{10a}-COO—

(p) an aspect where the divalent linking group represented by L¹⁰ is *—COO-L^{10a}-OCO—

(q) an aspect where the divalent linking group represented by L¹⁰ is *—OCO-L^{10a}-O—

(r) an aspect where the divalent linking group represented by L¹⁰ is *—OCO-L^{10a}-COO—

(s) an aspect where the divalent linking group represented by L¹⁰ is *—OCO-L₁₀ₐ-OCO—

(t) an aspect where the divalent linking group represented by L¹⁰ is *-heterocyclic group-O—

(u) an aspect where the divalent linking group represented by L¹⁰ is *-heterocyclic group-COO—

(v) an aspect where the divalent linking group represented by L¹⁰ is *-heterocyclic group-OCO—

(w) an aspect where the divalent linking group represented by L¹⁰ is *—O-L^{10a}-COO—

(y) an aspect where the divalent linking group represented by L¹⁰ is *—O-L^{10a}-OCO—

(z) an aspect where the divalent linking group represented by L¹⁰ is *—O-L^{10a}-O—

In the above description, “*” represents a linking portion to R⁹ in Formula (10). In addition, L^{10a} represents an alkylene group, an arylene group, a heterocyclic group, or a group including a combination thereof.

In Formula (10), X¹⁰ and Z¹⁰ each independently represent —SO₂—, —CO—, —B(OH)—, or —P(═O)(OH)—, it is preferable that at least one of X¹⁰ or Z¹⁰ represents —SO₂—, it is more preferable one of X¹⁰ or Z¹⁰ represents —SO₂— and another one of X¹⁰ or Z¹⁰ represents —SO₂— or —CO—, it is still more preferable one of X¹⁰ or Z¹⁰ represents —SO₂— and another one of X¹⁰ or Z¹⁰ represents —CO—, and it is still more preferable that X¹⁰ represents —CO— and Z¹⁰ represents —SO₂—.

In Formula (10), Y¹⁰ represents —NH—, —N—, or -NM¹-, and M¹ represents an atom or an atomic group forming a salt. Examples of the atom or the atomic group M¹ forming a salt include the atom or the atomic group described above regarding M¹ in Formula (1), and a preferable range is also the same.

In Formula (10), R⁹ represents a single bond or a hydrocarbon group having 1 or more carbon atoms which may include a substituent, and R¹⁰ represents a halogen atom, a hydroxyl group, or a hydrocarbon group having 1 or more carbon atoms which may include a substituent.

The hydrocarbon group represented by R⁹ and R¹⁰ may be an aliphatic hydrocarbon group or an aromatic hydrocarbon group. In addition, the aliphatic hydrocarbon group may be linear, branched, or cyclic. The number of carbon atoms in the aliphatic hydrocarbon group is preferably 1 to 30. The upper limit is preferably 25 or less, more preferably 20 or less, and still more preferably 15 or less. The lower limit is preferably 2 or more, more preferably 3 or more, and still more preferably 4 or more. The number of carbon atoms in the aromatic hydrocarbon group is preferably 6 to 20. The upper limit is preferably 18 or less, more preferably 15 or less, and still more preferably 12 or less. Examples of the substituent which may be included in the hydrocarbon group represented by R⁹ and R¹⁰ include a halogen atom, a hydroxyl group, a carboxyl group, an alkoxy group, a phenoxy group, an acyl group, and a sulfo group. In the alkoxy group, at least a part of the hydrogen atoms may be substituted with a halogen atom. The above-described substituent is preferably a halogen atom or an alkoxy group in which at least a part of the hydrogen atoms may be substituted with a halogen atom, more preferably a halogen atom or an alkoxy group in which at least a part of the hydrogen atoms is substituted with a halogen atom, and still more preferably a halogen atom. In addition, the halogen atom is preferably a chlorine atom, a fluorine atom, or a bromine atom and more preferably a fluorine atom.

In Formula (20), L²⁰ represents a single bond or a divalent linking group. Examples of the divalent linking group represented by L²⁰ include the group described above regarding L¹⁰ in Formula (10), and a preferable range thereof is also the same.

In Formula (20), X²⁰ to X²² each independently represent —SO₂—, —CO—, —B(OH)—, or —P(═O)(OH)—, and it is preferable that X²⁰ to X²² each independently represent —SO₂— or —CO—. In addition, it is preferable that at least one of X²⁰, . . . , or X²² represents —SO₂—, and it is more preferable that X²⁰ to X²² each independently represent —SO₂—.

In Formula (20), Y²⁰ represents —CH<, —C⁻<, or -CM²<, and M² represents an atom or an atomic group forming a salt. Examples of the atom or the atomic group M² forming a salt include the atom or the atomic group described above regarding M¹ in Formula (1), and a preferable range is also the same.

In Formula (20), R²⁰ represents a single bond or a hydrocarbon group having 1 or more carbon atoms which may include a substituent, and R²¹ and R²² each independently represent a halogen atom, a hydroxyl group, or a hydrocarbon group having 1 or more carbon atoms which may include a substituent. Examples of the hydrocarbon group represented by R²⁰ to R²² and the substituent which may be included in the hydrocarbon group represented by R²⁰ to R²² include the group described above regarding the hydrocarbon group represented by R⁹ and R¹⁰ and the substituent which may be included in the hydrocarbon group represented by R⁹ and R¹⁰ in Formula (10), and a preferable range thereof is also the same.

In Formula (30), L³⁰ represents a single bond or a divalent linking group. Examples of the divalent linking group represented by L³⁰ include the group described above regarding L¹⁰ in Formula (10), and a preferable range thereof is also the same.

In Formula (30), R³⁰ represents a hydrocarbon group having 1 or more carbon atoms which may include a substituent. Examples of the hydrocarbon group represented by R³⁰ and the substituent which may be included in the hydrocarbon group represented by R³⁰ include the group described above regarding the hydrocarbon group represented by R⁹ and R¹⁰ and the substituent which may be included in the hydrocarbon group represented by R⁹ and R¹⁰ in Formula (10), and a preferable range thereof is also the same.

In Formula (30), Y³⁰ represents —COOH, —COO⁻, —COOM³, —SO₃H, —SO₃—, —SO₃M³, or —B⁻(Rb1)(Rb2)(Rb3), M³ represents an atom or an atomic group forming a salt, and Rb1 to Rb3 each independently represent a halogen atom or a hydrocarbon group having 1 or more carbon atoms which may include a substituent. Examples of the atom or the atomic group M³ forming a salt include the atom or the atomic group described above regarding M¹ in Formula (1), and a preferable range is also the same. The halogen atom represented by Rb1 to Rb3 is preferably a chlorine atom, a fluorine atom, or a bromine atom and more preferably a fluorine atom. Examples of the hydrocarbon group represented by Rb1 to Rb3 and the substituent which may be included in the hydrocarbon group represented by Rb1 to Rb3 include the group described above regarding the hydrocarbon group represented by R⁹ and R¹⁰ and the substituent which may be included in the hydrocarbon group represented by R⁹ and R¹⁰ in Formula (10), and a preferable range thereof is also the same.

Specific examples of the functional group A include the following groups. In the following structural formulae, a wave line represents a direct bond. The pKa and the Clog P value are calculated by substituting a wave line portion with a methyl group. Among the following groups, a-1, a-2, a-3, a-6, a-10, a-11, a-17, a-20, a-21, a-23, a-31, a-32, a-33, a-35, a-36, or a-37 is preferable, and a-1, a-11, a-17, a-31, a-32, a-33, a-35, a-36, or a-37 is more preferable. It is preferable that the compound A includes the above-described group because the effect of the present invention tends to be particularly high.

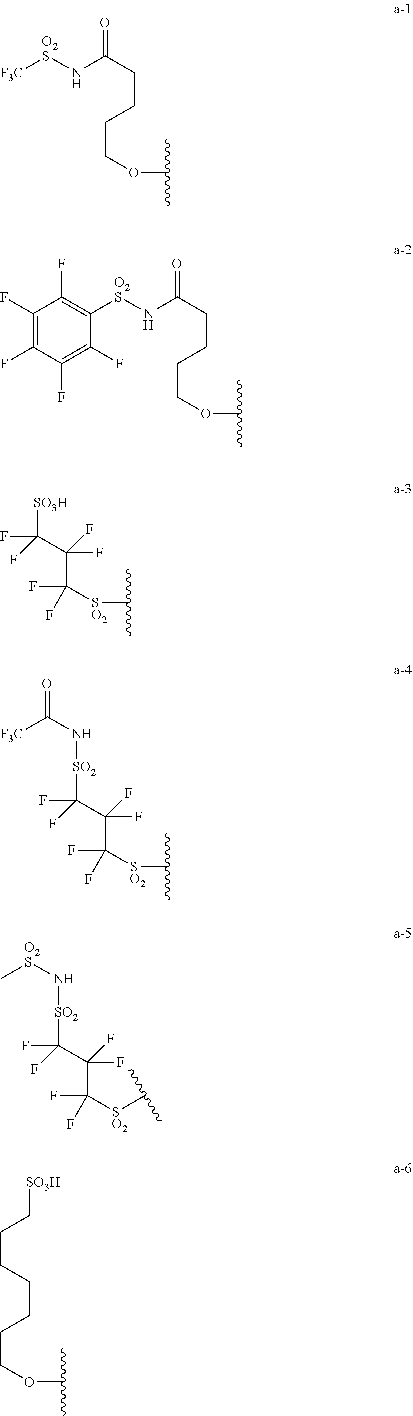 [SMILES: *OCCCCC(=O)NS(=O)(=O)C(F)(F)F ; *OCCCCC(=O)NS(=O)(=O)c1c(F)c(F)c(F)c(F)c1F ; *S(=O)(=O)C(F)(F)C(F)(F)C(F)(F)S(=O)(=O)O ; *S(=O)(=O)C(F)(F)C(F)(F)C(F)(F)S(=O)(=O)NC(=O)C(F)(F)F ; *S(=O)(=O)C(F)(F)C(F)(F)C(F)(F)S(=O)(=O)NS(C)(=O)=O ; *OCCCCCCCS(=O)(=O)O]

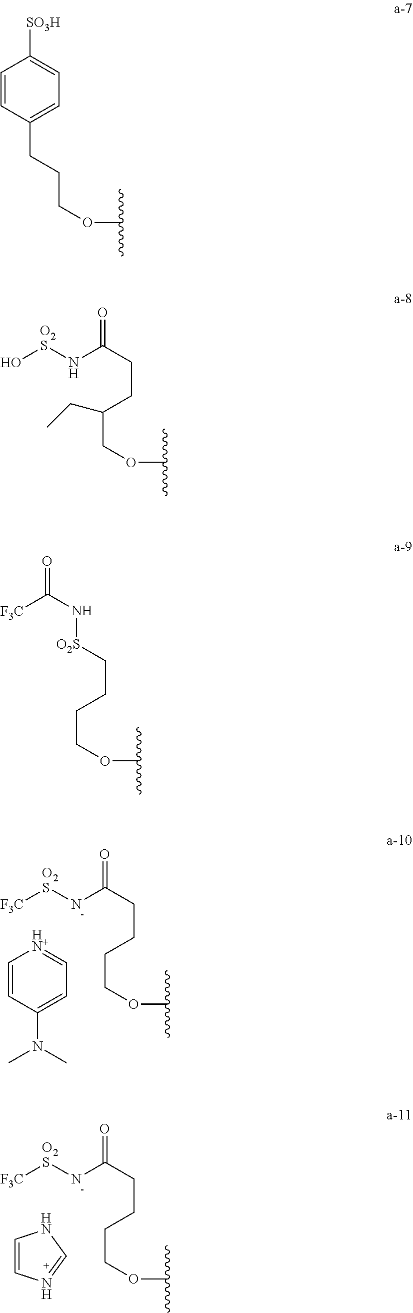

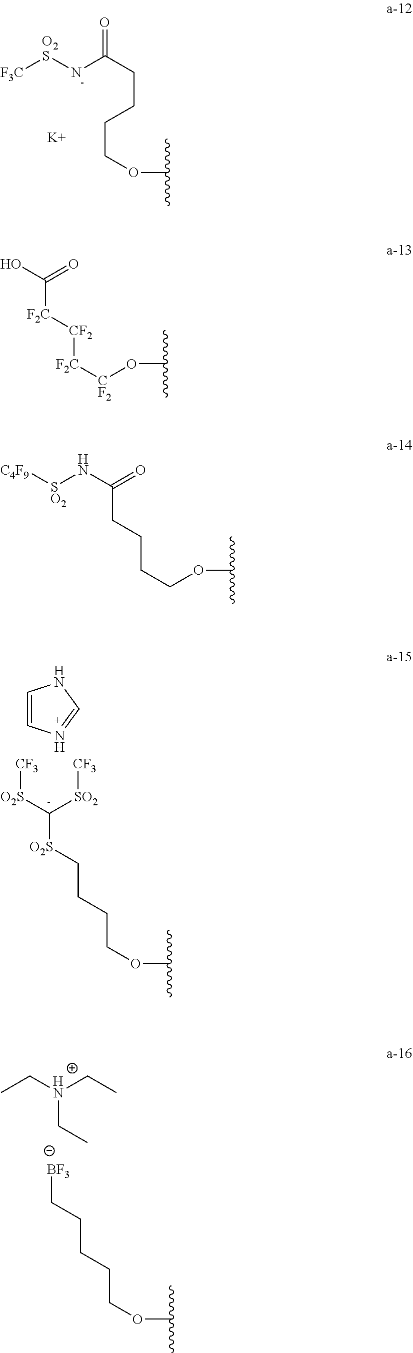

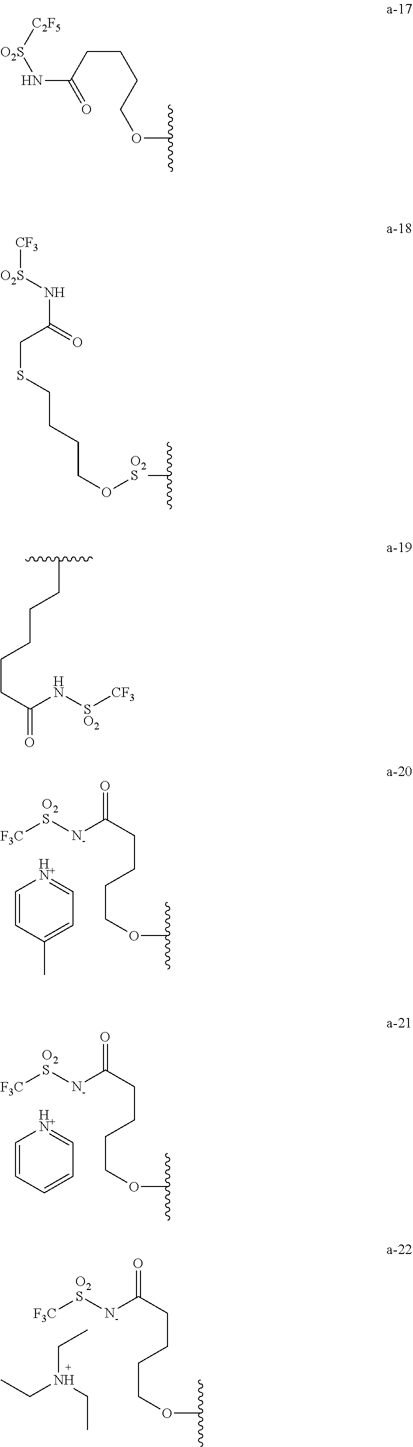

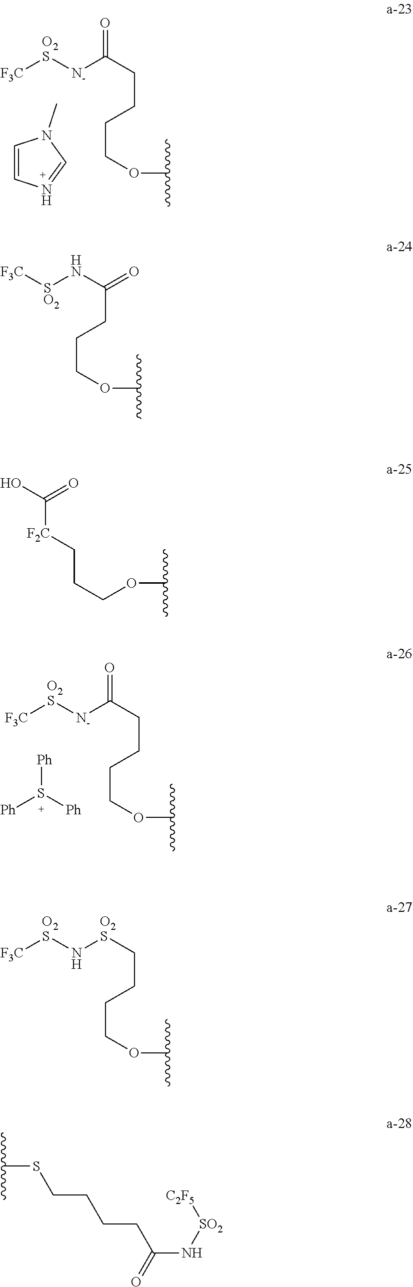

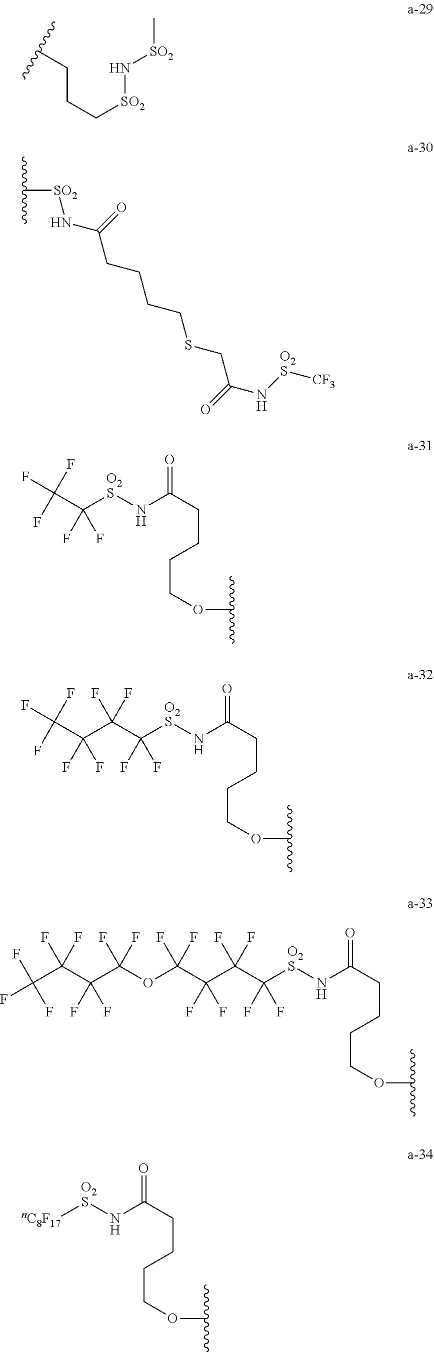

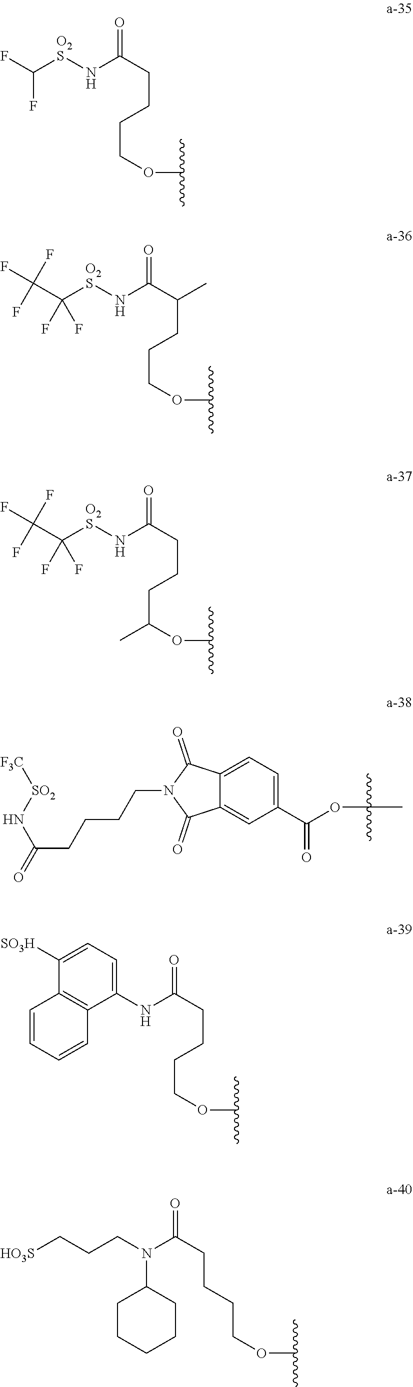 [SMILES: *OCCCCC(=O)NS(=O)(=O)C(F)F ; *OCCCC(C)C(=O)NS(=O)(=O)C(F)(F)C(F)(F)F ; *OC(C)CCCC(=O)NS(=O)(=O)C(F)(F)C(F)(F)F ; *OC(=O)c1ccc2c(c1)C(=O)N(CCCCC(=O)NS(=O)(=O)C(F)(F)F)C2=O ; *OCCCCC(=O)Nc1ccc(S(=O)(=O)O)c2ccccc12 ; *OCCCCC(=O)N(CCCS(=O)(=O)O)C1CCCCC1]

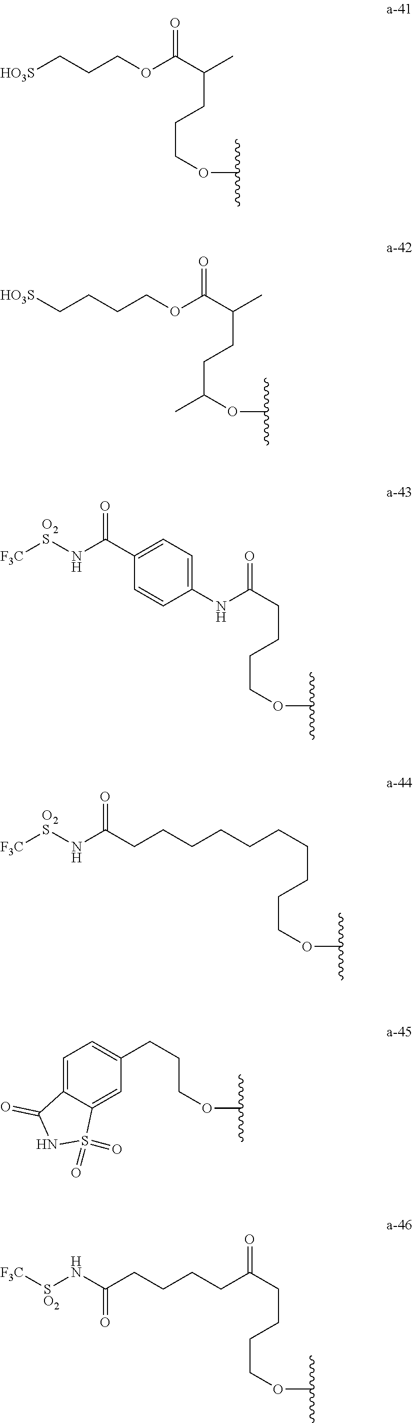 [SMILES: *OCCCC(C)C(=O)OCCCS(=O)(=O)O ; *OC(C)CCC(C)C(=O)OCCCCS(=O)(=O)O ; *OCCCCC(=O)Nc1ccc(C(=O)NS(=O)(=O)C(F)(F)F)cc1 ; *OCCCCCCCCCCC(=O)NS(=O)(=O)C(F)(F)F ; *OCCCc1ccc2c(c1)S(=O)(=O)NC2=O ; *OCCCCC(=O)CCCCC(=O)NS(=O)(=O)C(F)(F)F]

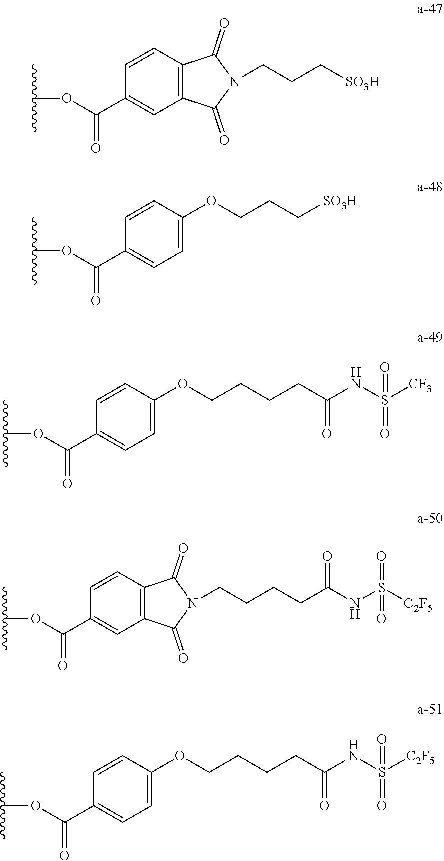

It is preferable that the compound A is a compound represented by Formula (A1).

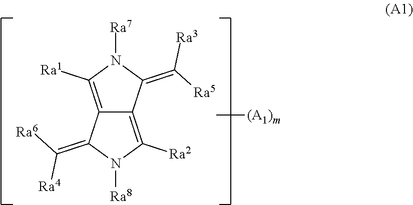

In Formula (A1), Ra¹ and Ra² each independently represent an alkyl group, an aryl group, or a heteroaryl group.

Ra³, Ra⁴, Ra⁵, and Ra⁶ each independently represent a cyano group, an acyl group, an alkoxycarbonyl group, an alkylsulfinyl group, an arylsulfinyl group, or a heteroaryl group.

Ra⁷ and Ra⁸ each independently represent a hydrogen atom, an alkyl group, an aryl group, a heteroaryl group, —BRa⁹Ra⁰, or a metal atom.

Ra⁷ may form a covalent bond or a coordinate bond with Ra¹, Ra³, or Ra⁵.

Ra⁸ may form a covalent bond or a coordinate bond with Ra², Ra⁴, or Ra⁶.

Ra⁹ and Ra¹⁰ each independently represent a hydrogen atom, a halogen atom, an alkyl group, an alkenyl group, an aryl group, a heteroaryl group, an alkoxy group, an aryloxy group, or a heteroaryloxy group, and Ra⁹ and Ra¹⁰ may be bonded to each other to form a ring.

A₁ represents the above-described functional group A.

m represents an integer of 1 to 10, and in a case where m represents 2 or more, a plurality of A₁'s may be the same as or different from each other.

In Formula (A1), Ra¹ and Ra² each independently represent an alkyl group, an aryl group, or a heteroaryl group, preferably an aryl group or a heteroaryl group, and more preferably an aryl group.

The number of carbon atoms in the alkyl group represented by Ra¹ and Ra² is preferably 1 to 30, more preferably 1 to 20, and still more preferably 1 to 10.

The number of carbon atoms in the aryl group represented by Ra¹ and Ra² is preferably 6 to 30, more preferably 6 to 20, and still more preferably 6 to 12.

The number of carbon atoms constituting the heteroaryl group represented by Ra¹ and Ra² is preferably 1 to 30 and more preferably 1 to 12. Examples of the kind of the heteroatom constituting the heteroaryl group include a nitrogen atom, an oxygen atom, and a sulfur atom. The number of heteroatoms constituting the heteroaryl group is preferably 1 to 3 and more preferably 1 or 2. The heteroaryl group is preferably a monocycle or a fused ring, more preferably a monocycle or a fused ring composed of 2 to 8 rings, and still more preferably a monocycle or a fused ring composed of 2 to 4 rings.

The alkyl group, the aryl group, and the heteroaryl group represented by Ra¹ to Ra² may be unsubstituted or may have a substituent. Examples of the substituent include the following substituent T. In addition, “-A₁” in Formula (A1) may be bonded to the alkyl group, the aryl group, or the heteroaryl group represented by Ra₁ and Ra₂ as a substituent, and it is preferable that “-A₁” in Formula (A1) may be bonded to the alkyl group, the aryl group, or the heteroaryl group represented by Ra₁ and Ra₂ as a substituent.

(Substituent T)

The substituent T includes an alkyl group (preferably an alkyl group having 1 to 30 carbon atoms), an alkenyl group (preferably an alkenyl group having 2 to 30 carbon atoms), an alkynyl group (preferably an alkynyl group having 2 to 30 carbon atoms), an aryl group (preferably an aryl group having 6 to 30 carbon atoms), an amino group (preferably an amino group having 0 to 30 carbon atoms), an alkoxy group (preferably an alkoxy group having 1 to 30 carbon atoms), an aryloxy group (preferably an aryloxy group having 6 to 30 carbon atoms), a heteroaryloxy group, an acyl group (preferably having an acyl group 1 to 30 carbon atoms), an alkoxycarbonyl group (preferably an alkoxycarbonyl group having 2 to 30 carbon atoms), an aryloxycarbonyl group (preferably an aryloxycarbonyl group having 7 to 30 carbon atoms), an acyloxy group (preferably an acyloxy group having 2 to 30 carbon atoms), an acylamino group (preferably an acylamino group having 2 to 30 carbon atoms), an alkoxycarbonylamino group (preferably an alkoxycarbonylamino group having 2 to 30 carbon atoms), an aryloxycarbonylamino group (preferably an aryloxycarbonylamino group having 7 to 30 carbon atoms), a sulfamoyl group (preferably a sulfamoyl group having 0 to 30 carbon atoms), a carbamoyl group (preferably a carbamoyl group having 1 to 30 carbon atoms), an alkylthio group (preferably an alkylthio group having 1 to 30 carbon atoms), an arylthio group (preferably an arylthio group having 6 to 30 carbon atoms), a heteroarylthio group (preferably a heteroarylthio group having 1 to 30 carbon atoms), an alkylsulfonyl group (preferably an alkylsulfonyl group having 1 to 30 carbon atoms), an arylsulfonyl group (preferably an arylsulfonyl group having 6 to 30 carbon atoms), a heteroarylsulfonyl group (preferably a heteroarylsulfonyl group having 1 to 30 carbon atoms), an alkylsulfinyl group (preferably an alkylsulfinyl group having 1 to 30 carbon atoms), an arylsulfinyl group (preferably an arylsulfinyl group having 6 to 30 carbon atoms), a heteroarylsulfinyl group (preferably a heteroarylsulfinyl group having 1 to 30 carbon atoms), a ureido group (preferably a ureido group having 1 to 30 carbon atoms), a phosphoric amide group (preferably a phosphoric amide group having 1 to 30 carbon atoms), a hydroxyl group, a carboxyl group, a sulfo group, a phosphate group, a mercapto group, a halogen atom, a cyano group, an alkylsulfino group, an arylsulfino group, a hydrazino group, an imino group, and a heteroaryl group (preferably a heteroaryl group having 1 to 30 carbon atoms). In a case where the above-described groups can be further substituted, the groups may further have a substituent. Examples of the substituent include the groups described regarding the substituent T.

In Formula (A1), Ra³, Ra⁴, Ra¹, and Ra⁶ each independently represent a cyano group, an acyl group, an alkoxycarbonyl group, an alkylsulfinyl group, an arylsulfinyl group, or a heteroaryl group.

It is preferable that one of Ra³ and Ra⁵ represents a cyano group, an acyl group, an alkoxycarbonyl group, an alkylsulfinyl group, or an arylsulfinyl group and another one of Ra³ and Ra⁵ represents a heteroaryl group, and it is more preferable that one of Ra³ and Ra⁵ represents a cyano group and another one of Ra³ and Ra⁵ represents a heteroaryl group. In addition, “-A₁” in Formula (A1) may be bonded to the heteroaryl group as a substituent.

It is preferable that one of Ra⁴ and Ra⁶ represents a cyano group, an acyl group, an alkoxycarbonyl group, an alkylsulfinyl group, or an arylsulfinyl group and another one of Ra⁴ and Ra⁶ represents a heteroaryl group, and it is more preferable that one of Ra⁴ and Ra⁶ represents a cyano group and another one of Ra⁴ and Ra⁶ represents a heteroaryl group. In addition, “-A₁” in Formula (A1) may be bonded to the heteroaryl group as a substituent.

It is preferable that the heteroaryl group represented by Ra³ to Ra⁶ is a group represented by the following Formula (A-1) or a group represented by the following Formula (A-2).

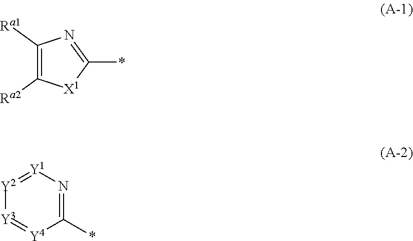

In Formula (A-1), X¹ represents O, S, NR^{X1}, or CR^{X2}R^{X3}, R^{X1} to R^{X3} each independently represent a hydrogen atom or a substituent, R^{a1} and R² each independently represent a hydrogen atom or a substituent, and Ra¹ and R^{a2} may be bonded to each other to form a ring. * represents a direct bond. Examples of the substituent represented by R^{a1}, R^{a2}, and R^{X1} to R^{x3} include the above-described substituent T.

The ring which is formed by Ra¹ and R^{a2} being bonded to each other is preferably an aromatic ring. In a case where R^{a1} and R^{a2} are bonded to each other to form a ring, for example, (A-1) represents a group represented by the following (A-1-1) or a group represented by the following (A-1-2).

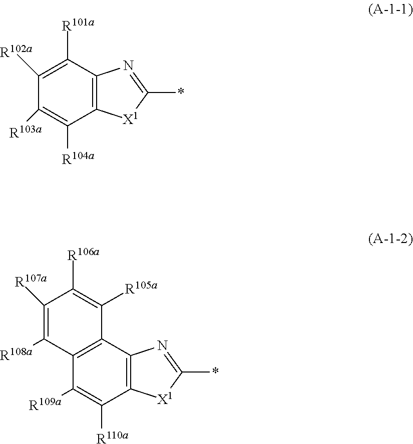

In the formula, X¹ represents O, S, NR^{X1}, or CR^{X2}R^{X3}, R^{X1} to R^{X3} each independently represent a hydrogen atom or a substituent, and R^{101a} to R^{110a} each independently represent a hydrogen atom or a substituent. * represents a direct bond. Examples of the substituent represented by R^{101a} to R^{110a} include the above-described substituent T.

In Formula (A-2), Y¹ to Y⁴ each independently represent N or CR^{Y1}, at least two of Y¹, Y², Y³, or Y⁴ represent CR^{Y1}, R^{Y1} represents a hydrogen atom or a substituent, and adjacent R^{Y1}'s may be bonded to each other to form a ring. * represents a direct bond. Examples of the substituent represented by R^{Y1} include the above-described substituent T. Among these, an alkyl group, an aryl group, or a halogen atom is preferable.

At least two of Y¹, or Y⁴ represent CR^{Y1}, and adjacent R^{y}'s may be bonded to each other to form a ring. The ring which is formed by adjacent R^{Y1}'s being bonded to each other is preferably an aromatic ring. In a case where adjacent R^{Y1}'s are bonded to each other to form a ring, examples of the group represented by (A-2) include a group represented by the following (A-2-1) and a group represented by the following (A-2-2).

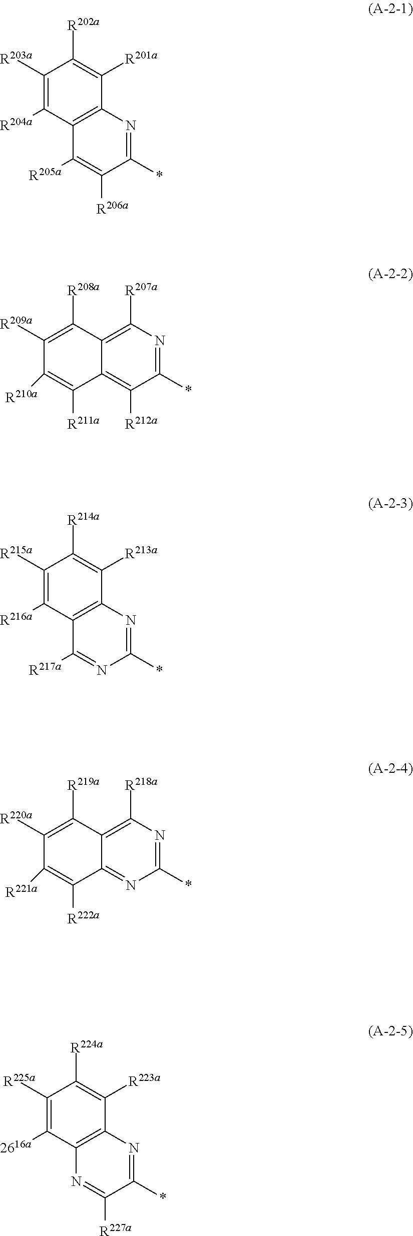

In the formulae, R^{201a} to R^{227a} each independently represent a hydrogen atom or a substituent, and * represents a direct bond. Examples of the substituent represented by R^{201a} to R^{227a} include the above-described substituent T.

In Formula (A1), Ra⁷ and Ra⁸ each independently represent a hydrogen atom, an alkyl group, an aryl group, a heteroaryl group, —BRa⁹Ra¹⁰, or a metal atom, and preferably —BRa⁹Ra¹⁰. Ra⁹ and Ra¹⁰ each independently represent a hydrogen atom, a halogen atom, an alkyl group, an alkenyl group, an aryl group, a heteroaryl group, an alkoxy group, an aryloxy group, or a heteroaryloxy group, preferably a halogen atom, an alkyl group, an aryl group, or a heteroaryl group, more preferably a halogen atom, an alkyl group, or an aryl group, and still more preferably an aryl group. Ra⁹ and Ra¹⁰ may be bonded to each other to form a ring.

In Formula (A1), Ra⁷ may form a covalent bond or a coordinate bond with Ra¹, Ra³, or Ra⁵, and Ra⁸ may form a covalent bond or a coordinate bond with Ra², Ra⁴, or Ra⁶.

In Formula (A1), A₁ represents the above-described functional group A. The details of the functional group A are as described above, and a preferable range thereof is also the same.

In Formula (A1), m represents an integer of 1 to 10, preferably 1 to 4, more preferably 1 to 3, still more preferably 1 or 2, and still more preferably 2.

It is preferable that the compound represented by Formula (A1) is a compound represented by the following Formula (A2). The compound represented by Formula (A2) is also the compound according to the present invention.

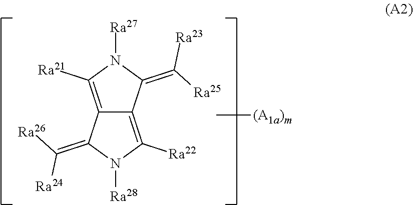

In Formula (A2), Ra²¹ and Ra²² each independently represent an alkyl group, an aryl group, or a heteroaryl group.

Ra²³, Ra²⁴, Ra²⁵, and Ra²⁶ each independently represent a cyano group, an acyl group, an alkoxycarbonyl group, an alkylsulfinyl group, an arylsulfinyl group, or a heteroaryl group.

Ra²⁷ and Ra²⁸ each independently represent —BRa²⁹Ra³⁰

Ra²⁹ and Ra³⁰ each independently represent a hydrogen atom, a halogen atom, an alkyl group, an alkenyl group, an aryl group, a heteroaryl group, an alkoxy group, an aryloxy group, or a heteroaryloxy group, and Ra²⁹ and Ra³⁰ may be bonded to each other to form a ring.

A₁ₐ represents the above-described functional group A.

m represents an integer of 1 to 10, and in a case where m represents 2 or more, a plurality of A₁a's may be the same as or different from each other.

Ra²¹ and Ra²² in Formula (A2) have the same definitions and the same preferable ranges as Ra¹ and Ra² in Formula (A1).

Ra²³, Ra²⁴, Ra²⁵, and Ra²⁶ in Formula (A2) have the same definitions and the same preferable ranges as Ra³, Ra⁴, Ra⁵, and Ra⁶ in Formula (A1).

Ra²⁹ and Ra³⁰ in Formula (A2) have the same definitions and the same preferable ranges as Ra⁹ and Ra¹⁰ in Formula (A1).

In Formula (A2), A₁a represents the above-described functional group A. The details of the functional group A are as described above, and a preferable range thereof is also the same.

In Formula (A2), m represents an integer of 1 to 10, preferably 1 to 4, more preferably 1 to 3, still more preferably 1 or 2, and still more preferably 2.

It is preferable that the compound represented by Formula (A1) is a compound represented by the following Formula (A10).

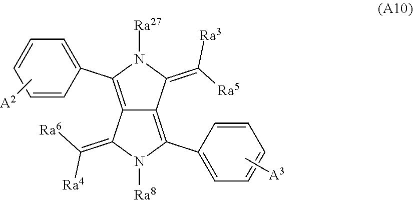

In Formula (A10), Ra³, Ra⁴, Ra⁵, and Ra⁶ each independently represent a cyano group, an acyl group, an alkoxycarbonyl group, an alkylsulfinyl group, an arylsulfinyl group, or a heteroaryl group.

Ra⁷ and Ra⁸ each independently represent a hydrogen atom, an alkyl group, an aryl group, a heteroaryl group, —BRa⁹Ra¹⁰, or a metal atom.

Ra⁷ may form a covalent bond or a coordinate bond with Ra³ or Ra⁵.

Ra⁸ may form a covalent bond or a coordinate bond with Ra⁴ or Ra⁶.

Ra⁹ and Ra¹⁰ each independently represent a hydrogen atom, a halogen atom, an alkyl group, an alkenyl group, an aryl group, a heteroaryl group, an alkoxy group, an aryloxy group, or a heteroaryloxy group, and R⁹ and R¹⁰ may be bonded to each other to form a ring.

A² and A³ each independently represent the above-described functional group A.

Ra³ to Ra⁸ in Formula (A10) have the same definitions and the same preferable ranges as Ra³ to Ra⁸ in Formula (A1). A² and A³ in Formula (A10) have the same definitions and the same preferable ranges as A¹ in Formula (A1).

Specific examples of the compound A include compounds having the following structures. In the following structural formulae and tables, Me represents a methyl group, and Ph represents a phenyl group.

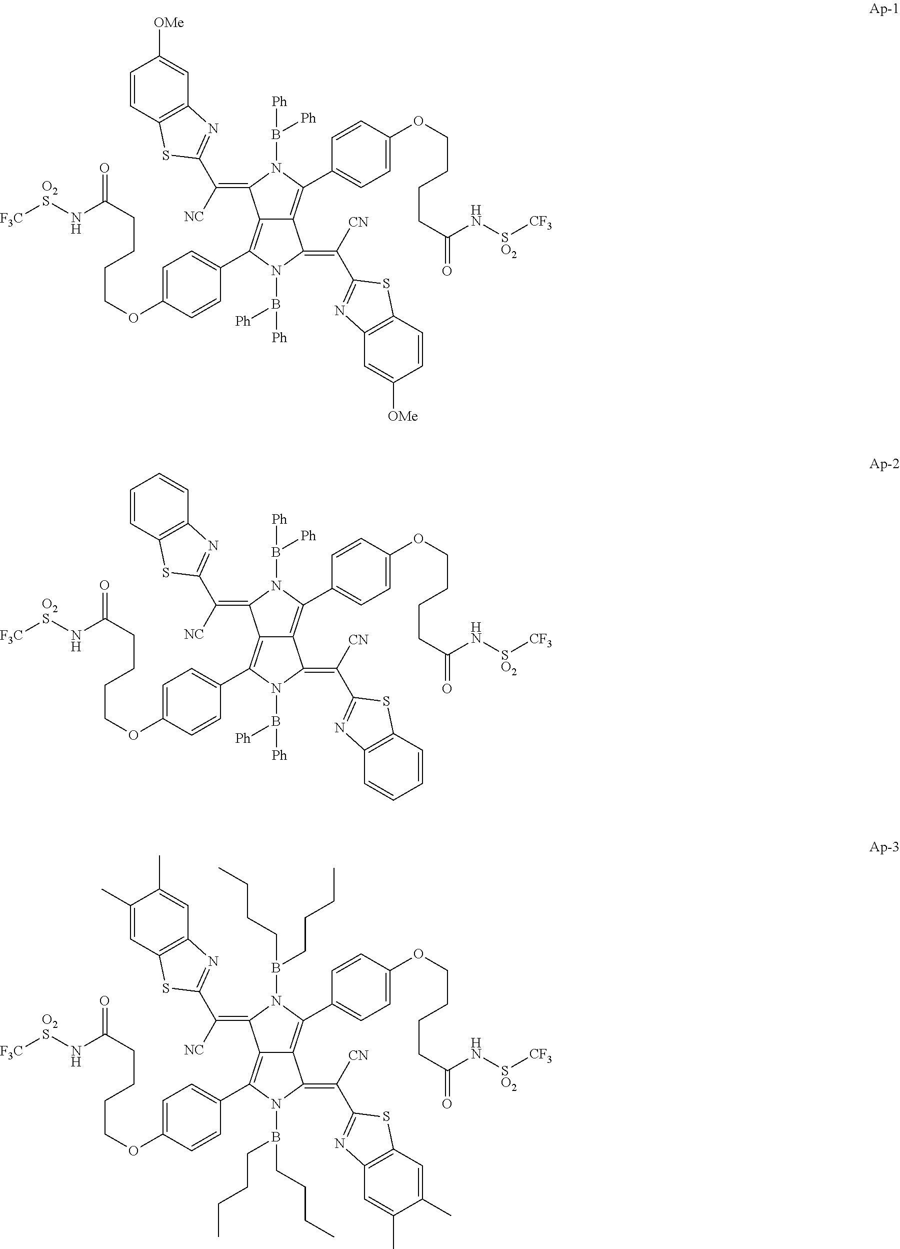

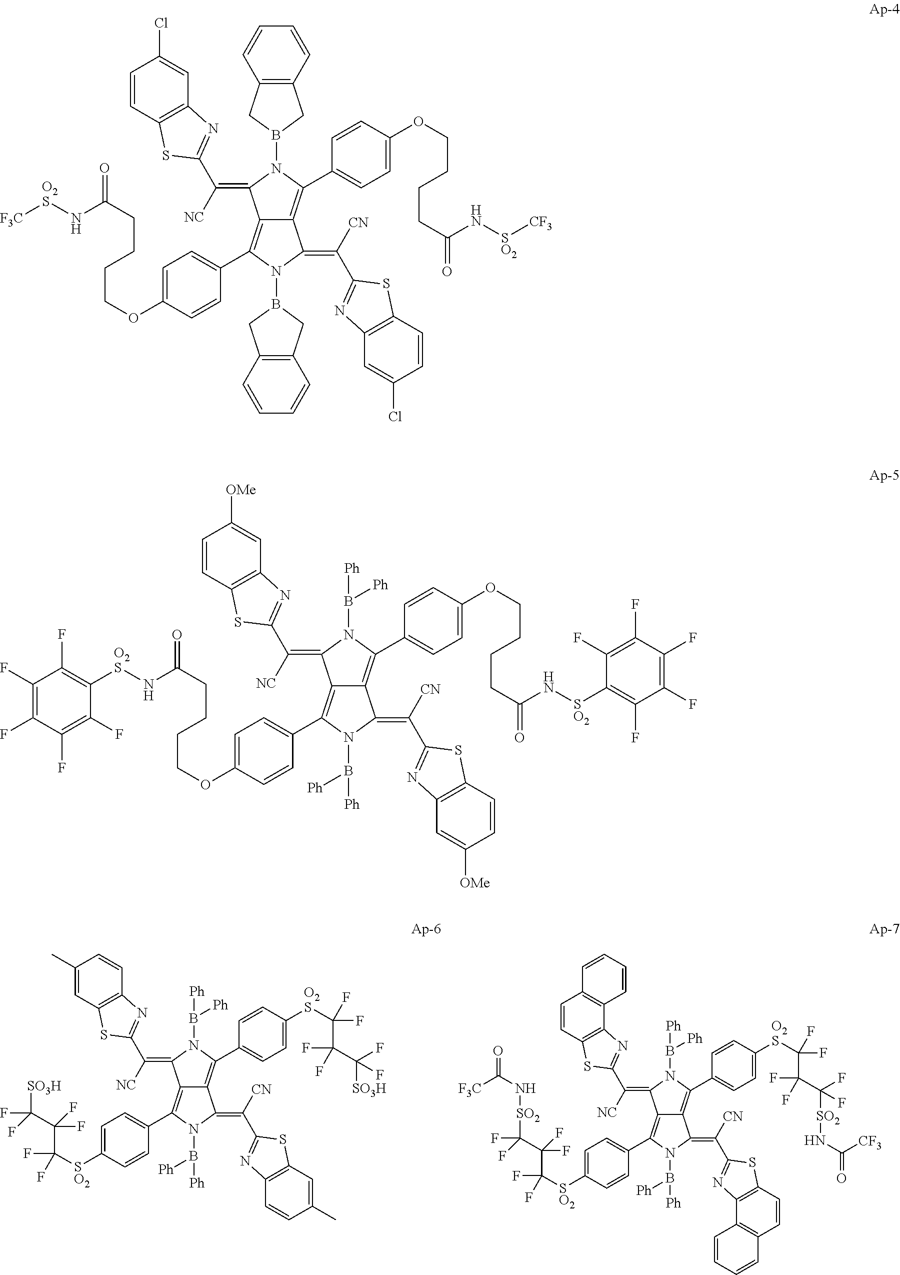

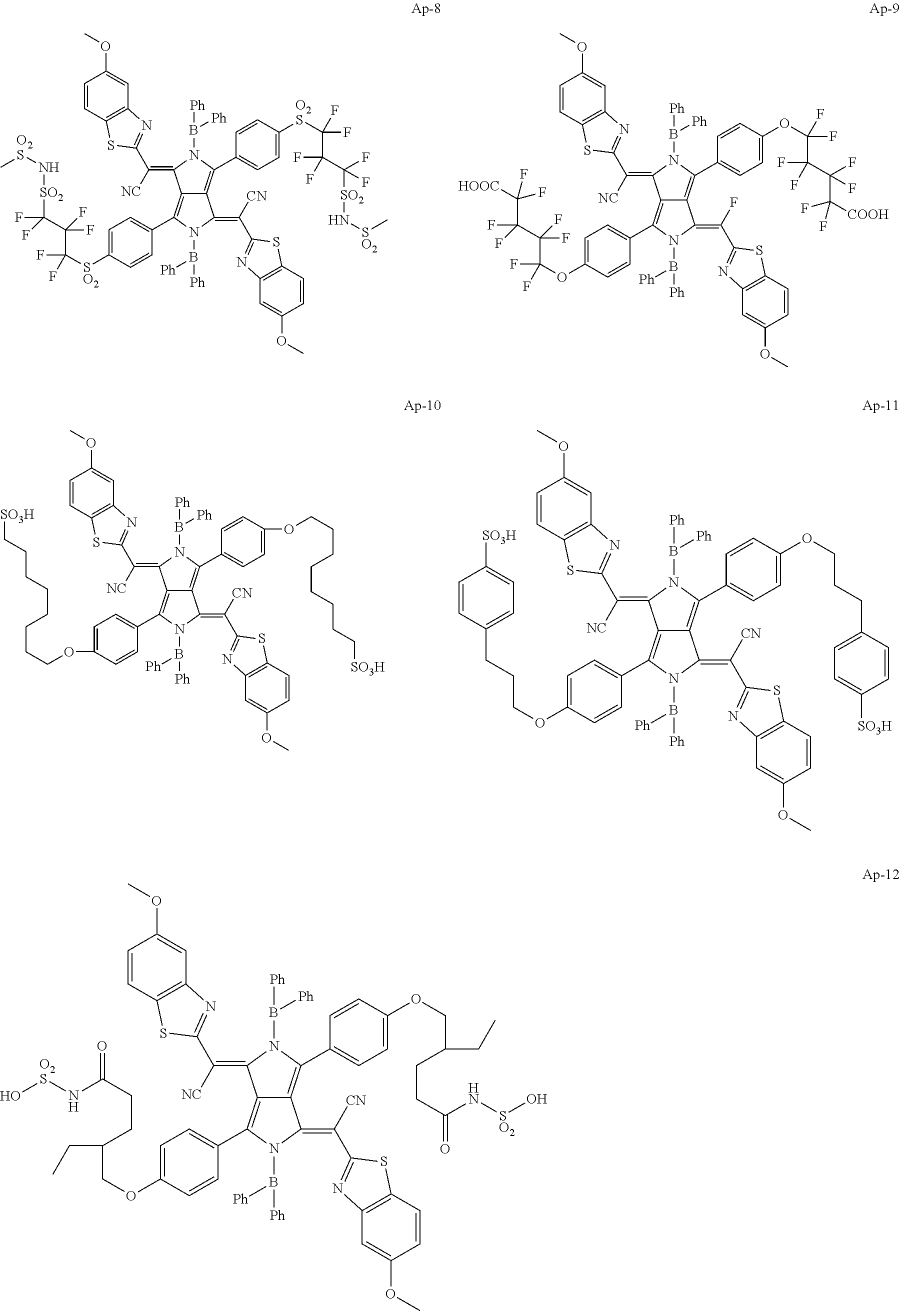

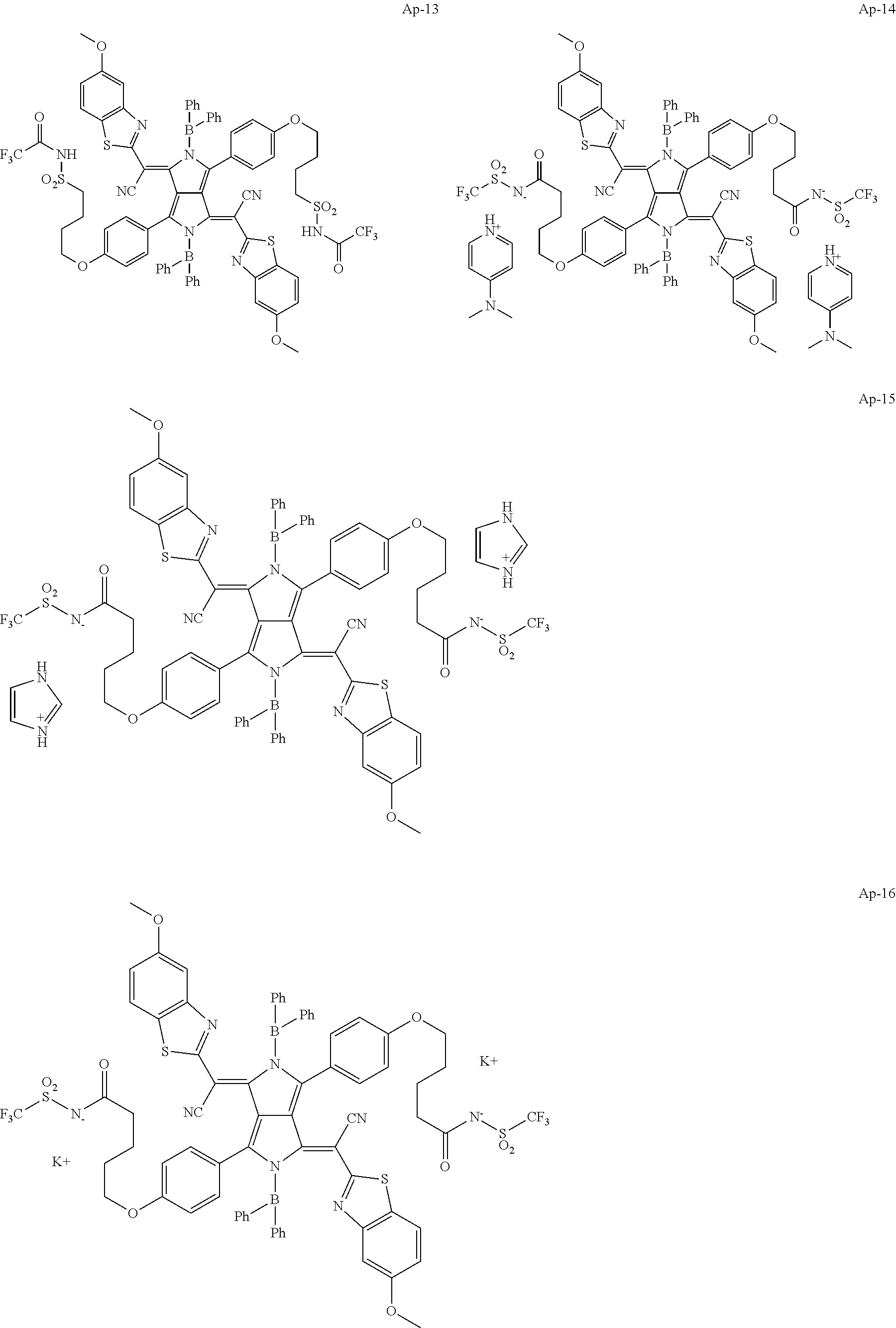

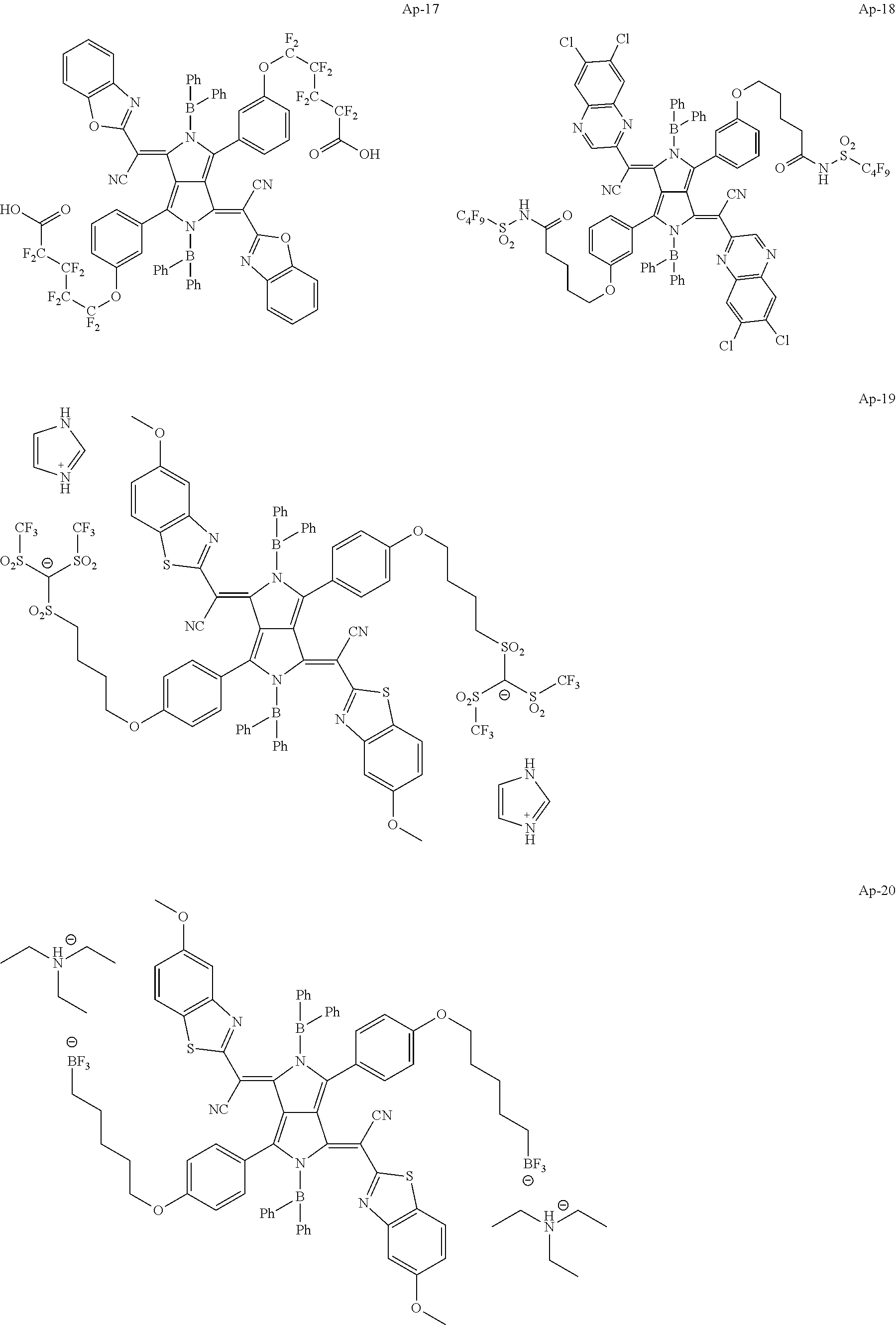

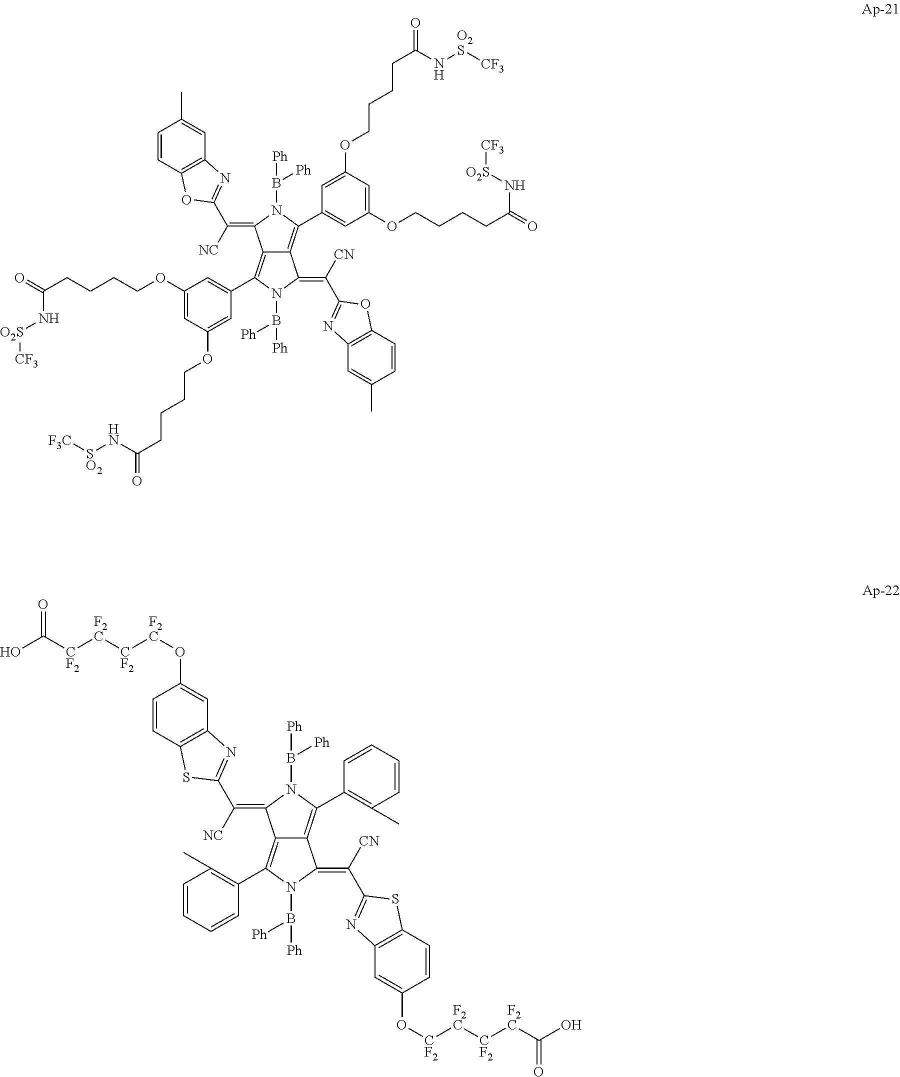 [SMILES: Cc1ccc2oc(/C(C#N)=c3/c4c(-c5cc(OCCCCC(=O)NS(=O)(=O)C(F)(F)F)cc(OCCCCC(=O)NS(=O)(=O)C(F)(F)F)c5)n(B(c5ccccc5)c5ccccc5)/c(=C(/C#N)c5nc6cc(C)ccc6o5)c4c(-c4cc(OCCCCC(=O)NS(=O)(=O)C(F)(F)F)cc(OCCCCC(=O)NS(=O)(=O)C(F)(F)F)c4)n3B(c3ccccc3)c3ccccc3)nc2c1 ; Cc1ccccc1-c1c2/c(=C(\C#N)c3nc4cc(OC(F)(F)C(F)(F)C(F)(F)C(F)(F)C(=O)O)ccc4s3)n(B(c3ccccc3)c3ccccc3)c(-c3ccccc3C)c2/c(=C(\C#N)c2nc3cc(OC(F)(F)C(F)(F)C(F)(F)C(F)(F)C(=O)O)ccc3s2)n1B(c1ccccc1)c1ccccc1]

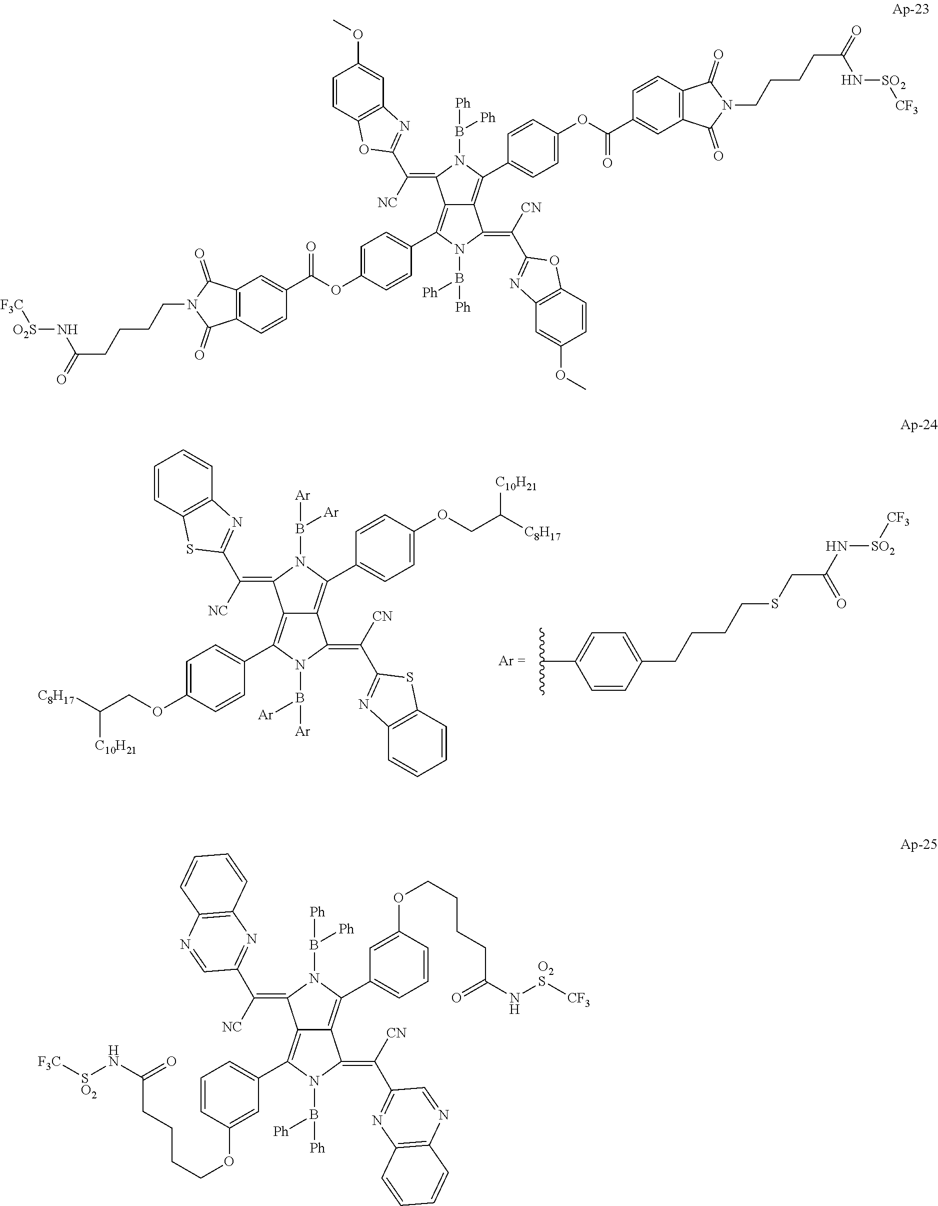

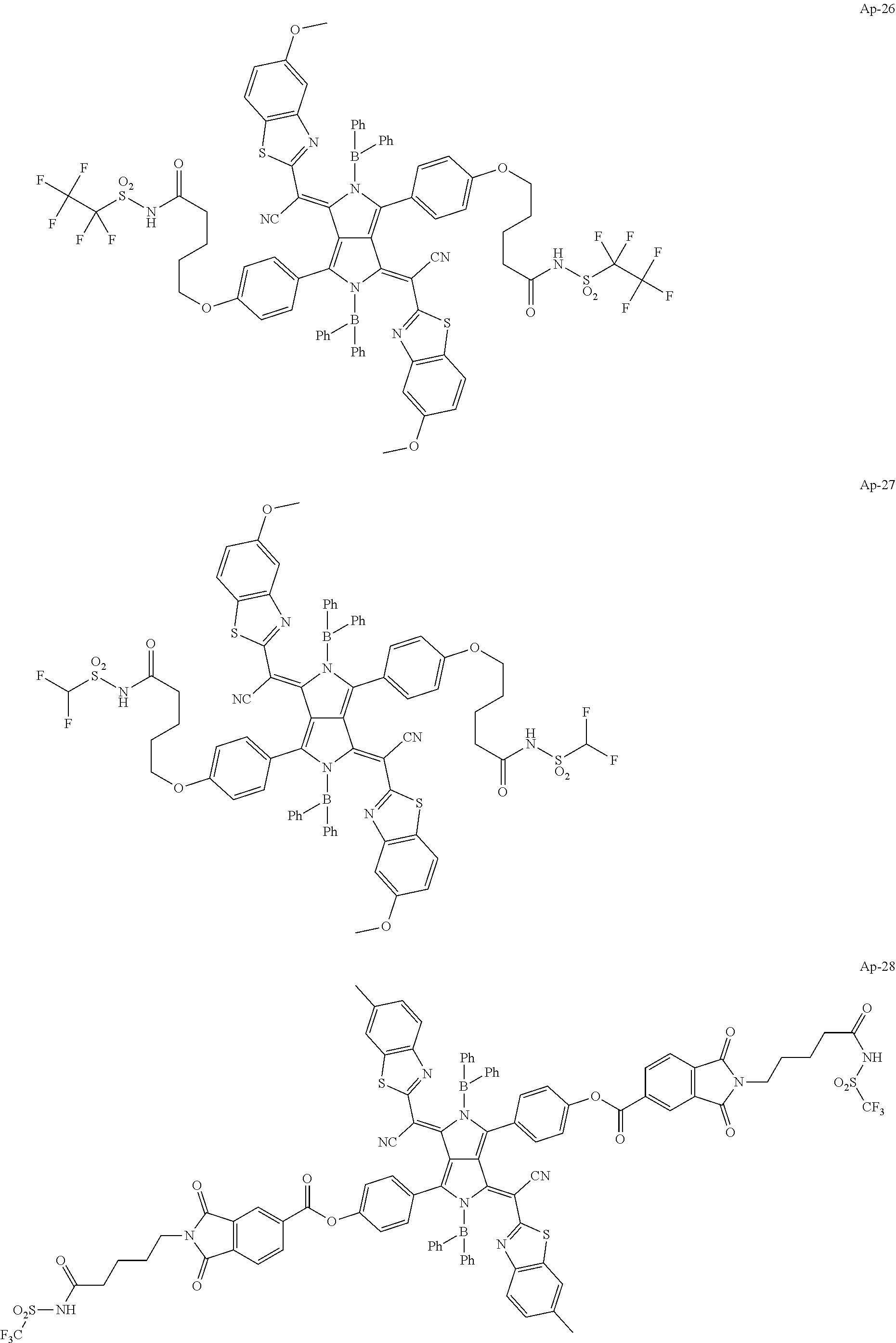

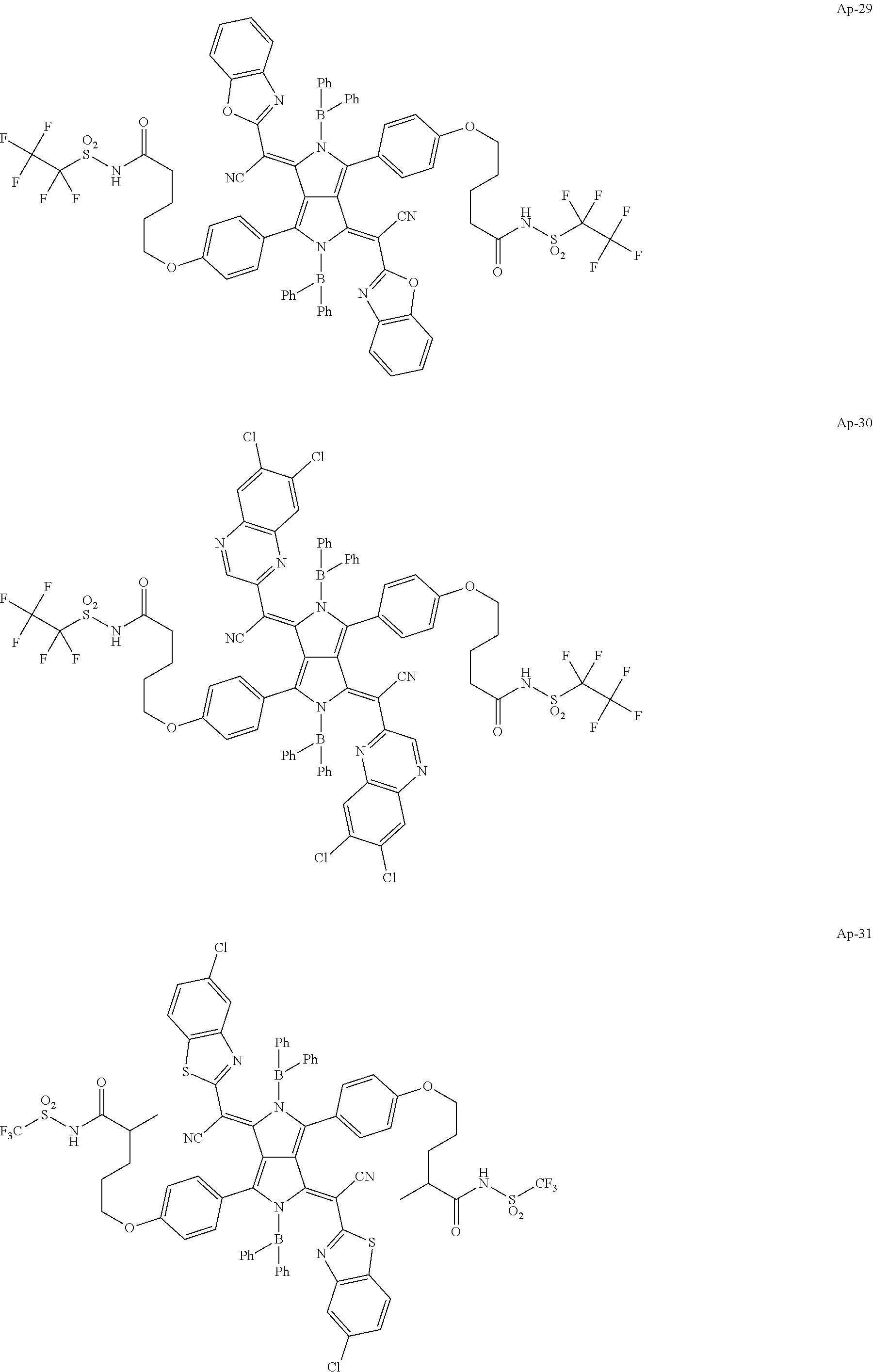

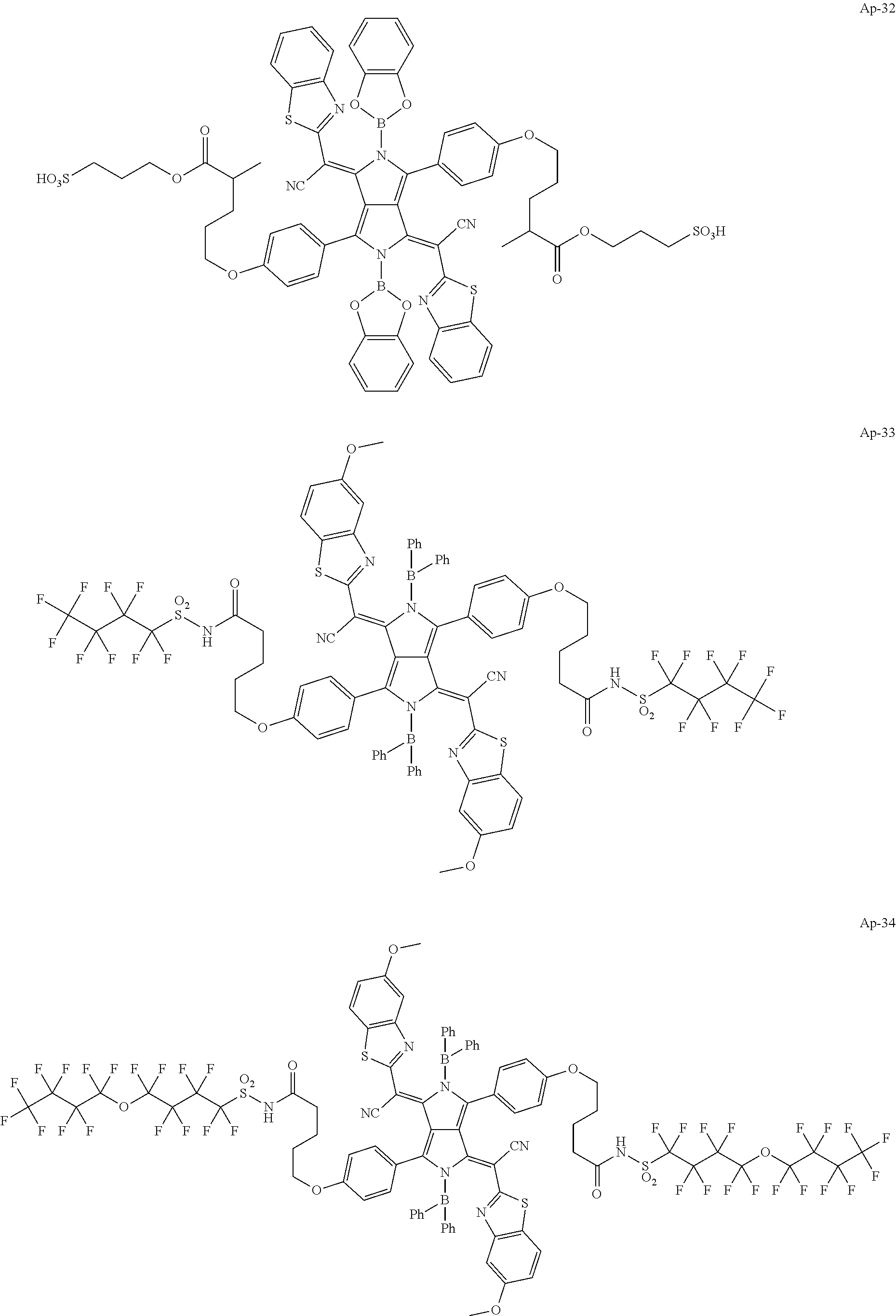

TABLE 1

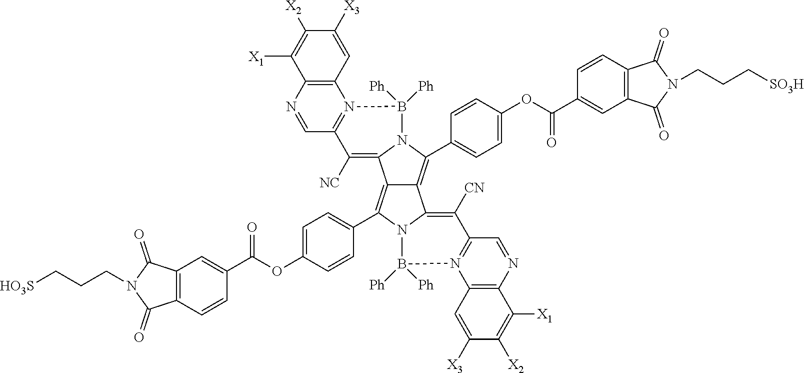

| Compound Name | X₁ | X₂ | X₃ |
|---|---|---|---|
| Ap-35 | H | CI | CI |
| Ap-36 | H | OMe | CI |
| Ap-37 | H | CI | H |
| Ap-38 | CI | CI | CI |
| Ap-39 | CI | OMe | H |
| Ap-40 | CI | CI | H |
| Ap-41 | CI | H | CI |
| Ap-42 | H | NO₂ | H |
| Ap-43 | H | CF₃ | H |
| Ap-44 | H | CN | H |
| Ap-45 | H | SMe | H |
| Ap-46 | H | SPh | H |
| Ap-47 | H | Ph | H |

TABLE 2

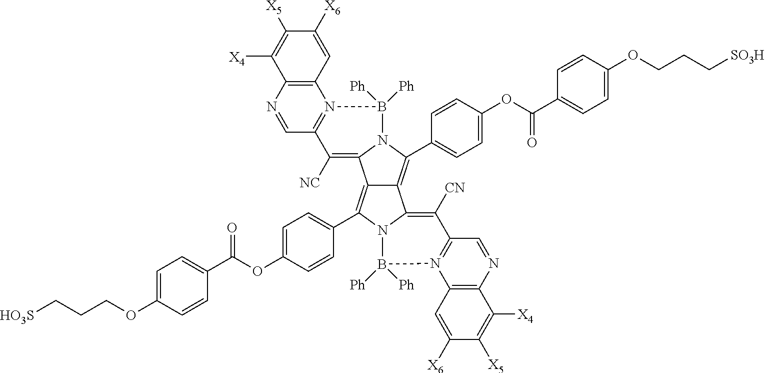

| Compound Name | X₄ | X₅ | X₆ |
|---|---|---|---|
| Ap-48 | H | CI | CI |
| Ap-49 | H | OMe | CI |
| Ap-50 | H | CI | H |
| Ap-51 | CI | CI | CI |
| Ap-52 | CI | OMe | H |
| Ap-53 | CI | CI | H |
| Ap-54 | CI | H | CI |
| Ap-55 | H | NO₂ | H |
| Ap-56 | H | CF₃ | H |
| Ap-57 | H | CN | H |
| Ap-58 | H | SMe | H |
| Ap-59 | H | SPh | H |
| Ap-60 | H | Ph | H |

TABLE 3

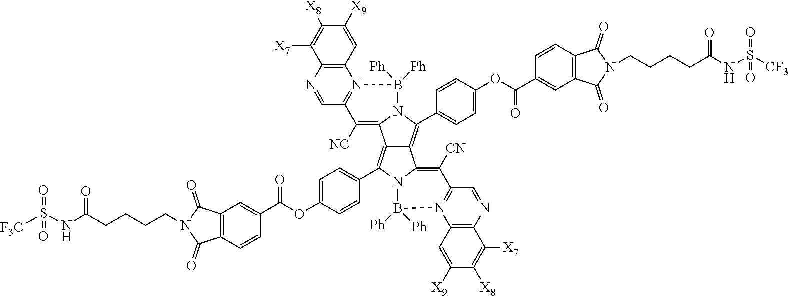

| Compound Name | X₇ | X₈ | X₉ |
|---|---|---|---|
| Ap-61 | H | CI | CI |
| Ap-62 | H | OMe | CI |
| Ap-63 | H | CI | H |
| Ap-64 | CI | CI | CI |
| Ap-65 | CI | OMe | H |
| Ap-66 | CI | CI | H |
| Ap-67 | CI | H | CI |
| Ap-68 | H | NO₂ | H |
| Ap-69 | H | CF₃ | H |
| Ap-70 | H | CN | H |
| Ap-71 | H | SMe | H |
| Ap-72 | H | SPh | H |
| Ap-73 | H | Ph | H |

TABLE 4

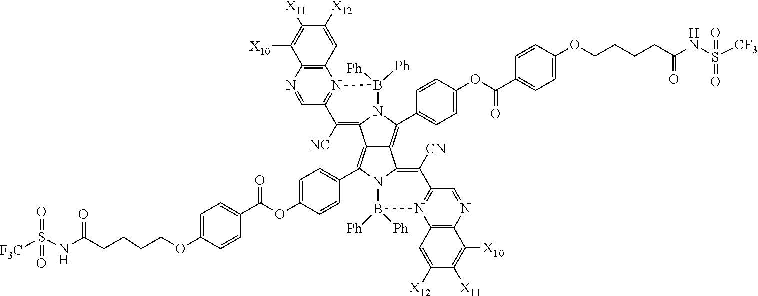

| Compound Name | X₁₀ | X₁₁ | X₁₂ |
|---|---|---|---|
| Ap-74 | H | CI | CI |
| Ap-75 | H | OMe | CI |
| Ap-76 | H | CI | H |
| Ap-77 | CI | CI | CI |
| Ap-78 | CI | OMe | H |
| Ap-79 | CI | CI | H |
| Ap-80 | CI | H | CI |
| Ap-81 | H | NO₂ | H |
| Ap-82 | H | CF₃ | H |
| Ap-83 | H | CN | H |
| Ap-84 | H | SMe | H |
| Ap-85 | H | SPh | H |
| Ap-86 | H | Ph | H |

TABLE 5

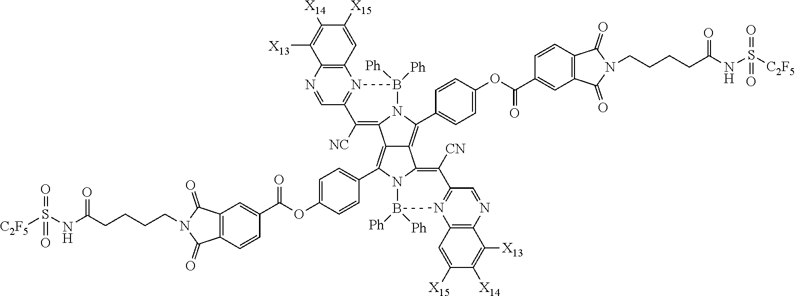

| Compound Name | X₁₃ | X₁₄ | X₁₅ |
|---|---|---|---|
| Ap-87 | H | CI | CI |
| Ap-88 | H | OMe | CI |
| Ap-89 | H | CI | H |
| Ap-90 | CI | CI | CI |
| Ap-91 | CI | OMe | H |
| Ap-92 | CI | CI | H |
| Ap-93 | CI | H | CI |
| Ap-94 | H | NO₂ | H |
| Ap-95 | H | CF₃ | H |
| Ap-96 | H | CN | H |
| Ap-97 | H | SMe | H |
| Ap-98 | H | SPh | H |
| Ap-99 | H | Ph | H |

TABLE 6

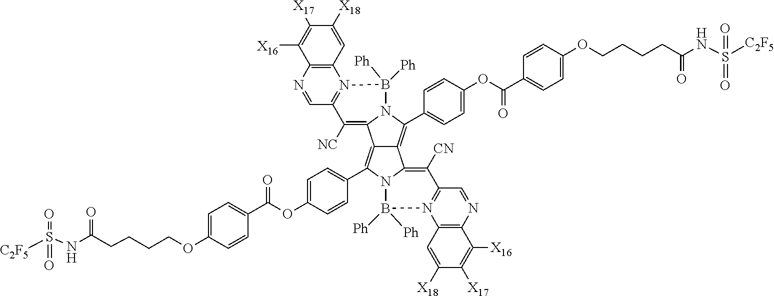

| Compound Name | X₁₆ | X₁₇ | X₁₈ |
|---|---|---|---|
| Ap-100 | H | CI | CI |
| Ap-101 | H | OMe | CI |
| Ap-102 | H | CI | H |
| Ap-103 | CI | CI | CI |
| Ap-104 | CI | OMe | H |
| Ap-105 | CI | Cl | H |
| Ap-106 | CI | H | CI |
| Ap-107 | H | NO₂ | H |
| Ap-108 | H | CF₃ | H |
| Ap-109 | H | CN | H |
| Ap-110 | H | SMe | H |
| Ap-111 | H | SPh | H |
| Ap-112 | H | Ph | H |

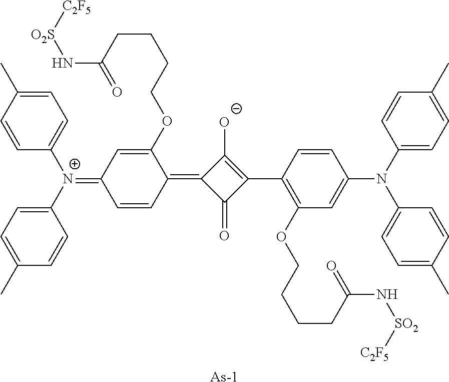

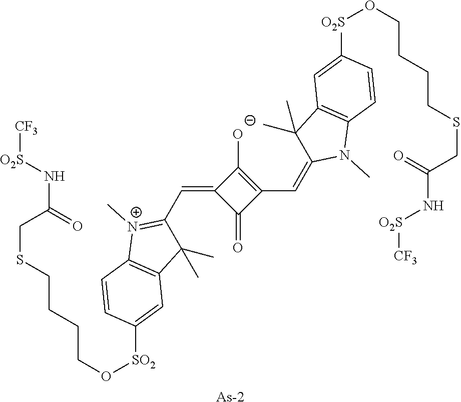

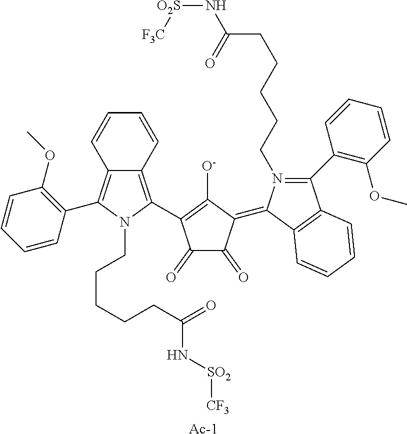

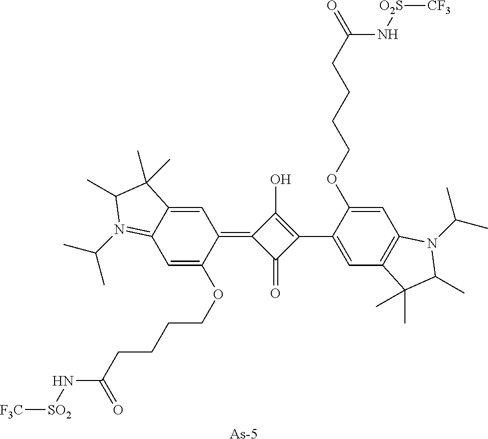

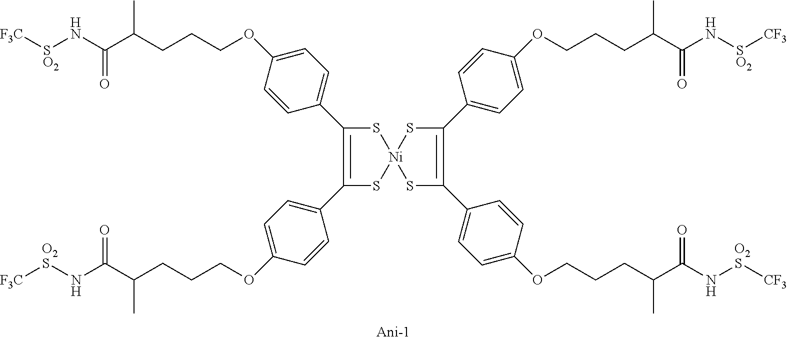

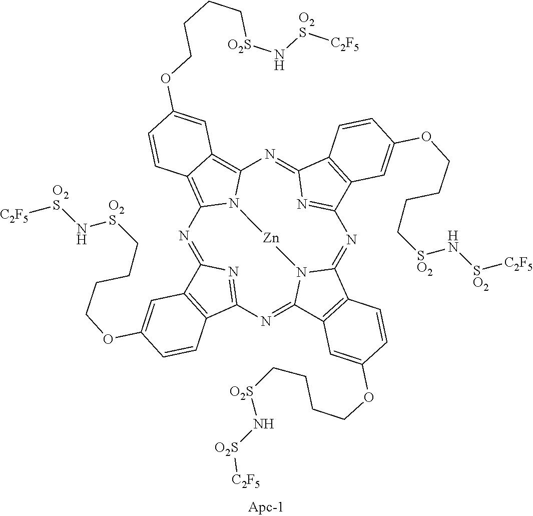

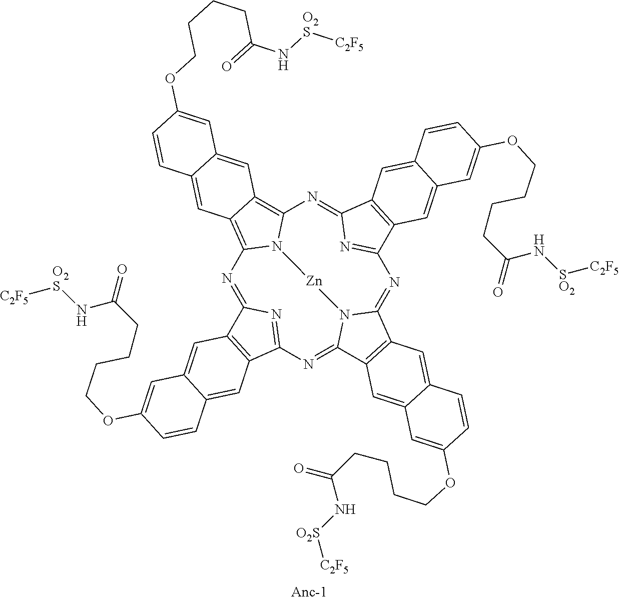

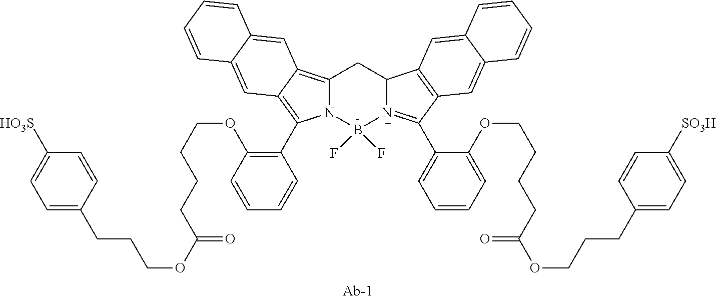

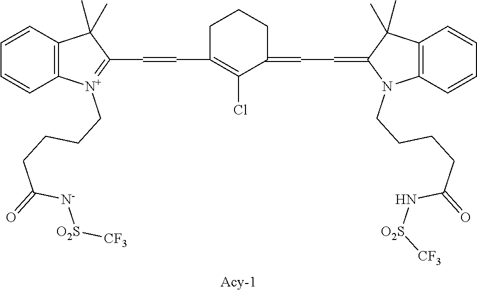 [SMILES: CC1(C)C(=CC=C2CCCC(C=CC3=[N+](CCCCC(=O)[N-]S(=O)(=O)C(F)(F)F)c4ccccc4C3(C)C)=C2Cl)N(CCCCC(=O)NS(=O)(=O)C(F)(F)F)c2ccccc21]

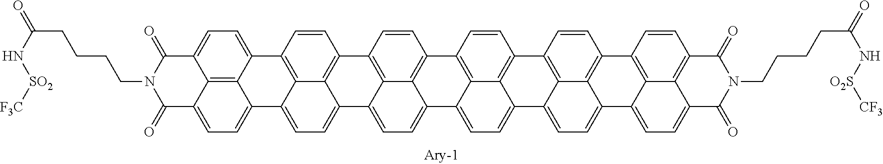 [SMILES: O=C(CCCCN1C(=O)c2ccc3c4ccc5c6ccc7c8ccc9c%10ccc%11c%12ccc%13c%14c(ccc(c%15ccc(c%16ccc(c%17ccc(c%18ccc(c%19ccc(c2c3%19)C1=O)c4c5%18)c6c7%17)c8c9%16)c%10c%11%15)c%14%12)C(=O)N(CCCCC(=O)NS(=O)(=O)C(F)(F)F)C%13=O)NS(=O)(=O)C(F)(F)F]

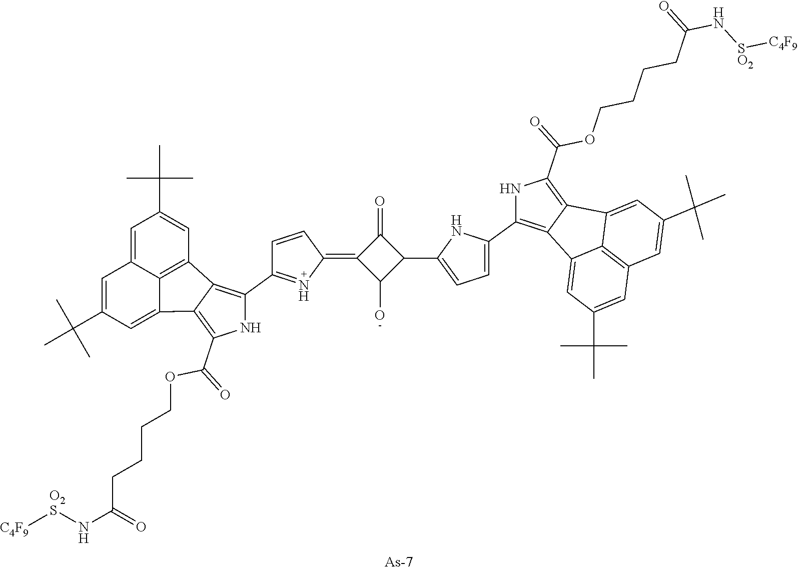

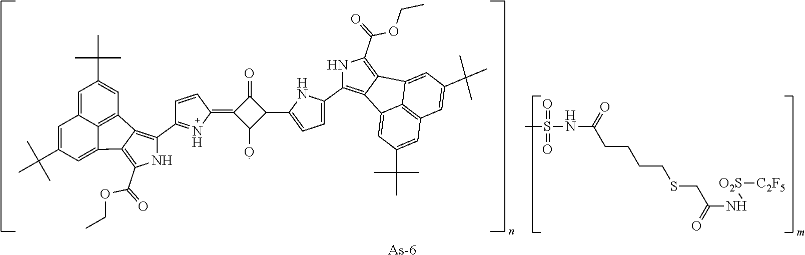

The compound A can be synthesized using a method described below in Examples. During synthesis of the compound A, a by-product may be formed. For example, in Ap-1, in a case where the compound A is synthesized using a method described below in Examples, the compound A may include a compound having a structure such as the following compound Y or compound Z.

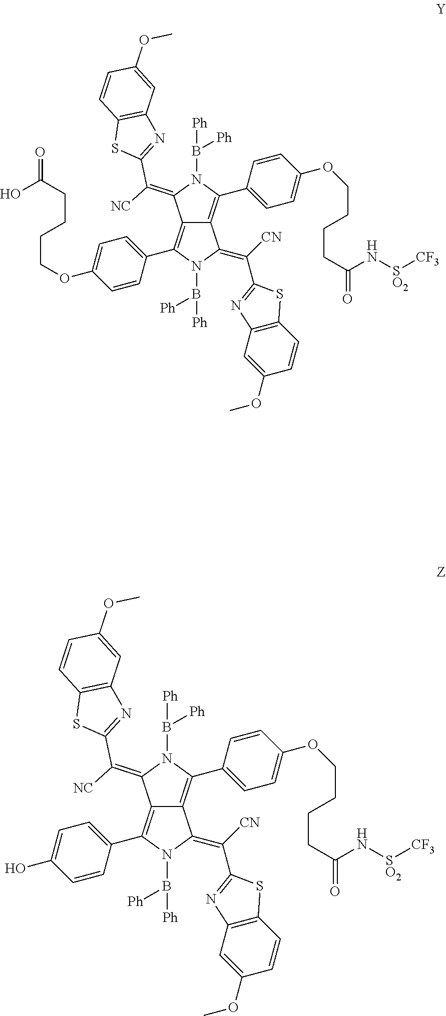 [SMILES: COc1ccc2sc(/C(C#N)=c3/c4c(-c5ccc(OCCCCC(=O)NS(=O)(=O)C(F)(F)F)cc5)n(B(c5ccccc5)c5ccccc5)/c(=C(/C#N)c5nc6cc(OC)ccc6s5)c4c(-c4ccc(OCCCCC(=O)O)cc4)n3B(c3ccccc3)c3ccccc3)nc2c1 ; COc1ccc2sc(/C(C#N)=c3/c4c(-c5ccc(OCCCCC(=O)NS(=O)(=O)C(F)(F)F)cc5)n(B(c5ccccc5)c5ccccc5)/c(=C(/C#N)c5nc6cc(OC)ccc6s5)c4c(-c4ccc(O)cc4)n3B(c3ccccc3)c3ccccc3)nc2c1]

In the present invention, the compound A may be used as a colorant or as a dispersing auxiliary agent. In a case where the compound A is used as a dispersing auxiliary agent, it is preferable that the curable composition according to the embodiment of the present invention includes another colorant described below other than the compound A. In addition, in a case where the compound A is used as colorant, the compound A may be a pigment or a dye. In the present invention, the pigment refers to a compound that is insoluble in a solvent. For example, a solubility of the pigment in the solvent (25° C.) included in the curable composition is preferably lower than 0.1 g/L and more preferably lower than 0.01 g/L. In addition, in the present invention, a dye refers to a compound that is likely to be soluble in a solvent. For example, a solubility of the dye in the solvent (25° C.) included in the curable composition is preferably 0.1 g/L or higher and more preferably 1 g/L or higher.

The compound A according to the present invention may include a metal selected from A1, Ca, Cu, Cr, Mg, Fe, Mn, Ni, Co, Cd, Li, Pb, Na, K, Zn, or Ti, the metal being free metal which is neither bonded nor coordinated to the compound A. The content of each of the metals other than Ti is preferably 20 ppm or lower. In addition, the content of free Ti is preferably 700 ppm or lower, more preferably 100 ppm or lower, and still more preferably 30 ppm or lower. According to this aspect, a color filter having reduced defects is likely to be manufactured. The content of the free metal in the compound A can be measured appropriately using the existing analysis methods and is preferably measured by inductively coupled plasma optical emission spectrometer (ICP-OES) as far as possible.

In the compound A according to the present invention, the content of free Br which is neither bonded nor coordinated to the compound A is preferably 20 ppm or lower. In addition, the content of free Cl is preferably 800 ppm or lower and more preferably 300 ppm or lower. According to this aspect, a color filter having reduced defects is likely to be manufactured. The content of free Br and the content of the free Cl in the compound A can be measured appropriately using the existing analysis methods. If possible, it is preferable that the measurement is performed using combustion ion chromatography according to BS EN 14582 (halogen content measurement).

The content of the compound A in the curable composition according to the embodiment of the present invention is preferably 0.01 to 50 mass % with respect to the total solid content of the curable composition according to the embodiment of the present invention. In addition, in a case where the compound A in the curable composition according to the embodiment of the present invention is used as a colorant, the content of the compound A is preferably 1 to 30 mass % with respect to the total solid content of the curable composition according to the embodiment of the present invention. The lower limit is preferably 2.5 mass % or higher and more preferably 5.0 mass % or higher. The upper limit is preferably 25 mass % or lower and more preferably 20 mass % or lower. In addition, in a case where the compound A in the curable composition according to the embodiment of the present invention is used as a dispersing auxiliary agent, the content of the compound A as the dispersing auxiliary agent is preferably 0.5 to 40 parts by mass with respect to 100 parts by mass of the pigment. The lower limit is preferably 1 part by mass or more and more preferably 5 parts by mass or more. The upper limit is preferably 35 parts by mass or less and more preferably 25 parts by mass or less.

<<Curable Compound>>

The curable composition according to the embodiment of the present invention includes a curable compound. Examples of the curable compound include a crosslinking compound and a resin. The resin may be a non-crosslinking resin (resin not having a crosslinking group) or a crosslinking resin (resin having a crosslinking group). Examples of the crosslinking group include a group having an ethylenically unsaturated bond, an epoxy group, a methylol group, and an alkoxymethyl group. Examples of the group having an ethylenically unsaturated bond include a vinyl group, a (meth)allyl group, and a (meth)acryloyl group. The crosslinking resin (resin having a crosslinking group) may be a crosslinking compound.

In the present invention, it is preferable that a compound including at least a resin is used as the curable compound, it is more preferable that a monomer type crosslinking compound including a resin is used as the curable compound, and it is still more preferable that a monomer type crosslinking compound including a resin and a group having an ethylenically unsaturated bond is used as the curable compound.

In the curable composition according to the embodiment of the present invention, the content of the curable compound is preferably 0.1 to 80 mass % with respect to the total solid content of the curable composition according to the embodiment of the present invention. The lower limit is more preferably 0.5 mass % or higher, still more preferably 1 mass % or higher, and still more preferably 5 mass % or higher. The upper limit is more preferably 75 mass % or lower and still more preferably 70 mass % or lower. As the curable compound, one kind may be used alone, or two or more kinds may be used. In a case where two or more curable compounds are used in combination, it is preferable that the total content of the two or more curable compounds is in the above-described range.

(Crosslinking Compound)

Examples of the crosslinking compound include a compound which has a group having an ethylenically unsaturated bond, a compound having an epoxy group, a compound having a methylol group, and a compound having an alkoxymethyl group. The crosslinking compound may be a monomer or a resin. The monomer type crosslinking compound that has a group having an ethylenically unsaturated bond can be preferably used as a radically polymerizable compound. In addition, the compound having an epoxy group, the compound having a methylol group, and the compound having an alkoxymethyl group can be preferably used as a cationically polymerizable compound.

The molecular weight of the monomer type crosslinking compound is preferably lower than 2000, more preferably 100 or higher and lower than 2000, and still more preferably 200 or higher and lower than 2000. The upper limit is, for example, preferably 1500 or lower. The weight-average molecular weight (Mw) of the resin type crosslinking compound is preferably 2000 to 2000000. The upper limit is 1000000 or lower and more preferably 500000 or lower. The lower limit is 3000 or higher and more preferably 5000 or higher.

Examples of the resin type crosslinking compound include an epoxy resin and a resin which includes a repeating unit having a crosslinking group. Examples of the repeating unit having a crosslinking group include the following (A2-1) to (A2-4).

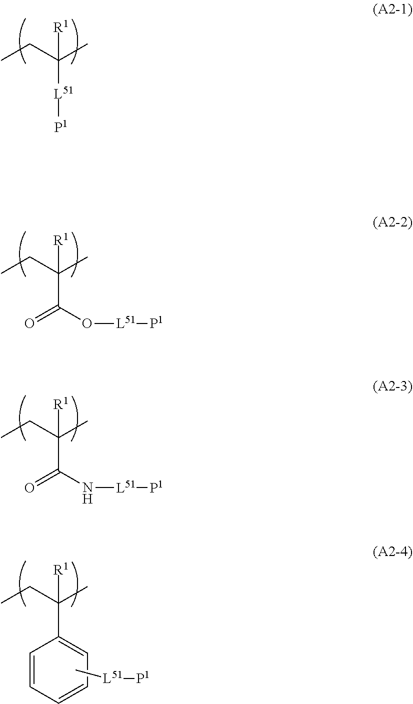

R¹ represents a hydrogen atom or an alkyl group. The number of carbon atoms in the alkyl group is preferably 1 to 5, more preferably 1 to 3, and still more preferably 1. It is preferable that R¹ represents a hydrogen atom or a methyl group.

L⁵¹ represents a single bond or a divalent linking group. Examples of the divalent linking group include an alkylene group, an arylene group, —O—, —S—, —CO—, —COO—, —OCO—, —SO₂—, —NR¹⁰— (R¹⁰ represents a hydrogen atom or an alkyl group and preferably a hydrogen atom), and a group including a combination thereof. The number of carbon atoms in the alkylene group is preferably 1 to 30, more preferably 1 to 15, and still more preferably 1 to 10.

The alkylene group may have a substituent but is preferably unsubstituted. The alkylene group may be linear, branched, or cyclic. In addition, the cyclic alkylene group may be monocyclic or polycyclic. The number of carbon atoms in the arylene group is preferably 6 to 18, more preferably 6 to 14, and still more preferably 6 to 10.

P¹ represents a crosslinking group. Examples of the crosslinking group include a group having an ethylenically unsaturated bond, an epoxy group, a methylol group, and an alkoxymethyl group.

The compound which has a group having an ethylenically unsaturated bond is preferably a (meth)acrylate compound having 3 to 15 functional groups and more preferably a (meth)acrylate compound having 3 to 6 functional groups. Examples of the compound which includes a group having an ethylenically unsaturated bond can be found in paragraphs “0033” and “0034” of JP2013-253224A, the content of which is incorporated herein by reference.

As specific examples, ethyleneoxy-modified pentaerythritol tetraacrylate (as a commercially available product, NK ESTER ATM-35E manufactured by Shin-Nakamura Chemical Co., Ltd.), dipentaerythritol triacrylate (as a commercially available product, KAYARAD D-330 manufactured by Nippon Kayaku Co., Ltd.), dipentaerythritol tetraacrylate (as a commercially available product, KAYARAD D-320 manufactured by Nippon Kayaku Co., Ltd.), dipentaerythritol penta(meth)acrylate (as a commercially available product, KAYARAD D-310 manufactured by Nippon Kayaku Co., Ltd.), dipentaerythritol hexa(meth)acrylate (as a commercially available product, KAYARAD DPHA manufactured by Nippon Kayaku Co., Ltd., A-DPH-12E, manufactured by Shin-Nakamura Chemical Co., Ltd.), or a structure in which the (meth)acryloyl group is bonded through an ethylene glycol residue or a propylene glycol residue is preferable. In addition, oligomers of the above-described examples can be used. In addition, the details can be found in paragraphs “0034” to “0038” of JP2013-253224A and paragraph “0477” of JP2012-208494A (corresponding to paragraph “0585” of US2012/0235099A), the contents of which are incorporated herein by reference. As specific examples of the compound which has a group having an ethylenically unsaturated bond, diglycerin ethylene oxide (EO)-modified (meth)acrylate (as a commercially available product, M-460 manufactured by Toagosei Co., Ltd.), pentaerythritol tetraacrylate (A-TMMT manufactured by Shin-Nakamura Chemical Co., Ltd.), or 1,6-hexanediol diacrylate (KAYARAD HDDA manufactured by Nippon Kayaku Co., Ltd.) can also be used. In addition, oligomers of the above-described examples can be used. For examples, RP-1040 (manufactured by Nippon Kayaku Co., Ltd.) is used. In addition, it is preferable that a compound substantially not including an environmentally regulated material such as toluene is also used as the compound which has a group having an ethylenically unsaturated bond. Examples of a commercially available product of the compound include KAYARAD DPHA LT and KAYARAD DPEA-12 LT (manufactured by Nippon Kayaku Co., Ltd.).

The compound which includes a group having an ethylenically unsaturated bond may further have an acid group such as a carboxyl group, a sulfo group, or a phosphate group. Examples of a commercially available product include ARONIX series (for example, M-305, M-510, or M-520, manufactured by Toagosei Co., Ltd.).

In addition, a compound having a caprolactone structure is also preferable as the compound which includes a group having an ethylenically unsaturated bond. Examples of the compound having a caprolactone structure can be found in paragraphs “0042” to “0045” of JP2013-253224A, the content of which is incorporated herein by reference. Examples of a commercially available product of the compound having a caprolactone structure include SR-494 (manufactured by Sartomer) which is a tetrafunctional acrylate having four ethyleneoxy chains, DPCA-60 (manufactured by Nippon Kayaku Co., Ltd.) which is a hexafunctional acrylate having six pentyleneoxy chains, and TPA-330 (manufactured by Nippon Kayaku Co., Ltd.) which is a trifunctional acrylate having three isobutyleneoxy chains. In addition, as the compound that includes the group having an ethylenically unsaturated bond, for example, 8UH-1006 or 8UH-1012 (manufactured by Taisei Fine Chemical Co., Ltd.) or LIGHT ACRYLATE POB-A0 (manufactured by Kyoeisha Chemical Co., Ltd.) is also preferably used.

In a case where the curable composition according to the embodiment of the present invention includes the compound which includes a group having an ethylenically unsaturated bond, the content of the compound which includes a group having an ethylenically unsaturated bond is preferably 0.1 mass % or higher, more preferably 0.5 mass % or higher, still more preferably 1 mass % or higher, and still more preferably 5 mass % or higher with respect to the total solid content of the curable composition. The upper limit is preferably 80 mass % or lower, more preferably 75 mass % or lower, and still more preferably 70 mass % or lower.

Examples of the compound having an epoxy group (hereinafter, also referred to as “epoxy compound”) include a monofunctional or polyfunctional glycidyl ether compound, and a polyfunctional aliphatic glycidyl ether compound. In addition, as the epoxy compound, a compound having an alicyclic epoxy group can also be used.

Examples of the epoxy compound include a compound having one or more epoxy groups in one molecule. It is preferable that the epoxy compound is a compound having 1 to 100 epoxy groups in one molecule. The upper limit of the number of epoxy groups is, for example, 10 or less or 5 or less. The lower limit of the number of epoxy groups is preferably 2 or more.

The epoxy compound may be a low molecular weight compound (for example, molecular weight: lower than 1000) or a high molecular weight compound (macromolecule; for example, molecular weight: 1000 or higher, and in the case of a polymer, weight-average molecular weight: 1000 or higher). The weight-average molecular weight of the epoxy compound is preferably 2000 to 100000. The upper limit of the weight-average molecular weight is preferably 10000 or lower, more preferably 5000 or lower, and still more preferably 3000 or lower.

Examples of a commercially available product of the epoxy compound include EHPE 3150 (manufactured by Daicel Corporation), EPICLON N-695 (manufactured by DIC Corporation), ADEKA GLYCILOL ED-505 (manufactured by Adeka Corporation, an epoxy group-containing monomer), and MARPROOF G-0150M, G-0105SA, G-0130SP, G-0250SP, G-1005S, G-1005SA, G-1010S, G-2050M, G-01100, or G-01758 (manufactured by NOF Corporation, an epoxy group-containing polymer). In addition, as the epoxy compound, compounds described in paragraphs “0034” to “0036” of JP2013-011869A, paragraphs “0147” to “0156” of JP2014-043556A, and paragraphs “0085” to “0092” of JP2014-089408A can also be used. The contents of this specification are incorporated herein by reference.

In a case where the curable composition according to the embodiment of the present invention includes the epoxy compound, the content of the epoxy compound is preferably 0.1 mass % or higher, more preferably 0.5 mass % or higher, still more preferably 1 mass % or higher, and still more preferably 5 mass % or higher with respect to the total solid content of the curable composition. The upper limit is preferably 80 mass % or lower, more preferably 75 mass % or lower, and still more preferably 70 mass % or lower.

In addition, in a case where the curable composition according to the embodiment of the present invention includes the radically polymerizable compound and the epoxy compound, a mass ratio radically polymerizable compound:epoxy compound is preferably 100:1 to 100:400 and more preferably 100:1 to 100:100.

Examples of the compound having a methylol group (hereinafter, also referred to as “methylol compound”) include a compound in which a methylol group is bonded to a nitrogen atom or a carbon atom which forms an aromatic ring. In addition, examples of the compound having an alkoxymethyl group (hereinafter, also referred to as “alkoxymethyl compound”) include a compound in which an alkoxymethyl group is bonded to a nitrogen atom or a carbon atom which forms an aromatic ring. As the compound in which an alkoxymethyl group or a methylol group is bonded to a nitrogen atom, for example, alkoxy methylated melamine, methylolated melamine, alkoxy methylated benzoguanamine, methylolated benzoguanamine, alkoxy methylated glycoluril, methylolated glycoluril, alkoxy methylated urea, or methylolated urea is preferable. In addition, the details can be found in paragraphs “0134” to “0147” of JP2004-295116A or paragraphs “0095” to “0126” of JP2014-089408A, the contents of which are incorporated herein by reference.

In a case where the curable composition according to the embodiment of the present invention includes the methylol compound, the content of the methylol compound is preferably 0.1 mass % or higher, more preferably 0.5 mass % or higher, still more preferably 1 mass % or higher, and still more preferably 5 mass % or higher with respect to the total solid content of the curable composition. The upper limit is preferably 80 mass % or lower, more preferably 75 mass % or lower, and still more preferably 70 mass % or lower.

In a case where the curable composition according to the embodiment of the present invention includes the alkoxymethyl compound, the content of the alkoxymethyl compound is preferably 0.1 mass % or higher, more preferably 0.5 mass % or higher, still more preferably 1 mass % or higher, and still more preferably 5 mass % or higher with respect to the total solid content of the curable composition. The upper limit is preferably 80 mass % or lower, more preferably 75 mass % or lower, and still more preferably 70 mass % or lower.

(Resin) The curable composition according to the embodiment of the present invention may include a resin as the curable compound. It is preferable that the curable compound includes at least a resin. The resin can also be used as a dispersant. The resin which is used to disperse the pigments and the like will also be referred to as a dispersant. However, the above-described uses of the resin are merely exemplary, and the resin can be used for purposes other than the uses. The resin having a crosslinking group also corresponds to the crosslinking compound.

The weight-average molecular weight (Mw) of the resin is preferably 2000 to 2000000. The upper limit is preferably 1000000 or lower and more preferably 500000 or lower. The lower limit is preferably 3000 or higher and more preferably 5000 or higher.

Examples of the resin include a (meth)acrylic resin, an epoxy resin, an enethiol resin, a polycarbonate resin, a polyether resin, a polyarylate resin, a polysulfone resin, a polyethersulfone resin, a polyphenylene resin, a polyarylene ether phosphine oxide resin, a polyimide resin, a polyamide imide resin, a polyolefin resin, a cyclic olefin resin, a polyester resin, and a styrene resin. Among these resins, one kind may be used alone, or a mixture of two or more kinds may be used. Examples of the epoxy resin include the polymer type compounds among the compounds described above as the examples of the epoxy compound regarding the crosslinking compound. In addition, as the resin, a resin described in Examples of WO2016/088645A or a resin described in Examples of JP2016-146619A can also be used.

The resin used in the present invention may have an acid group. Examples of the acid group include a carboxyl group, a phosphate group, a sulfo group, and a phenolic hydroxyl group. Among these acid groups, one kind may be used alone, or two or more kinds may be used in combination. The resin having an acid group can be preferably used as an alkali-soluble resin. By the curable composition according to the embodiment of the present invention including the alkali-soluble resin, a desired pattern can be formed by alkali development.

As the resin having an acid group, a polymer having a carboxyl group at a side chain is preferable. Specific examples of the resin include an alkali-soluble phenol resin such as a methacrylic acid copolymer, an acrylic acid copolymer, an itaconic acid copolymer, a crotonic acid copolymer, a maleic acid copolymer, a partially esterified maleic acid copolymer, or a novolac resin, an acidic cellulose derivative having a carboxyl group at a side chain thereof, and a resin obtained by adding an acid anhydride to a polymer having a hydroxyl group. In particular, a copolymer of (meth)acrylic acid and another monomer which is copolymerizable with the (meth)acrylic acid is preferable as the alkali-soluble resin. Examples of the monomer which is copolymerizable with the (meth)acrylic acid include an alkyl (meth)acrylate, an aryl (meth)acrylate, and a vinyl compound. Examples of the alkyl (meth)acrylate and the aryl (meth)acrylate include methyl (meth)acrylate, ethyl (meth)acrylate, propyl (meth)acrylate, butyl (meth)acrylate, isobutyl (meth)acrylate, pentyl (meth)acrylate, hexyl (meth)acrylate, octyl (meth)acrylate, phenyl (meth)acrylate, benzyl (meth)acrylate, tolyl (meth)acrylate, naphthyl (meth)acrylate, and cyclohexyl (meth)acrylate. Examples of the vinyl compound include styrene, α-methylstyrene, vinyl toluene, glycidyl methacrylate, acrylonitrile, vinyl acetate, N-vinylpyrrolidone, tetrahydrofurfuryl methacrylate, a polystyrene macromonomer, and a polymethyl methacrylate macromonomer. Examples of other monomers include a N-position-substituted maleimide monomer described in JP1998-300922A (JP-H10-300922A) such as N-phenylmaleimide or N-cyclohexylmaleimide. Among these monomers which are copolymerizable with the (meth)acrylic acid, one kind may be used alone, or two or more kinds may be used in combination.

The resin having an acid group may further include a repeating unit having a crosslinking group. In a case where the resin having an acid group further includes the repeating unit having a crosslinking group, the content of the repeating unit having a crosslinking group is preferably 10 to 90 mol %, more preferably 20 to 90 mol %, and still more preferably 20 to 85 mol % with respect to all the repeating units. In addition, the content of the repeating unit having an acid group is preferably 1 to 50 mol %, more preferably 5 to 40 mol %, and still more preferably 5 to 30 mol % with respect to all the repeating units.

As the resin having an acid group, a copolymer including benzyl (meth)acrylate and (meth)acrylic acid; a copolymer including benzyl (meth)acrylate, (meth)acrylic acid, and 2-hydroxyethyl (meth)acrylate; or a multi-component copolymer including benzyl (meth)acrylate, (meth)acrylic acid, and another monomer can be preferably used. In addition, copolymers described in JP1995-140654A (JP-H7-140654A) obtained by copolymerization of 2-hydroxyethyl (meth)acrylate can be preferably used, and examples thereof include: a copolymer including 2-hydroxypropyl (meth)acrylate, a polystyrene macromonomer, benzyl methacrylate, and methacrylic acid; a copolymer including 2-hydroxy-3-phenoxypropyl acrylate, a polymethyl methacrylate macromonomer, benzyl methacrylate, and methacrylic acid; a copolymer including 2-hydroxyethyl methacrylate, a polystyrene macromonomer, methyl methacrylate, and methacrylic acid; or a copolymer including 2-hydroxyethyl methacrylate, a polystyrene macromonomer, benzyl methacrylate, and methacrylic acid.

As the resin having an acid group, a polymer obtained by polymerization of monomer components including a compound represented by the following Formula (ED1) and/or a compound represented by the following Formula (ED2) (hereinafter, these compounds will also be referred to as “ether dimer”) is also preferable.

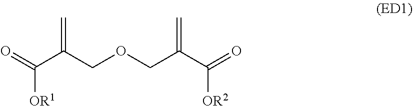

In Formula (ED1), R¹ and R² each independently represent a hydrogen atom or a hydrocarbon group having 1 to 25 carbon atoms which may have a substituent.

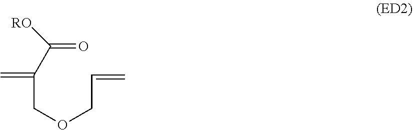

In Formula (ED2), R represents a hydrogen atom or an organic group having 1 to 30 carbon atoms. Specific examples of the compound represented by Formula (ED2) can be found in the description of JP2010-168539A.

Specific examples of the ether dimer can be found in paragraph “0317” of JP2013-029760A, the content of which is incorporated herein by reference. Among these ether dimers, one kind may be used alone, or two or more kinds may be used in combination.

The resin having an acid group may include a repeating unit which is derived from a compound represented by the following Formula (X).

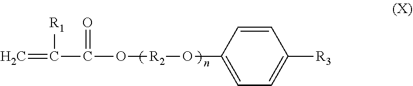

In Formula (X), R₁ represents a hydrogen atom or a methyl group, R₂ represents an alkylene group having 2 to 10 carbon atoms, and R₃ represents a hydrogen atom or an alkyl group having 1 to 20 carbon atoms which may have a benzene ring. n represents an integer of 1 to 15.

The details of the resin having an acid group can be found in paragraphs “0558” to “0571” of JP2012-208494A (paragraphs “0685” to “0700” of corresponding US2012/0235099A) and paragraphs “0076” to “0099” of JP2012-198408A, the contents of which are incorporated herein by reference.

The acid value of the resin having an acid group is preferably 30 to 200 mgKOH/g. The lower limit is preferably 50 mgKOH/g or higher and more preferably 70 mgKOH/g or higher. The upper limit is preferably 150 mgKOH/g or lower and more preferably 120 mgKOH/g or lower.

Examples of the resin having an acid group include resins having the following structures. In the following structural formulae, Me represents a methyl group.

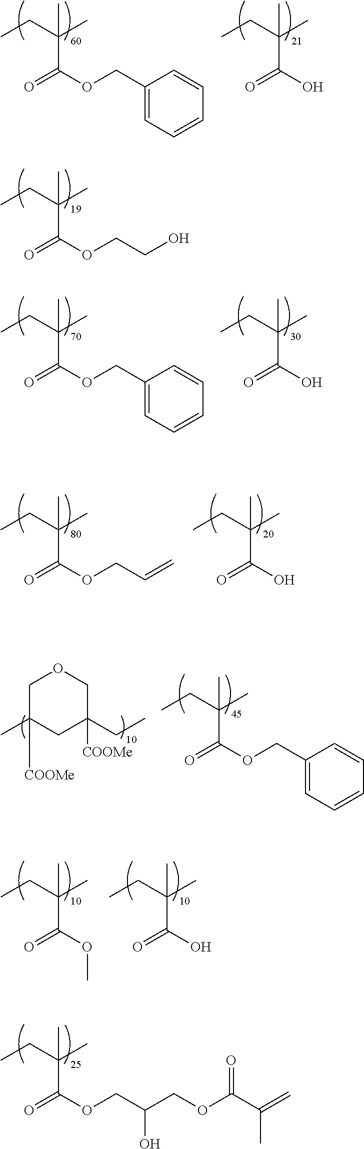

In the curable composition according to the embodiment of the present invention, as the resin, a resin having a repeating unit represented by any one of Formulae (A3-1) to (A3-7) is also preferably used.

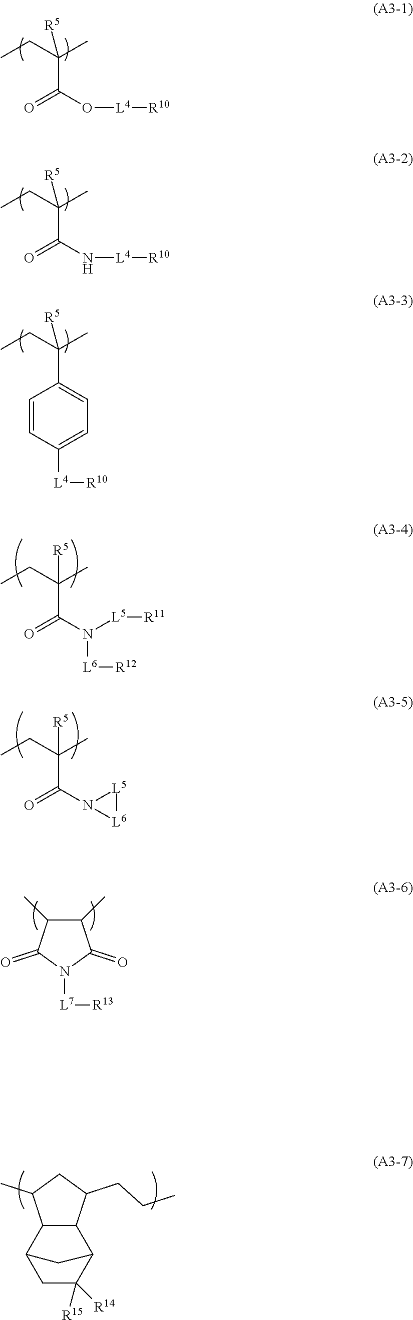

In the formulae, R⁵ represents a hydrogen atom or an alkyl group, L⁴ to L⁷ each independently represent a single bond or a divalent linking group, and R¹⁰ to R¹³ each independently represent an alkyl group or an aryl group. R¹⁴ and R¹⁵ each independently represent a hydrogen atom or a substituent.

The number of carbon atoms in the alkyl group represented by R⁵ is preferably 1 to 5, more preferably 1 to 3, and still more preferably 1. It is preferable that R⁵ represents a hydrogen atom or a methyl group.

Examples of the divalent linking group represented by L⁴ to L⁷ include an alkylene group, an arylene group, —O—, —S—, —CO—, —COO—, —OCO—, —SO₂—, —NR¹⁰— (R¹⁰ represents a hydrogen atom or an alkyl group and preferably a hydrogen atom), and a group including a combination thereof. Among these, a group including a combination an alkylene group, an arylene group, or an alkylene group and —O— is preferable. The number of carbon atoms in the alkylene group is preferably 1 to 30, more preferably 1 to 15, and still more preferably 1 to 10. The alkylene group may have a substituent but is preferably unsubstituted. The alkylene group may be linear, branched, or cyclic. In addition, the cyclic alkylene group may be monocyclic or polycyclic. The number of carbon atoms in the arylene group is preferably 6 to 18, more preferably 6 to 14, and still more preferably 6 to 10.

The alkyl group represented by R¹⁰ to R¹³ may be linear, branched, or cyclic and is preferably cyclic. The alkyl group may have a substituent or may be unsubstituted. The number of carbon atoms in the alkyl group is preferably 1 to 30, more preferably 1 to 20, and still more preferably 1 to 10. The number of carbon atoms in the aryl group represented by R¹⁰ to R¹³ is preferably 6 to 18, more preferably 6 to 12, and still more preferably 6. It is preferable that R¹⁰ represents a cyclic alkyl group or an aryl group. It is preferable that R¹¹ and R¹² represent a linear or branched alkyl group. It is preferable that R¹³ represents a linear alkyl group, a branched alkyl group, or an aryl group.

Examples of the substituent represented by R¹⁴ and R¹⁵ include a halogen atom, a cyano group, a nitro group, an alkyl group, an alkenyl group, an alkynyl group, an aryl group, a heteroaryl group, an aralkyl group, an alkoxy group, an aryloxy group, a heteroaryloxy group, an alkylthio group, an arylthio group, a heteroarylthio group, —NR^{a1}R^{a2}, —COR^{a3}, —COOR^{a4}, —OCOR^{a5}, —NHCOR^{a6}, —CONR^{a7}R^{a8}, —NHCONR^{a9}R^{a10}, —NHCOOR^{a11}, —SO₂R^{a12}, —SO₂OR^{a13}, —NHSO₂Ra¹⁴, and —SO₂NR^{a15}Ra¹⁶. R^{a1} to R^{a16} each independently represent a hydrogen atom, an alkyl group, an alkenyl group, an alkynyl group, an aryl group, or a heteroaryl group. In particular, it is preferable that at least one of R¹⁴ or R¹⁵ represents a cyano group or —COOR^{a4}. It is preferable that R^{a4} represents a hydrogen atom, an alkyl group, or an aryl group.

Examples of a commercially available product of the resin having a repeating unit represented by Formula (A3-7) include ARTON F4520 (manufactured by JSR Corporation). In addition, the details of the resin having a repeating unit represented by Formula (A3-7) can be found in paragraphs “0053” to “0075” and “0127” to “0130” of JP2011-100084A, the content of which is incorporated herein by reference.

The curable composition according to the embodiment of the present invention may include a dispersant as a resin. In particular, in a case where the compound A is a pigment or in a case where the curable composition further includes another pigment other than the compound A, it is preferable that the curable composition includes a dispersant. Examples of the dispersant include an acidic dispersant (acidic resin), a basic dispersant (basic resin), and an amphoteric dispersant (amphoteric resin). The dispersant is preferably a basic dispersant and/or an amphoteric dispersant and more preferably an amphoteric dispersant. By using a basic dispersant and/or an amphoteric dispersant as the dispersant, the dispersibility of the compound A or the pigment other than the compound A in the composition can be further improved. That is, in a case where a basic dispersant and/or an amphoteric dispersant is used as the dispersant, an interaction between the above-described functional group A in the compound A and the basic group in the basic dispersant or the amphoteric dispersant functions more strongly, and the dispersibility of the compound A in the curable composition can be further improved. In addition, in a case where the curable composition further includes a pigment (hereinafter, also referred to as “other pigment”) other than the compound A, the functional group A in the compound A also functions as the other pigment such that the compound A can also function as a dispersing auxiliary agent. Therefore, the dispersibility of the other pigment in the composition can also be improved.

In addition, by using a basic dispersant and/or an amphoteric dispersant as the dispersant, aggregation of the compound A or aggregation the pigment other than the compound A in the film can be suppressed, and a cured film in which the formation of an aggregate derived from a compound having a colorant skeleton is further suppressed can also be formed.

In a case where the amphoteric dispersant is used as the dispersant, not only the above-described effect but also developability can be further improved.

In the present invention, the acidic dispersant refers to a resin having an acid group which has an acid value of 5 mgKOH/g or higher and an amine value of lower than 5 mgKOH/g. It is preferable that the acidic dispersant does not have a basic group. The acid value of the acidic dispersant is preferably 5 to 200 mgKOH/g, more preferably 10 to 150 mgKOH/g, and still more preferably 30 to 150 mgKOH/g. In the present invention, the basic dispersant refers to a resin having an acid group which has an amine value of 5 mgKOH/g or higher and an acid value of lower than 5 mgKOH/g. It is preferable that the basic dispersant does not have an acid group. The amine value of the basic resin is preferably 5 to 200 mgKOH/g, more preferably 5 to 150 mgKOH/g, and still more preferably 5 to 100 mgKOH/g. In the present invention, the amphoteric dispersant refers to a resin having an acid group and a basic group which has an acid value of 5 mgKOH/g or higher and an amine value of 5 mgKOH/g or higher. The acid value of the amphoteric dispersant is preferably 5 to 200 mgKOH/g, more preferably 10 to 200 mgKOH/g, still more preferably 30 to 200 mgKOH/g, and still more preferably 30 to 180 mgKOH/g. The amine value of the amphoteric dispersant is preferably 5 to 200 mgKOH/g, more preferably 10 to 150 mgKOH/g, and still more preferably 10 to 130 mgKOH/g.

It is preferable that the resin used as the dispersant is a graft copolymer. Since the graft copolymer has affinity to the solvent due to the graft chain, the dispersibility of the pigment or the like and the dispersion stability over time are excellent. The details of the graft copolymer can be found in the description of paragraphs “0025” to “0094” of JP2012-255128A, the content of which is incorporated herein by reference.

In addition, in the present invention, as the resin (dispersant), an oligoimine dispersant having a nitrogen atom at at least either a main chain or a side chain is also preferably used. As the oligoimine dispersant, a resin, which includes a structural unit having a partial structure X with a functional group (pKa: 14 or lower) and a side chain including a side chain Y having 40 to 10000 atoms and has a basic nitrogen atom at at least either a main chain or a side chain, is preferable. The basic nitrogen atom is not particularly limited as long as it is a nitrogen atom exhibiting basicity.

The oligoimine dispersant can be found in the description of paragraphs “0102” to “0166” of JP2012-255128A, the content of which is incorporated herein by reference. Specific examples of the oligoimine dispersant are as follows. The following resin may also be a resin having an acid group (alkali-soluble resin). In addition, as the oligoimine dispersant, a resin described in paragraphs “0168” to “0174” of JP2012-255128A can also be used.

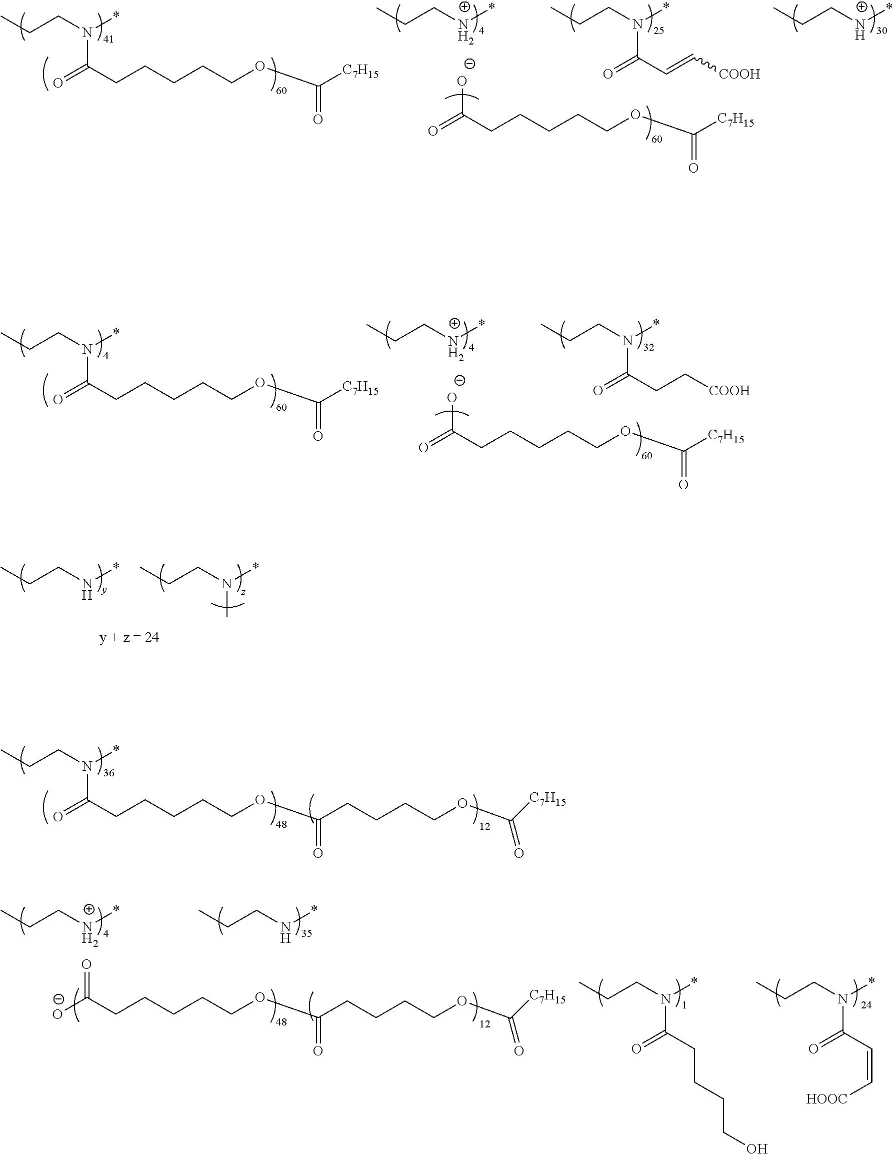

The dispersant is available as a commercially available product, and specific examples thereof include Disperbyk-111 (manufactured by BYK Chemie). In addition, a pigment dispersant described in paragraphs “0041” to “0130” of JP2014-130338A can also be used, the content of which is incorporated herein by reference. In addition, the resin having an acid group or the like can also be used as a dispersant.

In the curable composition according to the embodiment of the present invention, the content of the resin is preferably 1 mass % or higher, more preferably 5 mass % or higher, still more preferably 10 mass % or higher, and still more preferably 20 mass % or higher with respect to the total solid content of the curable composition. The upper limit is preferably 80 mass % or lower, more preferably 70 mass % or lower, and still more preferably 50 mass % or lower.

In a case where the curable composition according to the embodiment of the present invention includes the resin having an acid group, the content of the resin having an acid group is preferably 1 mass % or higher, more preferably 5 mass % or higher, still more preferably 10 mass % or higher, and still more preferably 20 mass % or higher with respect to the total solid content of the composition. The upper limit is preferably 80 mass % or lower, more preferably 70 mass % or lower, and still more preferably 50 mass % or lower.

In a case where the curable composition according to the embodiment of the present invention includes a dispersant, the content of the dispersant is preferably 50 to 1500 parts by mass with respect to 100 parts by mass of the compound A. In addition, in a case where the compound A is used as a pigment, the content of the dispersant is preferably 50 to 120 parts by mass with respect to 100 parts by mass of the compound A as the pigment. The lower limit is preferably 60 parts by mass or more and more preferably 70 parts by mass or more. The upper limit is preferably 110 parts by mass or less and more preferably 100 parts by mass or less. In addition, in a case where the compound A is used as a dispersing auxiliary agent, the content of the dispersant is preferably 500 to 1200 parts by mass with respect to 100 parts by mass of the compound A as the pigment as the dispersing auxiliary agent. The lower limit is preferably 600 parts by mass or more and more preferably 700 parts by mass or more. The upper limit is preferably 1100 parts by mass or less and more preferably 1000 parts by mass or less.

In addition, in a case where the curable composition according to the embodiment of the present invention includes the monomer type crosslinking compound that has a group having an ethylenically unsaturated bond and the resin, a mass ratio (monomer type crosslinking compound/resin) of the monomer type crosslinking compound that has a group having an ethylenically unsaturated bond to the resin is preferably 0.4 to 1.4. The lower limit of the mass ratio is preferably 0.5 or higher and more preferably 0.6 or higher. The upper limit of the mass ratio is preferably 1.3 or lower and more preferably 1.2 or lower. In a case where the mass ratio is in the above-described range, a pattern having excellent rectangularity can be easily formed.

In addition, a mass ratio (monomer type crosslinking compound/resin having an acid group) of the monomer type crosslinking compound that has a group having an ethylenically unsaturated bond to the resin having an acid group is preferably 0.4 to 1.4. The lower limit of the mass ratio is preferably 0.5 or higher and more preferably 0.6 or higher. The upper limit of the mass ratio is preferably 1.3 or lower and more preferably 1.2 or lower. In a case where the mass ratio is in the above-described range, a pattern having excellent rectangularity can be easily formed.

<<Solvent>>

The curable composition according to the embodiment of the present invention includes a solvent. Examples of the solvent include an organic solvent. Basically, the kind of the solvent is not particularly limited as long as it satisfies the solubility of the respective components and the application properties of the composition. Examples of the organic solvent include esters, ethers, ketones, and aromatic hydrocarbons. The details of the organic solvent can be found in paragraph “0223” of WO2015/166779A, the content of which is incorporated herein by reference. In addition, an ester solvent in which a cyclic alkyl group is substituted or a ketone solvent in which a cyclic alkyl group is substituted can also be preferably used. Specific examples of the organic solvent include dichloromethane, methyl 3-ethoxypropionate, ethyl 3-ethoxypropionate, ethyl cellosolve acetate, ethyl lactate, diethylene glycol dimethyl ether, butyl acetate, methyl 3-methoxypropionate, 2-heptanone, cyclohexanone, cyclohexyl acetate, cyclopentanone, ethyl carbitol acetate, butyl carbitol acetate, propylene glycol monomethyl ether, and propylene glycol monomethyl ether acetate. In addition, 3-methoxy-N,N-dimethylpropanamide or 3-butoxy-N,N-dimethylpropanamide is also preferable from the viewpoint of improving solubility. In the present invention, as the organic solvent, one kind may be used alone, or two or more kinds may be used in combination. In this case, it may be preferable that the content of the aromatic hydrocarbon (for example, benzene, toluene, xylene, or ethylbenzene) as the solvent is low (for example, 50 mass parts per million (ppm) or lower, 10 mass ppm or lower, or 1 mass ppm or lower with respect to the total mass of the organic solvent) in consideration of environmental aspects and the like.

In the present invention, a solvent having a low metal content is preferably used. For example, the metal content in the solvent is preferably 10 parts per billion (ppb) or lower. Optionally, a solvent having a metal content at a mass parts per trillion (ppt) level may be used. For example, such a high-purity solvent is available from Toyo Gosei Co., Ltd. (The Chemical Daily, Nov. 13, 2015).

Examples of a method of removing impurities such as metal from the solvent include distillation (for example, molecular distillation or thin-film distillation) and filtering using a filter. The pore size of a filter used for the filtering is preferably 10 m or less, more preferably 5 m or less, and still more preferably 3 μm or less. As a material of the filter, polytetrafluoroethylene, polyethylene, or nylon is preferable.

The solvent may include an isomer (a compound having the same number of atoms and a different structure). In addition, the organic solvent may include only one isomer or a plurality of isomers.

In the present invention, as the organic solvent, an organic solvent containing 0.8 mmol/L or lower of a peroxide is preferable, and an organic solvent containing substantially no peroxide is more preferable.

The content of the solvent is preferably 10 to 90 mass %, more preferably 20 to 80 mass %, and still more preferably 25 to 75 mass % with respect to the total mass of the curable composition. In a case where the composition includes two or more solvents, it is preferable that the total content of the solvents is in the above-described range.

In addition, it is also preferable that the curable composition according to the embodiment of the present invention does not substantially include an environmentally regulated material. In the present invention, not substantially including the environmentally regulated material represents that the content of the environmentally regulated material in the curable composition is 50 mass ppm or lower, preferably 30 mass ppm or lower, more preferably 10 mass ppm or lower, and still more preferably 1 mass ppm or lower. Examples of the environmentally regulated material include: benzene; an alkylbenzene such as toluene or xylene; and a halogenated benzene such as chlorobenzene. These compounds are registered as environmentally regulated materials based on Registration Evaluation Authorization and Restriction of Chemicals (REACH) regulation, Pollutant Release and Transfer Register (PRTR) method, Volatile Organic Compound (VOC) regulation, and the like, and the amount thereof used and a handling method thereof are strictly regulated. These compounds are used as solvents in a case where each of the components or the like used in the curable composition according to the embodiment of the present invention, and may be incorporated into the curable composition as residual solvents. From the viewpoints of safety for humans and consideration of the environment, it is preferable that these materials are reduced as much as possible. Examples of a method of reducing the environmentally regulated material include a method of distilling off the environmentally regulated material from the system by heating or depressurizing the system such that the temperature of the system is higher than or equal to a boiling point of the environmentally regulated material. In addition, in a case where a small amount of environmentally regulated material is removed by distillation, a method of azeotroping the environmentally regulated material with a solvent having the same boiling point as that of the corresponding solvent is also useful to increase the efficiency. In addition, in a case where a radically polymerizable compound is included, in order to suppress intermolecular crosslinking caused by the progress of a radical polymerization reaction during distillation under reduced pressure, a polymerization inhibitor or the like may be added for distillation under reduced pressure. This distillation method can be performed in, for example, any of a step of raw materials, a step of a reaction product (for example, a resin solution or a polyfunctional monomer solution after polymerization) obtained from a reaction of the raw materials, a step of a composition prepared by mixing these compounds with each other.

<<Other Colorants>>

The curable composition according to the embodiment of the present invention may further include a colorant other than the compound A (hereinafter, also referred to as “the other colorant”). The other colorant may be a pigment or a dye. A solubility of the pigment in the solvent (25° C.) included in the curable composition is preferably lower than 0.1 g/L and more preferably lower than 0.01 g/L. In addition, a solubility of the dye in the solvent (25° C.) included in the curable composition is preferably 0.1 g/L or higher and more preferably 1 g/L or higher. The other colorant may be a colorant having an absorption in a visible range (hereinafter, also referred to as “chromatic colorant”) or a colorant having an absorption in a near infrared range (hereinafter, also referred to as “near infrared absorbing colorant”). In particular, in a case where the other colorant is dispersed using the compound A, it is preferable that the other colorant is a near infrared absorbing colorant from the viewpoint of improving absorption properties in a near infrared range.

In addition, the other colorant may include a metal selected from A1, Ca, Cu, Cr, Mg, Fe, Mn, Ni, Co, Cd, Li, Pb, Na, K, Zn, or Ti, the metal being free metal which is neither bonded nor coordinated to the colorant. The content of each of the metals other than Ti is preferably 20 ppm or lower. In addition, the content of free Ti is preferably 700 ppm or lower. According to this aspect, a color filter having reduced defects is likely to be manufactured. In addition, in the other colorant, the content of free Br which is neither bonded nor coordinated to the colorant is preferably 20 ppm or lower. The content of free Cl is preferably 300 ppm or lower. According to this aspect, a color filter having reduced defects is likely to be manufactured.

The content of the other colorant is preferably 30 mass % or lower with respect to the total solid content of the curable composition. The lower limit is preferably higher than 0 mass %, more preferably 2.5 mass % or higher, and still more preferably 5 mass % or higher. The upper limit is preferably lower than 30 mass %, more preferably 25 mass % or lower, and still more preferably 20 mass % or lower.

In addition, the content of the other colorant is preferably 250 to 2000 parts by mass with respect to 100 parts by mass of the compound A. The lower limit is preferably more than 250 parts by mass, more preferably 300 parts by mass or more, and still more preferably 350 parts by mass or more. The upper limit is preferably less than 2000 parts by mass, more preferably 1750 parts by mass or less, and still more preferably 1500 parts by mass or less.

In addition, the total content of the compound A and the other colorant is preferably 1 to 50 mass % with respect to the total solid content of the curable composition. The lower limit is preferably 2 mass % or higher, more preferably 2.5 mass % or higher, and still more preferably 5.0 mass % or higher. The upper limit is preferably 45 mass % or lower, more preferably 40 mass % or lower, and still more preferably 30 mass % or lower.

(Chromatic Colorant)

The chromatic colorant is not particularly limited and is, for example, a colorant compound having an absorption in a visible range. Examples of the chromatic pigment include a diketo pyrrolo pyrrole compound, a phthalocyanine compound, a naphthalocyanine compound, an azo compound; an isoindoline compound, a quinophthalone compound, a benzimidazolone compound, and a perinone compound. Specific examples of the chromatic colorant include the following compounds.

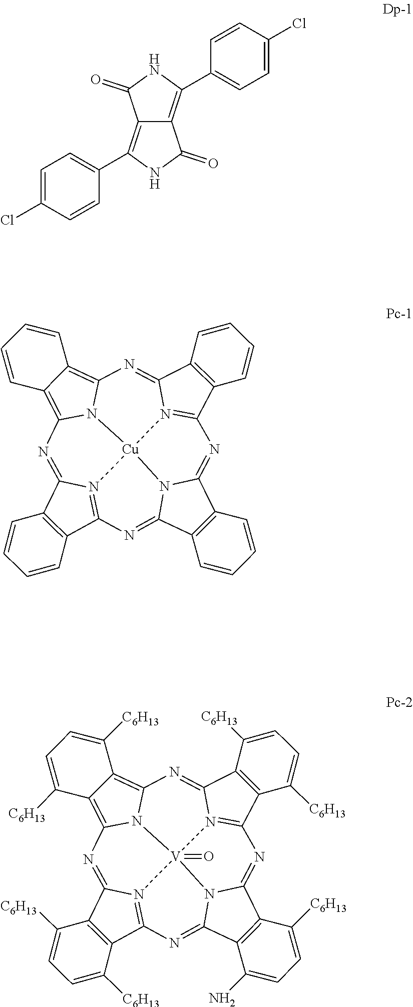

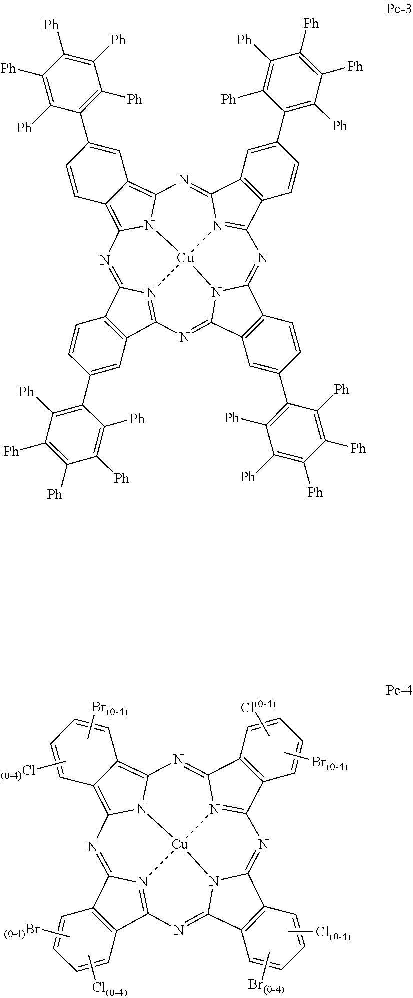

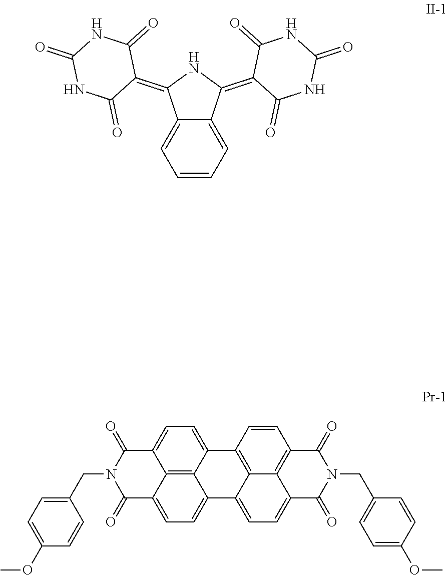

In addition, as the pigment type chromatic colorant, compounds having the following color index (C.I.) numbers can also be used.

Color Index (C.I.) Pigment Yellow 1, 2, 3, 4, 5, 6, 10, 11, 12, 13, 14, 15, 16, 17, 18, 20, 24, 31, 32, 34, 35, 35:1, 36, 36:1, 37, 37:1, 40, 42, 43, 53, 55, 60, 61, 62, 63, 65, 73, 74, 77, 81, 83, 86, 93, 94, 95, 97, 98, 100, 101, 104, 106, 108, 109, 110, 113, 114, 115, 116, 117, 118, 119, 120, 123, 125, 126, 127, 128, 129, 137, 138, 139, 147, 148, 150, 151, 152, 153, 154, 155, 156, 161, 162, 164, 166, 167, 168, 169, 170, 171, 172, 173, 174, 175, 176, 177, 179, 180, 181, 182, 185, 187, 188, 193, 194, 199, 213, 214, and 231 (all of which are yellow pigments);

C.I. Pigment Orange 2, 5, 13, 16, 17:1, 31, 34, 36, 38, 43, 46, 48, 49, 51, 52, 55, 59, 60, 61, 62, 64, 71, and 73 (all of which are orange pigments);

C.I. Pigment Red 1, 2, 3, 4, 5, 6, 7, 9, 10, 14, 17, 22, 23, 31, 38, 41, 48:1, 48:2, 48:3, 48:4, 49, 49:1, 49:2, 52:1, 52:2, 53:1, 57:1, 60:1, 63:1, 66, 67, 81:1, 81:2, 81:3, 83, 88, 90, 105, 112, 119, 122, 123, 144, 146, 149, 150, 155, 166, 168, 169, 170, 171, 172, 175, 176, 177, 178, 179, 184, 185, 187, 188, 190, 200, 202, 206, 207, 208, 209, 210, 216, 220, 224, 226, 242, 246, 254, 255, 264, 270, 272, and 279 (all of which are red pigments);

C.I. Pigment Green 7, 10, 36, 37, 58, 59, 62, and 63 (all of which are green pigments);

C.I. Pigment Violet 1, 19, 23, 27, 32, 37, and 42 (all of which are violet pigments); and

C.I. Pigment Blue 1, 2, 15, 15:1, 15:2, 15:3, 15:4, 15:6, 16, 22, 60, 64, 66, 79, and 80 (all of which are blue pigments).

Among these organic pigments, one kind may be used alone, or two or more kinds may be used in combination.

In addition, as a green colorant, a halogenated zinc phthalocyanine pigment having 10 to 14 halogen atoms, 8 to 12 bromine atoms, and 2 to 5 chlorine atoms in one molecule on average can also be used. Specific examples of the green colorant include a compound described in WO2015/118720A.

In addition, as a blue colorant, an aluminum phthalocyanine compound having a phosphorus atom can also be used. Specific examples of the blue colorant include a compound described in paragraphs “0022” to “0030” of JP2012-247591A and paragraph “0047” of JP2011-157478A.

In addition, as a yellow colorant, a colorant described in WO2012/128233A and JP2017-201003A can be used. In addition, as a red colorant, a colorant described in WO2012/102399A, WO02012/117965A, and JP2012-229344A can be used. In addition, as a green colorant, a colorant described in WO2012/102395A can be used. In addition, a salt-forming dye described in WO2011/037195A can also be formed.

In a case where the curable composition according to the embodiment of the present invention includes a chromatic colorant, it is preferable that the content of the chromatic colorant is 0.01 to 50 mass % with respect to the total solid content of the curable composition. The lower limit is preferably 0.1 mass % or higher and more preferably 0.5 mass % or higher. The upper limit is preferably 30 mass % or lower, and more preferably 15 mass % or lower. In addition, the total content of the chromatic colorant and the compound A is preferably 0.01 to 50 mass % with respect to the total solid content of the curable composition. The lower limit is preferably 0.1 mass % or higher and more preferably 0.5 mass % or higher. The upper limit is preferably 30 mass % or lower, and more preferably 15 mass % or lower. In the curable composition according to the embodiment of the present invention, as the chromatic colorant, one kind may be used alone, or two or more kinds may be used in combination. In a case where two or more chromatic colorants are used in combination, it is preferable that the total content of the chromatic colorants is in the above-described range.

(Near Infrared Absorbing Colorant)

As the near infrared absorbing colorant, a compound having a maximum absorption wavelength in a near infrared range can be preferably used. The near infrared absorbing colorant may be a pigment or a dye.

In the present invention, as the near infrared absorbing colorant, a near infrared absorbing compound that includes a π-conjugated structure having a monocyclic or fused aromatic ring can be preferably used. The number of atoms constituting the π-conjugated structure included in the near infrared absorbing compound other than hydrogen is preferably 14 or more, more preferably 20 or more, still more preferably 25 or more, and still more preferably 30 or more. For example, the upper limit is preferably 80 or less and more preferably 50 or less.

The number of monocyclic or fused aromatic rings in the in-conjugated structure included in the near infrared absorbing compound is preferably 2 or more, more preferably 3 or more, still more preferably 4 or more, and still more preferably 5 or more. The upper limit is preferably 100 or less, more preferably 50 or less, and still more preferably 30 or less. Examples of the aromatic ring include a benzene ring, a naphthalene ring, a pentalene ring, an indene ring, an azulene ring, a heptalene ring, an indacene ring, a perylene ring, a pentacene ring, a quaterrylene ring, an acenaphthene ring, a phenanthrene ring, an anthracene ring, a naphthacene ring, a chrysene ring, a triphenylene ring, a fluorene ring, a pyridine ring, a quinoline ring, an isoquinoline ring, an imidazole ring, a benzimidazole ring, a pyrazole ring, a thiazole ring, a benzothiazole ring, a triazole ring, a benzotriazole ring, an oxazole ring, a benzoxazole ring, an imidazoline ring, a pyrazine ring, a quinoxaline ring, a pyrimidine ring, a quinazoline ring, a pyridazine ring, a triazine ring, a pyrrole ring, an indole ring, an isoindole ring, a carbazole ring, and a fused ring including the above-described ring.

The near infrared absorbing compound has a maximum absorption wavelength preferably in a wavelength range of 700 to 1300 nm and more preferably in a wavelength range of 700 to 1000 nm.

In the present invention, as the near infrared absorbing compound, at least one selected from a pyrrolopyrrole compound, a cyanine compound, a squarylium compound, a phthalocyanine compound, a naphthalocyanine compound, a quaterrylene compound, a merocyanine compound, a croconium compound, an oxonol compound, a diimmonium compound, a dithiol compound, a triarylmethane compound, a pyrromethene compound, an azomethine compound, an anthraquinone compound, or a dibenzofuranone compound is preferable, at least one selected from a pyrrolopyrrole compound, a cyanine compound, a squarylium compound, a phthalocyanine compound, a naphthalocyanine compound, or a diimmonium compound is more preferable, at least one selected from a pyrrolopyrrole compound, a cyanine compound, and a squarylium compound is still more preferable, or a pyrrolopyrrole compound is still more preferable. Examples of the diimmonium compound include a compound described in JP2008-528706A, the content of which is incorporated herein by reference. Examples of the phthalocyanine compound include a compound described in paragraph “0093” of JP2012-077153A, oxytitaniumnphthalocyanine described in JP2006-343631A, and a compound described in paragraphs “0013” to “0029” of JP2013-195480A, the contents of which are incorporated herein by reference. Examples of the naphthalocyanine compound include a compound described in paragraph “0093” of JP2012-077153A, the content of which is incorporated herein by reference. In addition, as the cyanine compound, the phthalocyanine compound, the naphthalocyanine compound, the diim nonium compound, or the squarylium compound, for example, a compound described in paragraphs “0010” to “0081” of JP2010-111750A may be used, the content of which is incorporated herein by reference. In addition, the details of the cyanine compound can be found in, for example, “Functional Colorants by Makoto Okawara, Masaru Matsuoka, Teijiro Kitao, and Tsuneoka Hirashima, published by Kodansha Scientific Ltd.”, the content of which is incorporated herein by reference. In addition, a compound described in paragraphs JP2016-146619A can also be used as the near infrared absorbing compound, the content of which is incorporated herein by reference. In addition, it is also preferable that compounds having the following structures are used as the near infrared absorbing colorant.

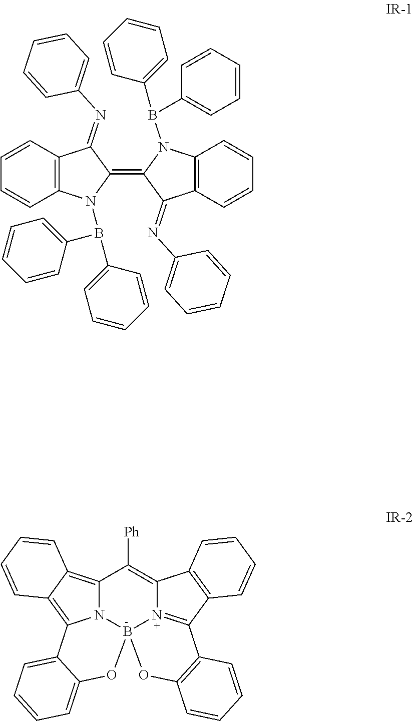

As the pyrrolopyrrole compound, a compound represented by Formula (PP) is preferable.

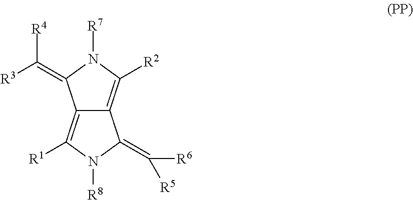

In Formula (PP), R¹ and R² each independently represent an alkyl group, an aryl group, or a heteroaryl group.

R³, R⁴, R⁵, and R⁶ each independently represent a cyano group, an acyl group, an alkoxycarbonyl group, an alkylsulfinyl group, an arylsulfinyl group, or a heteroaryl group.

R⁷ and R⁸ each independently represent a hydrogen atom, an alkyl group, an aryl group, a heteroaryl group, —BR⁹R¹⁰, or a metal atom.

R⁷ may form a covalent bond or a coordinate bond with R², R³, or R⁴.

R⁸ may form a covalent bond or a coordinate bond with R¹, R⁵, or R⁶.

R⁹ and R¹⁰ each independently represent a hydrogen atom, a halogen atom, an alkyl group, an aryl group, a heteroaryl group, an alkoxy group, an aryloxy group, or a heteroaryloxy group, and R⁹ and R¹⁰ may be bonded to each other to form a ring.

In Formula (PP), R¹ and R² each independently represent an alkyl group, an aryl group, or a heteroaryl group, preferably an aryl group or a heteroaryl group, and more preferably an aryl group. The number of carbon atoms in the alkyl group represented by R¹ and R² is preferably 1 to 30, more preferably 1 to 20, and still more preferably 1 to 10. The number of carbon atoms in the aryl group represented by R¹ and R² is preferably 6 to 30, more preferably 6 to 20, and still more preferably 6 to 12. The number of carbon atoms constituting the heteroaryl group represented by R¹ and R² is preferably 1 to 30 and more preferably 1 to 12. Examples of the kind of the heteroatom constituting the heteroaryl group include a nitrogen atom, an oxygen atom, and a sulfur atom. The number of heteroatoms constituting the heteroaryl group is preferably 1 to 3 and more preferably 1 or 2. The heteroaryl group is preferably a monocycle or a fused ring, more preferably a monocycle or a fused ring composed of 2 to 8 rings, and still more preferably a monocycle or a fused ring composed of 2 to 4 rings. The alkyl group, the aryl group, and the heteroaryl group may have a substituent or may be unsubstituted. It is preferable that the groups have a substituent. Examples of the substituent include the groups described regarding the substituent T.

In Formula (PP), R³, R⁴, R⁵, and R⁶ each independently represent a cyano group, an acyl group, an alkoxycarbonyl group, an alkylsulfinyl group, an arylsulfinyl group, or a heteroaryl group. It is preferable that one of R³ and R⁴ represents a cyano group, an acyl group, an alkoxycarbonyl group, an alkylsulfinyl group, or an arylsulfinyl group and another one of R³ and R⁴ represents a heteroaryl group, and it is more preferable that one of R³ and R⁴ represents a cyano group and another one of R³ and R⁴ represents a heteroaryl group. It is preferable that one of R⁵ and R⁶ represents a cyano group, an acyl group, an alkoxycarbonyl group, an alkylsulfinyl group, or an arylsulfinyl group and another one of R⁵ and R⁶ represents a heteroaryl group, and it is more preferable that one of R⁵ and R⁶ represents a cyano group and another one of R³ and R⁶ represents a heteroaryl group. Examples of the heteroaryl group include a group represented by Formula (A-1) of Formula (A1) and a group represented by Formula (A-2), and a preferable range thereof is also the same. In addition, the heteroaryl group may have a substituent or may be unsubstituted. Examples of the substituent include the groups described regarding the substituent T.

In Formula (PP), R⁷ and R⁸ each independently represent a hydrogen atom, an alkyl group, an aryl group, a heteroaryl group, —BR⁹R¹⁰, or a metal atom and preferably —BR⁹R. R⁹ and R¹⁰ each independently represent a hydrogen atom, a halogen atom, an alkyl group, an alkenyl group, an aryl group, a heteroaryl group, an alkoxy group, an aryloxy group, or a heteroaryloxy group, preferably a halogen atom, an alkyl group, an aryl group, or a heteroaryl group, more preferably a halogen atom, an alkyl group, or an aryl group, and still more preferably an aryl group. R⁹ and R¹⁰ may be bonded to each other to form a ring.

Specific examples of the compound represented by Formula (PP) include the following compounds. In the following structural formulae, Me represents a methyl group, and Ph represents a phenyl group. In addition, Examples of the pyrrolopyrrole compound include compounds described in paragraphs “0016” to “0058” of JP2009-263614A, compounds described in paragraphs “0037” to “0052” of JP2011-068731A, compounds described in paragraphs “0010” to “0033” of WO2015/166873A, the contents of which are incorporated herein by reference.

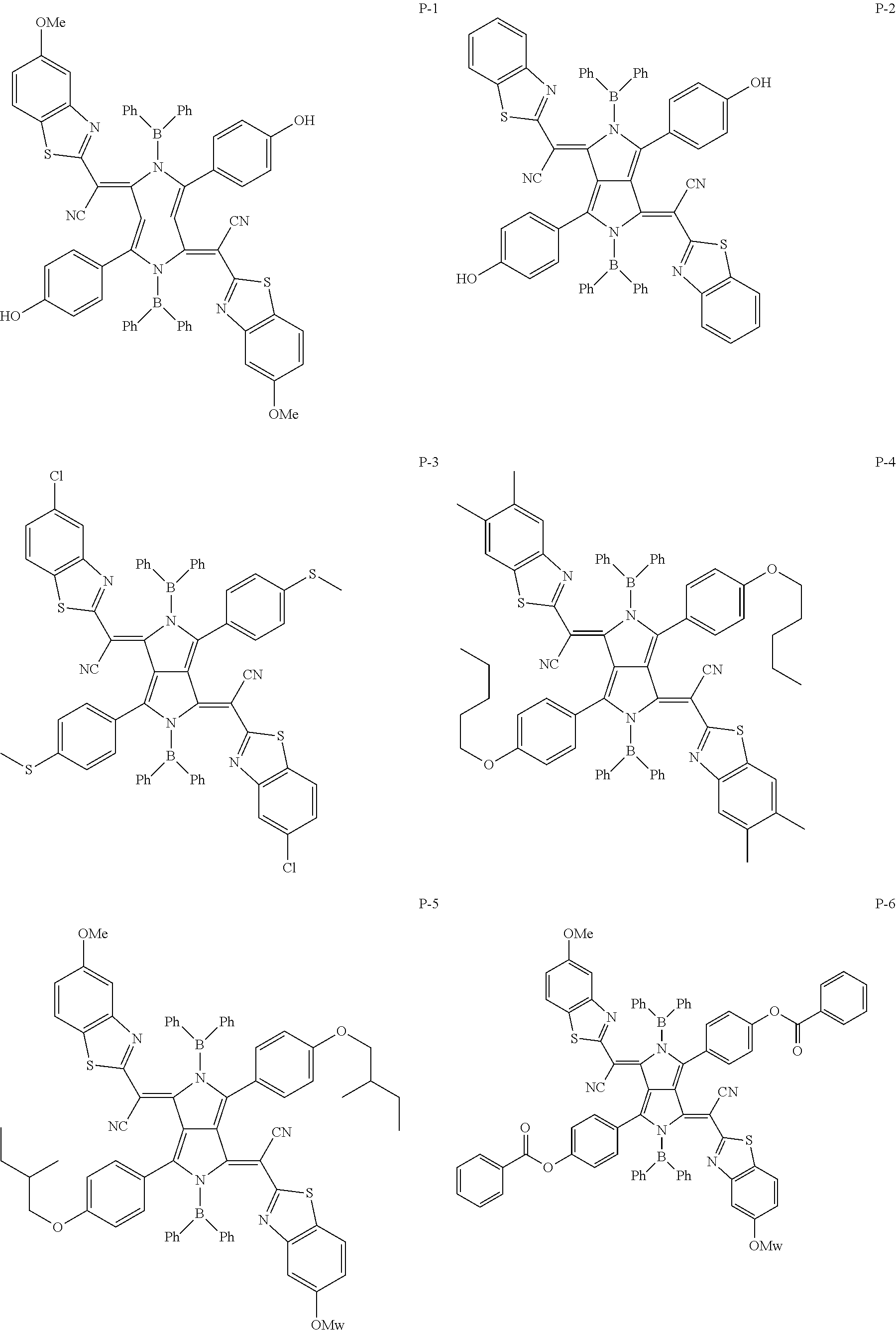

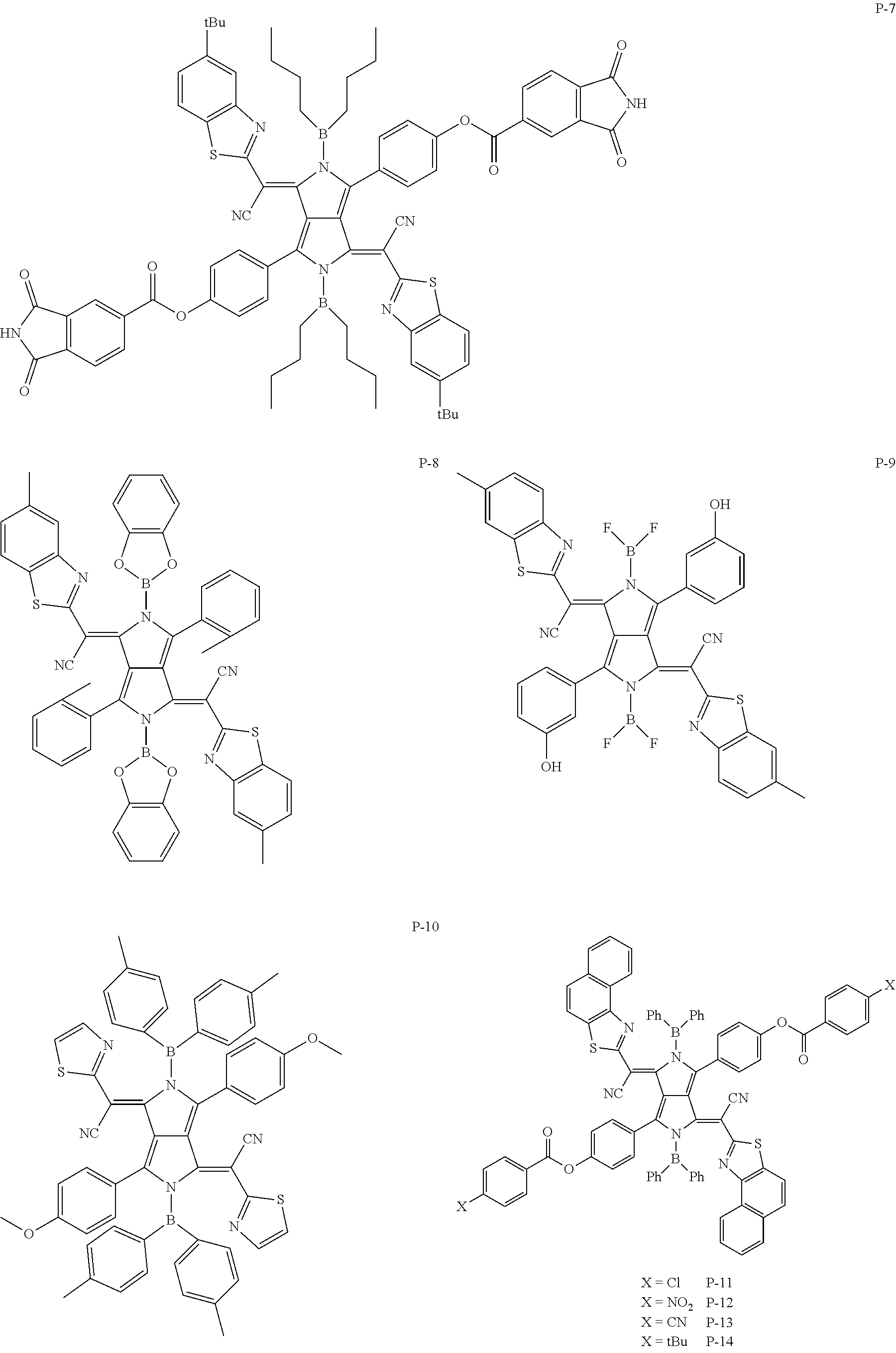

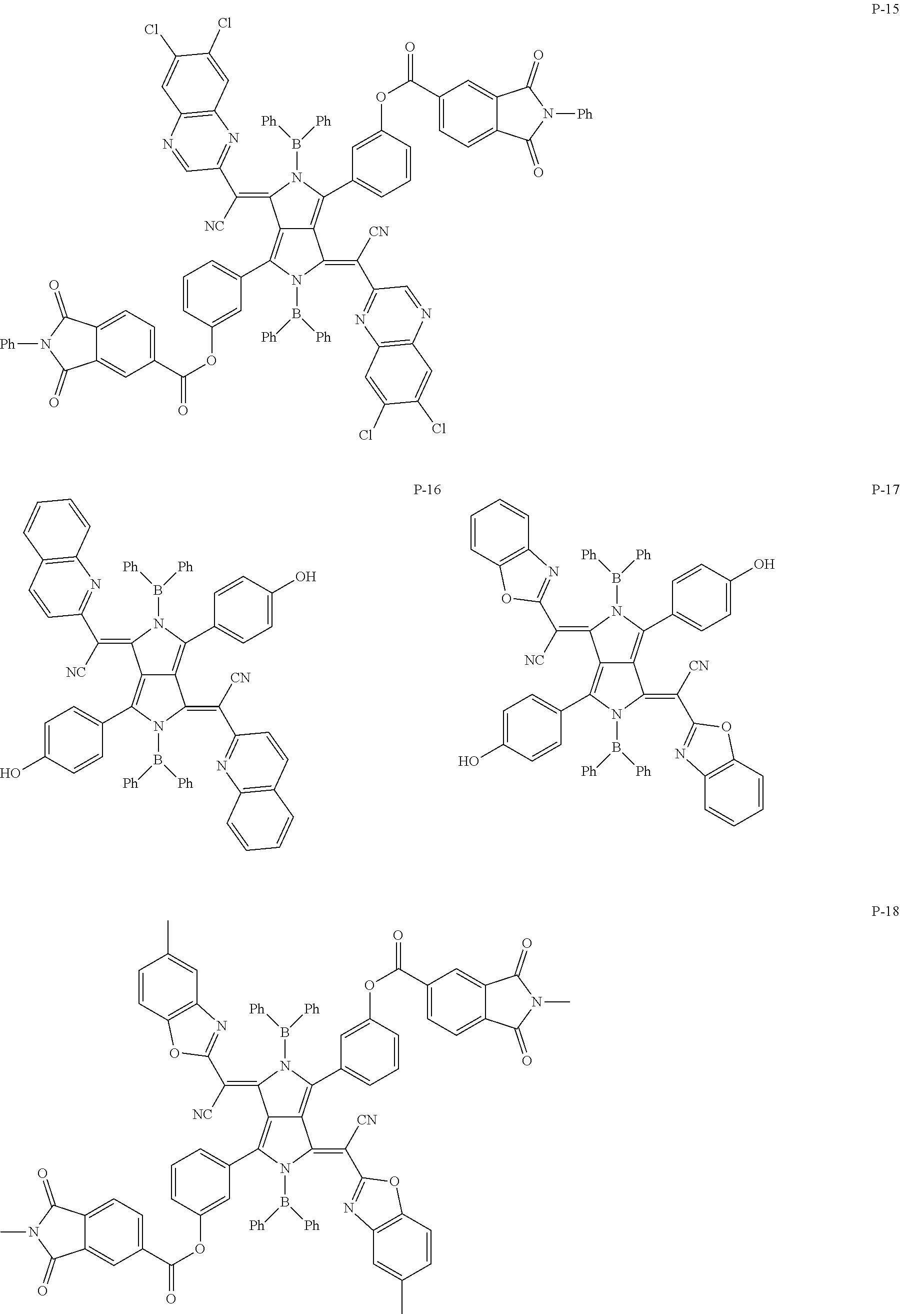

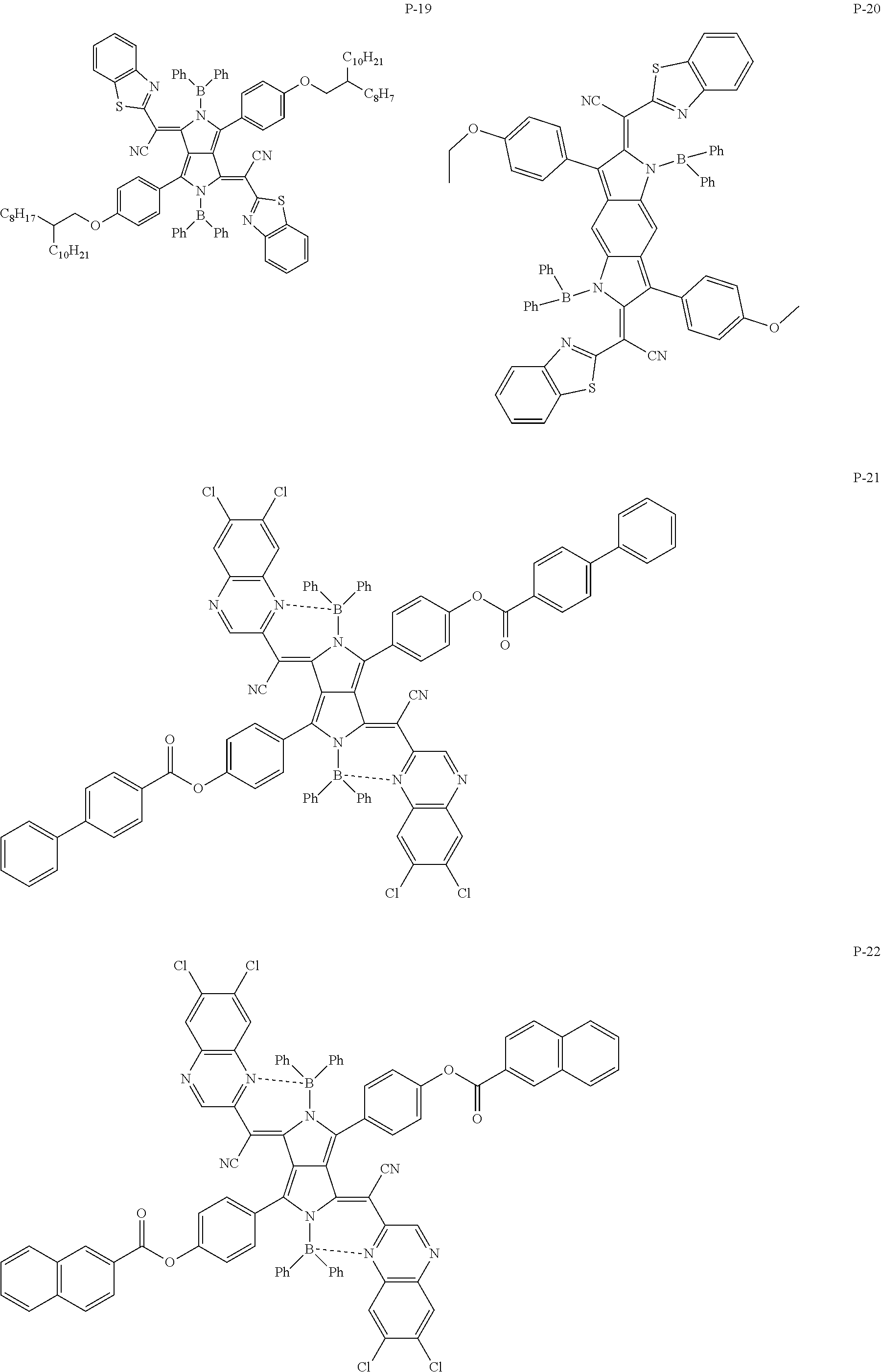

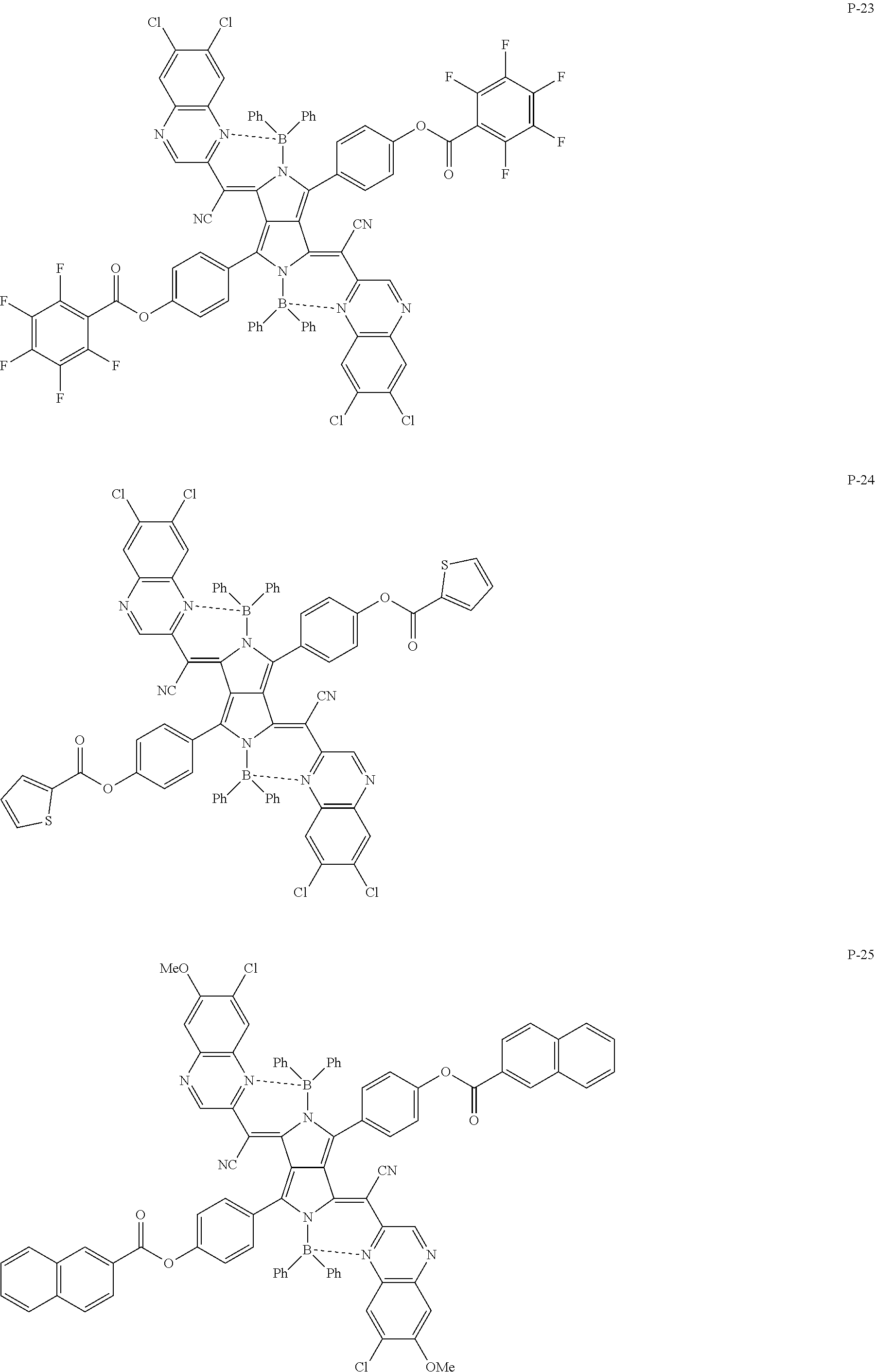

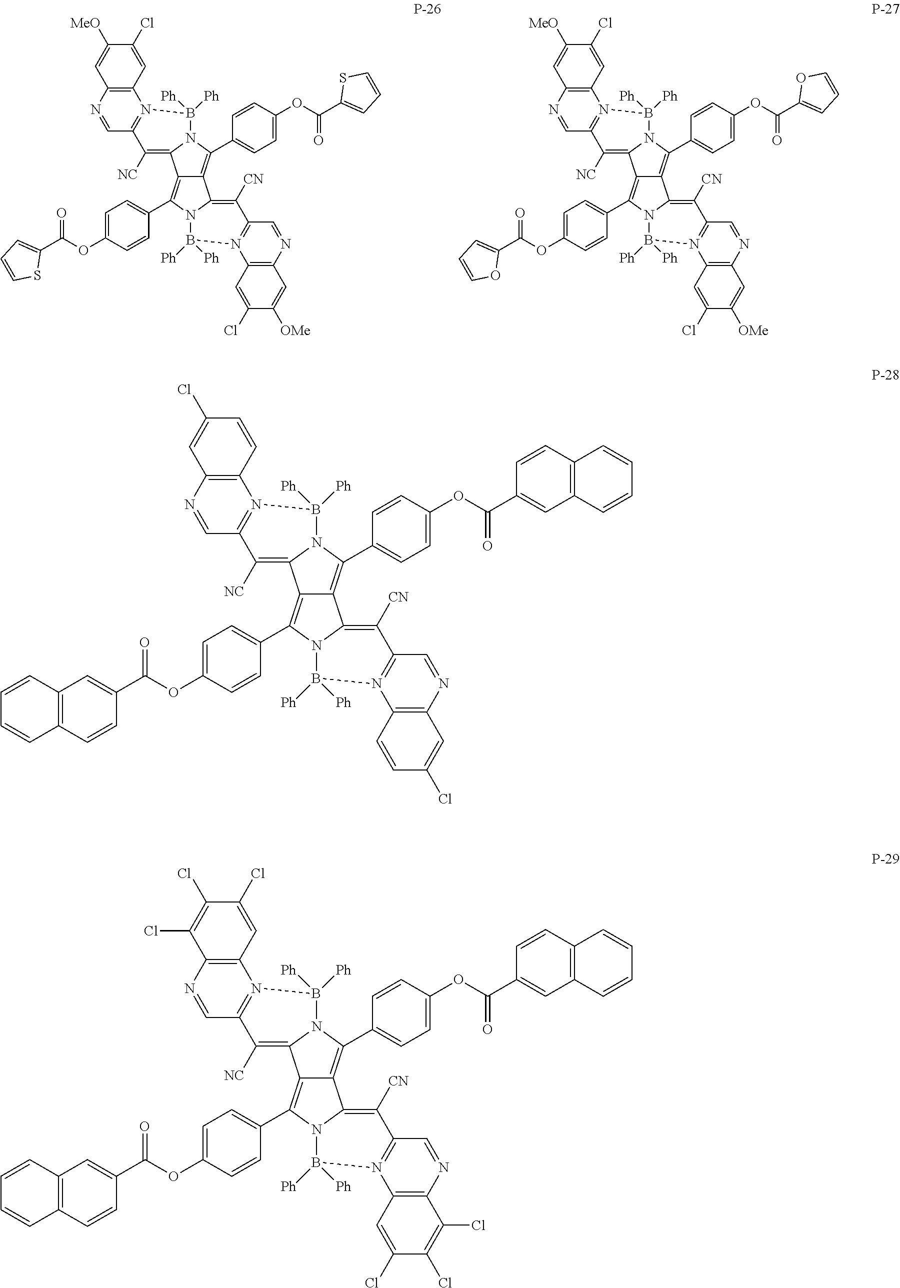

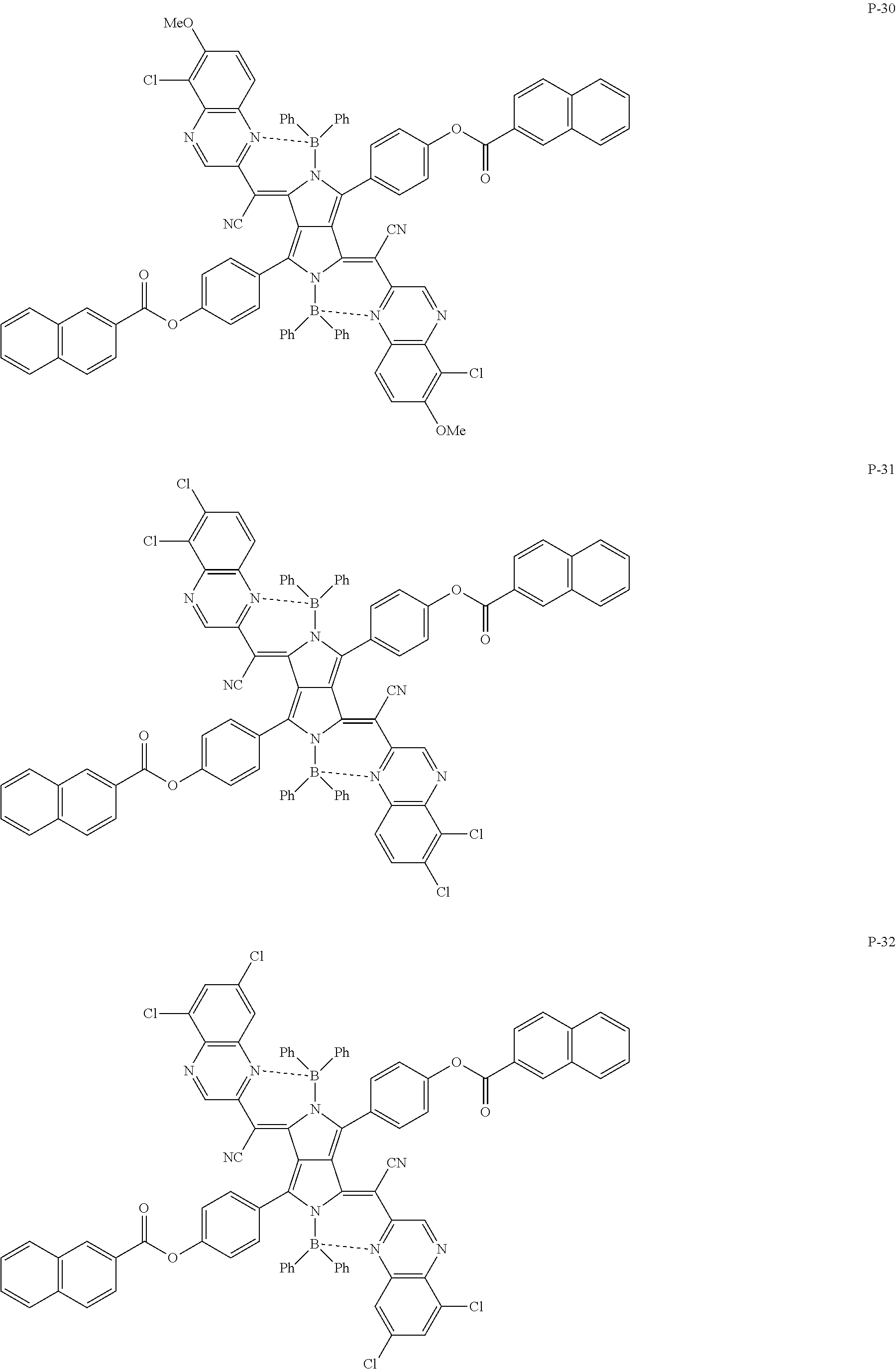

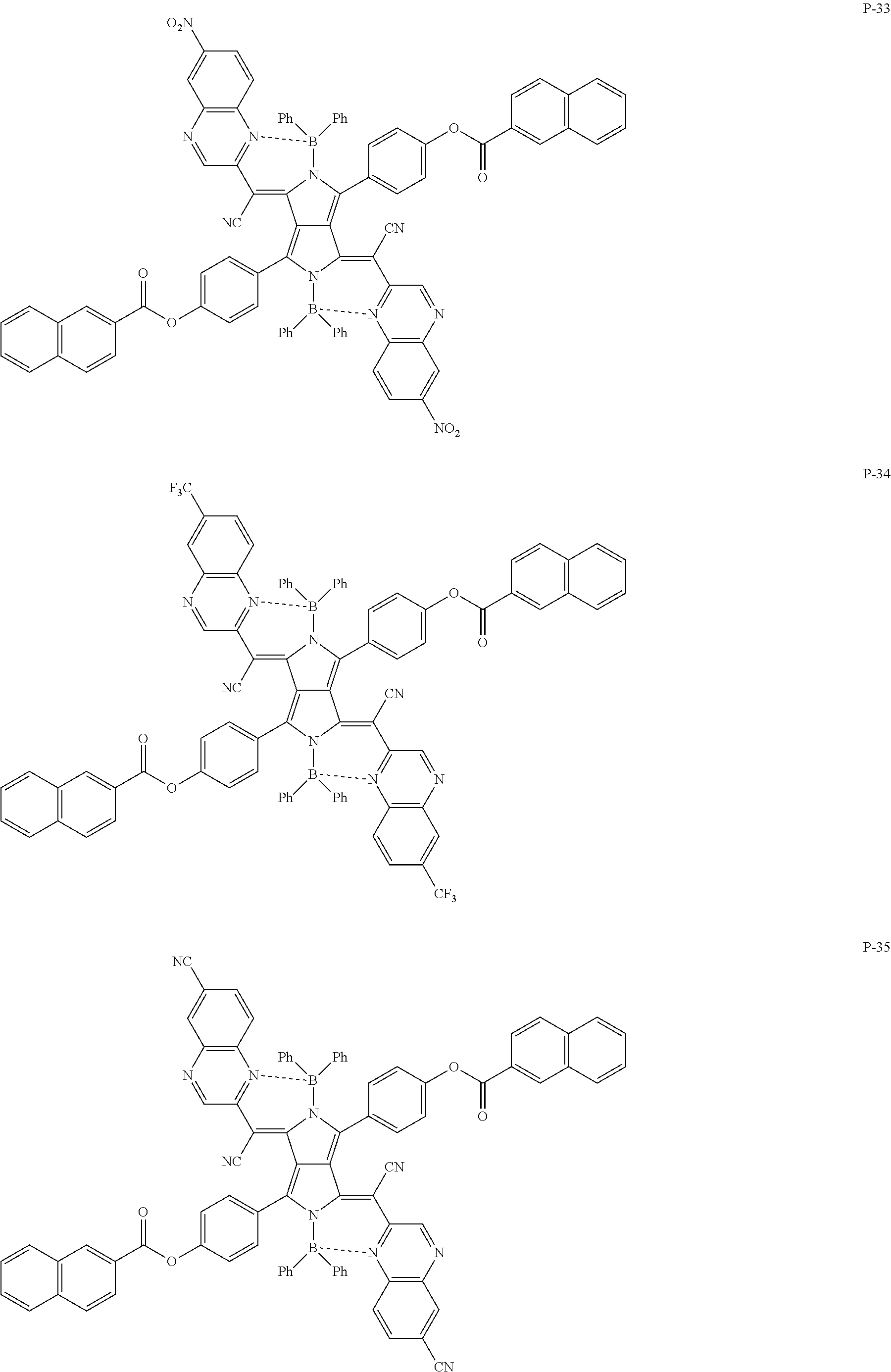

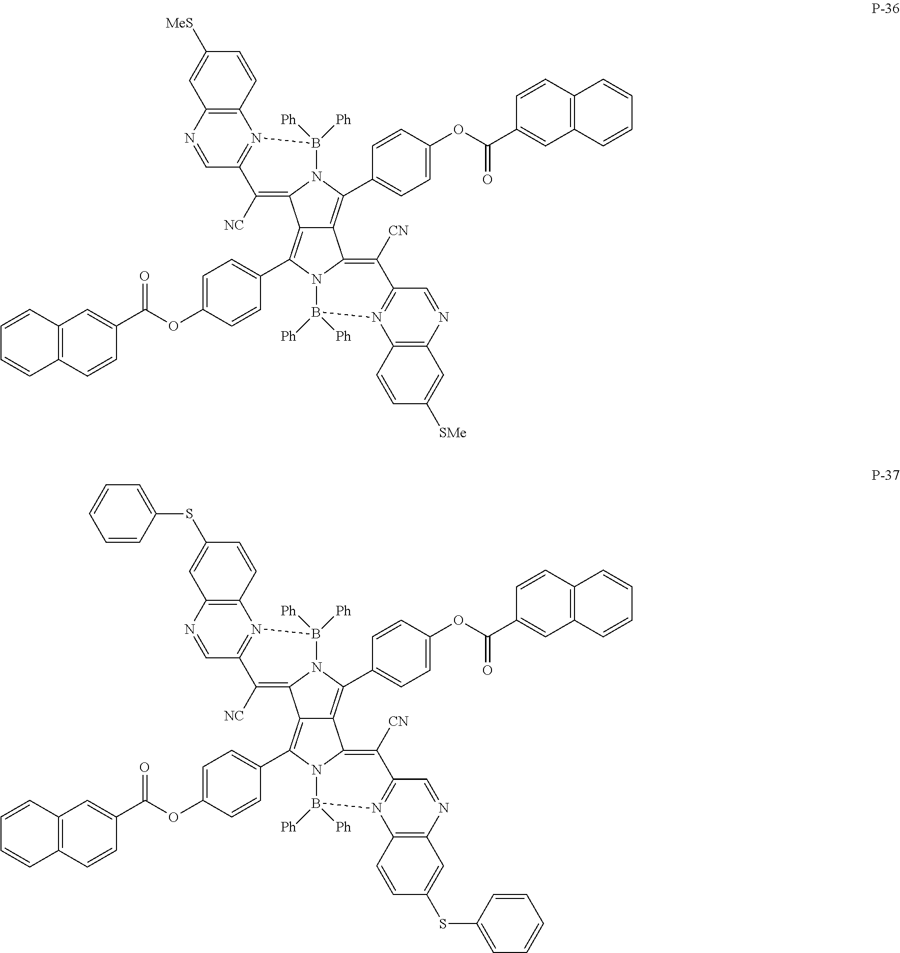

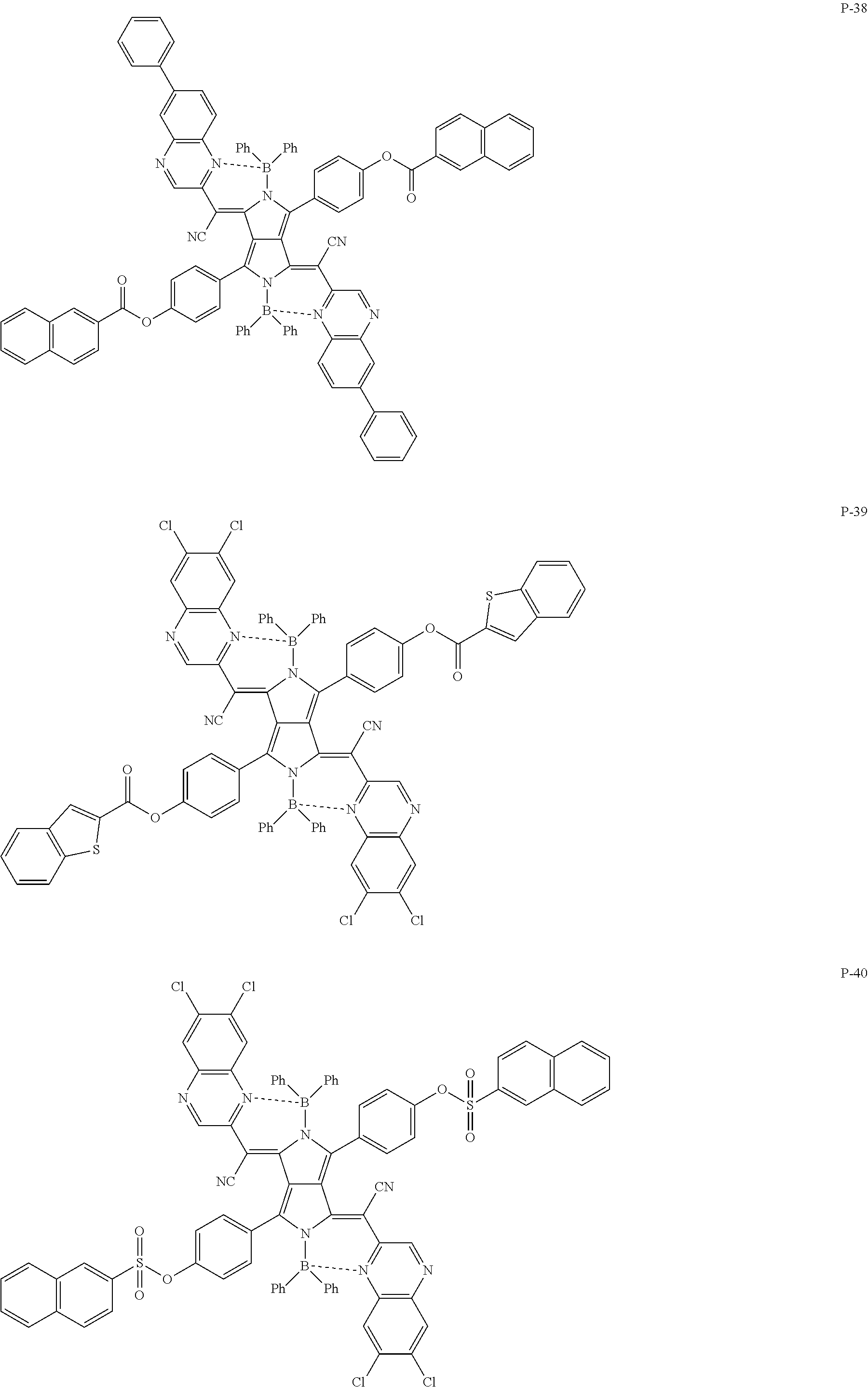

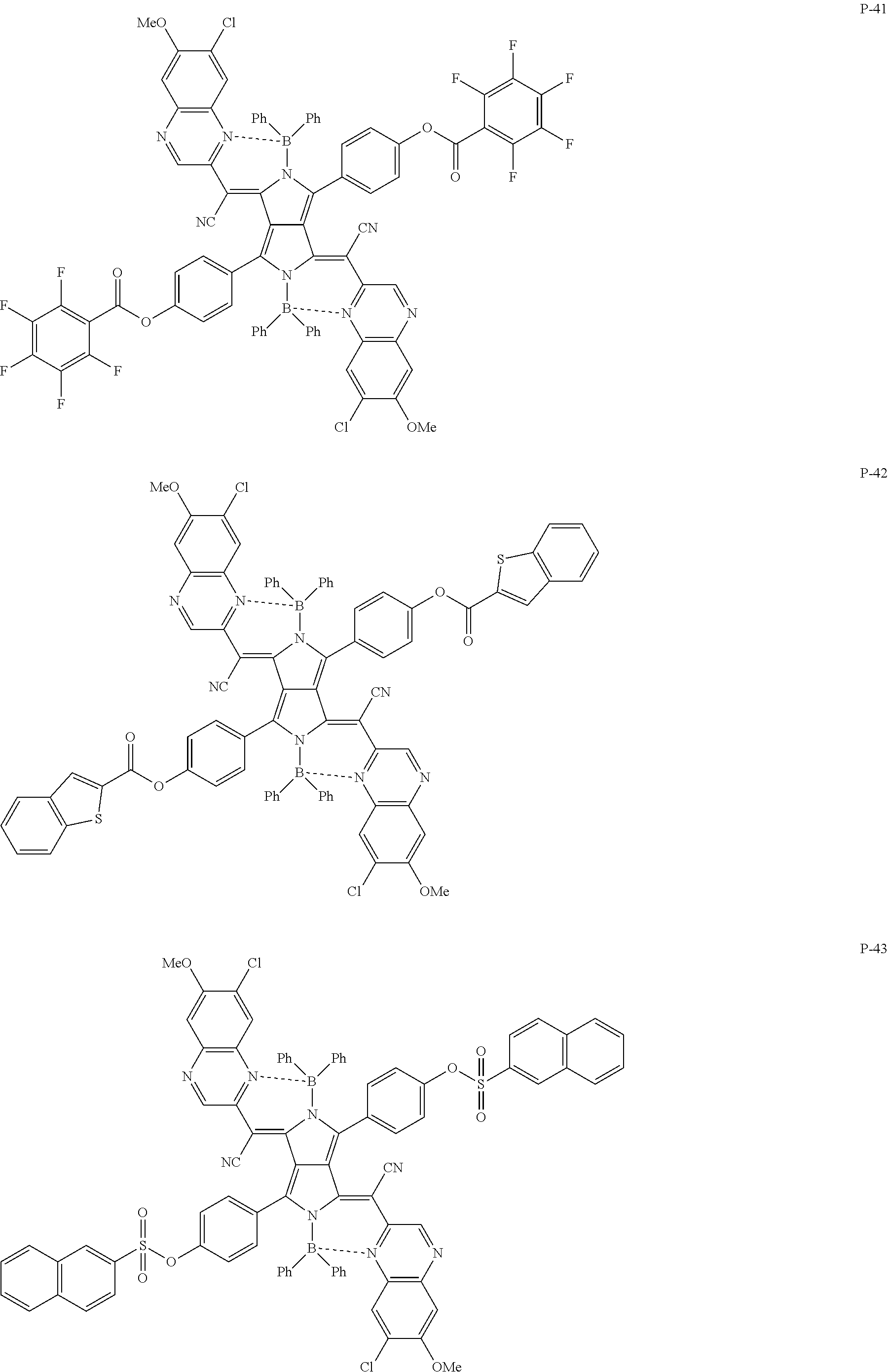

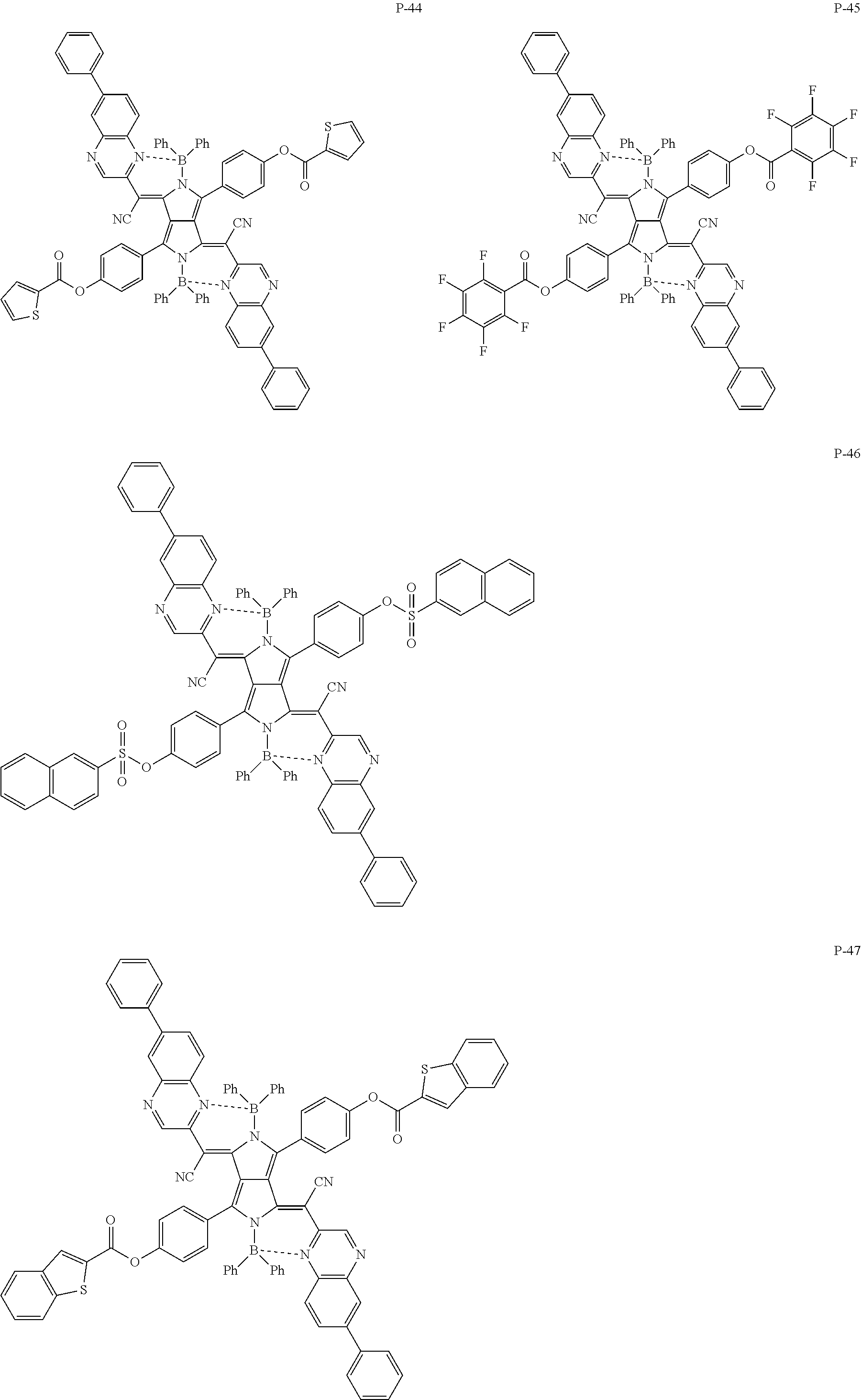

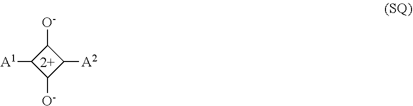

In Formula (SQ), A¹ and A² each independently represent an aryl group, a heteroaryl group, or a group represented by the following Formula (A-1).

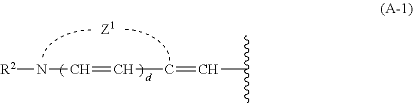

In Formula (A-1), Z¹ represents a non-metal atomic group for forming a nitrogen-containing heterocycle, R² represents an alkyl group, an alkenyl group, or an aralkyl group, d represents 0 or 1, and a wave line represents a direct bond.

The number of carbon atoms in the aryl group represented by A¹ and A² is preferably 6 to 48, more preferably 6 to 24, and still more preferably 6 to 12.

It is preferable that the heteroaryl group represented by A¹ and A² is a 5-membered or 6-membered ring. In addition, the heteroaryl group is preferably a monocycle or a fused ring composed of 2 to 8 rings, more preferably a monocycle or a fused ring composed of 2 to 4 rings, and still more preferably a monocycle or a fused ring composed of 2 or 3 rings. Examples of a heteroatom constituting the ring of the heteroaryl group include a nitrogen atom, an oxygen atom, and a sulfur atom. Among these, a nitrogen atom or a sulfur atom is preferable. The number of heteroatoms constituting the ring of the heteroaryl group is preferably 1 to 3 and more preferably 1 or 2.

The aryl group and the heteroaryl group may have a substituent. In a case where the aryl group and the heteroaryl group have two or more substituents, the plurality of substituents may be the same as or different from each other. Examples of the substituent include the above-described substituent T.

In Formula (A-1), R² represents an alkyl group, an alkenyl group, or an aralkyl group and preferably an alkyl group. In Formula (A-1), the nitrogen-containing heterocycle formed by Z¹ is preferably a 5- or 6-membered ring. In addition, the nitrogen-containing heterocycle is preferably a monocycle or a fused ring composed of 2 to 8 rings, more preferably a monocycle or a fused ring composed of 2 to 4 rings, and still more preferably a fused ring composed of 2 or 3 rings. In addition to a nitrogen atom, the nitrogen-containing heterocycle may include a sulfur atom. In addition, the nitrogen-containing heterocycle may have a substituent. Examples of the substituent include the substituents described above regarding the Formula (PP).

The details of Formula (SQ) can be found in paragraphs “0020” to “0049” of JP2011-208101A, paragraphs “0043” to “0062” of JP6065169B, and paragraphs “0024” to “0040” of WO2016/181987A, the contents of which are incorporated herein by reference.

As shown below, cations in Formula (SQ) are present without being localized.

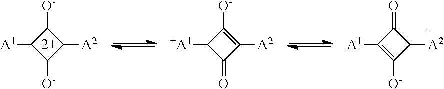

It is preferable that the squarylium compound is a compound represented by the following Formula (SQ-1).

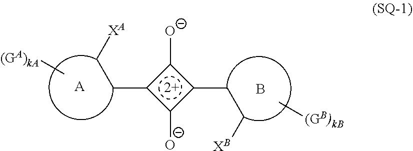

A Ring A and a ring B each independently represent an aromatic ring.

X^{A} and X^{B} each independently represent a substituent.

G^{A} and G^{B} each independently represent a substituent.

kA represents an integer of 0 to n_{A}, and kB represents an integer of 0 to n_{B}.

n_{A} and n_{B} represents integers representing the maximum numbers of G^{A}'s and G^{B}'s which may be substituted in the ring A and the ring B, respectively.

X^{A} and G^{A}, X^{B} and G^{B}, or X^{A} and X^{B} may be bonded to each other to form a ring, and in a case where a plurality of G^{A}'s and a plurality of G^{B}'s are present, G^{A}'s and G^{B}'s may be bonded to each other to form ring structures, respectively.

Examples of the substituent represented by G^{A} and G^{B} include the above-described substituent T.

As the substituent represented by X^{A} and X^{B}, a group having active hydrogen is preferable, —OH, —SH, —COOH, —SO₃H, —NR^{X1}R^{x}, —NHCOR^{X1}, —CONR^{X1}R^{X2}, —NHCONR^{X1}R^{X2}, —NHCOOR^{X1}, —NHSO₂R^{X1}, —B(OH)₂, or —PO(OH)₂ is more preferable, and —OH, —SH, or —NR^{X1}R^{X2} is still more preferable. R^{X1} and R^{X1} each independently represent a hydrogen atom or a substituent. Examples of the substituent include an alkyl group, an aryl group, and a heteroaryl group. Among these, an alkyl group is preferable.

The ring A and the ring B each independently represent an aromatic ring. The aromatic ring may be a monocycle or a fused ring. Specific examples of the aromatic ring include a benzene ring, a naphthalene ring, a pentalene ring, an indene ring, an azulene ring, a heptalene ring, an indacene ring, a perylene ring, a pentacene ring, an acenaphthene ring, a phenanthrene ring, an anthracene ring, a naphthacene ring, a chrysene ring, a triphenylene ring, a fluorene ring, a biphenyl ring, a pyrrole ring, a furan ring, a thiophene ring, an imidazole ring, an oxazole ring, a thiazole ring, a pyridine ring, a pyrazine ring, a pyrimidine ring, a pyridazine ring, an indolizine ring, an indole ring, a benzofuran ring, a benzothiophene ring, an isobenzofuran ring, a quinolizine ring, a quinoline ring, a phthalazine ring, a naphthyridine ring, a quinoxaline ring, a quinoxazoline ring, an isoquinoline ring, a carbazole ring, a phenanthridine ring, an acridine ring, a phenanthroline ring, a thianthrene ring, a chromene ring, a xanthene ring, a phenoxathiin ring, a phenothiazine ring, and a phenazine ring. Among these, a benzene ring or a naphthalene ring is preferable. The aromatic ring may be unsubstituted or may have a substituent. Examples of the substituent include the above-described substituent T.

X^{A} and G^{A}, X³ and G^{B}, or X^{A} and X^{B} may be bonded to each other to form a ring, and in a case where a plurality of GA's and a plurality of G^{B}'s are present, G^{A}'s and G^{B}'s may be bonded to each other to form rings, respectively. It is preferable that the ring is a 5- or 6-membered ring. The ring may be a monocycle or a fused ring. In a case where X^{A} and G^{A}, X^{B} and G^{B}, X^{A} and X^{B}, G^{A}'s, or G^{B}'s are bonded to each other to form a ring, the groups may be directly bonded to each other to form a ring, or may be bonded to each other through a divalent linking group selected from an alkylene group, —CO—, —O—, —NH—, —BR—, or a combination thereof to form a ring. R represents a hydrogen atom or a substituent. Examples of the substituent include the substituent T. Among these, an alkyl group or an aryl group is preferable.

kA represents an integer of 0 to n_{A}, kB represents an integer of 0 to n_{B}, n_{A} represents an integer representing the maximum number of G^{A}'s which may be substituted in the ring A, and n_{B} represents an integer representing the maximum number of G^{B}'s which may be substituted in the ring B. kA and kB each independently represent preferably an integer of 0 to 4, more preferably 0 to 2, and still more preferably 0 or 1.

It is also preferable that the squarylium compound is a compound represented by the following Formula (SQ-10), Formula (SQ-11), or Formula (SQ-12).

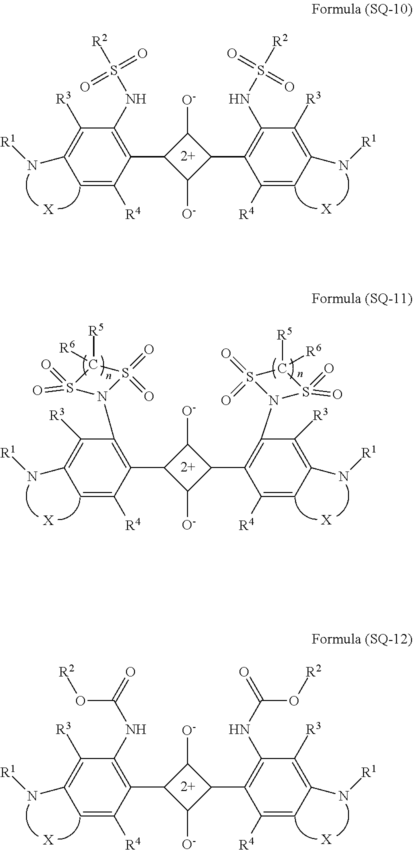

In Formulae (SQ-10) to (SQ-12), X's each independently represent a divalent organic group represented by Formula (1) or Formula (2) in which one or more hydrogen atoms are represented by a halogen atom, an alkyl group having 1 to 12 carbon atoms, or an alkoxy group.

—(CH₂)ₙ₁— (1)

In Formula (1), n1 represents 2 or 3.

—(CH₂)ₙ₂—O—(CH₂)ₙ₃— (2)

In Formula (2), n2 and n3 each independently represent an integer of 0 to 2, and n2+n3 represents 1 or 2.

R¹ and R² each independently represent an alkyl group or an aryl group. The alkyl group and the aryl group may have a substituent or may be unsubstituted. Examples of the substituent include the above-described substituent T.

R³ to R⁶ each independently represent a hydrogen atom, a halogen atom, an alkyl group, or an alkoxy group.

In Formula (SQ-11), n represents 2 or 3.

Examples of the squarylium compound include a compound having the following structure. In addition, for example, a compound described in paragraphs “0044” to “0049” of JP2011-208101A, a compound described in paragraphs “0060” and “0061” of JP6065169B, a compound described in paragraph “0040” of WO2016/181987A, or a compound described in JP2015-176046A can be used, the contents of which are incorporated herein by reference.

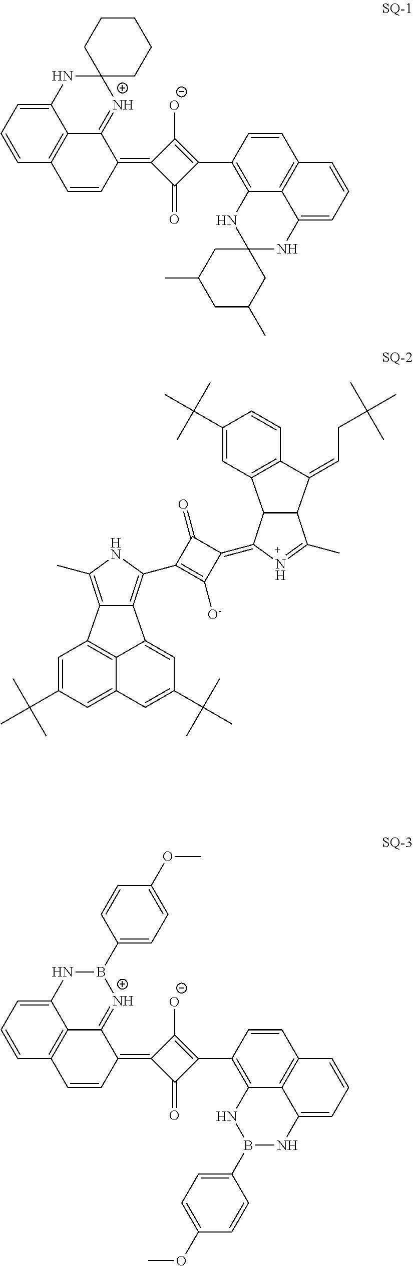 [SMILES: CC1CC(C)CC2(C1)Nc1cccc3ccc(C4=C([O-])/C(=c5/ccc6cccc7c6c5=[NH+]C5(CCCCC5)N7)C4=O)c(c13)N2 ; CC(C)=[NH+]/C(=C1/C(=O)C(c2[nH]c(C)c3c2-c2cc(C(C)(C)C)cc4cc(C(C)(C)C)cc-3c24)=C1[O-])C1C/C(=C/CC(C)(C)C)c2ccc(C(C)(C)C)cc21 ; COc1ccc(B2Nc3cccc4ccc(C5=C([O-])/C(=c6/ccc7cccc8c7c6=[NH+]B(c6ccc(OC)cc6)N8)C5=O)c(c34)N2)cc1]

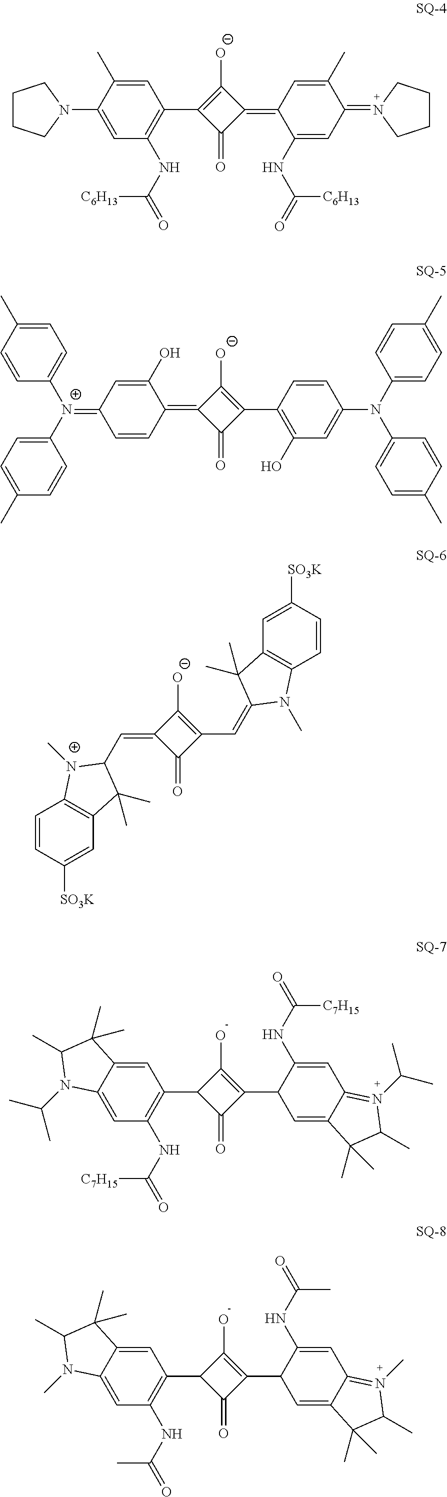

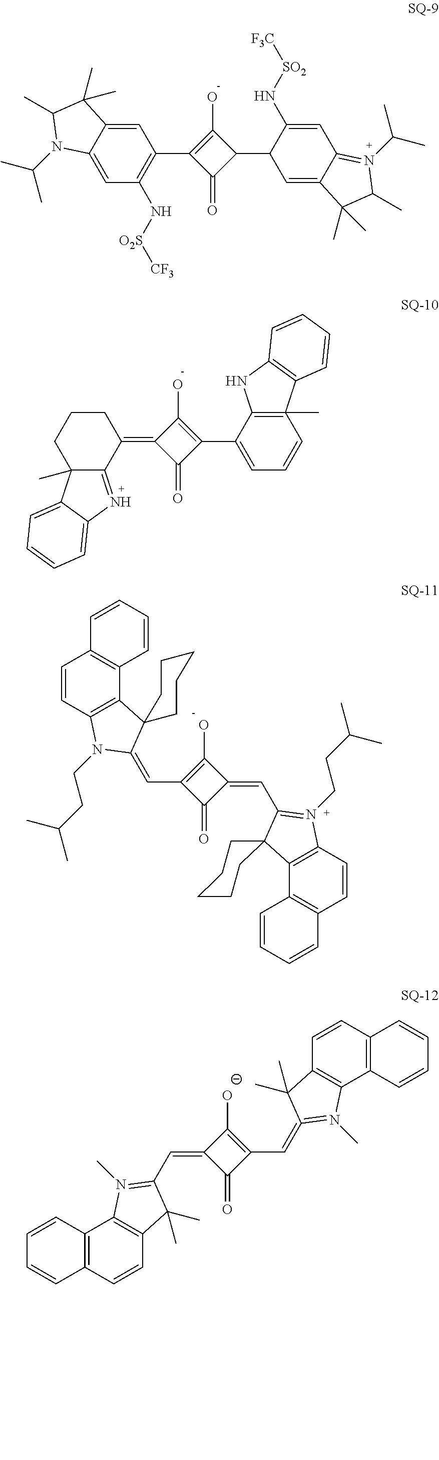

As the cyanine compound, a compound represented by Formula (C) is preferable.

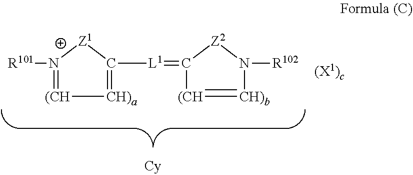

In the formula, Z¹ and Z² each independently represent a non-metal atomic group for forming a 5- or 6-membered nitrogen-containing heterocycle which may be fused.

R¹⁰¹ and R¹⁰² each independently represent an alkyl group, an alkenyl group, an alkynyl group, or an aryl group.

L¹ represents a methine chain including an odd number of methine groups.

a and b each independently represent 0 or 1.

In a case where a represents 0, a carbon atom and a nitrogen atom are bonded through a double bond. In a case where b represents 0, a carbon atom and a nitrogen atom are bonded through a single bond.

In a case where a site represented by Cy in the formula is a cation site, X¹ represents a counter anion, and c represents the number of X¹'s for balancing charge. In a case where a site represented by Cy in the formula is an anion site, X¹ represents a counter cation, and c represents the number of X¹'s for balancing charge. In a case where charge of a site represented by Cy in the formula is neutralized in a molecule, c represents 0.

In Formula (C), Z¹ and Z² each independently represent a non-metal atomic group for forming a 5- or 6-membered nitrogen-containing heterocycle which may be fused. Another heterocycle, an aromatic ring, or an aliphatic ring may be fused to the nitrogen-containing heterocycle. It is preferable that the nitrogen-containing heterocycle is a 5-membered ring. A structure in which a benzene ring or a naphthalene ring is fused to the 5-membered nitrogen-containing heterocycle is more preferable. The nitrogen-containing heterocycle and a ring fused thereto may have a substituent. Examples of the substituent include the above-described substituent T.

In Formula (C), R¹⁰¹ and R¹⁰² each independently represent an alkyl group, an alkenyl group, an alkynyl group, or an aryl group. These groups may further have a substituent or may be unsubstituted. Examples of the substituent include the above-described substituent T.

In Formula (C), L¹ represents a methine chain including an odd number of methine groups. It is preferable that L¹ represents a methine chain including 3, 5, or 7 methine groups. The methine group may have a substituent. It is preferable that the methine group having a substituent is a methine group positioned at the center (meso position). Specific examples of the substituent include the above-described substituent T. In addition, two substituents in the methine chain may be bonded to each other to form a 5- or 6-membered ring.

In Formula (C), a and b each independently represent 0 or 1. In a case where a represents 0, a carbon atom and a nitrogen atom are bonded through a double bond. In a case where b represents 0, a carbon atom and a nitrogen atom are bonded through a single bond. It is preferable that both a and b represent 0. In a case where both a and b represent 0, Formula (C) will be shown below.

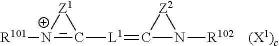

In a case where a site represented by Cy in Formula (C) is a cation site, X¹ represents a counter anion, and c represents the number of X¹'s for balancing charge. Examples of the counter anion include an halide ion (Cl⁻, Br⁻, or I⁻), a p-toluenesulfonate ion, an ethyl sulfate ion, PF₆⁻, BF₄⁻ClO₄⁻, a tris(halogenoalkylsufonyl)methide anion (for example, (CF₃SO₂)₃C⁻), a di(halogenoalkylsulfonyl)imide anion (for example, (CF₃SO₂)₂N⁻), and a tetracyanoborate anion. In addition, as the counter anion, a counter anion represented by Formula A can also be used.

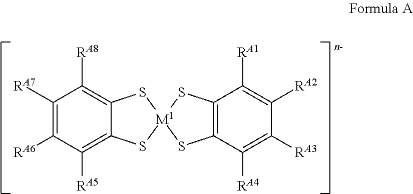

M¹ represents a transition metal, n represents an integer of 1 or 2, and R^{A1} to R^{A8} each independently represent a hydrogen atom or a substituent. The details of Formula A can be found in paragraphs “0030” to “0050” of JP2015-40895A, the content of which is incorporated herein by reference.

In a case where a site represented by Cy in Formula (C) is an anion site, X¹ represents a counter cation, and c represents the number of X¹'s for balancing charge. Examples of the counter cation include an alkali metal ion (for example, Li⁺, Na⁺, or K⁺), an alkali earth metal ion (Mg²⁺, Ca²⁺, Ba²⁺, or Sr²⁺), a transition metal ion (for example, Ag⁺, Fe²⁺, Co²⁺, Ni²⁺, Cu²⁺, or Zn²⁺), other metal ions (for example, A1⁺), an ammonium ion, a triethylammonium ion, a tributylammonium ion, a pyridinium ion, a tetrabutylammonium ion, a guanidinium ion, a tetramethylguanidinium ion, and a diazabicycloundecenium ion. As the cation, Na⁺, K⁺, Mg²⁺, Ca²⁺, Zn²⁺, or a diazabicycloundecenium ion is preferable.

In a case where charge of a site represented by Cy in Formula (C) is neutralized in a molecule, X¹ is not present. That is, c represents 0.

Examples of the cyanine compound include a compound having the following structure. In addition, examples of the cyanine compound include a compound described in paragraphs “0044” and “0045” of JP2009-108267A, a compound described in paragraphs “0026” to “0030” of JP2002-194040, a compound described in JP2015-172004A, a compound described in JP2015-172102A, and a compound described in JP2008-088426A, the contents of which are incorporated herein by reference.

TABLE 7

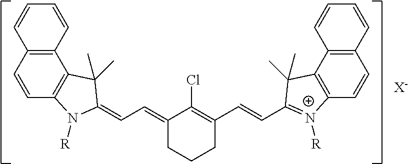

| | R | X- |
|---|---|---|
| CY-1 | —C₂H₄OCH₃ | SbF₆⁻ |
| CY-2 | —CH₃ | 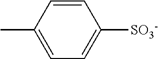 |
| CY-3 | —C₄H₉ | 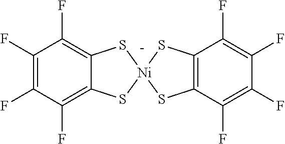 |
| CY-4 | —C₂H₄OCH₃ | 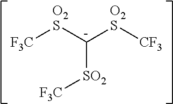 |
| CY-5 | —C₁₀H₂₁ | 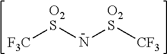 |

As the croconium compound, a compound represented by the following Formula (Cr) is preferable.

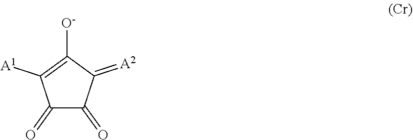

In Formula (Cr), A¹ and A² each independently represent an aryl group, a heteroaryl group, or a group represented by the following Formula (A-1).

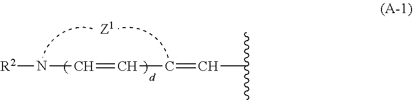

In Formula (A-1), Z¹ represents a non-metal atomic group for forming a nitrogen-containing heterocycle, R² represents an alkyl group, an alkenyl group, or an aralkyl group, d represents 0 or 1, and a wave line represents a direct bond.

The details of Formula (Cr) can be found in paragraphs “0007” to “0016” of JP1993-155145A (JP-H5-155145A) and paragraphs “0011” to “0038” of JP2007-031644A, the contents of which are incorporated herein by reference. Specific examples of the croconium compound include compounds having the following structures. In addition, other examples of the croconium compound include a compound described in JP1993-155145A (JP-H5-155145A) and JP2007-031644A, the contents of which are incorporated herein by reference.

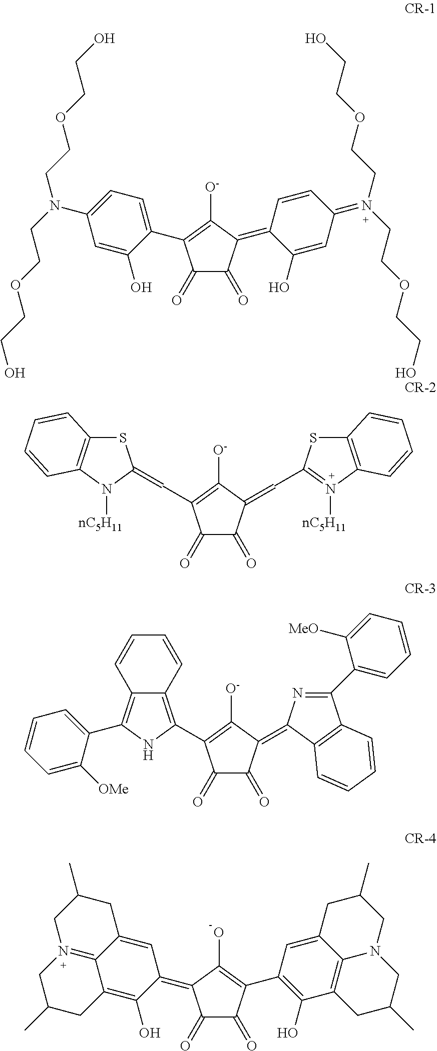

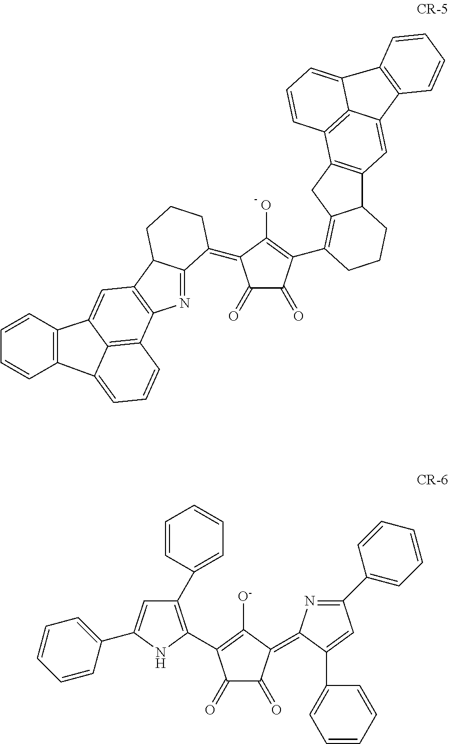 [SMILES: O=C1C(=O)/C(=C2/CCCC3C2=Nc2c3cc3c4c(cccc24)-c2ccccc2-3)C(O)=C1C1=C2Cc3c(cc4c5c(cccc35)-c3ccccc3-4)C2CCC1 ; O=C1C(=O)/C(=C2/N=C(c3ccccc3)C=C2c2ccccc2)C([O-])=C1c1[nH]c(-c2ccccc2)cc1-c1ccccc1]

As the diimmonium compound, a compound represented by the following Formula (Im) is preferable.

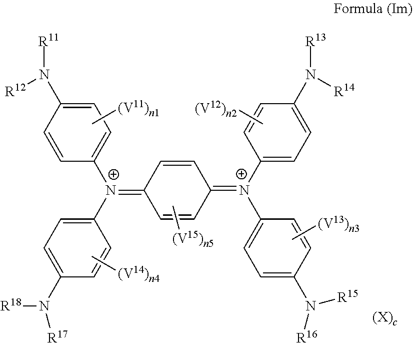

In the formula, R¹¹ to R¹⁸ each independently represent an alkyl group or an aryl group, V¹¹ to V¹⁵ each independently represent an alkyl group, an aryl group, a halogen atom, an alkoxy group, or a cyano group, X represents a counter anion, c represents the number of X's for balancing charge, and n1 to n5 each independently 0 to 4. Specific examples of the counter anion include the above-described counter anions.

Specific examples of the diimmonium compound include the following compounds. In addition, other examples of the croconium compound include a compound described in JP2012-012399A and JP2007-092060A, the contents of which are incorporated herein by reference. In the following structural formulae, Pr represents a propyl group, and Cy represents a cyclohexyl group.

TABLE 8

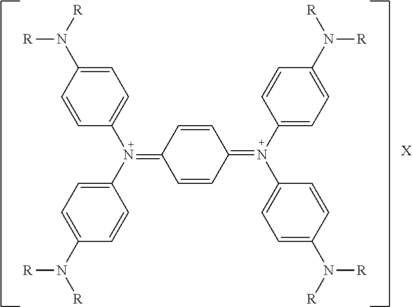

| | R | X |
|---|---|---|
| IM-1 | —C₆H₁₃ | 2(ClO₄⁻) |
| IM-2 | —C₄H₉ | 2(SbF₆⁻) |
| IM-3 | —CH₃ | 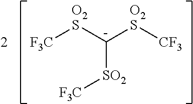 |
| IM-4 | —Cy | 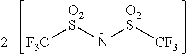 |
| IM-5 | —iPr | SO₄²⁻ |

In the present invention, as the near infrared absorbing colorant, a commercially available product can also be used. Examples of the commercially available product include SDO-C33 (manufactured by Arimoto Chemical Co., Ltd.); EXCOLOR IR-14, EXCOLOR IR-10A, EXCOLOR TX-EX-801B, and EXCOLOR TX-EX-805K (manufactured by Nippon Shokubai Co., Ltd.); Shigenox NIA-8041, Shigenox NIA-8042, Shigenox NIA-814, Shigenox NIA-820, and Shigenox NIA-839 (manufactured by Hakkol Chemical Co., Ltd.); Epolite V-63, Epolight 3801, and Epolight3036 (manufactured by Epolin Inc.); PRO-JET 825LDI (manufactured by Fujifilm Corporation); NK-3027 and NK-5060 (manufactured by Hayashibara Co., Ltd.); and YKR-3070 (manufactured by Mitsui Chemicals, Inc.).

In a case where the curable composition according to the embodiment of the present invention includes a near infrared absorbing colorant, the content of the near infrared absorbing colorant is preferably 0.01 to 50 mass % with respect to the total solid content of the curable composition. The lower limit is preferably 0.1 mass % or higher and more preferably 0.5 mass % or higher. The upper limit is preferably 30 mass % or lower, and more preferably 15 mass % or lower. In addition, the total content of the near infrared absorbing colorant and the compound A is preferably 0.01 to 50 mass % with respect to the total solid content of the curable composition. The lower limit is preferably 0.1 mass % or higher and more preferably 0.5 mass % or higher. The upper limit is preferably 30 mass % or lower, and more preferably 15 mass % or lower. In the curable composition according to the embodiment of the present invention, as the near infrared absorbing colorant, one kind may be used alone, or two or more kinds may be used in combination. In a case where two or more near infrared absorbing colorants are used in combination, it is preferable that the total content of the near infrared absorbing colorants is in the above-described range.

(Coloring Material that allows Transmission of Infrared Light and shields Visible Light)

The curable composition according to the embodiment of the present invention may also include, as a colorant, the coloring material that allows transmission of infrared light and shields visible light (hereinafter, also referred to as “coloring material that shields visible light”).

In the present invention, it is preferable that the coloring material that shields visible light is a coloring material that absorbs light in a wavelength range of violet to red. In addition, in the present invention, it is preferable that the coloring material that shields visible light is a coloring material that shields light in a wavelength range of 450 to 650 nm. In addition, it is preferable that the coloring material that shields visible light is a coloring material that allows transmission of light in a wavelength range of 900 to 1300 nm.

In the present invention, it is preferable that the coloring material that shields visible light satisfies at least one of the following requirement (A) or (B).

(A): The coloring material that shields visible light includes two or more chromatic colorants, and a combination of the two or more chromatic colorants forms black

(B): The coloring material that shields visible light includes an organic black colorant

Examples of the chromatic colorant are as described above. Examples of the organic black colorant include a bisbenzofuranone compound, an azomethine compound, a perylene compound, and an azo compound. Among these, a bisbenzofuranone compound or a perylene compound is preferable. Examples of the bisbenzofuranone compound include a compound described in JP2010-534726A, JP2012-515233A, and JP2012-515234A. For example, “Irgaphor Black” (manufactured by BASF SE) is available. Examples of the perylene compound include C.I. Pigment Black 31 and 32. Examples of the azomethine compound include a compound described in JP1989-170601A (JP-H1-170601A) and JP1990-034664A (JP-H2-034664A). For example, “CHROMOFINE BLACK A1103” (manufactured by Dainichiseika Color & Chemicals Mfg. Co., Ltd.) is available.

In a case where a combination of two or more chromatic colorants forms black, examples of the combination of chromatic colorants are as follows.

(1) An aspect where a yellow colorant, a blue colorant, a violet colorant, and a red colorant are included

(2) An aspect where a yellow colorant, a blue colorant, and a red colorant are included

(3) An aspect where a yellow colorant, a violet colorant, and a red colorant are included

(4) An aspect where a yellow colorant and a violet colorant are included

(5) An aspect where a green colorant, a blue colorant, a violet colorant, and a red colorant are included

(6) An aspect where a violet colorant and an orange colorant are included

(7) An aspect where a green colorant, a violet colorant, and a red colorant are included

(8) An aspect where a green colorant and a red colorant are included

In a case where the curable composition according to the embodiment of the present invention includes the coloring material that shields visible light, the content of the coloring material that shields visible light is preferably 60 mass % or lower, more preferably 50 mass % or lower, still more preferably 30 mass % or lower, still more preferably 20 mass % or lower, and still more preferably 15 mass % or lower with respect to the total solid content of the curable composition. The lower limit is, for example, 0.01 mass % or higher or 0.5 mass % or higher.

<<Other Near Infrared Absorbers>>

The curable composition according to the embodiment of the present invention may further include near infrared absorbers (also referred to as “other near infrared absorbers”) other than the near infrared absorbing colorant. Examples of the other near infrared absorber include an inorganic pigment (inorganic particles). The shape of the inorganic pigment is not particularly limited and may have a sheet shape, a wire shape, or a tube shape irrespective of whether or not the shape is spherical or non-spherical. As the inorganic pigment, metal oxide particles or metal particles are preferable. Examples of the metal oxide particles include indium tin oxide (ITO) particles, antimony tin oxide (ATO) particles, zinc oxide (ZnO) particles, A1-doped zinc oxide (A1-doped ZnO) particles, fluorine-doped tin dioxide (F-doped SnO₂) particles, and niobium-doped titanium dioxide (Nb-doped TiO₂) particles. Examples of the metal particles include silver (Ag) particles, gold (Au) particles, copper (Cu) particles, and nickel (Ni) particles. In addition, as the inorganic pigment, a tungsten oxide compound can also be used. As the tungsten oxide compound, cesium tungsten oxide is preferable. The details of the tungsten oxide compound can be found in paragraph “0080” of JP2016-006476A, the content of which is incorporated herein by reference.

In a case where the curable composition according to the embodiment of the present invention includes the other near infrared absorbers, the content of the other near infrared absorbers is preferably 0.01 to 50 mass % with respect to the total solid content of the curable composition. The lower limit is preferably 0.1 mass % or higher and more preferably 0.5 mass % or higher. The upper limit is preferably 30 mass % or lower, and more preferably 15 mass % or lower.

<<Photoinitiator>>

The curable composition according to the embodiment of the present invention may include a photoinitiator. Examples of the photoinitiator include a photoradical polymerization initiator and a photocationic polymerization initiator. It is preferable that the photoinitiator is selected and used according to the kind of the curable compound. In a case where the radically polymerizable compound is used as the curable compound, it is preferable that the photoradical polymerization initiator is used as the photoinitiator. In a case where the cationically polymerizable compound is used as the curable compound, it is preferable that the photocationic polymerization initiator is used as the photoinitiator. The photoinitiator is not particularly limited and can be appropriately selected from well-known photoinitiators. For example, a compound having photosensitivity to light in a range from an ultraviolet range to a visible range is preferable.

The content of the photoinitiator is preferably 0.1 to 50 mass %, more preferably 0.5 to 30 mass %, and still more preferably 1 to 20 mass % with respect to the total solid content of the curable composition. In a case where the content of the photoinitiator is in the above-described range, higher sensitivity and pattern formability can be obtained. The curable composition according to the embodiment of the present invention may include one photoinitiator or two or more photoinitiators. In a case where the composition includes two or more photoinitiators, it is preferable that the total content of the photopolymerization initiators is in the above-described range.

(Photoradical Polymerization Initiator)

Examples of the photoradical polymerization initiator include a halogenated hydrocarbon derivative (for example, a compound having a triazine skeleton or a compound having an oxadiazole skeleton), an acylphosphine compound, a hexaarylbiimidazole, an oxime compound, an organic peroxide, a thio compound, a ketone compound, an aromatic onium salt, an α-hydroxyketone compound, and an α-aminoketone compound. In addition, from the viewpoint of exposure sensitivity, as the photoradical polymerization initiator, a trihalomethyltriazine compound, a benzyldimethylketal compound, an α-hydroxyketone compound, an α-aminoketone compound, an acylphosphine compound, a phosphine oxide compound, a metallocene compound, an oxime compound, a triarylimidazole dimer, an onium compound, a benzothiazole compound, a benzophenone compound, an acetophenone compound, a cyclopentadiene-benzene-iron complex, a halomethyl oxadiazole compound, or a 3-aryl-substituted coumarin compound is preferable, a compound selected from the group consisting of an oxime compound, an α-hydroxy ketone compound, an α-aminoketone compound, and an acylphosphine compound is more preferable, and an oxime compound is still more preferable. The details of the photopolymerization initiator can be found in paragraphs “0065” to “0111” of JP2014-130173A, the content of which is incorporated herein by reference.

Examples of a conmmercially available product of the α-hydroxyketone compound include IRGACURE-184, DAROCUR-1173, IRGACURE-500, IRGACURE-2959, and IRGACURE-127 (all of which are manufactured by BASF SE). Examples of a commercially available product of the α-aminoketone compound include IRGACURE-907, IRGACURE-369, IRGACURE-379, and IRGACURE-379EG (all of which are manufactured by BASF SE). Examples of a commercially available product of the acylphosphine compound include IRGACURE-819, and DAROCUR-TPO (all of which are manufactured by BASF SE).

As the oxime compound, a compound described in JP2001-233842A, a compound described in JP2000-080068A, a compound described in JP2006-342166A, or a compound described in JP2016-021012A can be used. Examples of the oxime compound which can be preferably used in the present invention include 3-benzoyloxyiminobutane-2-one, 3-acetoxyiminobutane-2-one, 3-propionyloxyiminobutane-2-one, 2-acetoxyiminopentane-3-one, 2-acetoxyimino-1-phenylpropane-1-one, 2-benzoyloxyimino-1-phenylpropane-1-one, 3-(4-toluene sulfonyloxy)iminobutane-2-one, and 2-ethoxycarbonyloxyimino-1-phenylpropane-1-one. In addition, examples of the oxime compound include a compound described in J.C.S. Perkin II (1979), pp. 1653-1660, J.C.S. Perkin II (1979), pp. 156-162 and Journal of Photopolymer Science and Technology (1995), pp. 202-232, JP2000-066385A, JP2000-080068A, JP2004-534797A, or JP2006-342166A. As a commercially available product of the oxime compound, IRGACURE-OXE01, IRGACURE-OXE02, IRGACURE-OXE03, or IRGACURE-OXE04 (all of which are manufactured by BASF SE) can also be preferably used. In addition, TR-PBG-304 (manufactured by Changzhou Tronly New Electronic Materials Co., Ltd.), ADEKA OPTOMER N-1919 (manufactured by Adeka Corporation, a photopolymerization initiator 2 described in JP2012-014052A) can also be used. As the oxime compound, a compound having no colorability or a compound having high transparency that is not likely to be discolored can also be preferably used. Examples of a commercially available product of the oxime compound include ADEKA ARKLS NCI-730, NCI-831, and NCI-930 (all of which are manufactured by Adeka Corporation).

In the present invention, an oxime compound having a fluorene ring can also be used as the photoradical polymerization initiator. Specific examples of the oxime compound having a fluorene ring include a compound described in JP2014-137466A. The content of this specification is incorporated herein by reference.

In the present invention, an oxime compound having a fluorine atom can also be used as the photoradical polymerization initiator. Specific examples of the oxime compound having a fluorine atom include a compound described in JP2010-262028A, Compound 24 and 36 to 40 described in JP2014-500852A, and Compound (C-3) described in JP2013-164471A. The contents of this specification are incorporated herein by reference.

In the present invention, as the photoradical polymerization initiator, an oxime compound having a nitro group can be used. It is preferable that the oxime compound having a nitro group is a dimer. Specific examples of the oxime compound having a nitro group include a compound described in paragraphs “0031” to “0047” of JP2013-114249A and paragraphs “0008” to “0012” and “0070” to “0079” of JP2014-137466A, a compound described in paragraphs “0007” to 0025” of JP4223071B, and ADEKA ARKLS NCI-831 (manufactured by Adeka Corporation).

Specific examples of the oxime compound which are preferably used in the present invention are shown below, but the present invention is not limited thereto.

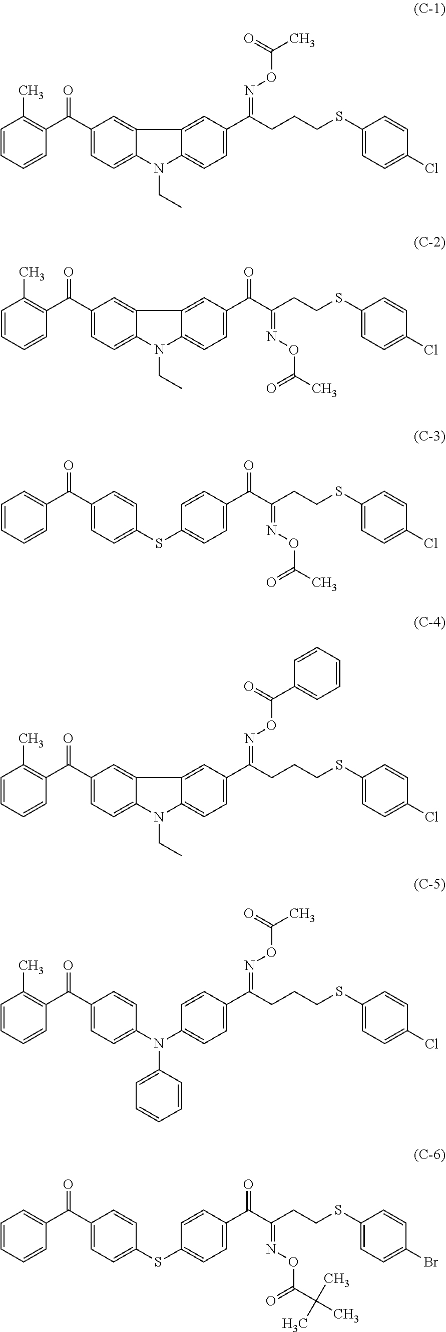 [SMILES: CCn1c2ccc(C(=O)c3ccccc3C)cc2c2cc(/C(CCCSc3ccc(Cl)cc3)=N/OC(C)=O)ccc21 ; CCn1c2ccc(C(=O)/C(CCSc3ccc(Cl)cc3)=N/OC(C)=O)cc2c2cc(C(=O)c3ccccc3C)ccc21 ; CC(=O)O/N=C(\CCSc1ccc(Cl)cc1)C(=O)c1ccc(Sc2ccc(C(=O)c3ccccc3)cc2)cc1 ; CCn1c2ccc(C(=O)c3ccccc3C)cc2c2cc(/C(CCCSc3ccc(Cl)cc3)=N/OC(=O)c3ccccc3)ccc21 ; CC(=O)O/N=C(\CCCSc1ccc(Cl)cc1)c1ccc(N(c2ccccc2)c2ccc(C(=O)c3ccccc3C)cc2)cc1 ; CC(C)(C)C(=O)O/N=C(\CCSc1ccc(Br)cc1)C(=O)c1ccc(Sc2ccc(C(=O)c3ccccc3)cc2)cc1]

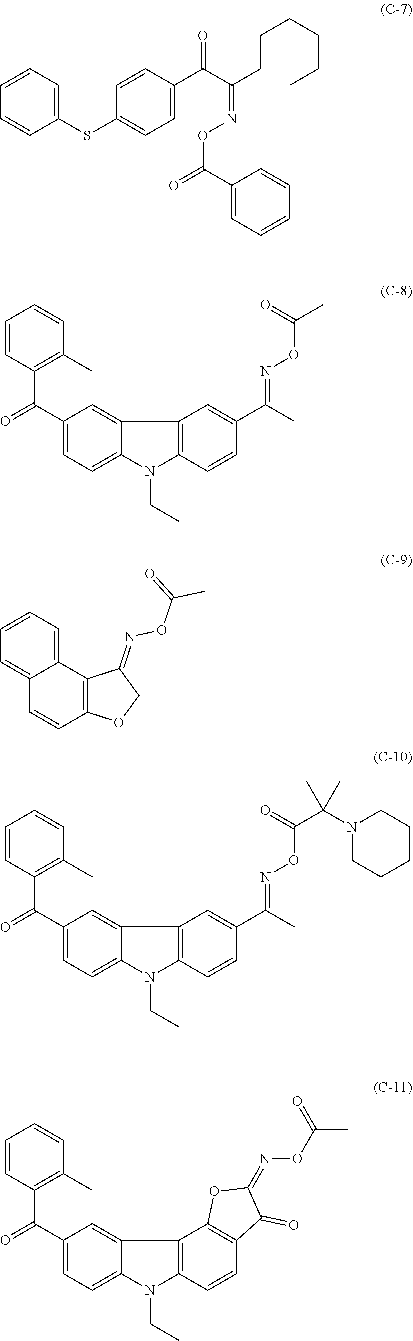 [SMILES: CCCCCC/C(=N/OC(=O)c1ccccc1)C(=O)c1ccc(Sc2ccccc2)cc1 ; CCn1c2ccc(C(=O)c3ccccc3C)cc2c2cc(/C(C)=N/OC(C)=O)ccc21 ; CC(=O)O/N=C1\COc2ccc3ccccc3c21 ; CCn1c2ccc(C(=O)c3ccccc3C)cc2c2cc(/C(C)=N/OC(=O)C(C)(C)N3CCCCC3)ccc21 ; CCn1c2ccc(C(=O)c3ccccc3C)cc2c2c3c(ccc21)C(=O)/C(=N\OC(C)=O)O3]

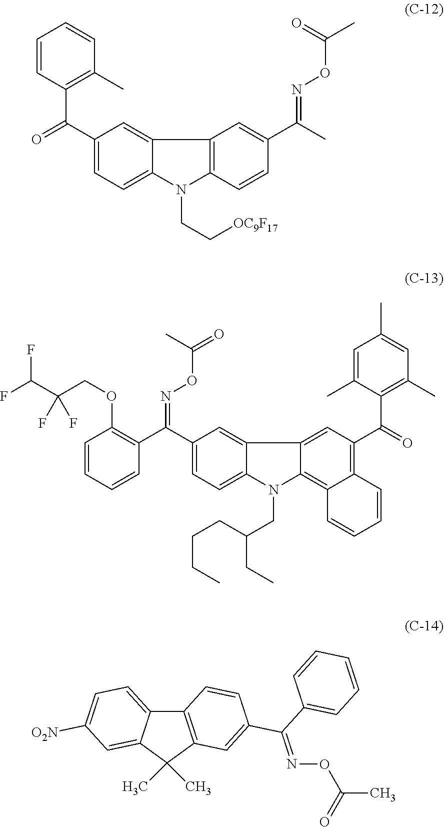

The oxime compound is preferably a compound having an absorption maximum in a wavelength range of 350 to 500 nm and more preferably a compound having an absorption maximum in a wavelength range of 360 to 480 nm. In addition, the oxime compound is preferably a compound having a high absorbance to light having a wavelength of 365 nm and 405 nm.

The molar absorption coefficient of the oxime compound at 365 nm or 405 nm is preferably 1000 to 300000, more preferably 2000 to 300000, and still more preferably 5000 to 200000 from the viewpoint of sensitivity.

The molar absorption coefficient of the compound can be measured using a well-known method. For example, it is preferable that the molar absorption coefficient can be measured using a spectrophotometer (Gary-S spectrophotometer, manufactured by Varian Medical Systems, Inc.) and ethyl acetate as a solvent at a concentration of 0.01 g/L.

It is preferable that the photoradical polymerization initiator includes an oxime compound and an α-aminoketone compound. By using the oxime compound and the α-aminoketone compound in combination, the developability is improved, and a pattern having excellent rectangularity is likely to be formed. In a case where the oxime compound and the α-aminoketone compound are used in combination, the content of the α-aminoketone compound is preferably 50 to 600 parts by mass and more preferably 150 to 400 parts by mass with respect to 100 parts by mass of the oxime compound.

The content of the photoradical polymerization initiator is preferably 0.1 to 50 mass %, more preferably 0.5 to 30 mass %, and still more preferably 1 to 20 mass % with respect to the total solid content of the curable composition. In a case where the content of the photoradical polymerization initiator is in the above-described range, higher sensitivity and pattern formability can be obtained. The curable composition according to the embodiment of the present invention may include one photoradical polymerization initiator or two or more photoradical polymerization initiators. In a case where the composition includes two or more photoradical polymerization initiators, it is preferable that the total content of the photoradical polymerization initiators is in the above-described range.

(Photocationic Polymerization Initiator) Examples of the photocationic polymerization initiator include a photoacid generator.

Examples of the photoacid generator include compounds which are decomposed by light irradiation to generate an acid including: an onium salt compound such as a diazonium salt, a phosphonium salt, a sulfonium salt, or an iodonium salt; and a sulfonate compound such as imidosulfonate, oximesulfonate, diazodisulfone, disulfone, or o-nitrobenzyl sulfonate. The details of the photocationic polymerization initiator can be found in paragraphs “0139” to “0214” of JP2009-258603A, the content of which is incorporated herein by reference.

As the photocationic polymerization initiator, a commercially available product can also be used. Examples of the commercially available product of the photocationic polymerization initiator include ADEKA ARKLS SP series manufactured by Adeka Corporation (for example, ADEKA ARKLS SP-606) and IRGACURE 250, IRGACURE 270, and IRGACURE 290 manufactured by BASF SE.

The content of the photocationic polymerization initiator is preferably 0.1 to 50 mass %, more preferably 0.5 to 30 mass %, and still more preferably 1 to 20 mass % with respect to the total solid content of the curable composition. In a case where the content of the photocationic polymerization initiator is in the above-described range, higher sensitivity and pattern formability can be obtained. The curable composition according to the embodiment of the present invention may include one photocationic polymerization initiator or two or more photocationic polymerization initiators. In a case where the composition includes two or more photocationic polymerization initiators, it is preferable that the total content of the two or more photocationic polymerization initiators is in the above-described range.

<<Acid Anhydride, Polycarboxylic Acid>>

In a case where the curable composition according to the embodiment of the present invention includes the epoxy compound, it is preferable that the composition further includes at least one selected from an acid anhydride or a polycarboxylic acid.

Specific examples of the acid anhydride include phthalic anhydride, trimellitic anhydride, pyromellitic anhydride, maleic acid anhydride, tetrahydrophthalic anhydride, methyltetrahydrophthalic anhydride, methylnadic anhydride, nadic anhydride, hexahydrophthalic anhydride, methylhexahydrophthalic anhydride, glutaric anhydride, 2,4-diethylglutaric anhydride, 3,3-dimethylglutaric anhydride, butanetetracarboxylic anhydride, bicyclo[2,2,1]heptane-2,3-dicarboxylic anhydride, methylbicyclo [2,2,1]heptane-2,3-dicarboxylic anhydride, and cyclohexane-1,3,4-tricarboxylic acid-3,4-anhydride. In particular, methyltetrahydrophthalic anhydride, methylnadic anhydride, nadic anhydride, hexahydrophthalic anhydride, methylhexahydrophthalic anhydride, 2,4-diethylglutaric anhydride, butanetetracarboxylic anhydride, bicyclo[2,2,1]heptane-2,3-dicarboxylic anhydride, or methylbicyclo[2,2,1]heptane-2,3-dicarboxylic anhydride, or cyclohexane-1,3,4-tricarboxylic acid-3,4-anhydride is preferable from the viewpoints of light fastness, transparency, and workability.

The polycarboxylic acid is a compound having at least two carboxyl groups. The polycarboxylic acid is not particularly limited as long as a geometric isomer or an optical isomer is present in the following compound. As the polycarboxylic acid, a bifunctional to hexafunctional carboxylic acid is preferable. For example, an alkyltricarboxylic acid such as 1,2,3,4-butanetetracarboxylic acid, 1,2,3-propanetricarboxylic acid, 1,3,5-pentanetricarboxylic acid, or citric acid; an alicyclic polycarboxylic acid such as phthalic acid, hexahydrophthalic acid, methylhexahydrophthalic acid, tetrahydrophthalic acid, methyltetrahydrophthalic acid, cyclohexanetricarboxylic acid, nadic acid, or methylnadic acid; a polymer of an unsaturated fatty acid such as linolenic acid or oleic acid and a dimer which is a reduction product thereof; or a linear alkyldioic acid such as butanedioic acid, malic acid, hexanedioic acid, pentanedioic acid, heptanedioic acid, octanedioic acid, nonanedioic acid, decanedioic acid is preferable, butanedioic acid, hexanedioic acid, pentanedioic acid, heptanedioic acid, octanedioic acid, nonanedioic acid, or decanedioic acid is more preferable, and butanedioic acid is still more preferable from the viewpoint of heat resistance and film transparency.

The content of the acid anhydride and the polycarboxylic acid is preferably 0.01 to 20 parts by mass, more preferably 0.01 to 10 parts by mass, and still more preferably 0.1 to 6.0 parts by mass with respect to 100 parts by mass of the epoxy compound.

<<Ultraviolet Absorber>>

The curable composition according to the embodiment of the present invention may include an ultraviolet absorber. As the ultraviolet absorber, for example, a conjugated diene compound, an aminobutadiene compound, a methyldibenzoyl compound, a coumarin compound, a salicylate compound, a benzophenone compound, a benzotriazole compound, an acrylonitrile compound, or a hydroxyphenyltriazine compound can be used. The details can be found in paragraphs “0052” to “0072” of JP2012-208374A and paragraphs “0317” to “0334” of JP2013-068814A, the contents of which are incorporated herein by reference. Examples of a commercially available product of the conjugated diene compound include UV-503 (manufactured by Daito Chemical Co., Ltd.). In addition, as the benzotriazole compound, MYUA series (manufactured by Miyoshi Oil&Fat Co., Ltd.; The Chemical Daily, Feb. 1, 2016) may be used.

In the curable composition according to the embodiment of the present invention, the content of the ultraviolet absorber is preferably 0.01 to 10 mass % and more preferably 0.01 to 5 mass % with respect to the total solid content of the curable composition according to the embodiment of the present invention. In the present invention, as the ultraviolet absorber, one kind may be used alone, or two or more kinds may be used. In a case where two or more ultraviolet absorbers are used in combination, it is preferable that the total content of the two or more ultraviolet absorbers is in the above-described range.

<<Polymerization Inhibitor>>

The curable composition according to the embodiment of the present invention may include a polymerization inhibitor. Examples of the polymerization inhibitor include hydroquinone, p-methoxyphenol, di-tert-butyl-p-cresol, pyrogallol, tert-butylcatechol, benzoquinone, 4,4′-thiobis(3-methyl-6-tert-butylphenol), 2,2′-methylenebis(4-methyl-6-tert-butylphenol), and N-nitrosophenylhydroxyamine salt (for example, an ammonium salt or a cerium (III) salt). Among these, p-methoxyphenol is preferable. The content of the polymerization inhibitor is preferably 0.001 to 5 mass % with respect to the total solid content of the curable composition.

<<Silane Coupling Agent>>

The curable composition according to the embodiment of the present invention may include a silane coupling agent. In the present invention, the silane coupling agent refers to a silane compound having a functional group other than a hydrolyzable group. In addition, the hydrolyzable group refers to a substituent directly linked to a silicon atom and capable of forming a siloxane bond due to at least one of a hydrolysis reaction or a condensation reaction. Examples of the hydrolyzable group include a halogen atom, an alkoxy group, and an acyloxy group. Among these, an alkoxy group is preferable. That is, it is preferable that the silane coupling agent is a compound having an alkoxysilyl group. Examples of the functional group other than a hydrolyzable group include a vinyl group, a styrene group, a (meth)acryloyl group, a mercapto group, an epoxy group, an oxetanyl group, an amino group, an ureido group, a sulfide group, an isocyanate group, and a phenyl group. Among these, a (meth)acryloyl group or an epoxy group is preferable. Examples of the silane coupling agent include a compound described in paragraphs “0018” to “0036” of JP2009-288703A and a compound described in paragraphs “0056” to “0066” of JP2009-242604A, the contents of which are incorporated herein by reference.

The content of the silane coupling agent is preferably 0.01 to 15.0 mass % and more preferably 0.05 to 10.0 mass % with respect to the total solid content of the curable composition. As the silane coupling agent, one kind may be used alone, or two or more kinds may be used. In a case where two or more silane coupling agents are used in combination, it is preferable that the total content of the two or more silane coupling agents is in the above-described range.

<<Surfactant>>

The curable composition according to the embodiment of the present invention may include a surfactant. As the surfactants, various surfactants such as a fluorine surfactant, a nonionic surfactant, a cationic surfactant, an anionic surfactant, or a silicone surfactant can be used. The details of the surfactant can be found in paragraphs “0238” to “0245” of WO2015/166779A, the content of which is incorporated herein by reference.

It is preferable that the surfactant is a fluorine surfactant. By the curable composition according to the embodiment of the present invention containing a fluorine surfactant, liquid characteristics (in particular, fluidity) are further improved, and liquid saving properties can be further improved. In addition, a film having reduced thickness unevenness can be formed.

The fluorine content in the fluorine surfactant is preferably 3 to 40 mass %, more preferably 5 to 30 mass %, and still more preferably 7 to 25 mass %. The fluorine surfactant in which the fluorine content is in the above-described range is effective from the viewpoints of the uniformity in the thickness of the coating film and liquid saving properties. In addition, the solubility of the fluorine surfactant in the composition is also excellent.

Specific examples of the fluorine surfactant include a surfactant described in paragraphs “0060” to “0064” of JP2014-041318A (paragraphs “0060” to “0064” of corresponding WO02014/017669A) and a surfactant described in paragraphs “0117” to “0132” of JP2011-132503A, the contents of which are incorporated herein by reference. Examples of a commercially available product of the fluorine surfactant include: MEGAFACE F171, F172, F173, F176, F177, F141, F142, F143, F144, R30, F437, F475, F479, F482, F554, and F780 (all of which are manufactured by DIC Corporation); FLUORAD FC430, FC431, and FC171 (all of which are manufactured by Sumitomo 3M Ltd.); SURFLON S-382, SC-101, SC-103, SC-104, SC-105, SC-1068, SC-381, SC-383, S-393, and KH-40 (all of which are manufactured by Asahi Glass Co., Ltd.); and POLYFOX PF636, PF656, PF6320, PF6520, and PF7002 (all of which are manufactured by OMNOVA Solutions Inc.).

In addition, as the fluorine surfactant, a polymer of a fluorine-containing vinyl ether compound having a fluorinated alkyl group or a fluorinated alkylene ether group and a hydrophilic vinyl ether compound is also preferable. The details of this fluorine surfactant can be found in JP2016-216602A, the content of which is incorporated herein by reference

In addition, as the fluorine surfactant, an acrylic compound in which, in a case where heat is applied to a molecular structure which has a functional group having a fluorine atom, the functional group having a fluorine atom is cut and a fluorine atom is volatilized can also be preferably used. Examples of the fluorine surfactant include MEGAFACE DS series (manufactured by DIC Corporation, The Chemical Daily, Feb. 22, 2016, Nikkei Business Daily, Feb. 23, 2016), for example, MEGAFACE DS-21.

As the fluorine surfactant, a block polymer can also be used. Examples of the block polymer include a compound described in JP2011-089090A. As the fluorine surfactant, a fluorine-containing polymer compound can be preferably used, the fluorine-containing polymer compound including: a repeating unit derived from a (meth)acrylate compound having a fluorine atom; and a repeating unit derived from a (meth)acrylate compound having 2 or more (preferably 5 or more) alkyleneoxy groups (preferably an ethyleneoxy group and a propyleneoxy group). For example, the following compound can also be used as the fluorine surfactant used in the present invention.

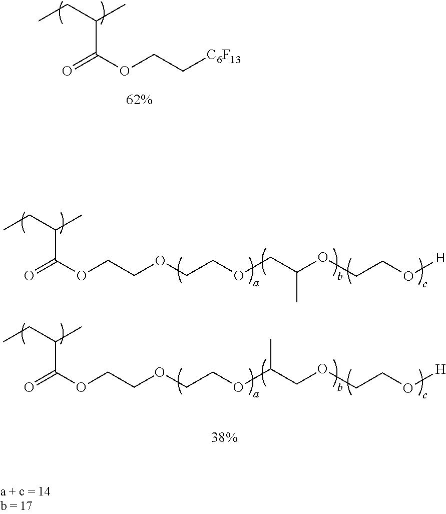

The weight-average molecular weight of the compound is preferably 3000 to 50000 and, for example, 14000. In the compound, “%” representing the proportion of a repeating unit is mol %.

In addition, as the fluorine surfactant, a fluorine-containing polymer having an ethylenically unsaturated group at a side chain can also be used. Specific examples include a compound described in paragraphs “0050” to “0090” and paragraphs “0289” to “0295” of JP2010-164965A, for example, MEGAFACE RS-101, RS-102, RS-718K, and RS-72-K manufactured by DIC Corporation. As the fluorine surfactant, a compound described in paragraphs “0015” to “0158” of JP2015-117327A can also be used.

Examples of the nonionic surfactant include glycerol, trimethylolpropane, trimethylolethane, an ethoxylate and a propoxylate thereof (for example, glycerol propoxylate or glycerol ethoxylate), polyoxyethylene lauryl ether, polyoxyethylene stearyl ether, polyoxyethylene oleyl ether, polyoxyethylene octylphenyl ether, polyoxyethylene nonylphenyl ether, polyethylene glycol dilaurate, polyethylene glycol distearate, sorbitan fatty acid esters, PLURONIC L10, L31, L61, L62, 10R5, 17R2, and 25R2 (manufactured by BASF SE), TETRONIC 304, 701, 704, 901, 904, and 150R1 (manufactured by BASF SE), SOLSPERSE 20000 (manufactured by Lubrication Technology Inc.), NCW-101, NCW-1001, and NCW-1002 (all of which are manufactured by Wako Pure Chemical Industries, Ltd.), PIONIN D-6112, D-6112-W, and D-6315 (all of which are manufactured by Takemoto Oil&Fat Co., Ltd.), and OLFINE E1010 and SURFYNOL 104, 400, and 440 (all of which are manufactured by Nissin Chemical Co., Ltd.).

The content of the surfactant is preferably 0.001 mass % to 5.0 mass % and more preferably 0.005 to 3.0 mass % with respect to the total solid content of the curable composition according to the embodiment of the present invention. As the surfactant, one kind may be used alone, or two or more kinds may be used. In a case where two or more surfactants are used in combination, it is preferable that the total content of the two or more surfactants is in the above-described range.

<<Other Components>>

Optionally, the curable composition according to the embodiment of the present invention may further include a sensitizer, a curing accelerator, a filler, a thermal curing accelerator, a thermal polymerization inhibitor, a plasticizer, and other auxiliary agents (for example, conductive particles, a filler, an antifoaming agent, a flame retardant, a leveling agent, a peeling accelerator, an antioxidant, an aromatic chemical, a surface tension adjuster, or a chain transfer agent). By the curable composition appropriately including the components, properties such as film properties can be adjusted. The details of the components can be found in, for example, paragraph “0183” of JP2012-003225A (corresponding to paragraph “0237” of US2013/0034812A) and paragraphs “0101” to “0104” and “0107” to “0109” of JP2008-250074A, the contents of which are incorporated herein by reference. As the antioxidant, for example, a phenol compound, a phosphorus compound, (for example, a compound described in paragraph “0042” of JP2011-90147A), or a thioether compound can be used. Examples of a commercially available product of the antioxidant include ADEKA STAB series (AO-20, AO-30, AO-40, AO-50, AO-50F, AO-60, AO-60G, AO-80, and AO-330, manufactured by Adeka Corporation). The content of the antioxidant is preferably 0.01 to 20 mass % and more preferably 0.3 to 15 mass % with respect to the mass of the total solid content of the curable composition according to the embodiment of the present invention. As the antioxidant, one kind may be used alone, or two or more kinds may be used. In a case where two or more antioxidants are used in combination, it is preferable that the total content of the antioxidants is in the above-described range.

For example, in a case where a film is formed by coating, the viscosity (23° C.) of the curable composition according to the embodiment of the present invention is preferably 1 to 100 mPa·s. The lower limit is more preferably 2 mPa·s or higher and still more preferably 3 mPa·s or higher. The upper limit is more preferably 50 mPa·s or lower, still more preferably 30 mPa·s or lower, and still more preferably 15 mPa·s or lower.

The solid content concentration of the curable composition according to the embodiment the present invention changes depending on a coating method or the like and, for example, is preferably 1 to 50 mass %. The lower limit is more preferably 10 mass % or higher. The upper limit is more preferably 30 mass % or lower.

A storage container of the curable composition according to the embodiment of the present invention is not particularly limited, and a well-known storage container can be used. In addition, as the storage container, in order to suppress infiltration of impurities into the raw materials or the composition, a multilayer bottle in which a container inner wall having a six-layer structure is formed of six kinds of resins or a bottle in which a container inner wall having a seven-layer structure is formed of six kinds of resins is preferably used. Examples of the container include a container described in JP2015-123351A.

The use of the curable composition according to the embodiment of the present invention is not particularly limited. The composition according to the embodiment of the present invention can be preferably used to form a near infrared cut filter or the like. In addition, by the curable composition according to the embodiment of the present invention including the coloring material that shields visible light, an infrared transmitting filter that can allow transmission of only near infrared light at a specific wavelength or higher can also be formed.

<Method of Preparing Curable Composition>

The curable composition according to the embodiment of the present invention can be prepared by mixing the above-described components with each other. During the preparation of the curable composition, all the components may be dissolved or dispersed in a solvent at the same time to prepare the curable composition. Optionally, two or more solutions or dispersions to which the respective components are appropriately added may be prepared, and the solutions or dispersions may be mixed with each other during use (during application) to prepare the curable composition.

In addition, in a case where the curable composition according to the embodiment of the present invention includes particles of a pigment or the like, it is preferable that a process of dispersing the particles is provided. Examples of a mechanical force used for dispersing the particles in the process of dispersing the particles include compression, squeezing, impact, shearing, and cavitation. Specific examples of the process include a beads mill, a sand mill, a roll mill, a ball mill, a paint shaker, a Microfluidizer, a high-speed impeller, a sand grinder, a flow jet mixer, high-pressure wet atomization, and ultrasonic dispersion. During the pulverization of the particles using a sand mill (beads mill), it is preferable that the process is performed under conditions for increasing the pulverization efficiency, for example, by using beads having a small size and increasing the filling rate of the beads. In addition, it is preferable that rough particles are removed by filtering, centrifugal separation, and the like after pulverization. In addition, as the process and the disperser for dispersing the particles, a process and a disperser described in “Complete Works of Dispersion Technology, Johokiko Co., Ltd., Jul. 15, 2005”, “Dispersion Technique focusing on Suspension (Solid/Liquid Dispersion) and Practical Industrial Application, Comprehensive Reference List, Publishing Department of Management Development Center, Oct. 10, 1978”, and paragraph “0022” JP2015-157893A can be suitably used. In addition, in the process of dispersing the particles, particles may be refined in a salt milling step. A material, a device, process conditions, and the like used in the salt milling step can be found in, for example, JP2015-194521A and JP2012-046629A.

During the preparation of the curable composition, it is preferable that the curable composition is filtered through a filter, for example, in order to remove foreign matter or to reduce defects. As the filter, any filter which is used in the related art for filtering or the like can be used without any particular limitation. Examples of a material of the filter include: a fluororesin such as polytetrafluoroethylene (PTFE); a polyamide resin such as nylon (for example, nylon-6 or nylon-6,6); and a polyolefin resin (including a polyolefin resin having a high density and an ultrahigh molecular weight) such as polyethylene or polypropylene (PP). Among these materials, polypropylene (including high-density polypropylene) or nylon is preferable.

The pore size of the filter is suitably about 0.01 to 7.0 μm and is preferably about 0.01 to 3.0 m and more preferably about 0.05 to 0.5 μm. In a case where the pore size of the filter is in the above-described range, fine foreign matter can be reliably removed. In addition, it is preferable that a fibrous filter material is used. Examples of the fibrous filter material include polypropylene fiber, nylon fiber, and glass fiber. Specific examples include a filter cartridge of SBP type series (for example, SBP008), TPR type series (for example, TPR002 or TPR005), and SHPX type series (for example, SHPX003) all of which are manufactured by Roki Techno Co., Ltd.

In a case where a filter is used, a combination of different filters (for example, a first filter and a second filter) may be used. At this time, the filtering using each of the filters may be performed once, or twice or more.

In addition, a combination of filters having different pore sizes in the above-described range may be used. Here, the pore size of the filter can refer to a nominal value of a manufacturer of the filter. A commercially available filter can be selected from various filters manufactured by Pall Corporation (for example, DFA4201NXEY), Toyo Roshi Kaisha, Ltd., Entegris Japan Co., Ltd. (former Mykrolis Corporation), or Kits Microfilter Corporation.

The second filter may be formed of the same material as that of the first filter.

In addition, the filtering using the first filter may be performed only on the dispersion, and the filtering using the second filter may be performed on a mixture of the dispersion and other components.

<Dispersing Auxiliary Agent>

Next, a dispersing auxiliary agent according to the embodiment of the present invention will be described. The dispersing auxiliary agent according to the embodiment of the present invention includes a compound (hereinafter, also referred to as “compound Ax”) having a structure in which at least one functional group selected from an acid group having a pKa of 3 or lower and a Clog P value of −1.1 or higher, an anionic group obtained by dissociating one or more hydrogen atoms from the acid group, or a salt of the acid group is bonded to a π-conjugated structure of a colorant skeleton. The compound Ax will also be referred to as “colorant derivative”. Examples of the functional group in the compound Ax include the functional group A described above regarding the compound A, and a preferable range thereof is also the same.

The colorant skeleton in the compound Ax may be a colorant skeleton derived from a colorant compound having an absorption in a visible range or a colorant skeleton derived from a colorant compound having an absorption in a near infrared range. Among these, from the viewpoint of improving absorption properties of a near infrared range, a colorant skeleton derived from a colorant compound having an absorption in a near infrared range is preferable. Specific examples of the colorant skeleton in the compound Ax include a pyrrolopyrrole colorant skeleton, a diimonium colorant skeleton, a phthalocyanine colorant skeleton, a naphthalocyanine colorant skeleton, a polymethine colorant skeleton, a xanthene colorant skeleton, a pyrromethene colorant skeleton, a quinacridone colorant skeleton, an azo colorant skeleton, a diketo pyrrolo pyrrole colorant skeleton, an anthraquinone colorant skeleton, a benzimidazolone colorant skeleton, a triazine colorant skeleton, an isophthalic acid colorant skeleton, an isoindoline colorant skeleton, a quinoline colorant skeleton, a benzothiazole colorant skeleton, a quinoxaline colorant skeleton, and a benzoxazole colorant skeleton. Among these, a pyrrolopyrrole colorant skeleton, a diimonium colorant skeleton, a phthalocyanine colorant skeleton, a naphthalocyanine colorant skeleton, a polymethine colorant skeleton, a pyrromethene colorant skeleton, or a perylene colorant skeleton is preferable, at least one selected from a pyrrolopyrrole colorant skeleton, a phthalocyanine colorant skeleton, a naphthalocyanine colorant skeleton, or a polymethine colorant skeleton is more preferable, a pyrrolopyrrole colorant skeleton or a polymethine colorant skeleton is still more preferable, and a pyrrolopyrrole colorant skeleton is still more preferable. In addition, examples of the polymethine colorant skeleton depending on the kind of an atomic group to be bonded include a cyanine colorant skeleton, a merocyanine colorant skeleton, a squarylium colorant skeleton, a croconium colorant skeleton, and an oxonol colorant skeleton. Among these, a cyanine colorant skeleton, a squarylium colorant skeleton, or an oxonol colorant skeleton is preferable, and a cyanine colorant skeleton or a squarylium colorant skeleton is more preferable.

The compound Ax may be a compound having an absorption in a visible range or a compound having an absorption in a near infrared range. In addition, the compound Ax may also be a colorless compound. It is preferable that the compound Ax is a compound having a maximum absorption wavelength in a wavelength range of 650 to 1200 nm.

Specific examples of the compound Ax include the compounds described above as the specific examples of the compound A and compounds having the following structures.

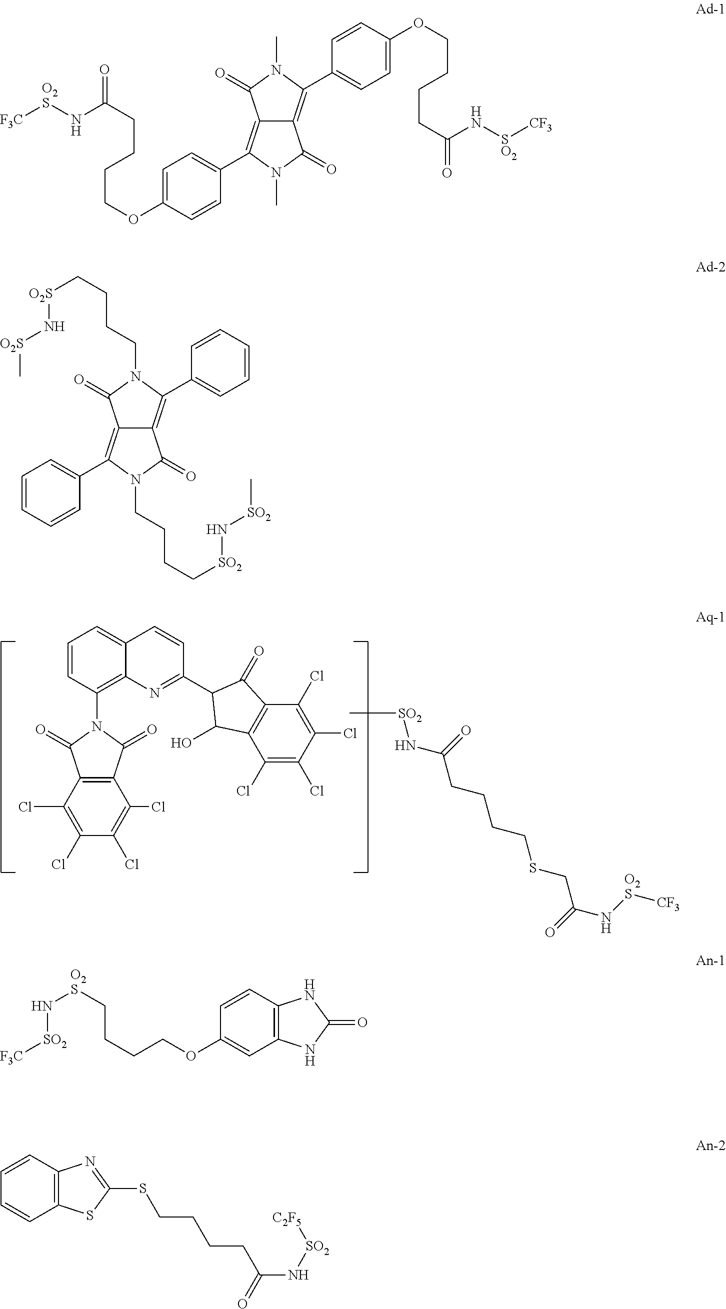

<Dispersion>

Next, a dispersion according to the embodiment of the present invention will be described. The dispersion according to the embodiment of the present invention comprises a pigment, the dispersing auxiliary agent according to the embodiment of the present invention, a dispersant, and a solvent. Examples of the pigment, the dispersant, and the solvent include the pigments, the dispersants, and the solvents described above as the components that can be used in the curable composition, and preferable ranges thereof are also the same. In addition, as the dispersing auxiliary agent, the compound A described above as the component that can be used in the curable composition can also be used. In particular, in a case where the pigment is a near infrared absorbing colorant and the dispersing auxiliary agent is a compound that has a colorant skeleton derived from a colorant compound having an absorption in a near infrared range, interactivity between the pigment and the dispersing auxiliary agent can be improved, dispersibility of the pigment can be further improved, and absorption properties of the near infrared range can be further improved.

In the dispersion according to the embodiment of the present invention, the content of the pigment is preferably 10 to 80 mass % with respect to the total solid content of the dispersion. The upper limit is preferably 70 mass %% or lower, more preferably 60 mass % or lower, and still more preferably 50 mass % or lower. The lower limit is preferably 15 mass % or higher, more preferably 20 mass % or higher, and still more preferably 25 mass % or higher.

In the dispersion according to the embodiment of the present invention, the content of the dispersant is preferably 10 to 80 mass % with respect to the total solid content of the dispersion. The upper limit is preferably 70 mass %% or lower, more preferably 60 mass % or lower, and still more preferably 50 mass % or lower. The lower limit is preferably 15 mass % or higher, more preferably 20 mass % or higher, and still more preferably 25 mass % or higher. In addition, the content of the dispersant is preferably 20 to 150 parts by mass with respect to 100 parts by mass of the pigment. The upper limit is preferably 140 parts by mass or less, more preferably 130 parts by mass or less, and still more preferably 120 parts by mass or less. The lower limit is preferably 30 parts by mass or more, more preferably 35 parts by mass or more, and still more preferably 40 parts by mass or more.

In the dispersion according to the embodiment of the present invention, the content of the dispersing auxiliary agent is preferably 0.1 to 30 mass % with respect to the total solid content of the dispersion. The upper limit is preferably 25 mass % or lower, more preferably 20 mass % or lower, and still more preferably 15 mass % or lower. The lower limit is preferably 0.1 mass % or higher, more preferably 1.0 mass % or higher, and still more preferably 2.0 mass % or higher. In addition, the content of the dispersing auxiliary agent is preferably 1 to 50 parts by mass with respect to 100 parts by mass of the pigment. The upper limit is preferably 45 parts by mass or less, more preferably 40 parts by mass or less, and still more preferably 35 parts by mass or less. The lower limit is preferably 2.5 parts by mass or more, more preferably 5.0 parts by mass or more, and still more preferably 7.5 parts by mass or more.

The content of the solvent in the dispersion according to the embodiment of the present invention is preferably 1 to 50 mass % with respect to the solid content concentration of the dispersion. The lower limit of the solid content concentration of the dispersion is preferably 2.5 mass % or higher, 5.0 mass % or higher, and still more preferably 7.5 mass % or higher. The upper limit is preferably 45 mass % or lower, more preferably 40 mass % or lower, and still more preferably 35 mass % or lower.

The curable composition according to the embodiment of the present invention may include the above-described dispersion according to the embodiment of the present invention.

<Method of Manufacturing Dispersion>

Next, a method of manufacturing a dispersion according to the embodiment of the present invention will be described.

The method of manufacturing a dispersion according to the embodiment of the present invention includes a step of dispersing a pigment in the presence of the dispersing auxiliary agent according to the embodiment of the present invention, a dispersant, and a solvent. Examples of a mechanical force used for dispersing the pigment include compression, squeezing, impact, shearing, and cavitation. Specific examples of the process include a beads mill, a sand mill, a roll mill, a ball mill, a paint shaker, a Microfluidizer, a high-speed impeller, a sand grinder, a project mixer, high-pressure wet atomization, and ultrasonic dispersion. The details of the process are the same as the contents described above regarding the method of preparing the curable composition. In addition, in the method of manufacturing a dispersion, it is preferable that the pigment is filtered through a filter, for example, in order to remove foreign matter or to reduce defects. The details of the filtering are the same as the contents described above regarding the method of preparing the curable composition.

<Cured Film>

Next, a cured film according to the embodiment of the present invention will be described. The cured film according to the embodiment of the present invention is obtained from the above-described curable composition according to the embodiment of the present invention. The cured film according to the embodiment of the present invention can be preferably used as various optical filters such as a near infrared cut filter, an infrared transmitting filter, or a color filter. In particular, the cured film according to the embodiment of the present invention can be preferably used as a near infrared cut filter. The cured film according to the embodiment of the present invention may be a film having a pattern or a film (flat film) not having a pattern. In addition, the cured film according to the embodiment of the present invention may be used in a state where it is laminated on a support, or the cured film according to the embodiment of the present invention may be peeled off from a support.

In a case where the cured film according to the embodiment of the present invention is used as a near infrared cut filter, the curable composition according to the embodiment of the present invention may further include a near infrared absorbing colorant in addition to the above-described compound A. In addition, in a case where the cured film according to the embodiment of the present invention is used as an infrared transmitting filter, it is preferable that the curable composition according to the embodiment of the present invention further includes a coloring material that shields visible light in addition to the compound A. In addition, in a case where the cured film according to the embodiment of the present invention is used as a color filter, it is preferable that the curable composition according to the embodiment of the present invention further includes a chromatic colorant in addition to the compound A.

The thickness of the cured film according to the embodiment of the present invention can be adjusted according to the purpose. The thickness is preferably 20 μm or less, more preferably 10 μm or less, and still more preferably 5 μm or less. For example, the lower limit of the thickness is preferably 0.1 μm or more, more preferably 0.2 μm or more, and still more preferably 0.3 μm or more.

In the present invention, “near infrared cut filter” refers to a filter that allows transmission of light (visible light) in the visible range and shields at least a part of light (near infrared light) in the near infrared range. The near infrared cut filter may be a filter that allows transmission of light in the entire wavelength range of the visible range, or may be a filter that allows transmission of light in a specific wavelength range of the visible range and shields light in another specific wavelength range of the visible range. In addition, in the present invention, a color filter refers to a filter that allows transmission of light in a specific wavelength range of the visible range and shields light in another specific wavelength range of the visible range. In addition, “infrared transmitting filter” refers to a filter that shields light in the visible range and allows transmission of at least a part of light (near infrared light) in the near infrared range.

In a case where the cured film according to the embodiment of the present invention is used as a near infrared cut filter, it is preferable that the cured film according to the embodiment of the present invention has an absorption maximum in a wavelength range of 650 to 1200 nm (preferably in a wavelength range of 700 to 1000 nm). The average transmittance in a wavelength range of 400 to 550 nm is preferably 70% or higher, more preferably 80% or higher, still more preferably 85% or higher, and still more preferably 90% or higher. In addition, a transmittance of in the entire wavelength range of 400 to 550 nm is preferably 70% or higher, more preferably 80% or higher, and still more preferably 90% or higher. In addition, a preferable range of the near infrared shielding properties of the near infrared cut filter varies depending on the use. For example, a transmittance at at least one point in a wavelength range of 650 to 1200 nm (preferably in a wavelength range of 700 to 1000 nm) is preferably 20% or lower, more preferably 15% or lower, and still more preferably 10% or lower.

In a case where the cured film according to the embodiment of the present invention is used as a near infrared cut filter, the cured film according to the embodiment of the present invention can be used in combination with a color filter that includes a chromatic colorant. For example, the cured film according to the embodiment of the present invention and the color filter can be laminated to be used as a laminate. In the laminate, the cured film according to the embodiment of the present invention and the color filter may be or may not be adjacent to each other in a thickness direction. In a case where the cured film according to the embodiment of the present invention is not adjacent to the color filter in the thickness direction, the cured film according to the embodiment of the present invention may be formed on another support other than a support on which the color filter is formed, or another member (for example, a microlens or a planarizing layer) constituting a solid image pickup element may be interposed between the cured film according to the embodiment of the present invention and the color filter.

In a case where the cured film according to the embodiment of the present invention is used as an infrared transmitting filter, it is preferable that the cured film according to the embodiment of the present invention has at least one of the following spectral characteristics (1) to (4).

(1): a film in which a maximum value of a light transmittance of the film in a thickness direction in a wavelength range of 400 to 640 nm is 20% or lower (preferably 15% or lower and more preferably 10% or lower), and a minimum value of a light transmittance of the film in the thickness direction in a wavelength range of 800 to 1300 nm is 70% or higher (preferably 75% or higher and more preferably 80% or higher). This cured film can shield light in a wavelength range of 400 to 640 nm and can allow transmission of light having a wavelength of longer than 670 nm can be formed.

(2): a film in which a maximum value of a light transmittance of the film in a thickness direction in a wavelength range of 400 to 750 nm is 20% or lower (preferably 15% or lower and more preferably 10% or lower), and a minimum value of a light transmittance of the film in the thickness direction in a wavelength range of 900 to 1300 nm is 70% or higher (preferably 75% or higher and more preferably 80% or higher). This film can shield light in a wavelength range of 400 to 750 nm and can allow transmission of light having a wavelength of longer than 850 nm can be formed.

(3): a film in which a maximum value of a light transmittance of the film in a thickness direction in a wavelength range of 400 to 830 nm is 20% or lower (preferably 15% or lower and more preferably 10% or lower), and a minimum value of a light transmittance of the film in the thickness direction in a wavelength range of 1000 to 1300 nm is 70% or higher (preferably 75% or higher and more preferably 80% or higher). This film can shield light in a wavelength range of 400 to 830 nm and can allow transmission of light having a wavelength of longer than 940 nm can be formed.

(4): a film in which a maximum value of a light transmittance of the film in a thickness direction in a wavelength range of 400 to 950 nm is 20% or lower (preferably 15% or lower and more preferably 10% or lower), and a minimum value of a light transmittance of the film in the thickness direction in a wavelength range of 1100 to 1300 nm is 70% or higher (preferably 75% or higher and more preferably 80% or higher). This film can shield light in a wavelength range of 400 to 950 run and can allow transmission of light having a wavelength of longer than 1040 nm can be formed.

In a case where the cured film according to the embodiment of the present invention is used as a color filter, it is preferable that the cured film according to the embodiment of the present invention has a color such as green, red, blue, cyan, magenta, or yellow.

The cured film according to the embodiment of the present invention can be used in various devices including a solid image pickup element such as a charge coupled device (CCD) or a complementary metal-oxide semiconductor (CMOS), an infrared sensor, or an image display device.

<Method of Forming Cured Film>

The cured film according to the embodiment of the present invention can be formed through a step of applying the curable composition according to the embodiment of the present invention.

In the method of forming the cured film, it is preferable that the curable composition is applied to a support. Examples of the support include a substrate formed of a material such as silicon, non-alkali glass, soda glass, PYREX (registered trade name) glass, or quartz glass. For example, an organic film or an inorganic film may be formed on the substrate. Examples of a material of the organic film include the above-described transparent resin. In addition, as the support, a substrate formed of the resin can also be used. In addition, a charge coupled device (CCD), a complementary metal-oxide semiconductor (CMOS), a transparent conductive film, or the like may be formed on the support. In addition, a black matrix that separates pixels from each other may be formed on the support. In addition, optionally, an undercoat layer may be provided on the support to improve adhesiveness with a layer above the support, to prevent diffusion of materials, or to make a surface of the substrate flat. In addition, in a case where a glass substrate is used as the support, it is preferable that an inorganic film is formed on the glass substrate or the glass substrate may be dealkalized to be used.

As a method of applying the curable composition, a well-known method can be used. Examples of the well-known method include: a drop casting method; a slit coating method; a spray coating method; a roll coating method; a spin coating method; a cast coating method; a slit and spin method; a pre-wetting method (for example, a method described in JP2009-145395A); various printing methods including jet printing such as an ink jet method (for example, an on-demand method, a piezoelectric method, or a thermal method) or a nozzle jet method, flexographic printing, screen printing, gravure printing, reverse offset printing, and metal mask printing; a transfer method using a mold or the like; and a nanoimprint lithography method. The application method using an ink jet method is not particularly limited, and examples thereof include a method (in particular, pp. 115 to 133) described in “Extension of Use of Ink Jet—Infinite Possibilities in Patent-” (February, 2005, S.B. Research Co., Ltd.) and methods described in JP2003-262716A, JP2003-185831A, JP2003-261827A, JP2012-126830A, and JP2006-169325A.

A composition layer formed by applying the curable composition may be dried (pre-baked). In a case where a pattern is formed through a low-temperature process, pre-baking is not necessarily performed. In a case where pre-baking is performed, the pre-baking temperature is preferably 150° C. or lower, more preferably 120° C. or lower, and still more preferably 110° C. or lower. The lower limit is, for example, 50° C. or higher or 80° C. or higher. By setting the pre-baking temperature to be 150° C. or lower, for example, in a case where a photoelectric conversion film of an image sensor is formed of an organic material, the characteristics of the organic material can be effectively maintained. The pre-baking time is preferably 10 to 3000 seconds, more preferably 40 to 2500 seconds, and still more preferably 80 to 220 seconds. Drying can be performed using a hot plate, an oven, or the like.

The method of forming the cured film according to the embodiment of the present invention may further include a step of forming a pattern. Examples of a pattern forming method include a pattern forming method using a photolithography method and a pattern forming method using a dry etching method. In a case where the cured film according to the embodiment of the present invention is used as a flat film, the step of forming a pattern is not necessarily performed. Hereinafter, the step of forming a pattern will be described in detail.

(Case where Pattern is Formed Using Photolithography Method)

It is preferable that the pattern forming method using a photolithography method includes: a step (exposure step) of exposing the composition layer, which is formed by applying the curable composition according to the embodiment of the present invention, in a pattern shape; and a step (development step) of forming a pattern by removing a non-exposed portion of the composition layer by development. Optionally, the pattern forming method may further include a step (post-baking step) of baking the developed pattern. Hereinafter, the respective steps will be described.

<<Exposure Step>>

In the exposure step, the composition layer is exposed in a pattern shape. For example, the composition layer can be exposed in a pattern shape using an exposure device such as a stepper through a mask having a predetermined mask pattern. As a result, an exposed portion can be cured. As radiation (light) used during the exposure, in particular, ultraviolet rays such as g-rays or i-rays are preferable, and i-rays are more preferable. The irradiation dose (exposure dose) is preferably 0.03 to 2.5 J/cm², more preferably 0.05 to 1.0 J/cm², and most preferably 0.08 to 0.5 J/cm². The oxygen concentration during exposure can be appropriately selected. The exposure may be performed not only in air but also in a low-oxygen atmosphere having an oxygen concentration of 19 vol % or lower (for example, 15 vol %, 5 vol %, or substantially 0 vol %) or in a high-oxygen atmosphere having an oxygen concentration of higher than 21 vol % (for example, 22 vol %, 30 vol %, or 50 vol %). In addition, the exposure illuminance can be appropriately set and typically can be selected in a range of 1000 W/m² to 100000 W/m² (for example, 5000 W/m², 15000 W/m², or 35000 W/m²).

Conditions of the oxygen concentration and conditions of the exposure illuminance may be appropriately combined. For example, conditions are oxygen concentration: 10 vol % and illuminance: 10000 W/m², or oxygen concentration: 35 vol % and illuminance: 20000 W/m².

<<Development Step>>

Next, a pattern is formed by removing a non-exposed portion of the exposed composition layer by development. The non-exposed portion of the composition layer can be removed by development using a developer. As a result, a non-exposed portion of the composition layer in the exposure step is eluted into the developer, and only the photocured portion remains on the support. As the developer, an alkali developer which does not cause damages to a solid image pickup element as an underlayer, a circuit or the like is desired. For example, the temperature of the developer is preferably 20° C. to 30° C. The development time is preferably 20 to 180 seconds. In addition, in order to further improve residue removing properties, a step of shaking the developer off per 60 seconds and supplying a new developer may be repeated multiple times.

Examples of the alkaline agent used as the developer include: an organic alkaline compound such as ammonia water, ethylamine, diethylamine, dimethylethanolamine, diglycolamine, diethanolamine, hydroxyamine, ethylenediamine, tetramethylammonium hydroxide, tetraethylarnuonium hydroxide, tetrapropylammonium hydroxide, tetrabutylammonium hydroxide, ethyltrimethylammonium hydroxide, benzyltrimethylammonium hydroxide, dimethyl bis(2-hydroxyethyl)ammonium hydroxide, choline, pyrrole, piperidine, or 1,8-diazabicyclo[5.4.0]-7-undecene; and an inorganic alkaline compound such as sodium hydroxide, potassium hydroxide, sodium carbonate, sodium bicarbonate, sodium silicate, or sodium metasilicate. From the viewpoints of environment and safety, it is preferable that the alkaline agent is a compound having a high molecular weight. As the developer, an alkaline aqueous solution in which the above alkaline agent is diluted with pure water is preferably used. A concentration of the alkaline agent in the alkaline aqueous solution is preferably 0.001 to 10 mass % and more preferably 0.01 to 1 mass %. In addition, the developer may include a surfactant to be used. Examples of the surfactant include the above-described surfactants. Among these, a nonionic surfactant is preferable. From the viewpoint of easiness of transport, storage, and the like, the developer may be obtained by temporarily preparing a concentrated solution and diluting the concentrated solution to a necessary concentration during use. The dilution factor is not particularly limited and, for example, can be set to be in a range of 1.5 to 100 times. In a case where a developer including the alkaline aqueous solution is used, it is preferable that the layer is rinsed with pure water after development.

After the development, the film can also be dried and then heated (post-baking). Post-baking is a heat treatment which is performed after development to completely cure the film. In a case where post-baking is performed, for example, the post-baking temperature is preferably 100° C. to 240° C. From the viewpoint of curing the film, the post-baking temperature is more preferably 200° C. to 230° C. In addition, in a case where an organic electroluminescence (organic EL) element is used as a light-emitting light source, or in a case where a photoelectric conversion film of an image sensor is formed of an organic material, the post-baking temperature is preferably 150° C. or lower, more preferably 120° C. or lower, still more preferably 100° C. or lower, and still more preferably 90° C. or lower. The lower limit is, for example, 50° C. or higher. The film after the development is post-baked continuously or batchwise using heating means such as a hot plate, a convection oven (hot air circulation dryer), a high-frequency heater under the above-described conditions.

(Case where Pattern is Formed Using Dry Etching Method)

The formation of a pattern using a dry etching method can be performed using a method including: applying the curable composition to a support or the like to form a composition layer; curing the composition layer to form a cured composition layer; forming a patterned photoresist layer on the cured composition layer; and dry-etching the cured composition layer with etching gas by using the patterned photoresist layer as a mask. It is preferable that pre-baking is further performed in order to form the photoresist layer. In particular, in a preferable aspect, as a process of forming the photoresist, baking after exposure or baking after development (post-baking) is performed. The details of the pattern formation using the dry etching method can be found in paragraphs “0010” to “0067” of JP2013-064993A, the content of which is incorporated herein by reference.

<Optical Filter>

Next, an optical filter according to the embodiment of the present invention will be described. The optical filter according to the embodiment of the present invention includes the cured film according to the embodiment of the present invention. The optical filter according to the embodiment of the present invention can be preferably used as at least one selected from a near infrared cut filter, an infrared transmitting filter, or a color filter, can be more preferably used as a near infrared cut filter or an infrared transmitting filter, and can be still more preferably used as a near infrared cut filter.

In a case where the cured film according to the embodiment of the present invention is used as a near infrared cut filter, the near infrared cut filter may further include, for example, a layer containing copper, a dielectric multi-layer film, or an ultraviolet absorbing layer in addition to the cured film according to the embodiment of the present invention. By further including the layer containing copper and/or the dielectric multi-layer film, the near infrared cut filter having a viewing angle and excellent near infrared shielding properties can be easily obtained. In addition, by including the ultraviolet absorbing layer, the near infrared cut filter having excellent ultraviolet shielding properties can be obtained. The details of the ultraviolet absorbing layer can be found in, for example, the description of an absorbing layer described in paragraphs “0040” to “0070” and paragraphs “0119” to “0145” of WO2015/099060, the content of which is incorporated herein by reference. The details of the dielectric multi-layer film can be found in paragraphs “0255” to “0259” of JP2014-041318A, the content of which is incorporated herein by reference. As the layer containing copper, a glass substrate (copper-containing glass substrate) formed of glass containing copper, or a layer (copper complex-containing layer) containing a copper complex may also be used. Examples of the glass containing copper include a phosphate glass including copper and a fluorophosphate glass including copper. Examples of a commercially available product of the glass containing copper include NF-50 (manufactured by AGC Techno Glass Co., Ltd.), BG-60 and BG-61 (both of which are manufactured by Schott AG), and CD5000 (manufactured by Hoya Corporation). Specific examples of the copper complex include compounds described in paragraphs “0009” to “0049” of WO2016/068037A, the content of which is incorporated herein by reference.

The optical filter according to the embodiment of the present invention can be used by using a near infrared cut filter and an infrared transmitting filter in combination. By using a near infrared cut filter and an infrared transmitting filter in combination, the optical filter can be preferably used for an infrared sensor that detects near infrared light at a specific wavelength. In a case where both an infrared cut filter and an infrared transmitting filter are used in combination, either or both of the near infrared cut filter and the infrared transmitting filter can be formed using the curable composition according to the embodiment of the present invention.

<Solid Image Pickup Element>

A solid image pickup element according to the embodiment of the present invention includes the cured film according to the embodiment of the present invention. The configuration of the solid image pickup element is not particularly limited as long as it includes the cured film according to the embodiment of the present invention and functions as a solid image pickup element. For example, the following configuration can be adopted.

The solid image pickup element includes a plurality of photodiodes and transfer electrodes on the support, the photodiodes constituting a light receiving area of the solid image pickup element, and the transfer electrode being formed of polysilicon or the like. In the solid image pickup element, a light shielding film formed of tungsten or the like which has openings through only light receiving sections of the photodiodes is provided on the photodiodes and the transfer electrodes, a device protective film formed of silicon nitride or the like is formed on the light shielding film so as to cover the entire surface of the light shielding film and the light receiving sections of the photodiodes, and the cured film according to the embodiment of the present invention is formed on the device protective film. Further, a configuration in which light collecting means (for example, a microlens; hereinafter, the same shall be applied) is provided above the device protective film and below the cured film according to the embodiment of the present invention (on a side thereof close the support), or a configuration in which light collecting means is provided on the cured film according to the embodiment of the present invention may be adopted. In addition, the color filter may have a structure in which it is embedded in a space which is partitioned in, for example, a lattice shape by a partition wall. In this case, it is preferable that the partition wall has a lower refractive index than each pixel. Examples of an imaging device having such a structure include a device described in JP2012-227478A and JP2014-179577A.

<Image Display Device>

An image display device according to the embodiment of the present invention comprises the cured film according to the embodiment of the present invention. Examples of the image display device include a liquid crystal display device or an organic electroluminescence (organic EL) display device. The definition and details of the image display device can be found in, for example, “Electronic Display Device (by Akiya Sasaki, Kogyo Chosakai Publishing Co., Ltd., 1990)” or “Display Device (Sumiaki Ibuki, Sangyo Tosho Co., Ltd.). In addition, the details of a liquid crystal display device can be found in, for example, “Next-Generation Liquid Crystal Display Techniques (Edited by Tatsuo Uchida, Kogyo Chosakai Publishing Co., Ltd., 1994)”. The liquid crystal display device to which the present invention is applicable is not particularly limited. For example, the present invention is applicable to various liquid crystal display devices described in “Next-Generation Liquid Crystal Display Techniques”. The image display device may include a white organic EL element. It is preferable that the white organic EL element has a tandem structure. The tandem structure of the organic EL element is described in, for example, JP2003-045676A, or pp. 326-328 of “Forefront of Organic EL Technology Development—Know-How Collection of High Brightness, High Precision, and Long Life” (Technical Information Institute, 2008). It is preferable that a spectrum of white light emitted from the organic EL element has high maximum emission peaks in a blue range (430 nm to 485 nm), a green range (530 nm to 580 nm), and a yellow range (580 nm to 620 nm). It is more preferable that the spectrum has a maximum emission peak in a red range (650 nm to 700 nm) in addition to the above-described emission peaks.

<Infrared Sensor>

An infrared sensor according to the embodiment of the present invention includes the cured film according to the embodiment of the present invention. The configuration of the infrared sensor is not particularly limited as long as it functions as an infrared sensor.

Hereinafter, an embodiment of the infrared sensor used in the present invention will be described using the drawings.

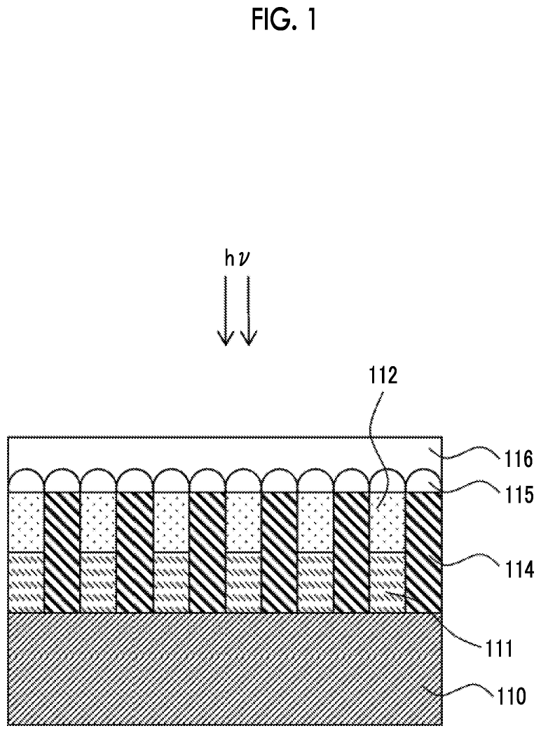

In FIG. 1, reference numeral 110 represents a solid image pickup element. In an imaging region provided on a solid image pickup element 110, near infrared cut filters 111 and infrared transmitting filters 114 are provided. In addition, color filters 112 are laminated on the near infrared cut filters 111. Microlenses 115 are disposed on an incidence ray hυ side of the color filters 112 and the infrared transmitting filters 114. A planarizing layer 116 is formed so as to cover the microlenses 115.

Spectral characteristics of the near infrared cut filters 111 can be selected according to the emission wavelength of an infrared light emitting diode (infrared LED) to be used. The near infrared cut filter 111 can be formed using, for example, the curable composition according to the embodiment of the present invention.

The color filters 112 is not particularly limited as long as pixels which allow transmission of light having a specific wavelength in a visible range and absorbs the light are formed therein, and well-known color filters of the related art for forming a pixel can be used. For example, pixels of red (R), green (G), and blue (B) are formed in the color filters. For example, the details of the color filters can be found in paragraphs “0214” to “0263” of JP2014-043556A, the content of which is incorporated herein by reference.

Characteristics of the infrared transmitting filters 114 can be selected according to the emission wavelength of the infrared LED to be used. For example, in a case where an infrared LED has an emission wavelength of 850 nm, it is preferable that the infrared transmitting filter 114 is a film in which a maximum value of a light transmittance of the film in a thickness direction in a wavelength range of 400 to 750 nm is 20% or lower (preferably 15% or lower and more preferably 10% or lower), and a minimum value of a light transmittance of the film in the thickness direction in a wavelength range of 900 to 1300 nm is 70% or higher (preferably 75% or higher and more preferably 80% or higher)

In addition, for example, in a case where the emission wavelength of the infrared LED is 940 nm, it is preferable that the infrared transmitting filter 114 is a film in which a maximum value of a light transmittance of the film in a thickness direction in a wavelength range of 400 to 830 nm is 20% or lower (preferably 15% or lower and more preferably 10% or lower), and a minimum value of a light transmittance of the film in the thickness direction in a wavelength range of 1000 to 1300 nm is 70% or higher (preferably 75% or higher and more preferably 80% or higher).

In the infrared sensor shown in FIG. 1, a near infrared cut filter (other near infrared cut filter) other than the near infrared cut filter 111 may be further disposed on the planarizing layer 116. As the other near infrared cut filter, for example, a layer containing copper and/or a dielectric multi-layer film may be provided. The details of the groups are as described above. In addition, as the other near infrared cut filter, a dual band pass filter may be used.

<Compound>

A compound according to the embodiment of the present invention is the above-described compound represented by the following Formula (A2).

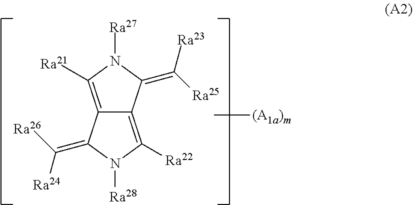

In Formula (A2), Ra²¹ and Ra²² each independently represent an alkyl group, an aryl group, or a heteroaryl group.

Ra²³, Ra²⁴, Ra²⁵, and Ra²⁶ each independently represent a cyano group, an acyl group, an alkoxycarbonyl group, an alkylsulfinyl group, an arylsulfinyl group, or a heteroaryl group.

Ra²⁷ and Ra²⁸ each independently represent —BRa²⁹Ra³⁰.

Ra²⁹ and Ra³⁰ each independently represent a hydrogen atom, a halogen atom, an alkyl group, an alkenyl group, an aryl group, a heteroaryl group, an alkoxy group, an aryloxy group, or a heteroaryloxy group, and Ra²⁹ and Ra³⁰ may be bonded to each other to form a ring.

A₁a represents the above-described functional group A.

m represents an integer of 1 to 10, and in a case where m represents 2 or more, a plurality of A₁a's may be the same as or different from each other.

Ra²¹ and Ra²² in Formula (A2) have the same definitions and the same preferable ranges as Ra¹ and Ra² in Formula (A1). Ra²³, Ra²⁴, Ra²⁵, and Ra²⁶ in Formula (A2) have the same definitions and the same preferable ranges as Ra³, Ra⁴, Ra⁵, and Ra⁶ in Formula (A1). Ra²⁹ and Ra³⁰ in Formula (A2) have the same definitions and the same preferable ranges as Ra⁹ and Ra¹⁰ in Formula (A1).

In Formula (A2), A₁ₐ represents the above-described functional group A. The details of the functional group A are as described above, and a preferable range thereof is also the same.

In Formula (A2), m represents an integer of 1 to 10, preferably 1 to 4, more preferably 1 to 3, still more preferably 1 or 2, and still more preferably 2.

### EXAMPLES

Hereinafter, the present invention will be described in detail using examples. Materials, used amounts, ratios, treatment details, treatment procedures, and the like shown in the following examples can be appropriately changed within a range not departing from the scope of the present invention. Accordingly, the scope of the present invention is not limited to the following specific examples. Unless specified otherwise, “part(s)” and “%” represent “part(s) by mass” and “mass %”.

<Synthesis of Compound (Ap-1)>

A compound (Ap-1) was synthesized according to the following scheme.

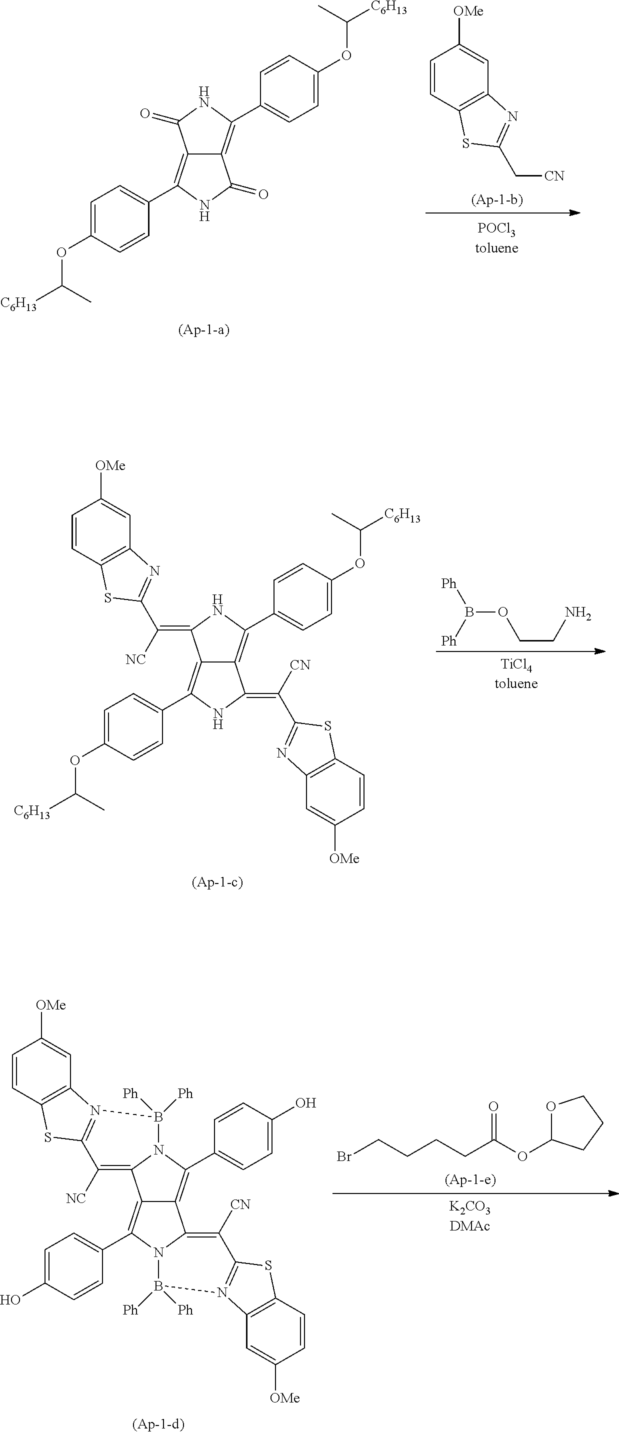

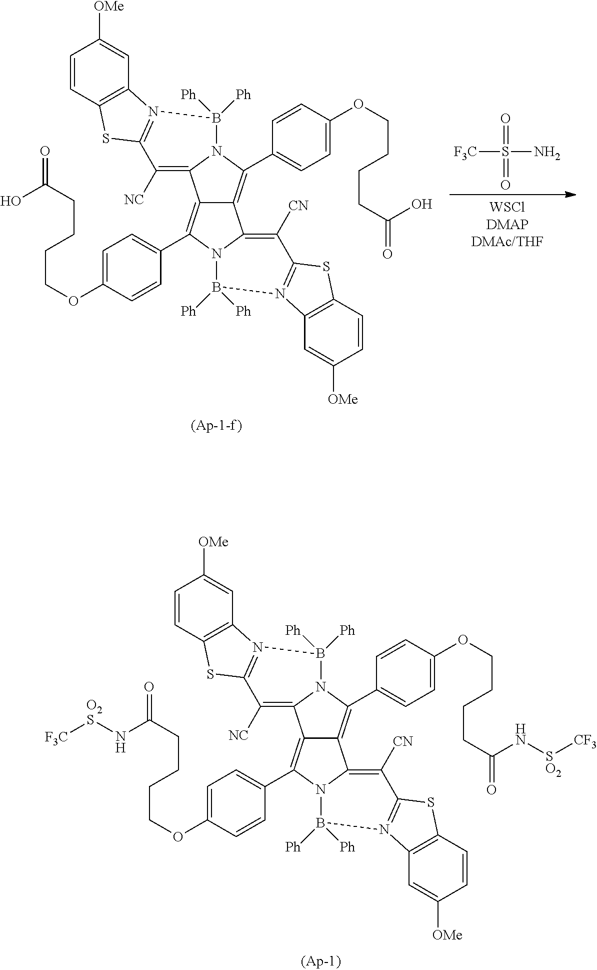

A compound (Ap-1-a) was synthesized from 4-(1-methylheptoxy)benzonitrile using a method described in U.S. Pat. No. 5,969,154A.

125.0 parts by mass of 5-methoxy-2-methylbenzothiazole and 234.8 parts by mass of potassium hydroxide were heated to reflux in 468 parts by mass of water and 468 parts by mass of ethylene glycol for 21 hours, and the solution was cooled to 10° C. or lower. While maintaining the temperature at 10° C. or lower, 6 mol/L hydrochloric acid was added such that the pH of the reaction solution was 6. Precipitated crystals were separated by filtration and were cleaned with 500 parts by mass of water. The entire amount of the obtained crystals, 46.1 parts by mass of malononitrile, and 49 parts by mass of acetic acid were stirred in 780 parts by mass of methanol at 60° C. for 1 hour, the solution was diluted with 250 parts by mass of methanol, and then the dilute solution was filtered while hot. The obtained filtrate was cooled to 10° C. or lower, and precipitated crystals were separated by filtration and were cleaned with 375 parts by mass of cold methanol. The obtained crystals were dried by blowing air at 50° C. for 12 hours. As a result, 117.2 parts by mass of a compound (Ap-1-b) was obtained.

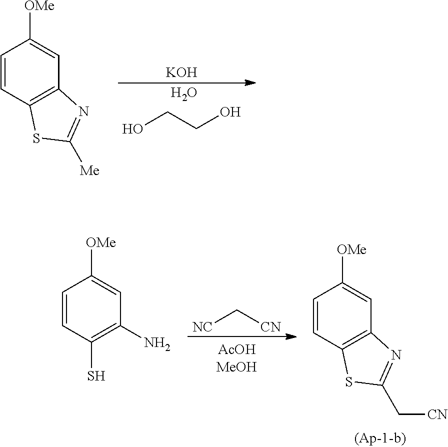

125.0 parts by mass of the compound (Ap-1-a) and 112.5 parts by mass of the compound (Ap-1-b) were stirred in 1400 parts by mass of toluene, 281.5 parts by mass of phosphorus oxychloride was added dropwise at 95° C., and the solution was stirred at 95° C. for 1 hour. After completion of the reaction, the solution was cooled to an internal temperature of 25° C., and 2500 parts by mass of methanol was added dropwise for 30 minutes while maintaining the internal temperature at 30° C. or lower. After completion of the dropwise addition, the solution was stirred at 25° C. for 30 minutes. Precipitated crystals were separated by filtration and were cleaned with 1250 parts by mass of methanol. 1250 parts by mass of methanol was added to the obtained crystals, the solution was heated to reflux for 30 minutes and was allowed to cool to 30° C., and crystals were separated by filtration. This operation was repeated twice. The obtained crystals were dried by blowing air at 50° C. for 12 hours. As a result, 140.2 parts by mass of a compound (Ap-1-c) was obtained.

116.0 parts by mass of diphenylborinic acid 2-aminoethyl ester and 135.0 parts by mass of the compound (Ap-1-c) were stirred in 2160 parts by mass of toluene, and 251.3 parts by mass of titanium tetrachloride was added dropwise for 15 minutes at an outside temperature of 95° C. The solution was heated to an outside temperature of 130° C. and was heated to reflux for 1 hour. The solution was allowed to cool to an internal temperature of 30° C., and 2160 parts by mass of methanol was added dropwise while maintaining the internal temperature at 30° C. or lower. After the dropwise addition, the solution was stirred for 30 minutes, and precipitated crystals were separated by filtration and were cleaned with 1080 parts by mass of methanol. 2160 parts by mass of methanol was added to the obtained crystals, the solution was heated to reflux for 30 minutes and was allowed to cool to 30° C., and crystals were separated by filtration. The obtained crystals were dried by blowing air at 50° C. for 12 hours, 2025 parts by mass of N-methylpyrrolidone was added, the solution was stirred at 120° C. for 2 hours and was allowed to cool to 30° C., and crystals were separated by filtration and were sequentially cleaned with 675 parts by mass of N-methylpyrrolidone and 1350 parts by mass of methanol. 2025 parts by mass of dimethylacetamide were added to the obtained crystals, the solution was stirred at 85° C. for 1 hour and was allowed to cool to 30° C., and crystals were separated by filtration and were sequentially cleaned with 675 parts by mass of dimethylacetamide and 1350 parts by mass of methanol. 2025 parts by mass of methanol was added to the obtained crystals, the solution was heated to reflux for 30 minutes and was allowed to cool to 30° C., and crystals were separated by filtration. The obtained crystals were dried by blowing air at 50° C. for 12 hours. As a result, 130.0 parts by mass of a compound (Ap-1-d) was obtained.

25 parts by mass of 5-bromovaleric acid and 96.8 parts by mass of dihydrofuran were added and were heated to reflux for 1 hour. Next, the solution was allowed to cool to an internal temperature of 30° C. or lower, and the solvent was removed by distillation under reduced pressure at an outside temperature of 30° C. As a result, 36.7 parts by mass of a compound (Ap-1-e) was obtained.

¹H-NMR (CDCl₃): δ1.70-2.10 (m, 8H), δ2.33 (t, 2H), δ3.41 (t, 2H), δ3.90-4.08 (m, 2H), δ6.31 (d, 1H)

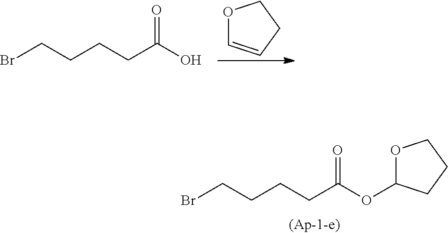

4 parts by mass of the compound (Ap-1-d) and 4.33 parts by mass of potassium carbonate were stirred in 75.2 parts by mass of dimethylacetamide, 7.87 parts by mass of the compound (Ap-1-e) and 15.0 parts by mass of dimethylacetamide were added, and the solution was stirred at room temperature for 10 minutes. The solution was heated to an internal temperature of 85° C. and was stirred for 1 hour. Next, the solution was allowed to cool to an internal temperature of 30° C., and 126.7 parts by mass of methanol was added dropwise. Precipitated crystals were separated by filtration and were cleaned with 64.0 parts by mass of methanol, 64.0 parts by mass of deionized water, and 64.0 parts by mass of methanol in this order. The obtained crystals were added to 160 parts by mass of 1 mol/L hydrogen chloride/ethyl acetate solution, and the solution was stirred at room temperature for 1 hour. Crystals were separated by filtration and were cleaned with 160 parts by mass of ethyl acetate. The obtained crystals were added to 80 parts by mass of ethyl acetate, and the solution was heated to reflux for 30 minutes. Next, crystals were separated by filtration and were cleaned with 160 parts by mass of ethyl acetate. The obtained crystals were dried by blowing air at 50° C. for 12 hours. As a result, 3.3 parts by mass of a compound (Ap-1-f) was obtained.

5 parts by mass of the compound (Ap-1-f), 3.26 parts by mass of trifluoromethanesulfonamide, 2.00 parts by mass of dimethylaminopyridine, 23.5 parts by mass of dimethylacetamide, and 22.23 parts by mass of tetrahydrofuran were added and stirred at room temperature for 5 minutes. Next, 3.1 parts by mass of 1-ethyl-3-(3-dimethylaminopropyl)carbodiimide hydrochloride was added, and the solution was heated to 40° C. and was stirred for 4 hours. Next, the reaction solution was allowed to cool to an internal temperature of 30° C. and was added dropwise to 134 parts by mass of ethyl acetate. Precipitated crystals were separated by filtration and were cleaned with 89.7 parts by mass of ethyl acetate. The obtained crystals were added to 150 parts by mass of distilled water, and a reslurry operation was performed for 30 minutes. The suspension was filtered and was cleaned with 150 parts by mass of distilled water. The obtained crystals were added to 44.45 parts by mass of tetrahydrofuran, and the obtained THF suspension was added to 100 parts by mass of 1 N hydrochloric acid aqueous solution. Precipitated crystals were separated by filtration and were cleaned with 100 parts by mass of water. The obtained crystals were dried by blowing air at 50° C. for 12 hours. As a result, 4.0 parts by mass of the compound (Ap-1) was obtained. A peak of molecular weight of 1483.2 was observed by matrix-assisted laser desorption/ionization-mass spectrometry (MALDI-MS), and the obtained compound was identified as the compound (Ap-1). In addition, the measurement result of nuclear magnetic resonance (NMR) of the compound (Ap-1) is as follows.

¹H-NMR (dimethyl sulfoxide): δ1.65-1.68 (m, 8H), 2.23 (t, 4H), 3.29 (s, 6H), 3.90 (t, 4H), 6.39-6.45 (m, 10H), 6.84 (d, 2H), 7.14-7.25 (m, 20H), 7.78 (d, 2H) ¹⁹F-NMR (dimethyl sulfoxide): δ −76.9

<Synthesis of Compound (Ap-26)>

A compound (Ap-26) was synthesized using the same synthesis method as that of the compound (Ap-1), except that perfluoroethanesulfonamide was used instead of trifluoromethanesulfonamide. A peak of molecular weight of 1583.2 was observed by MALDI-MS, and the obtained compound was identified as the compound (Ap-26). In addition, the measurement result of NMR of the compound (Ap-26) is as follows.

¹H-NMR (dimethyl sulfoxide): δ1.65-1.76 (m, 8H), 2.23 (t, 4H), 3.29 (s, 6H), 3.90 (t, 4H), 6.40-6.45 (m, 10H), 6.84 (d, 2H), 7.14-7.25 (m, 20H), 7.78 (d, 2H)

¹⁹F-NMR (dimethyl sulfoxide): δ −78.6 (6F), −116.6 (4F)

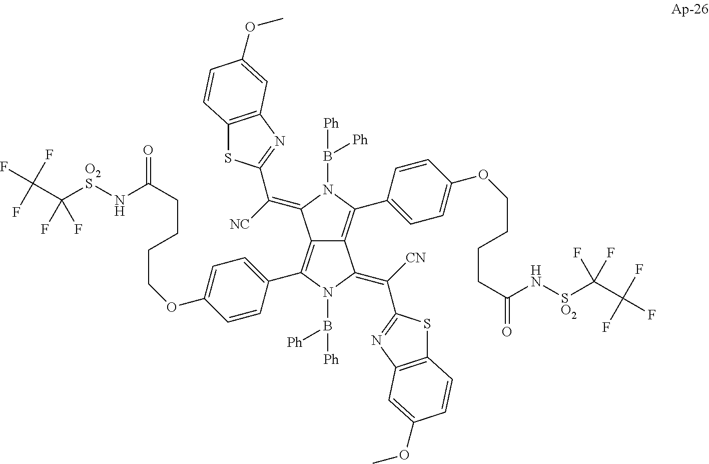 [SMILES: COc1ccc2sc(/C(C#N)=c3/c4c(-c5ccc(OCCCCC(=O)NS(=O)(=O)C(F)(F)C(F)(F)F)cc5)n(B(c5ccccc5)c5ccccc5)/c(=C(/C#N)c5nc6cc(OC)ccc6s5)c4c(-c4ccc(OCCCCC(=O)NS(=O)(=O)C(F)(F)C(F)(F)F)cc4)n3B(c3ccccc3)c3ccccc3)nc2c1]

<Synthesis of Compound (Ap-27)>

A compound (Ap-27) was synthesized using the same synthesis method as that of the compound (Ap-1), except that difluoromethanesulfonamide was used instead of trifluoromethanesulfonamide. A peak of molecular weight of 1446.3 was observed by MALDI-MS, and the obtained compound was identified as the compound (Ap-27).

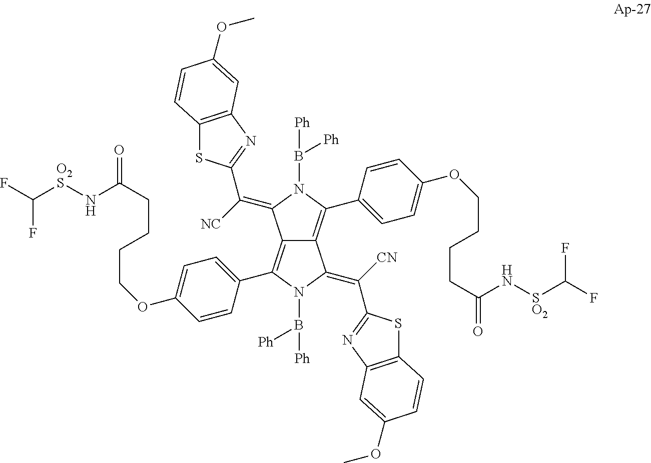 [SMILES: COc1ccc2sc(/C(C#N)=c3/c4c(-c5ccc(OCCCCC(=O)NS(=O)(=O)C(F)F)cc5)n(B(c5ccccc5)c5ccccc5)/c(=C(/C#N)c5nc6cc(OC)ccc6s5)c4c(-c4ccc(OCCCCC(=O)NS(=O)(=O)C(F)F)cc4)n3B(c3ccccc3)c3ccccc3)nc2c1]

<Synthesis of Compound (Ap-33)>

A compound (Ap-33) was synthesized using the same synthesis method as that of the compound (Ap-1), except that perfluorobutanesulfonamide was used instead of trifluoromethanesulfonamide. A peak of molecular weight of 1782.3 was observed by MALDI-MS, and the obtained compound was identified as the compound (Ap-33).

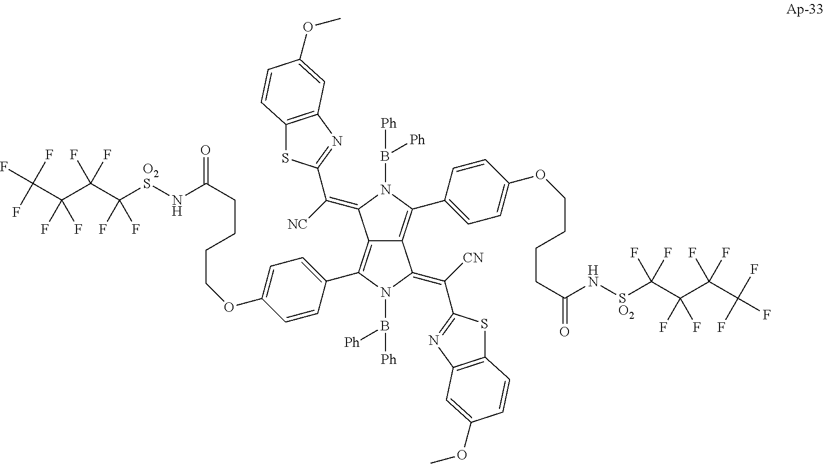 [SMILES: COc1ccc2sc(/C(C#N)=c3/c4c(-c5ccc(OCCCCC(=O)NS(=O)(=O)C(F)(F)C(F)(F)C(F)(F)C(F)(F)F)cc5)n(B(c5ccccc5)c5ccccc5)/c(=C(/C#N)c5nc6cc(OC)ccc6s5)c4c(-c4ccc(OCCCCC(=O)NS(=O)(=O)C(F)(F)C(F)(F)C(F)(F)C(F)(F)F)cc4)n3B(c3ccccc3)c3ccccc3)nc2c1]

<Synthesis of Compound (Ap-34)>

A compound (Ap-34) was synthesized using the same synthesis method as that of the compound (Ap-1), except that 4-(perfluro butoxy)perfluorobutanesulfonamide was used instead of trifluoromethanesulfonamide. A peak of molecular weight of 2214.3 was observed by MALDI-MS, and the obtained compound was identified as the compound (Ap-34).

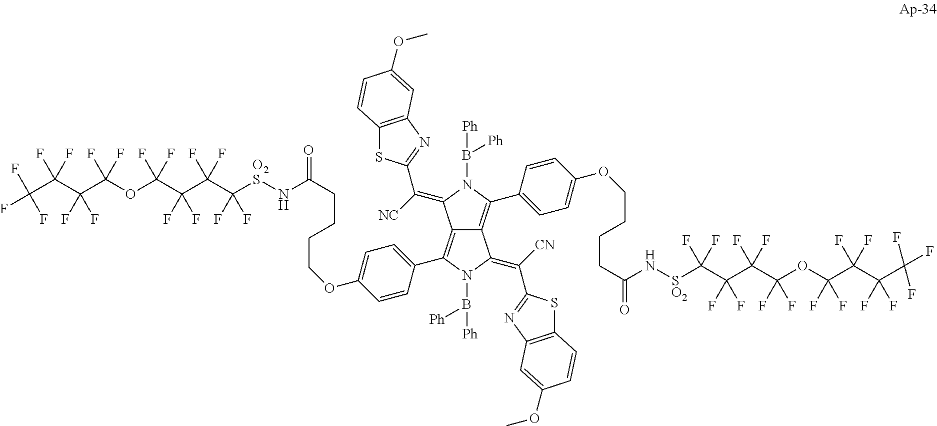 [SMILES: COc1ccc2sc(/C(C#N)=c3/c4c(-c5ccc(OCCCCC(=O)NS(=O)(=O)C(F)(F)C(F)(F)C(F)(F)C(F)(F)OC(F)(F)C(F)(F)C(F)(F)C(F)(F)F)cc5)n(B(c5ccccc5)c5ccccc5)/c(=C(/C#N)c5nc6cc(OC)ccc6s5)c4c(-c4ccc(OCCCCC(=O)NS(=O)(=O)C(F)(F)C(F)(F)C(F)(F)C(F)(F)OC(F)(F)C(F)(F)C(F)(F)C(F)(F)F)cc4)n3B(c3ccccc3)c3ccccc3)nc2c1]

### Test Example 1

<Manufacturing of Dispersion>

3 parts by mass of a specific compound A shown in the following table, 10 parts by mass of a colorant B shown in the following table, 150 parts by mass of a solvent C shown in the following table, 7.8 parts by mass of a resin D shown in the following Table, and 230 parts by mass of zirconia beads having a diameter of 0.3 mm were mixed with each other, the mixture was dispersed using a paint shaker for 5 hours, and the beads were separated by filtration. As a result, a dispersion was manufactured. In Example 4, as the solvent C, 140 parts by mass of C-1 and 10 parts by mass of C-9 were used. In addition, in Example 9, as the specific compound A, 1.5 parts by mass of Ap-1 and 1.5 parts by mass of Ap-2 were used. In addition, in Example 10, as the colorant B, 5 parts by mass of P-1 and 5 parts by mass of P-17 were used. In addition, in Example 11, as the colorant B, 6 parts by mass of P-1 and 4 parts by mass of P-18 were used. In addition, in Example 12, as the colorant B, 8 parts by mass of P-1 and 2 parts by mass of SQ-1 were used. In addition, in Example 13, as the colorant B, 8 parts by mass of P-1 and 2 parts by mass of SQ-7 were used. In addition, in Example 14, as the colorant B, 6 parts by mass of P-1 and 4 parts by mass of SQ-9 were used. In addition, in Example 15, as the colorant B, 6 parts by mass of P-1 and 6 parts by mass of CY-2 were used. In addition, in Example 34, as the colorant B, 6 parts by mass of Dp-1, 2 parts by mass of Pc-1, and 2 parts by mass of II-1 were used.

<Evaluation of Dispersibility>

(Viscosity)

Using an E-type viscometer, the viscosity of the dispersion at 25° C. was measured at a rotation speed of 1000 rpm and was evaluated based on the following criteria.

A: 1 mPa·s to 15 mPa·s

B: higher than 15 mPa·s and 100 mPa·s or lower

C: higher than 100 mPa·s

(Thixotropy)

Using an E-type viscometer, the viscosity of the dispersion at 25° C. was measured at a rotation speed of 20 rpm and at a rotation speed of 50 rpm, the viscosity at a rotation speed of 20 rpm/the viscosity at a rotation speed of 50 rpm was defined as a thixotropy index (TI value), and the TI value was evaluated based on the following criteria.

A: the TI value was 1 to 1.3

B: the TI value was higher than 1.3 and 2 or lower

C: the TI value was higher than 2

(Particle Size)

The volume average particle size of a pigment in the dispersion immediately after manufacturing was measured using MICROTRAC UPA 150 (manufactured by Nikkiso Co., Ltd.). The average particle size of particles of the specific compound A and the colorant B as the pigment were measured.

A: 5 nm to 50 nm

B: higher than 50 nm and 500 nm or lower

C: higher than 500 nm

TABLE 9

| | | | | | Dispersibility | | |
|---|---|---|---|---|---|---|---|
| | Specific | | | | | | Particle |
| | Compound A | Colorant B | Solvent C | Resin D | Viscosity | Thixotropy | Size |
| Example 1 | Ap-1 | P-1 | C-1 | D-4 | A | A | A |
| Example 2 | Ap-1 | P-1 | C-3 | D-2 | A | A | A |
| Example 3 | Ap-1 | P-1 | C-8 | D-2 | A | A | A |
| Example 4 | Ap-1 | P-1 | C-1 + C-9 | D-2 | A | A | A |
| Example 5 | Ap-1 | P-2 | C-1 | D-4 | A | A | A |
| Example 6 | Ap-1 | P-6 | C-1 | D-3 | A | B | A |
| Example 7 | Ad-1 | P-1 | C-1 | D-2 | B | B | B |
| Example 8 | An-2 | P-1 | C-1 | D-2 | B | B | B |
| Example 9 | Ap-1 + Ap-2 | P-1 | C-1 | D-2 | A | A | A |
| Example 10 | Ad-17 | P-1 | C-1 | D-2 | B | B | B |
| Example 11 | Ap-1 | P-1 + P-17 | C-1 | D-2 | A | A | A |
| Example 12 | Ap-1 | P-1 + P-18 | C-1 | D-2 | A | A | A |
| Example 13 | Ap-5 | P-1 + SQ-1 | C-1 | D-2 | B | B | B |
| Example 14 | Ap-5 | P-1 + SQ-7 | C-1 | D-2 | B | B | B |
| Example 15 | Ap-5 | P-1 + SQ-9 | C-1 | D-2 | B | B | B |
| Example 16 | Ap-5 | P-1 + CY-2 | C-9 | D-2 | B | B | B |
| Example 17 | Ap-5 | P-15 | C-1 | D-2 | B | B | B |
| Example 18 | Ap-18 | P-15 | C-1 | D-2 | B | B | B |
| Example 19 | Ap-25 | P-15 | C-1 | D-2 | A | A | A |
| Example 20 | Ap-5 | P-18 | C-1 | D-2 | B | B | B |
| Example 21 | Ap-17 | P-18 | C-1 | D-2 | A | A | A |
| Example 22 | Ap-5 | SQ-1 | C-1 | D-2 | B | B | B |
| Example 23 | Ap-5 | SQ-8 | C-3 | D-2 | B | B | B |
| Example 24 | Ap-5 | SQ-9 | C-8 | D-2 | B | B | B |
| Example 25 | As-5 | SQ-9 | C-3 | D-2 | A | A | A |
| Example 26 | Ap-5 | SQ-10 | C-3 | D-2 | B | B | B |
| Example 27 | Ap-5 | SQ-11 | C-3 | D-2 | B | B | B |
| Example 28 | Ap-5 | CY-1 | C-4 | D-4 | B | B | B |
| Example 29 | Ap-5 | CR-3 | C-3 | D-2 | B | B | B |
| Example 30 | Ac-1 | CR-3 | C-3 | D-2 | A | A | A |
| Example 31 | Ap-5 | Dp-1 | C-5 | D-4 | B | B | B |
| Example 32 | Ap-5 | Pc-1 | C-1 | D-4 | B | B | B |
| Example 33 | Ap-5 | Pc-4 | C-7 | D-4 | B | B | B |
| Example 34 | Ap-5 | Pc-2 | C-1 | D-4 | B | B | B |
| Example 35 | Ap-5 | Pc-3 | C-6 | D-4 | B | B | B |
| Example 36 | Ap-5 | Dp-1 + Pc-1 + II-1 | C-1 | D-4 | B | B | B |
| Example 37 | Ap-5 | Pr-1 | C-1 | D-4 | B | B | B |
| Example 38 | Ap-1 | — | C-3 | D-4 | A | A | A |
| Example 39 | Ap-1 | P-1 | C-2 | D-2 | A | A | B |
| Example 40 | Ap-14 | P-1 | C-1 | D-4 | A | A | A |
| Example 41 | Ap-10 | P-1 | C-1 | D-4 | B | B | B |
| Example 42 | Ap-5 | P-1 | C-1 | D-4 | B | B | B |
| Example 43 | Ap-26 | P-1 | C-1 | D-4 | A | A | A |
| Example 44 | Ap-27 | P-1 | C-1 | D-4 | A | A | A |
| Example 45 | Ap-33 | P-1 | C-1 | D-4 | A | A | A |
| Example 46 | Ap-34 | P-1 | C-1 | D-4 | A | A | A |
| Comparative | a-1 | P-1 | C-1 | D-4 | B | B | B |
| Example 1 | | | | | | | |
| Comparative | a-2 | P-1 | C-1 | D-4 | C | C | C |
| Example 2 | | | | | | | |

TABLE 10

| | Specific | | | | Dispersibility | | |
|---|---|---|---|---|---|---|---|
| | Compound | | | | | | Particle |
| | A | Colorant B | Solvent C | Resin D | Viscosity | Thixotropy | Size |
| Example 47 | Ap-35 | P-21 | C-1 | D-4 | B | A | B |
| Example 48 | Ap-35 | P-22 | C-1 | D-4 | B | A | B |
| Example 49 | Ap-48 | P-22 | C-1 | D-4 | B | A | B |
| Example 50 | Ap-61 | P-22 | C-1 | D-4 | A | A | A |
| Example 51 | Ap-74 | P-22 | C-1 | D-4 | A | A | A |
| Example 52 | Ap-87 | P-22 | C-1 | D-4 | A | A | A |
| Example 53 | Ap-100 | P-22 | C-1 | D-4 | A | A | A |
| Example 54 | Ap-35 | P-23 | C-1 | D-4 | B | A | B |
| Example 55 | Ap-48 | P-23 | C-1 | D-4 | B | A | B |
| Example 56 | Ap-61 | P-23 | C-1 | D-4 | A | A | A |
| Example 57 | Ap-74 | P-23 | C-1 | D-4 | A | A | A |
| Example 58 | Ap-87 | P-23 | C-1 | D-4 | A | A | A |
| Example 59 | Ap-100 | P-23 | C-1 | D-4 | A | A | A |
| Example 60 | Ap-35 | P-24 | C-1 | D-4 | B | A | B |
| Example 61 | Ap-48 | P-24 | C-1 | D-4 | B | A | B |
| Example 62 | Ap-61 | P-24 | C-1 | D-4 | A | A | A |
| Example 63 | Ap-74 | P-24 | C-1 | D-4 | A | A | A |
| Example 64 | Ap-87 | P-24 | C-1 | D-4 | A | A | A |
| Example 65 | Ap-100 | P-24 | C-1 | D-4 | A | A | A |
| Example 66 | Ap-36 | P-25 | C-1 | D-4 | B | A | B |
| Example 67 | Ap-49 | P-25 | C-1 | D-4 | B | A | B |
| Example 68 | Ap-62 | P-25 | C-1 | D-4 | A | A | A |
| Example 69 | Ap-75 | P-25 | C-1 | D-4 | A | A | A |
| Example 70 | Ap-88 | P-25 | C-1 | D-4 | A | A | A |
| Example 71 | Ap-101 | P-25 | C-1 | D-4 | A | A | A |
| Example 72 | Ap-36 | P-26 | C-1 | D-4 | B | A | B |
| Example 73 | Ap-49 | P-26 | C-1 | D-4 | B | A | B |
| Example 74 | Ap-62 | P-26 | C-1 | D-4 | A | A | A |
| Example 75 | Ap-75 | P-26 | C-1 | D-4 | A | A | A |
| Example 76 | Ap-88 | P-26 | C-1 | D-4 | A | A | A |
| Example 77 | Ap-101 | P-26 | C-1 | D-4 | A | A | A |
| Example 78 | Ap-36 | P-27 | C-1 | D-4 | B | A | B |
| Example 79 | Ap-49 | P-27 | C-1 | D-4 | B | A | B |
| Example 80 | Ap-62 | P-27 | C-1 | D-4 | A | A | A |
| Example 81 | Ap-75 | P-27 | C-1 | D-4 | A | A | A |
| Example 82 | Ap-88 | P-27 | C-1 | D-4 | A | A | A |
| Example 83 | Ap-101 | P-27 | C-1 | D-4 | A | A | A |
| Example 84 | Ap-63 | P-28 | C-1 | D-4 | A | A | A |
| Example 85 | Ap-64 | P-29 | C-1 | D-4 | A | A | A |

TABLE 11

| | | | | | Dispersibility | | |
|---|---|---|---|---|---|---|---|
| | Specific | | | | | | Particle |
| | Compound A | Colorant B | Solvent C | Resin D | Viscosity | Thixotropy | Size |
| Example 86 | Ap-39 | P-30 | C-1 | D-4 | B | A | B |
| Example 87 | Ap-52 | P-30 | C-1 | D-4 | B | A | B |
| Example 88 | Ap-65 | P-30 | C-1 | D-4 | A | A | A |
| Example 89 | Ap-78 | P-30 | C-1 | D-4 | A | A | A |
| Example 90 | Ap-91 | P-30 | C-1 | D-4 | A | A | A |
| Example 91 | Ap-104 | P-30 | C-1 | D-4 | A | A | A |
| Example 92 | Ap-79 | P-31 | C-1 | D-4 | A | A | A |
| Example 93 | Ap-80 | P-32 | C-1 | D-4 | A | A | A |
| Example 94 | Ap-81 | P-33 | C-1 | D-4 | A | A | A |
| Example 95 | Ap-82 | P-34 | C-1 | D-4 | A | A | A |
| Example 96 | Ap-83 | P-35 | C-1 | D-4 | A | A | A |
| Example 97 | Ap-45 | P-36 | C-1 | D-4 | B | A | B |
| Example 98 | Ap-58 | P-36 | C-1 | D-4 | B | A | B |
| Example 99 | Ap-71 | P-36 | C-1 | D-4 | A | A | A |
| Example 100 | Ap-84 | P-36 | C-1 | D-4 | A | A | A |
| Example 101 | Ap-97 | P-36 | C-1 | D-4 | A | A | A |
| Example 102 | Ap-110 | P-36 | C-1 | D-4 | A | A | A |
| Example 103 | Ap-46 | P-37 | C-1 | D-4 | B | A | B |
| Example 104 | Ap-59 | P-37 | C-1 | D-4 | B | A | B |
| Example 105 | Ap-72 | P-37 | C-1 | D-4 | A | A | A |
| Example 106 | Ap-85 | P-37 | C-1 | D-4 | A | A | A |
| Example 107 | Ap-98 | P-37 | C-1 | D-4 | A | A | A |
| Example 108 | Ap-111 | P-37 | C-1 | D-4 | A | A | A |
| Example 109 | Ap-47 | P-38 | C-1 | D-4 | B | A | B |
| Example 110 | Ap-60 | P-38 | C-1 | D-4 | B | A | B |
| Example 111 | Ap-73 | P-38 | C-1 | D-4 | A | A | A |
| Example 112 | Ap-86 | P-38 | C-1 | D-4 | A | A | A |
| Example 113 | Ap-99 | P-38 | C-1 | D-4 | A | A | A |
| Example 114 | Ap-112 | P-38 | C-1 | D-4 | A | A | A |
| Example 115 | Ap-35 | P-39 | C-1 | D-4 | B | A | B |
| Example 116 | Ap-35 | P-40 | C-1 | D-4 | B | A | B |
| Example 117 | Ap-36 | P-41 | C-1 | D-4 | B | A | B |
| Example 118 | Ap-36 | P-42 | C-1 | D-4 | B | A | B |
| Example 119 | Ap-36 | P-43 | C-1 | D-4 | B | A | B |
| Example 120 | Ap-47 | P-44 | C-1 | D-4 | B | A | B |
| Example 121 | Ap-47 | P-45 | C-1 | D-4 | B | A | B |
| Example 122 | Ap-47 | P-46 | C-1 | D-4 | B | A | B |
| Example 123 | Ap-47 | P-47 | C-1 | D-4 | B | A | B |

As shown in the tables, the dispersibility of the dispersions according to Examples was excellent

The raw materials shown above in the table are as follows.

(Specific Compound A)

Ap-1, Ap-2, Ap-5, Ap-10, Ap-14, Ap-17, Ap-18, Ap-25, Ap-26, Ap-27, Ap-33, Ap-34, Ad-1, An-2, As-5, Ac-1: compounds having the following structures These compounds are compounds having a structure in which the functional group A is bonded to a π-conjugated structure of a colorant skeleton. In addition, Ap-1, Ap-2, Ap-5, Ap-10, Ap-14, Ap-17, Ap-18, Ap-25, Ap-26, Ap-27, Ap-33, Ap-34, Ac-1, and As-5 are compounds having a structure in which the functional group A is bonded to a r-conjugated structure of a colorant skeleton and having a maximum absorption wavelength in a wavelength range of 650 to 1200 nm. In Ap-1, Ap-2, Ap-25, Ad-1, and As-5, the functional group A is a group represented by the following (a-1) (an acid group having a pKa of −1.43 and a Clog P value of 1.09). In Ap-17, the functional group A is a group represented by the following (a-13) (an acid group having a pKa of 0.43 and a Clog P value of 2.92). In Ap-18, the functional group A is a group represented by the following (a-14) (an acid group having a pKa of 0.27 and a Clog P value of 2.74). In Ap-26, the functional group A is a group represented by the following (a-31) (an acid group having a pKa of 0.27 and a Clog P value of 1.71). In Ap-27, the functional group A is a group represented by the following (a-35) (an acid group having a pKa of 1.35 and a Clog P value of −0.08). In Ap-33, the functional group A is a group represented by the following (a-32) (an acid group having a pKa of 0.38 and a Clog P value of 2.74). In Ap-33, the functional group A is a group represented by the following (a-34) (an acid group having a pKa of 0.92 and a Clog P value of 6.14). In Ac-1, the functional group A is a group represented by the following (a-19) (an acid group having a pKa of −1.37 and a Clog P value of 3.42). In Ap-14, the functional group A is a group represented by the following (a-10) (an acid group having a pKa of 1.43 and a Clog P value of 1.09). In Ap-10, the functional group A is a group represented by the following (a-6) (an acid group having a pKa of 1.84 and a Clog P value of −0.52). In Ap-5, the functional group A is a group represented by the following (a-2) (an acid group having a pKa of 2.88 and a Clog P value of 1.20). In An-2, the functional group A is a group represented by the following (a-28) (an acid group having a pKa of −0.26 and a Clog P value of 2.597).

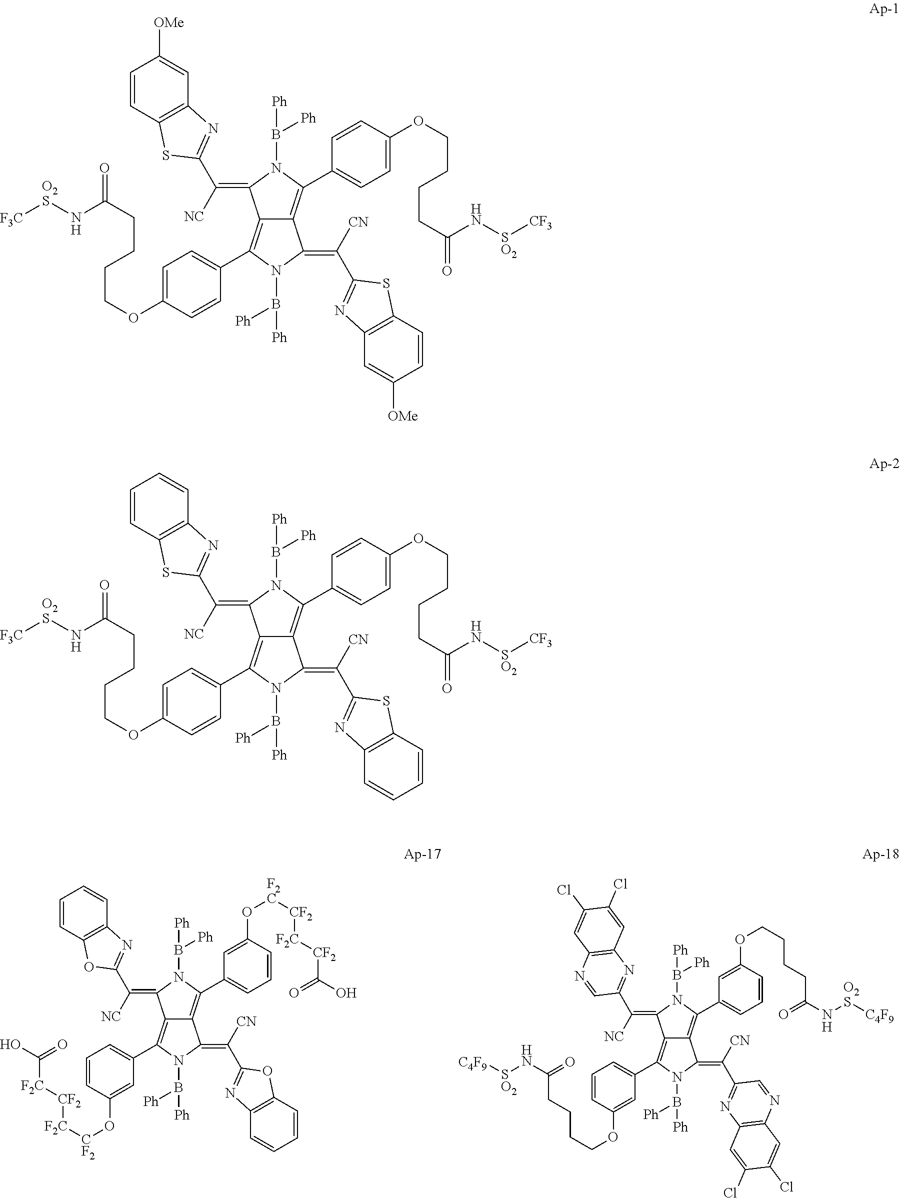

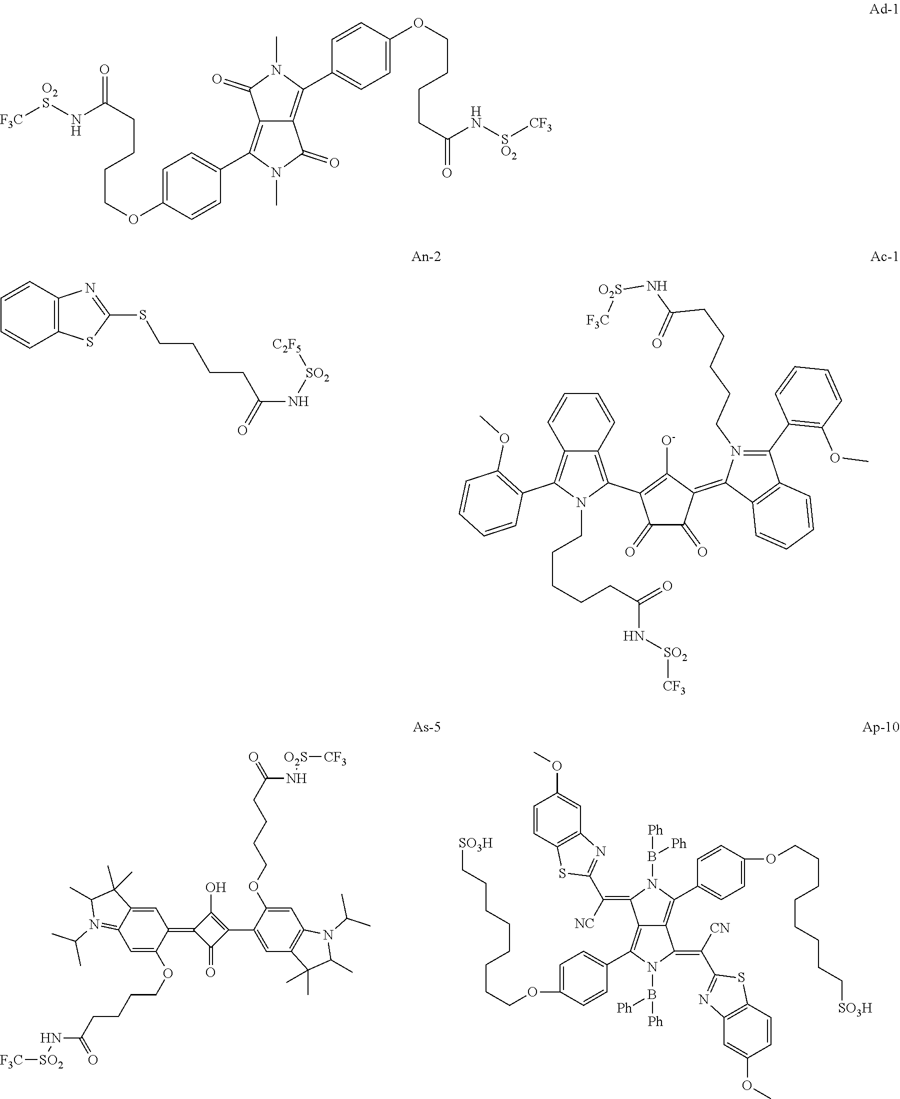

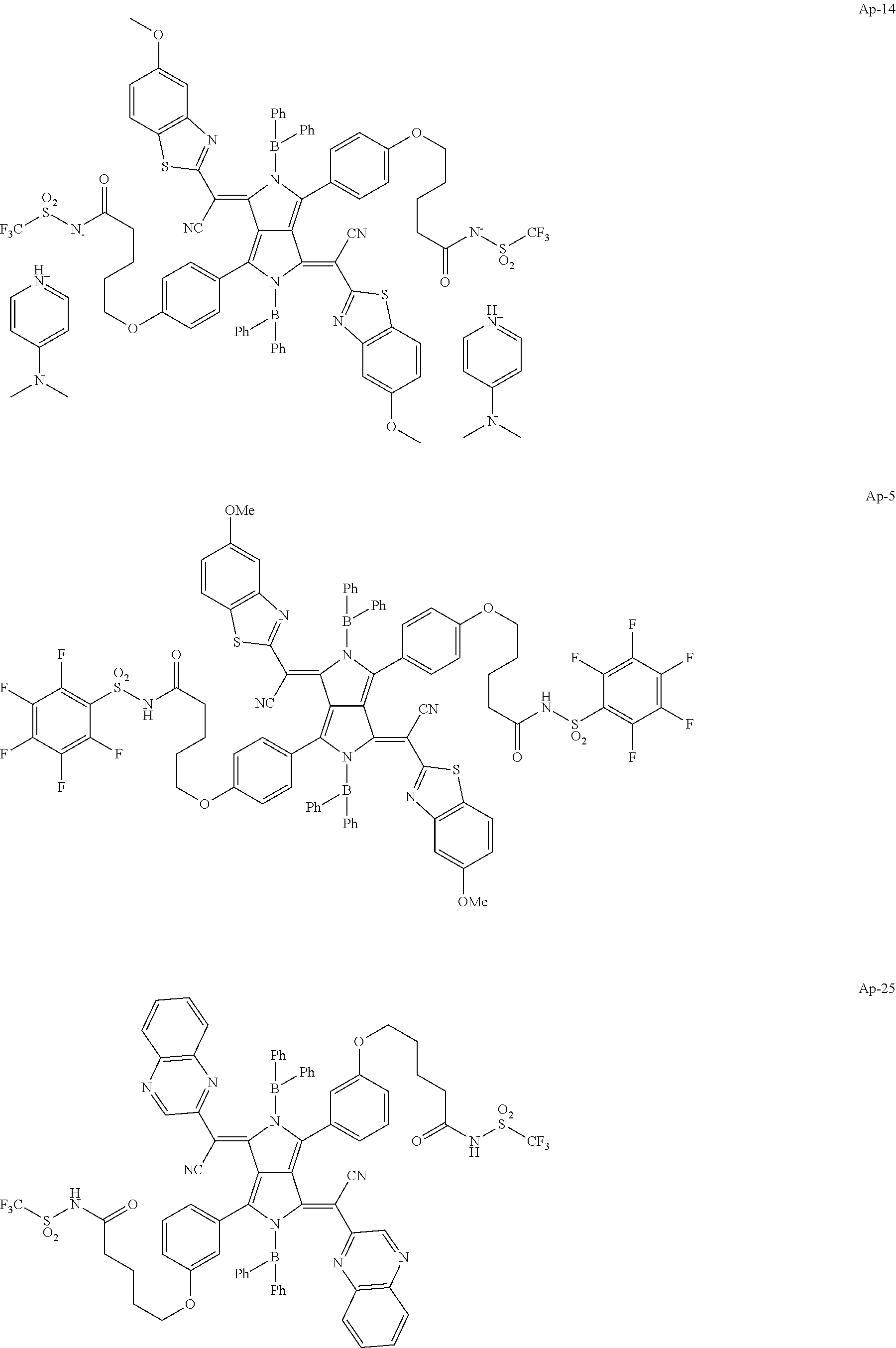

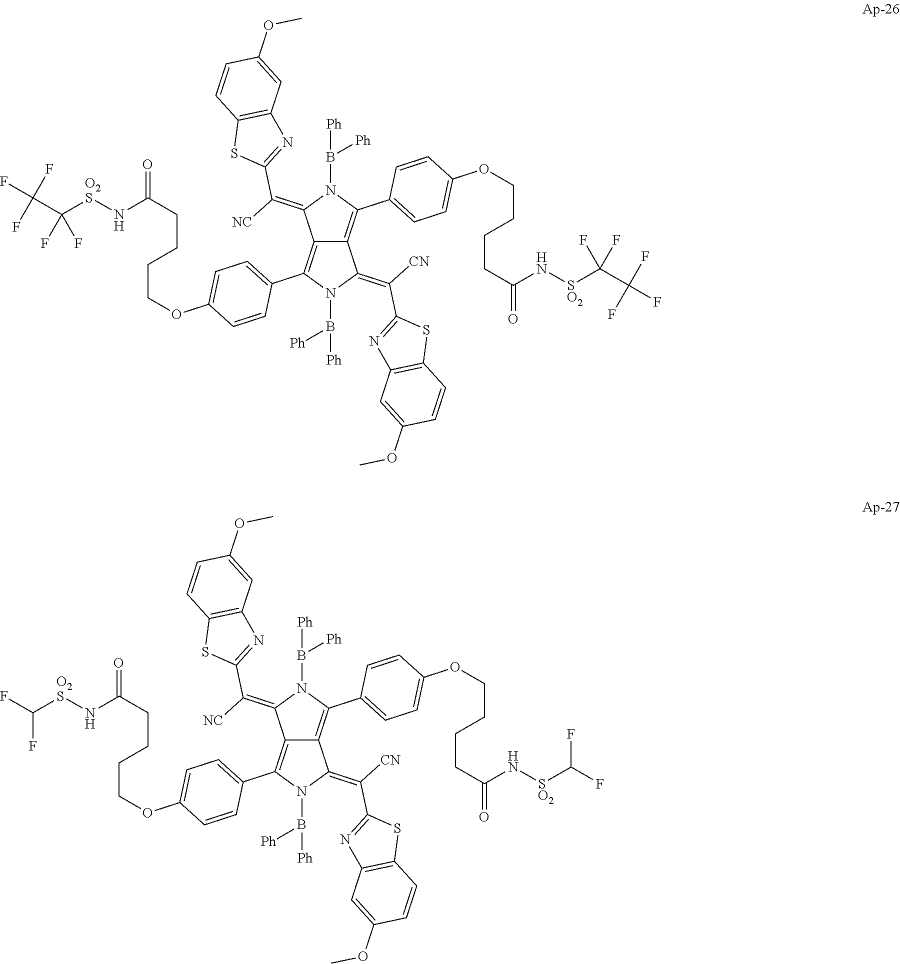 [SMILES: COc1ccc2sc(/C(C#N)=c3/c4c(-c5ccc(OCCCCC(=O)NS(=O)(=O)C(F)(F)C(F)(F)F)cc5)n(B(c5ccccc5)c5ccccc5)/c(=C(/C#N)c5nc6cc(OC)ccc6s5)c4c(-c4ccc(OCCCCC(=O)NS(=O)(=O)C(F)(F)C(F)(F)F)cc4)n3B(c3ccccc3)c3ccccc3)nc2c1 ; COc1ccc2sc(/C(C#N)=c3/c4c(-c5ccc(OCCCCC(=O)NS(=O)(=O)C(F)F)cc5)n(B(c5ccccc5)c5ccccc5)/c(=C(/C#N)c5nc6cc(OC)ccc6s5)c4c(-c4ccc(OCCCCC(=O)NS(=O)(=O)C(F)F)cc4)n3B(c3ccccc3)c3ccccc3)nc2c1]

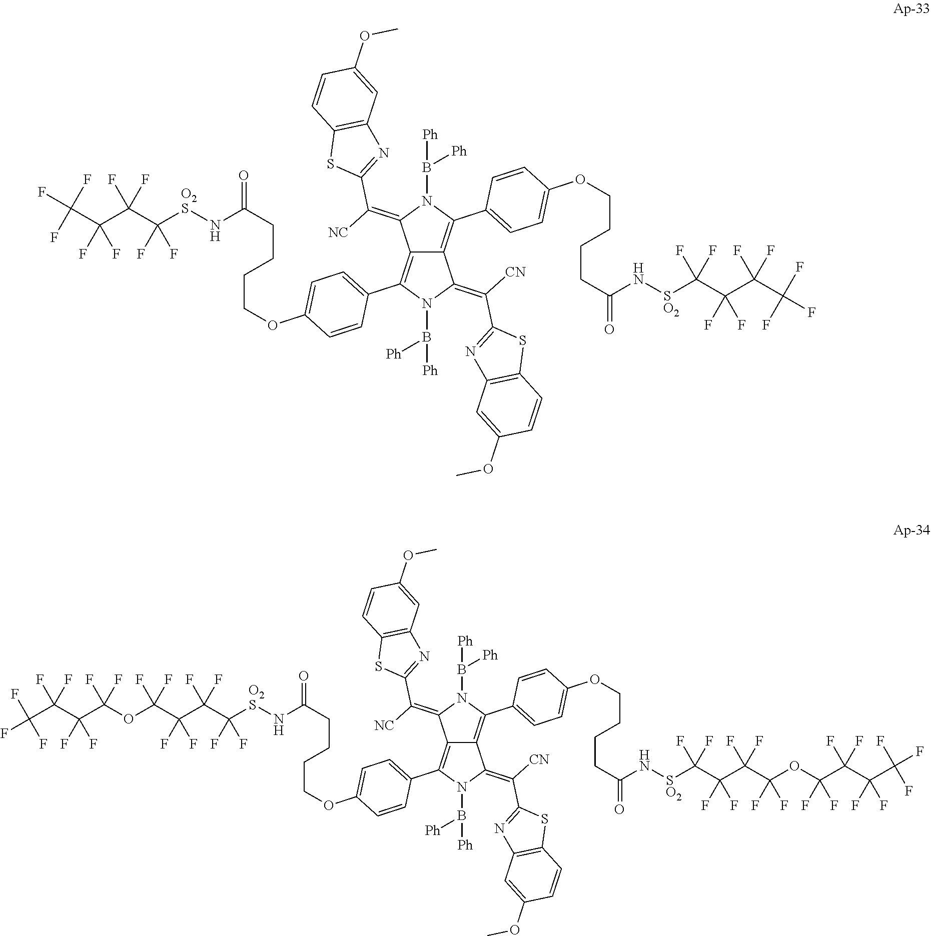

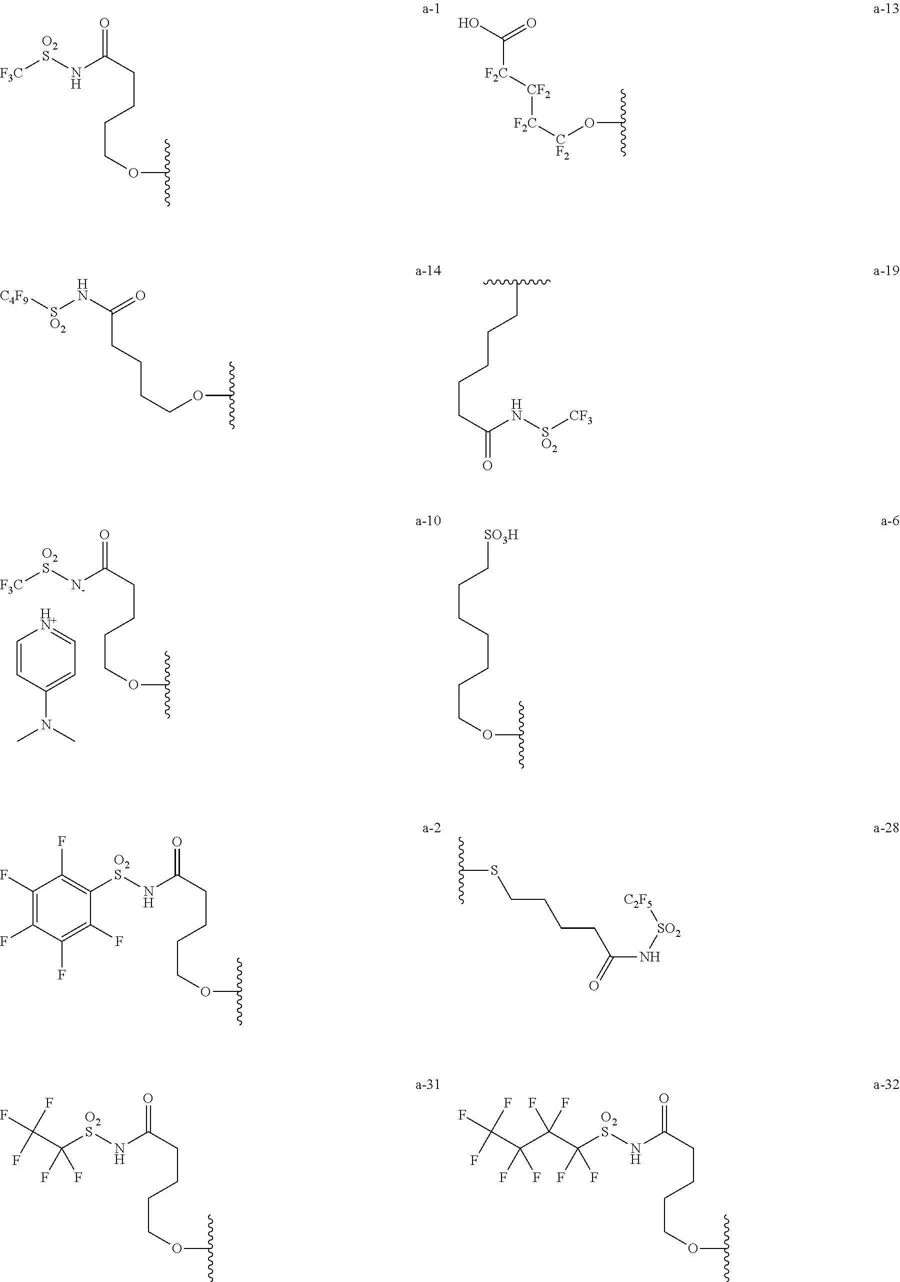

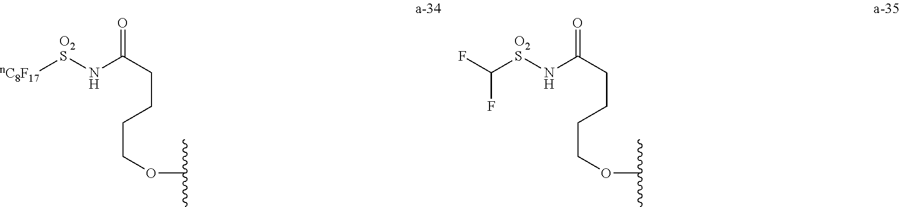

Ap-35, Ap-36, Ap-39, Ap-45, Ap-46, Ap-47, Ap-48, Ap-49, Ap-52, Ap-58, Ap-59, Ap-60, Ap-61, Ap-62, Ap-63, Ap-64, Ap-65, Ap-71, Ap-72, Ap-73, Ap-74, Ap-75, AP-78, Ap-79, Ap-80, Ap-81, Ap-82, Ap-83, Ap-84, Ap-85, Ap-86, Ap-87, Ap-88, Ap-91, Ap-97, Ap-98, Ap-99, Ap-100, Ap-101, Ap-104, Ap-110, Ap-111, Ap-112: compounds having the structures shown in the specific examples of the compound A. These compounds are compounds having a structure in which the functional group A is bonded to a it-conjugated structure of a colorant skeleton and having a maximum absorption wavelength in a wavelength range of 650 to 1200 nm. In Ap-35, Ap-36, Ap-39, Ap-45, Ap-46, and Ap-47, the functional group A is a group represented by the following (a-47) (an acid group having a pKa of 1.64 and a Clog P value of −0.32). In Ap-48, Ap-49, Ap-52, Ap-58, Ap-59, and Ap-60, the functional group A is a group represented by the following (a-48) (an acid group having a pKa of 1.60 and a Clog P value of 0.48). In Ap-61, Ap-62, Ap-63, Ap-64, Ap-65, Ap-71, Ap-72, and Ap-73, the functional group A is a group represented by the following (a-38) (an acid group having a pKa of −1.44 and a Clog P value of 2.76). In Ap-74, Ap-75, Ap-78, Ap-79, Ap-80, Ap-81, Ap-82, Ap-83, Ap-84, Ap-85, and Ap-86, the functional group A is a group represented by the following (a-49) (an acid group having a pKa of −1.46 and a Clog P value of 3.42). In Ap-87, Ap-88, Ap-91, Ap-97, Ap-98, and Ap-99, the functional group A is a group represented by the following (a-50) (an acid group having a pKa of 0.26 and a Clog P value of 3.38). In Ap-100, Ap-101, Ap-104, Ap-110, Ap-111, and Ap-112, the functional group A is a group represented by the following (a-51) (an acid group having a pKa of 0.25 and a Clog P value of 4.04).

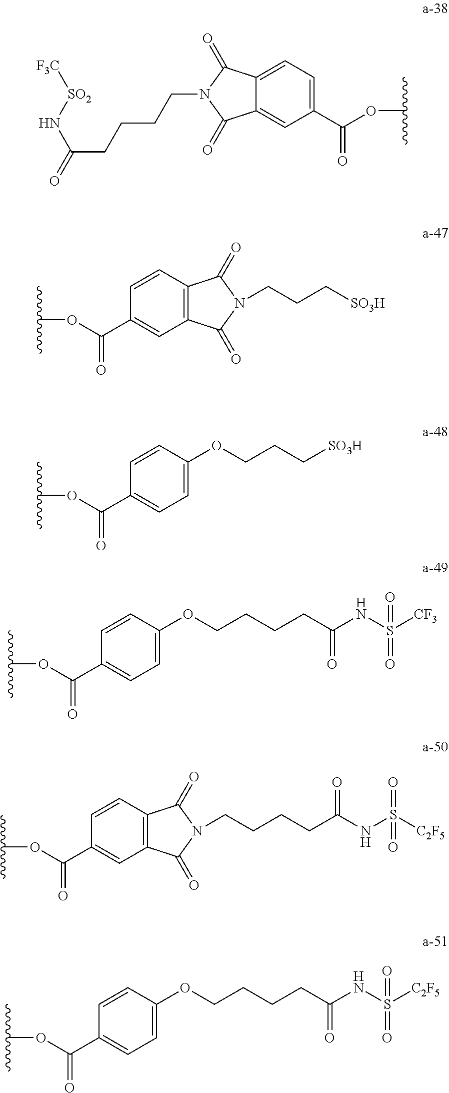

a-1, a-2: compounds having the following structures A compound a-1 is a compound having a structure in which a —SO₃H group (an acid group having a pKa of 1.75 and a Clog P value of −2.42) is bonded to a π-conjugated structure of a colorant skeleton. In addition, a compound a-2 is a compound having a structure in which a —C₆H₁₂—COOH group (an acid group having a pKa of 4.78 and a Clog P value of 2.98) is bonded to a it-conjugated structure of a colorant skeleton.

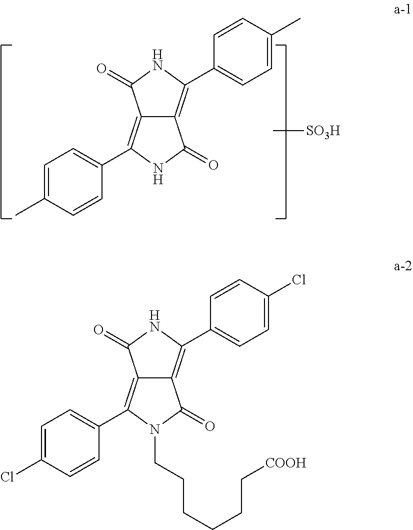

(Colorant B)

P-1, P-2, P-6, P-15, P-17, P-18, P-21 to P-47, SQ-1, SQ-7, SQ-8, SQ-9, SQ-10, SQ-11, CY-1, CY-2, CR-3, Dp-1, Pc-1, Pc-2, Pc-3, Pc-4, II-1, Pr-1: P-1, P-2, P-6, P-15, P-17, P-18, P-21 to P-47, SQ-1, SQ-7, SQ-8, SQ-9, SQ-10, SQ-11, CY-1, CY-2, CR-3, Dp-1, Pc-1, Pc-2, Pc-3, Pc-4, II-1, and Pr-1 described as the specific examples of the other colorant

(Solvent C)

C-1: propylene glycol monomethyl ether acetate (PGMEA)

C-2: butanol

C-3: propylene glycol monomethyl ether (PGME)

C-4: lactic acid

C-5: butyl acetate

C-6: cyclopentanone

C-7: cyclohexanone

C-8: 3-methoxy-N,N-dimethylpropanamide

C-9: 3-butoxy-N,N-dimethylpropanamide

(Resin D)

In the following structure, a numerical value added to a main chain represents a molar ratio of a repeating unit, and a numerical value added to a side chain represents the number of repeating units.

D-2: a resin having the following structure (acid value=32.3 mgKOH/g, amine value=45.0 mgKOH/g, weight-average molecular weight=22900)

D-3: a resin having the following structure (acid value=44.3 mgKOH/g, amine value=40.0 mgKOH/g, weight-average molecular weight=10424)

D-4: a resin having the following structure (acid value=36.0 mgKOH/g, amine value=47.0 mgKOH/g, weight-average molecular weight=20903)

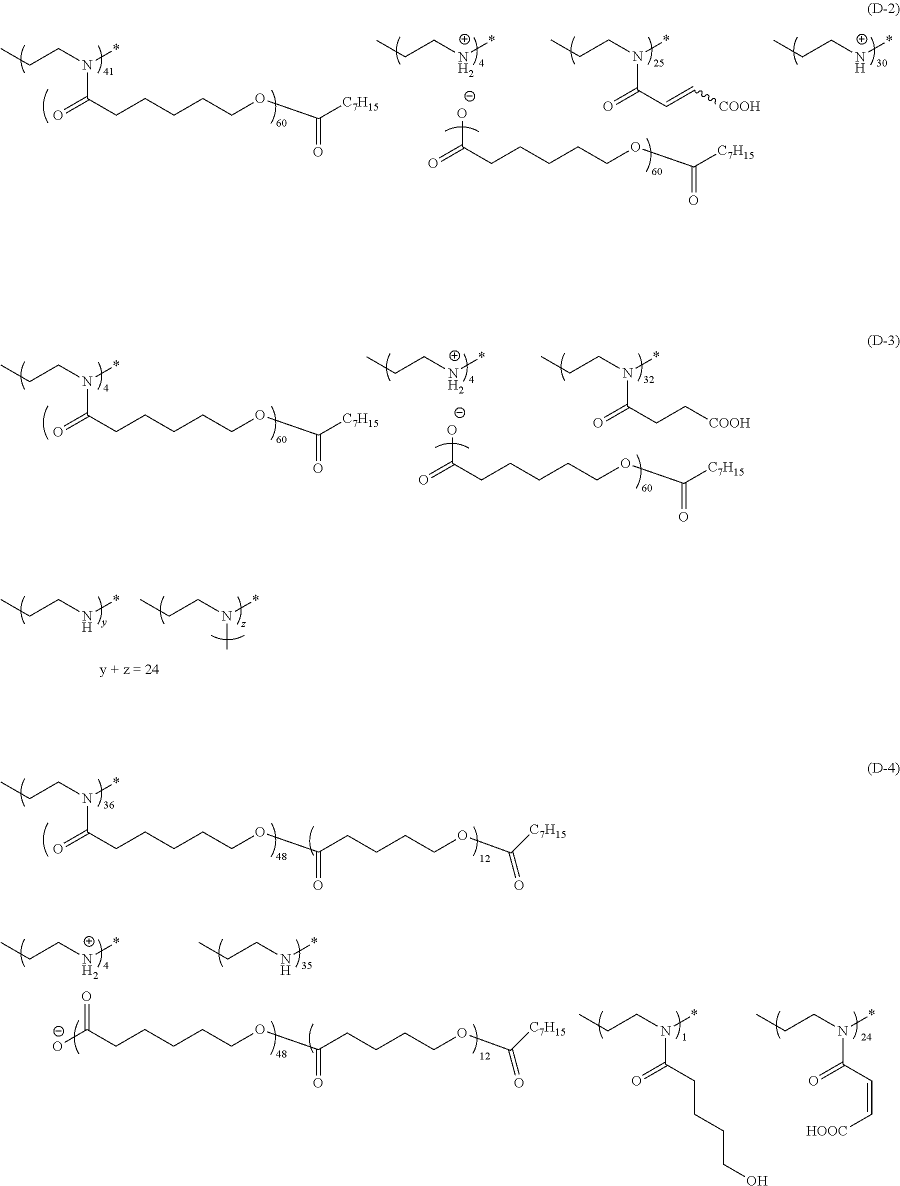

### Test Example 2

<Manufacturing of Curable Composition>

(Manufacturing Example 101)

The following components were mixed with each other to prepare a curable composition according to Manufacturing Example 101.
Dispersion of Example 1: 55 parts by mass
Alkali-Soluble Resin (ACRYBASE FF-426, manufactured by Nippon Shokubai Co., Ltd.): 7.0 parts by mass
Curable compound (ARONIX M-305, a mixture of pentaerythritol triacrylate and pentaeiythritol tetraacrylate, containing 55 to 63 mass % of pentaerythritol triacrylate, manufactured by Toagosei Co., Ltd.): 4.5 parts by mass
Photoradical polymerization initiator (IRGACURE-OXE02, manufactured by BASF SE): 0.8 parts by mass
Polymerization inhibitor (p-methoxyphenol): 0.001 parts by mass
Surfactant (the following mixture (Mw=14000); in the following formula, “%” representing the proportion of a repeating unit is mol %): 0.03 parts by mass

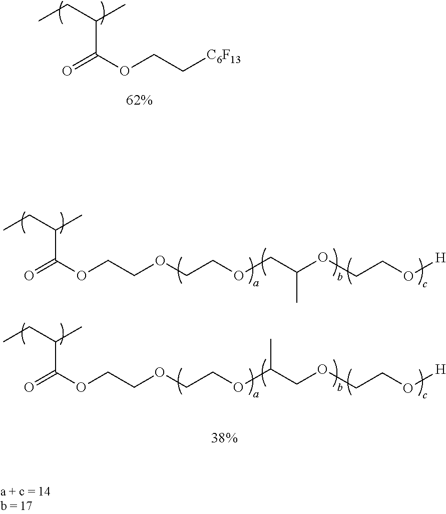

Ultraviolet absorber (UV-503, manufactured by Daito Chemical Co., Ltd.): 1.3 parts by mass
Solvent (propylene glycol monomethyl ether acetate): 31 parts by mass

<Manufacturing Examples 102 to 105 and 108 to 225>

Each of curable compositions was manufactured using the same method as that of the curable composition according to Manufacturing Example 101, except that a dispersion shown in the following table was used instead of the dispersion.

<Manufacturing Example 106>

A curable composition was manufactured using the same method as that of Manufacturing Example 101, except that a dispersion according to Example 6 was used instead of the dispersion and 25 parts by mass of propylene glycol monomethyl ether acetate and 6 parts by mass of 3-methoxy-N,N-dimethylpropanamide were used as the solvent.

<Manufacturing Example 107>

A curable composition was manufactured using the same method as that of Manufacturing Example 101, except that a dispersion according to Example 7 was used instead of the dispersion and 20 parts by mass of propylene glycol monomethyl ether acetate and 11 parts by mass of 3-butoxy-N,N-dimethylpropanamide were used as the solvent.

<Preparation of Cured Film>

The curable composition was applied to a glass substrate using a spin coating method and then was heated using a hot plate at 100° C. for 2 minutes. As a result, a composition layer was obtained. The obtained composition layer was exposed using an i-ray stepper at an exposure dose of 500 mJ/cm². Next, the exposed coating layer was cured using a hot plate at 220° C. for 5 minutes. As a result, a cured film having a thickness of 0.7 m was obtained.

<Evaluation of Moisture Resistance>

Each of the curable compositions was applied to a glass substrate using a spin coater (manufactured by Mikasa Co., Ltd.) such that the thickness after pre-baking was 0.8 μm. As a result, a coating film was formed. Next, the coating film was heated (pre-baked) using a hot plate at 100° C. for 120 seconds. Next, the entire surface of the coating film was exposed using an i-ray stepper exposure device FPA-3000 i5+(manufactured by Canon Corporation) at an exposure dose of 1000 mJ/cm² and then was heated (post-baked) again using a hot plate at 200° C. for 300 seconds. As a result, a film was obtained. Regarding the obtained film, the transmittance of light in a wavelength range of 700 to 1000 nm was measured. Next, this film was put into a constant-temperature tank at 85° C. and a humidity of 95% and was stored therein for 6 months to perform a moisture-resistance test. Regarding the film after the moisture-resistance test, the transmittance of light at each wavelength in a wavelength range of 700 to 1000 nm was measured. The transmittance of the film was measured using a spectrophotometer (manufactured by Hitachi High-Technologies Corporation, U-4100).

A maximum value (ΔT) of a change in transmittance at each wavelength in a wavelength range of 700 to 1000 nm before and after the moisture-resistance test was measured and was set as an index indicating moisture resistance.

Change (ΔT) in Transmittance=|Transmittance (%) of Film before Moisture-Resistance Test-Transmittance (%) of Film after Moisture-Resistance Test|

A: ΔT %<4%

B: 4%<ΔT %<10%

C: 10%<ΔT %

<Evaluation of Aggregate derived from Compound having Colorant Skeleton>

Each of the curable compositions was applied to a glass substrate using a spin coater (manufactured by Mikasa Co., Ltd.) such that the thickness after pre-baking was 0.8 m. As a result, a coating film was formed. Next, the coating film was heated (pre-baked) using a hot plate at 100° C. for 120 seconds. Next, the entire surface of the coating film was exposed using an i-ray stepper exposure device FPA-3000 i5+(manufactured by Canon Corporation) at an exposure dose of 1000 mJ/cm² and then was heated (post-baked) again using a hot plate at 200° C. for 300 seconds. As a result, a film was obtained. The obtained film was observed (measurement magnification=10000 times) using a scanning electron microscope, and the number of foreign matter particles present in a range of 10 μm×15 μm was measured.

A: no foreign matter was present in a range of 10 μm×15 μm

B: the number of foreign matter particles present in a range of 10 μm×15 μm was more than 0 and 100 or less

C: the number of foreign matter particles present in a range of 10 mx15 μm was more than 100

<Evaluation of Developability>

Each of the curable compositions was applied to a silicon wafer with an undercoat layer using a spin coating method such that the thickness after the application was 0.7 μm, and then was heated using a hot plate at 100° C. for 2 minutes. As a result, a curable composition layer was obtained. Next, using an i-ray stepper exposure device FPA-3000 i5+(manufactured by Canon Corporation), the obtained curable composition layer was exposed (an optimum exposure dose was selected such that the line width was 1.1 μm) through a mask having a 1.1 μm×1.1 μm Bayer pattern. Next, puddle development was performed on the exposed curable composition layer at 23° C. for 60 seconds using a tetramethylammonium hydroxide (TMAH) 0.3 mass % aqueous solution. Next, the silicon wafer was rinsed by spin showering and was cleaned with pure water. As a result, a pattern was obtained. The amount of residues remaining on the underlayer of the obtained pattern was evaluated by binarization of the image based on the following criteria.

A: the amount of the residues was lower than 1% with respect to the total area of the underlayer

B: the amount of the residues was higher than 1% and 3% or lower with respect to the total area of the underlayer

C: the amount of the residues was higher than 3% with respect to the total area of the underlayer

TABLE 12

| | Dispersion | | Moisture | |
|---|---|---|---|---|
| | Used | Developability | Resistance | Aggregate |
| Manufacturing Example 101 | Example 1 | A | A | A |
| Manufacturing Example 102 | Example 2 | A | A | A |
| Manufacturing Example 103 | Example 3 | A | A | A |
| Manufacturing Example 104 | Example 4 | A | A | A |
| Manufacturing Example 105 | Example 5 | A | A | A |
| Manufacturing Example 106 | Example 6 | A | A | A |
| Manufacturing Example 107 | Example 7 | A | A | A |
| Manufacturing Example 108 | Example 8 | A | A | A |
| Manufacturing Example 109 | Example 9 | A | A | A |
| Manufacturing Example 110 | Example 10 | B | A | A |
| Manufacturing Example 111 | Example 11 | A | A | A |
| Manufacturing Example 112 | Example 12 | A | A | A |
| Manufacturing Example 113 | Example 13 | B | A | A |
| Manufacturing Example 114 | Example 14 | B | A | B |
| Manufacturing Example 115 | Example 15 | B | A | B |
| Manufacturing Example 116 | Example 16 | B | A | B |
| Manufacturing Example 117 | Example 17 | B | A | B |
| Manufacturing Example 118 | Example 18 | B | A | B |
| Manufacturing Example 119 | Example 19 | A | A | A |
| Manufacturing Example 120 | Example 20 | A | A | A |
| Manufacturing Example 121 | Example 21 | A | A | A |
| Manufacturing Example 122 | Example 22 | B | A | A |
| Manufacturing Example 123 | Example 23 | B | A | A |
| Manufacturing Example 124 | Example 24 | B | A | A |
| Manufacturing Example 125 | Example 25 | A | A | A |
| Manufacturing Example 126 | Example 26 | B | A | A |
| Manufacturing Example 127 | Example 27 | B | A | A |
| Manufacturing Example 128 | Example 28 | B | A | A |
| Manufacturing Example 129 | Example 29 | B | A | A |
| Manufacturing Example 130 | Example 30 | A | A | A |
| Manufacturing Example 131 | Example 31 | B | A | B |
| Manufacturing Example 132 | Example 32 | B | A | A |
| Manufacturing Example 133 | Example 33 | B | A | A |
| Manufacturing Example 134 | Example 34 | B | A | B |
| Manufacturing Example 135 | Example 35 | B | A | B |
| Manufacturing Example 136 | Example 36 | B | A | A |
| Manufacturing Example 137 | Example 37 | B | A | A |
| Manufacturing Example 138 | Example 38 | A | A | A |
| Manufacturing Example 139 | Example 39 | A | A | A |
| Manufacturing Example 140 | Example 40 | A | A | A |
| Manufacturing Example 141 | Example 41 | B | B | B |
| Manufacturing Example 142 | Example 42 | B | A | B |
| Manufacturing Example 143 | Example 43 | A | A | A |
| Manufacturing Example 144 | Example 44 | A | A | A |
| Manufacturing Example 145 | Example 45 | A | A | A |
| Manufacturing Example 146 | Example 46 | A | A | A |
| Manufacturing Example 147 | Comparative | B | C | B |
| | Example 1 | | | |
| Manufacturing Example 148 | Comparative | C | A | C |
| | Example 2 | | | |

TABLE 13

| | | | Moisture | |
|---|---|---|---|---|
| | Dispersion Used | Developability | Resistance | Aggregate |
| Manufacturing Example 149 | Example 47 | A | A | B |
| Manufacturing Example 150 | Example 48 | A | A | B |
| Manufacturing Example 151 | Example 49 | B | A | B |
| Manufacturing Example 152 | Example 50 | A | A | A |
| Manufacturing Example 153 | Example 51 | A | A | A |
| Manufacturing Example 154 | Example 52 | A | A | A |
| Manufacturing Example 155 | Example 53 | A | A | A |
| Manufacturing Example 156 | Example 54 | A | A | B |
| Manufacturing Example 157 | Example 55 | A | A | B |
| Manufacturing Example 158 | Example 56 | A | A | A |
| Manufacturing Example 159 | Example 57 | A | A | A |
| Manufacturing Example 160 | Example 58 | A | A | A |
| Manufacturing Example 161 | Example 59 | A | A | A |
| Manufacturing Example 162 | Example 60 | A | A | B |
| Manufacturing Example 163 | Example 61 | A | A | B |
| Manufacturing Example 164 | Example 62 | A | A | A |
| Manufacturing Example 165 | Example 63 | A | A | A |
| Manufacturing Example 166 | Example 64 | A | A | A |
| Manufacturing Example 167 | Example 65 | A | A | A |
| Manufacturing Example 168 | Example 66 | A | A | B |
| Manufacturing Example 169 | Example 67 | A | A | B |
| Manufacturing Example 170 | Example 68 | A | A | A |
| Manufacturing Example 171 | Example 69 | A | A | A |
| Manufacturing Example 172 | Example 70 | A | A | A |
| Manufacturing Example 173 | Example 71 | A | A | A |
| Manufacturing Example 174 | Example 72 | A | A | B |
| Manufacturing Example 175 | Example 73 | A | A | B |
| Manufacturing Example 176 | Example 74 | A | A | A |
| Manufacturing Example 177 | Example 75 | A | A | A |
| Manufacturing Example 178 | Example 76 | A | A | A |
| Manufacturing Example 179 | Example 77 | A | A | A |
| Manufacturing Example 180 | Example 78 | A | A | B |
| Manufacturing Example 181 | Example 79 | A | A | B |
| Manufacturing Example 182 | Example 80 | A | A | A |
| Manufacturing Example 183 | Example 81 | A | A | A |
| Manufacturing Example 184 | Example 82 | A | A | A |
| Manufacturing Example 185 | Example 83 | A | A | A |
| Manufacturing Example 186 | Example 84 | A | A | A |
| Manufacturing Example 187 | Example 85 | A | A | A |

TABLE 14

| | | | Moisture | |
|---|---|---|---|---|
| | Dispersion Used | Developability | Resistance | Aggregate |
| Manufacturing Example 188 | Example 86 | A | A | B |
| Manufacturing Example 189 | Example 87 | A | A | B |
| Manufacturing Example 190 | Example 88 | A | A | A |
| Manufacturing Example 191 | Example 89 | A | A | A |
| Manufacturing Example 192 | Example 90 | A | A | A |
| Manufacturing Example 193 | Example 91 | A | A | A |
| Manufacturing Example 194 | Example 92 | A | A | A |
| Manufacturing Example 195 | Example 93 | A | A | A |
| Manufacturing Example 196 | Example 94 | A | A | A |
| Manufacturing Example 197 | Example 95 | A | A | A |
| Manufacturing Example 198 | Example 96 | A | A | A |
| Manufacturing Example 199 | Example 97 | A | A | B |
| Manufacturing Example 200 | Example 98 | A | A | B |
| Manufacturing Example 201 | Example 99 | A | A | A |
| Manufacturing Example 202 | Example 100 | A | A | A |
| Manufacturing Example 203 | Example 101 | A | A | A |
| Manufacturing Example 204 | Example 102 | A | A | A |
| Manufacturing Example 205 | Example 103 | A | A | B |
| Manufacturing Example 206 | Example 104 | A | A | B |
| Manufacturing Example 207 | Example 105 | A | A | A |
| Manufacturing Example 208 | Example 106 | A | A | A |
| Manufacturing Example 209 | Example 107 | A | A | A |
| Manufacturing Example 210 | Example 108 | A | A | A |
| Manufacturing Example 211 | Example 109 | A | A | B |
| Manufacturing Example 212 | Example 110 | A | A | B |
| Manufacturing Example 213 | Example 111 | A | A | A |
| Manufacturing Example 214 | Example 112 | A | A | A |
| Manufacturing Example 215 | Example 113 | A | A | A |
| Manufacturing Example 216 | Example 114 | A | A | A |
| Manufacturing Example 217 | Example 115 | A | A | B |
| Manufacturing Example 218 | Example 116 | A | A | B |
| Manufacturing Example 219 | Example 117 | A | A | B |
| Manufacturing Example 220 | Example 118 | A | A | B |
| Manufacturing Example 221 | Example 119 | A | A | B |
| Manufacturing Example 222 | Example 120 | A | A | B |
| Manufacturing Example 223 | Example 121 | A | A | B |
| Manufacturing Example 224 | Example 122 | A | A | B |
| Manufacturing Example 225 | Example 123 | A | A | B |

Each of the curable compositions 101 according to Manufacturing Examples 106, 109 to 146, and 149 to 225 among the curable compositions shown in the tables is a curable composition including a dispersion that is formed by using, as the specific compound A, Ap-1, Ap-2, Ap-5, Ap-10, Ap-14, Ap-17, Ap-18, Ap-25, Ap-26, Ap-27, Ap-33, Ap-34, Ap-35, Ap-36, Ap-39, Ap-45, Ap-46, Ap-47, Ap-48, Ap-49, Ap-52, Ap-58, Ap-59, Ap-60, Ap-61, Ap-62, Ap-63, Ap-64, Ap-65, Ap-71, Ap-72, Ap-73, Ap-74, Ap-75, AP-78, Ap-79, Ap-80, Ap-81, Ap-82, Ap-83, Ap-84, Ap-85, Ap-86, Ap-87, Ap-88, Ap-91, Ap-97, Ap-98, Ap-99, Ap-100, Ap-101, Ap-104, Ap-110, Ap-111, Ap-112, Ac-1, or As-5 that is a compound having a structure in which the functional group A is bonded to a π-conjugated structure of a colorant skeleton and having a maximum absorption wavelength in a wavelength range of 650 to 1200 nm. In addition, curable compositions according to Manufacturing Examples 147 and 148 are curable compositions according to Comparative Examples of the present invention.

As shown in the tables, in each of Manufacturing Examples 101 to 146 and 149 to 225, a cured film having excellent moisture resistance in which the formation of an aggregate derived from a compound having a colorant skeleton was suppressed was able to be formed. In addition, in the curable compositions according to Manufacturing Examples 101 to 106, 109 to 146, and 149 to 225, near infrared absorbing properties were excellent. In addition, in the curable compositions according to Manufacturing Examples 101 to 106, 109 to 130, 138 to 146, and 149 to 225, higher near infrared absorbing properties were excellent.

In Manufacturing Examples 101 to 146 and 149 to 225, even in a case where ARONIX M-510 (manufactured by Toagosei Co., Ltd.), KAYARAD DPHA (manufactured by Nippon Kayaku Co., Ltd.), or ARTON F4520 (manufactured by JSR Corporation) was used as the curable compound instead of ARONIX M-305 (manufactured by Toagosei Co., Ltd.), the same effects were obtained.

In Manufacturing Examples 101 to 146 and 149 to 225, even in a case where a combination including KAYARAD DPHA (manufactured by Nippon Kayaku Co., Ltd.) and NK ESTER A-DPH-12E (manufactured by Shin-Nakamura Chemical Co., Ltd.) at a mass ratio of 1:1 a combination including NK ESTER A-TMMT (manufactured by Shin-Nakamura Chemical Co., Ltd.) and NK ESTER A-DPH-12E (manufactured by Shin-Nakamura Chemical Co., Ltd.) at a mass ratio of 1:1, or a combination including ARONIX TO-2349 (manufactured by Toagosei Co., Ltd.) and NK ESTER A-DPH-12E (manufactured by Shin-Nakamura Chemical Co., Ltd. at a mass ratio of 1:1 was used as the curable compound instead of ARONIX M-305 (manufactured by Toagosei Co., Ltd.), the same effects were obtained.

In Manufacturing Examples 101 to 146 and 149 to 225, even in a case where ACRYCURE RD-F8 (manufactured by Nippon Shokubai Co., Ltd.) or ACRYBASE FFS-6752 (manufactured by Nippon Shokubai Co., Ltd.) was used as the alkali-soluble resin instead of ACRYBASE FF-426 (manufactured by Nippon Shokubai Co., Ltd.), the same effects were obtained.

In Manufacturing Examples 101 to 137, 139 to 146, and 149 to 225, even in a case where the colorant B on which kneading and polishing was performed using the following method was used, the same effects were obtained.

5.3 parts by mass of the colorant B after the synthesis, 74.7 parts by mass of a milling agent, and 14 parts by mass of a binder were added to a LABO PLASTOMILL (manufactured by Toyo Seiki Seisaku-sho, Ltd.) and were kneaded for 2 hours while controlling the temperature such that the temperature of a kneaded material in the device was 70° C. As the milling agent, a neutral anhydrous mirabilite E (average particle size (a 50% diameter (D50) in terms of volume))=20 μm, manufactured by Mitajiri Chemical Industry Co., Ltd.) was used. As the binder, diethylene glycol was used. The kneaded material having undergone kneading and polishing was cleaned with 10 L of water at 24° C. to remove the milling agent and the binder and then was treated using a heating oven at 80° C. for 24 hours.

### Test Example 3

<Manufacturing of Curable Composition>

### Example 201

The following components were mixed with each other to prepare a curable composition.

Compound A (the compound (Ap-1) having the structure): 0.5 parts by mass

Curable compound (EHPE 3150, manufactured by Daicel Corporation): 32.94 parts by mass

Curing agent (pyromellitic anhydride): 3.50 parts by mass
Surfactant 1 (a compound having the following structure, weight-average molecular weight=14000, “%” representing the proportion of a repeating unit is mol %): 0.02 parts by mass

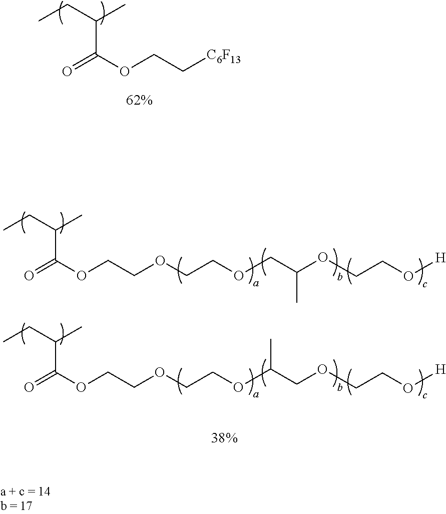

PGMEA: 63.04 parts by mass

### Example 202

The following components were mixed with each other to prepare a curable composition.

Compound A (the compound (Ap-1) having the structure): 0.33 parts by mass

Colorant (SQ-7 described above): 0.17 parts by mass

Curable compound (CYCLOMER P(ACA)230AA, manufactured by Daicel Corporation): 6.78 parts by mass

Curable compound (KAYARAD DPHA, manufactured by Nippon Kayaku Co., Ltd.): 2.54 parts by mass

Curable compound (EHPE 3150, manufactured by Daicel Corporation): 2.54 parts by mass

Photoradical polymerization initiator (IRGACURE-OXE01, manufactured by BASF SE): 1.46 parts by mass

Curing agent (pyromellitic anhydride): 0.72 parts by mass

Polymerization inhibitor (para-methoxyphenol): 0.10 parts by mass

PGMEA: 50.00 parts by mass

Butyl acetate: 5.36 parts by mass

Cyclopentanone: 30.00 parts by mass

### Example 203

The following components were mixed with each other to prepare a curable composition.

Compound A (the compound (Ac-1) having the structure): 2.40 parts by mass

Curable compound (CYCLOMER P(ACA)230AA, manufactured by Daicel Corporation): 9.32 parts by mass

Photoradical polymerization initiator (IRGACURE-OXE01, manufactured by BASF SE): 1.46 parts by mass

Curing agent (RIKACID MTA-15, manufactured by New Japan Chemical Co., Ltd.): 2.54 parts by mass

Polymerization inhibitor (para-methoxyphenol): 0.10 parts by mass

3-butoxy-N,N-dimethylpropanamide: 84.16 parts by mass

Surfactant 1: 0.02 parts by mass

### Example 204

The following components were mixed with each other to prepare a curable composition.

Compound A (the compound (As-5) having the structure): 0.2 parts by mass

Colorant (SQ-7 described above): 0.05 parts by mass

Curable compound (ARTON F4520, manufactured by JSR Corporation): 39.2 parts by mass

Surfactant 1: 0.02 parts by mass

Cyclohexanone: 60.53 parts by mass

### Example 205

The following components were mixed with each other to prepare a curable composition.

Compound A (the compound (Ap-26) having the structure): 0.5 parts by mass

Curable compound (EHPE 3150, manufactured by Daicel Corporation): 32.94 parts by mass

Curing agent (pyromellitic anhydride): 3.50 parts by mass

Surfactant 1: 0.02 parts by mass

PGMEA: 63.04 parts by mass

### Comparative Example 201

The following components were mixed with each other to prepare a curable composition.

Colorant (SQ-7 described above): 0.44 parts by mass

Curable compound (JER157S65, manufactured by Mitsubishi Chemical Corporation): 39.2 parts by mass

Surfactant 1: 0.02 parts by mass

Cyclohexanone: 60.34 parts by mass

<Preparation of Cured Film>

Each of the curable compositions according to Examples 201, 204, and 205 and Comparative Example 201 was applied to a glass substrate using a spin coating method and then was cured using a hot plate at 100° C. for 2 minutes at 230° C. for 5 minutes. As a result, a cured film having a thickness of about 2.0 μm was obtained. In addition, each of the curable compositions according to Examples 202 and 203 was applied to a glass substrate using a spin coating method and then was heated using a hot plate at 100° C. for 2 minutes. As a result, a composition layer was obtained. The obtained composition layer was exposed using an i-ray stepper at an exposure dose of 1000 mJ/cm². Next, the exposed composition layer was further heated using a hot plate at 230° C. for 5 minutes. As a result, a cured film having a thickness of about 2.0 μm was obtained.

<Evaluation of Moisture Resistance>

Regarding the obtained cured film, the transmittance of light in a wavelength range of 700 to 1000 nm was measured. Next, this cured film was put into a constant-temperature tank at 85° C. and a humidity of 95% and was stored therein for 6 months to perform a moisture-resistance test. Regarding the cured film after the moisture-resistance test, the transmittance of light at each wavelength in a wavelength range of 700 to 1000 nm was measured. The transmittance of the cured film was measured using a spectrophotometer (manufactured by Hitachi High-Technologies Corporation, U-4100).

A maximum value (ΔT) of a change in transmittance at each wavelength in a wavelength range of 700 to 1000 nm before and after the moisture-resistance test was measured and was set as an index indicating moisture resistance.

Change (ΔT) in Transmittance=Transmittance (%) of Cured Film before Moisture-Resistance Test-Transmittance (%) of Cured Film after Moisture-Resistance Test

A: ΔT %<4%

B: 4%≤ΔT %<10%

C: 10%≤ΔT %

<Evaluation of Aggregate Derived from Compound Having Colorant Skeleton>

The obtained cured film was observed (measurement magnification=10000 times) using a scanning electron microscope, and the number of foreign matter particles present in a range of 10 μm×15 μm was measured.

A: no foreign matter was present in a range of 10 μm×15 μm

B: the number of foreign matter particles present in a range of 10 μm×15 μm was more than 0 and 100 or less

C: the number of foreign matter particles present in a range of 10 μm×15 μm was more than 100

TABLE 15

| | Moisture | |
|---|---|---|
| | Resistance | Aggregate |
| Example 201 | A | A |
| Example 202 | A | B |
| Example 203 | A | B |
| Example 204 | A | A |
| Example 205 | A | A |
| Comparative | C | C |
| Example 201 | | |

As shown in the tables, in each of Examples 201 to 205, a cured film having excellent moisture resistance in which the formation of an aggregate derived from a compound having a colorant skeleton was suppressed was able to be formed.

### Test Example 4

(Preparation of Curable Composition for Forming Infrared Transmitting Filter)

The following raw materials were mixed and stirred, and the obtained mixture was filtered through a nylon filter (manufactured by Pall Corporation) having a pore size of 0.45 μm to prepare a curable composition 101 for forming an infrared transmitting filter.

(Curable Composition 101 for Forming Infrared Transmitting Filter)

| | |
|---|---|
| Dispersion of Example 1 | 16.21 parts by mass |
| Pigment Dispersion 1-1 | 11.33 parts by mass |
| Pigment Dispersion 1-2 | 22.67 parts by mass |
| Pigment Dispersion 1-3 | 10.34 parts by mass |
| Pigment Dispersion 1-4 | 6.89 parts by mass |
| Curable compound (ARONIX M-305, | 1.37 parts by mass |
| manufactured by Toagosei Co., Ltd.) | |
| Resin 101 | 3.52 parts by mass |
| Photoradical polymerization | 0.86 parts by mass |
| initiator (IRGACURE-OXE01, | |
| manufactured by BASF SE) | |
| Surfactant 101 | 0.42 parts by mass |
| Polymerization inhibitor | 0.001 parts by mass |
| (p-methoxyphenol) | |
| PGMEA | 19.93 parts by mass |

Resin 101: a resin having the following structure (Mw=40000, a numerical value added to a main chain represents a mass ratio of a repeating unit)

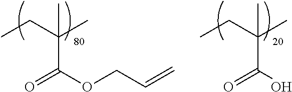

Surfactant 101: a 1 mass % PGMEA solution of the following mixture (Mw: 14000; in the following formula, “%” representing the proportion of a repeating unit is mol %)

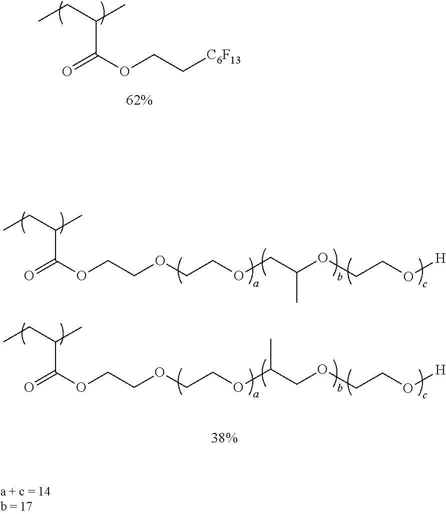

(Pigment Dispersion 1-1)

A mixed solution having a composition shown below was mixed and dispersed using a beads mill (a high-pressure disperser with a pressure reducing mechanism, NANO-3000-10 (manufactured by Nippon BEE Chemical Co., Ltd.)) with zirconia beads having a diameter of 0.3 mm. This way, pigment dispersions were prepared.

| | |
|---|---|
| C.I. Pigment Red 254 | 13.5 parts by mass |
| Resin 11 | 2 parts by mass |
| Resin 12 | 2 parts by mass |
| PGMEA | 82.5 parts by mass |

Resin 11: a resin having the following structure (Mw=7950, a numerical value added to a main chain represents a molar ratio of a repeating unit, a numerical value added to a side chain represents the number of repeating units)

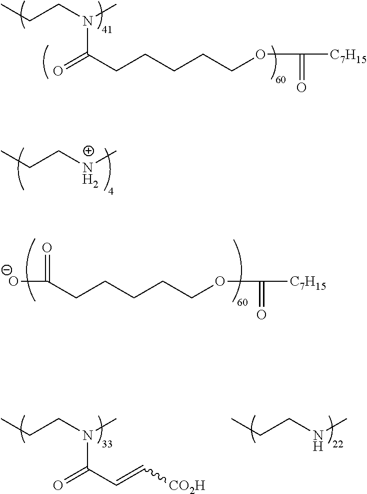

Resin 12: a resin having the following structure (Mw=12000, a numerical value added to a main chain represents a molar ratio of a repeating unit)

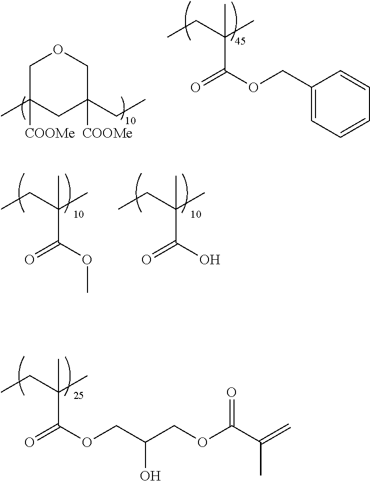

(Pigment Dispersion 1-2)

A mixed solution having a composition shown below was mixed and dispersed using a beads mill (a high-pressure disperser with a pressure reducing mechanism, NANO-3000-10 (manufactured by Nippon BEE Chemical Co., Ltd.)) with zirconia beads having a diameter of 0.3 mm. This way, pigment dispersions were prepared.

| | |
|---|---|
| C.I. Pigment Blue 15:6 | 13.5 parts by mass |
| Resin 13 | 4 parts by mass |
| PGMEA | 82.5 parts by mass |

Resin 13: a resin having the following structure (Mw=30000, a numerical value added to a main chain represents a molar ratio of a repeating unit, a numerical value added to a side chain represents the number of repeating units)

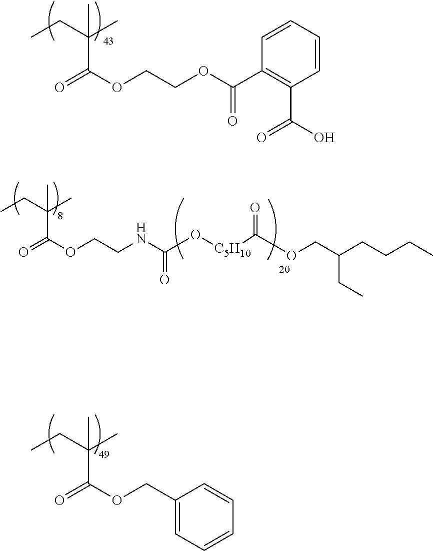

(Pigment Dispersion 1-3)

A mixed solution having a composition shown below was mixed and dispersed using a beads mill (a high-pressure disperser with a pressure reducing mechanism, NANO-3000-10 (manufactured by Nippon BEE Chemical Co., Ltd.)) with zirconia beads having a diameter of 0.3 mm. This way, pigment dispersions were prepared.

| | |
|---|---|
| C.I. Pigment Yellow 139 | 14.8 parts by mass |
| Resin (Disperbyk-111, manufactured | 3 parts by mass |
| by BYK Chemie) | |
| Resin 12 | 2.2 parts by mass |
| PGMEA | 80 parts by mass |

(Pigment Dispersion 1-4)

A mixed solution having a composition shown below was mixed and dispersed using a beads mill (a high-pressure disperser with a pressure reducing mechanism, NANO-3000-10 (manufactured by Nippon BEE Chemical Co., Ltd.)) with zirconia beads having a diameter of 0.3 mm. This way, pigment dispersions were prepared.

| | |
|---|---|
| C.I. Pigment Violet 23 | 14.8 parts by mass |
| Resin (Disperbyk-111, manufactured by BYKX | 3 parts by mass |
| by BYK Chemie) | |
| Resin 12 | 2.2 parts by mass |
| PGMEA | 80 parts by mass |

(Preparation of Red Composition)

9.6 parts by mass of C.I. Pigment Red 254, 4.3 parts by mass of C.I. Pigment Yellow 139, 6.8 parts by mass of a dispersant (Disperbyk-161, manufactured by BYK Chemie), and 79.3 parts by mass of PGMEA were mixed with each other to obtain a mixed solution, and the mixed solution was mixed and dispersed using a beads mill (zirconia beads; diameter: 0.3 mm) for 3 hours. As a result, a pigment dispersion was prepared. Next, using a high-pressure disperser NANO-3000-10 (manufactured by Nippon BEE Chemical Co., Ltd.) equipped with a pressure reducing mechanism, the pigment dispersion was further dispersed under a pressure of 2000 kg/cm³ at a flow rate of 500 g/min. This dispersing treatment was repeated 10 times. As a result, a Red pigment dispersion was obtained.

The following raw materials were mixed and stirred, and the obtained mixture was filtered through a nylon filter (manufactured by Pall Corporation) having a pore size of 0.45 m to prepare a Red composition.

| | |
|---|---|
| Red Pigment Dispersion | 51.7 parts by mass |
| Resin 102 (40% PGMEA solution) | 0.6 parts by mass |
| Curable compound 102 | 0.6 parts by mass |
| Photoradical polymerization initiator | 0.3 parts by mass |
| (IRGACURE-OXE01, manufactured | |
| by BASF SE) | |
| Surfactant 101 | 4.2 parts by mass |
| PGMEA | 42.6 parts by mass |

Curable Compound 102: a compound having the following structure

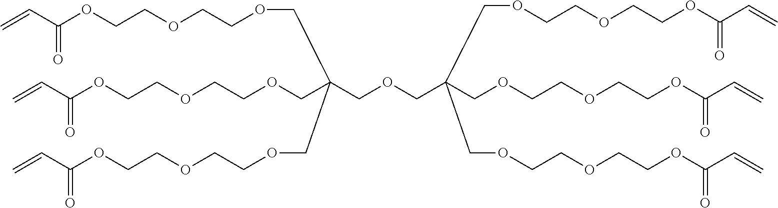 [SMILES: C=CC(=O)OCCOCCOCC(COCCOCCOC(=O)C=C)(COCCOCCOC(=O)C=C)COCC(COCCOCCOC(=O)C=C)(COCCOCCOC(=O)C=C)COCCOCCOC(=O)C=C]

Resin 102: a resin having the following structure (acid value=70 mgKOH/g, Mw=11000, a numerical value added to a main chain represents a mass ratio of a repeating unit)

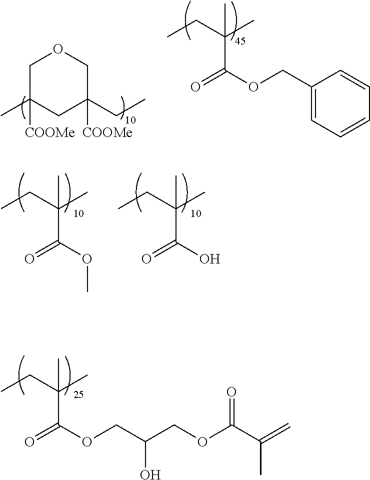

(Preparation of Green Composition)

6.4 parts by mass of C.I. Pigment Green 36, 5.3 parts by mass of C.I. Pigment Yellow 150, 5.2 parts by mass of a dispersant (Disperbyk-161, manufactured by BYK Chemie), and 83.1 parts by mass of PGMEA were mixed with each other to obtain a mixed solution, and the mixed solution was mixed and dispersed using a beads mill (zirconia beads; diameter: 0.3 mm) for 3 hours. As a result, a pigment dispersion was prepared. Next, using a high-pressure disperser NANO-3000-10 (manufactured by Nippon BEE Chemical Co., Ltd.) equipped with a pressure reducing mechanism, the pigment dispersion was further dispersed under a pressure of 2000 kg/cm³ at a flow rate of 500 g/min. This dispersing treatment was repeated 10 times. As a result, a Green pigment dispersion was obtained.

The following raw materials were mixed and stirred, and the obtained mixture was filtered through a nylon filter (manufactured by Pall Corporation) having a pore size of 0.45 μm to prepare a Green composition.

| | |
|---|---|
| Green Pigment Dispersion | 73.7 parts by mass |
| Resin 102 (40% PGMEA solution) | 0.3 parts by mass |
| Curable compound (KAYARAD DPHA, | 1.2 parts by mass |
| manufactured by Nippon Kayaku Co., Ltd.): | |
| Photoradical polymerization initiator | 0.6 parts by mass |
| (IRGACURE-OXE01, manufactured by BASF SE) | |
| Surfactant 101 | 4.2 parts by mass |
| Ultraviolet absorber (UV-503, manufactured by | 0.5 parts by mass |
| Daito Chemical Co., Ltd.) | |
| PGMEA | 19.5 parts by mass |

(Preparation of Blue Composition)

9.7 parts by mass of C.I. Pigment Blue 15:6, 2.4 parts by mass of C.I. Pigment Violet 23, 5.5 parts by mass of a dispersant (Disperbyk-161 (manufactured by BYK Chemie)), 82.4 parts by mass of PGMEA were mixed with each other to obtain a mixed solution, and the mixed solution was mixed and dispersed using a beads mill (zirconia beads; diameter: 0.3 mm) for 3 hours. As a result, a pigment dispersion was prepared. Next, using a high-pressure disperser NANO-3000-10 (manufactured by Nippon BEE Chemical Co., Ltd.) equipped with a pressure reducing mechanism, the pigment dispersion was further dispersed under a pressure of 2000 kg/cm³ at a flow rate of 500 g/min. This dispersing treatment was repeated 10 times. As a result, a Blue pigment dispersion was obtained.

The following raw materials were mixed and stirred, and the obtained mixture was filtered through a nylon filter (manufactured by Pall Corporation) having a pore size of 0.45 μm to prepare a Blue composition.

| | |
|---|---|
| Blue Pigment Dispersion | 44.9 parts by mass |
| Resin 102 (40% PGMEA solution) | 2.1 parts by mass |
| Curable compound (KAYARAD DPHA, | 1.5 parts by mass |
| manufactured by Nippon Kayaku Co., Ltd.): | |
| Curable compound 102 | 0.7 parts by mass |
| Photoradical polymerization initiator | 0.8 parts by mass |
| (IRGACURE-OXE01, manufactured by BASF SE) | |
| Surfactant 101 | 4.2 parts by mass |
| PGMEA | 45.8 parts by mass |

(Pattern Formation)

The curable composition according to Manufacturing Example 118 was applied to a silicon wafer using a spin coating method such that the thickness of the formed film was 1.0 μm, was heated using a hot plate at 100° C. for 2 minutes, and then was heated using a hot plate at 200° C. for 5 minutes. Next, a 2 μm Bayer pattern (near infrared cut filter) was formed using a dry etching method.

Next, the Red composition was applied to the Bayer pattern of the near infrared cut filter using a spin coating method such that the thickness of the formed film was 1.0 μm. Next, the coating film was heated using a hot plate at 100° C. for 2 minutes. Next, using an i-ray stepper exposure device FPA-3000 i5+(manufactured by Canon Corporation) at 1000 mJ/cm², a 2 μm dot pattern was exposed through a mask at 1000 mJ/cm². Next, puddle development was performed at 23° C. for 60 seconds using a tetramethylammonium hydroxide (TMAH) 0.3 mass % aqueous solution. Next, the silicon wafer was rinsed by spin showering, was cleaned with pure water, and was heated using a hot plate at 200° C. for 5 minutes. As a result, a cured film of the Red composition was patterned on the Bayer pattern of the near infrared cut filter. Likewise, a cured film of the Green composition and a cured film of the Blue composition were sequentially patterned to form red, blue, and green color patterns.

Next, the curable composition 101 for forming an infrared transmitting filter was applied to the pattern-formed film using a spin coating method such that the thickness of the formed film was 2.0 μm. Next, the coating film was heated using a hot plate at 100° C. for 2 minutes. Next, using an i-ray stepper exposure device FPA-3000 i5+(manufactured by Canon Corporation) at 1000 mJ/cm², a 2 μm Bayer pattern was exposed through a mask at 1000 mJ/cm². Next, puddle development was performed at 23° C. for 60 seconds using a tetramethylammonium hydroxide (TMAH) 0.3 mass % aqueous solution. Next, the silicon wafer was rinsed by spin showering, was cleaned with pure water, and was heated using a hot plate at 200° C. for 5 minutes. As a result, the infrared transmitting filter was patterned on a portion where the Bayer pattern of the near infrared cut filter was not formed. This filter was incorporated into a solid image pickup element using a well-known method

The obtained solid image pickup element was irradiated with light having an emission wavelength of 940 nm emitted from an infrared light emitting diode (infrared LED) light source in a low-illuminance environment (0.001 Lux) to obtain an image. The subject was able to be clearly recognized on the image.

### Test Example 5

(Preparation of Curable Composition for Forming Infrared Transmitting Filter)

The following raw materials were mixed and stirred, and the obtained mixture was filtered through a nylon filter (manufactured by Pall Corporation) having a pore size of 0.45 m to prepare a curable composition 102 for forming an infrared transmitting filter.

(Curable Composition 102 for Forming Infrared Transmitting Filter)

| | |
|---|---|
| Pigment Dispersion 10-1 | 46.5 parts by mass |
| Pigment Dispersion 10-2 | 37.1 parts by mass |
| Curable compound 201 | 1.8 parts by mass |
| Resin 201 | 1.1 parts by mass |
| Photoradical polymerization initiator 201 | 0.9 parts by mass |
| Surfactant 101 | 4.2 parts by mass |
| Polymerization inhibitor (p-methoxyphenol) | 0.001 parts by mass |
| Silane coupling agent 201 | 0.6 parts by mass |
| PGMEA | 7.8 parts by mass |

(Pigment Dispersion 10-1)

A mixed solution having a composition shown below was mixed and dispersed for 3 hours using a beads mill (a high-pressure disperser with a pressure reducing mechanism, NANO-3000-10 (manufactured by Nippon BEE Chemical Co., Ltd.)) in which zirconia beads having a diameter of 0.3 mm were used. As a result, a pigment dispersion 10-1 was prepared.

| | |
|---|---|
| A mixed pigment including a red pigment | 11.8 parts by mass |
| (C.I. Pigment Red 254) and a yellow | |
| pigment (C.I. Pigment Yellow 139) | |
| Resin (Disperbyk-111, manufactured by BYK | 9.1 parts by mass |
| Chemie) | |
| PGMEA | 79.1 parts by mass |

(Pigment Dispersion 10-2)

A mixed solution having a composition shown below was mixed and dispersed for 3 hours using a beads mill (a high-pressure disperser with a pressure reducing mechanism, NANO-3000-10 (manufactured by Nippon BEE Chemical Co., Ltd.)) in which zirconia beads having a diameter of 0.3 mm were used. As a result, a pigment dispersion 10-2 was prepared.

| | |
|---|---|
| A mixed pigment including a blue pigment | 12.6 parts by mass |
| (C.I. Pigment Blue 15:6) and a violet | |
| pigment (C.I. Pigment Violet 23) | |
| Resin (Disperbyk-111, manufactured by BYK | 2.0 parts by mass |
| Chemie) | |
| Resin 202 | 3.3 parts by mass |
| Cyclohexanone | 31.2 parts by mass |
| PGMEA | 50.9 parts by mass |

Curable Compound 201: the following structures (a mixture in which a molar ratio between a left compound and a right compound is 7:3)

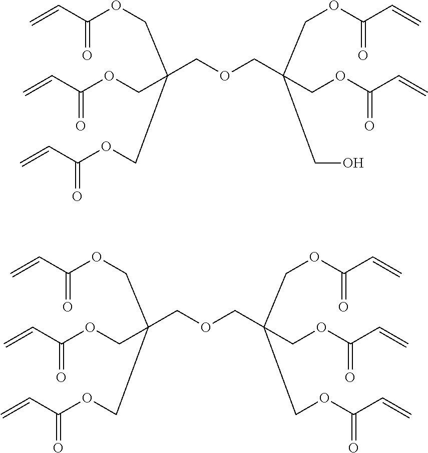

Resin 201: a resin having the following structure (acid value=70 mgKOH/g, Mw=11000, a numerical value added to a main chain represents a molar ratio of a repeating unit)

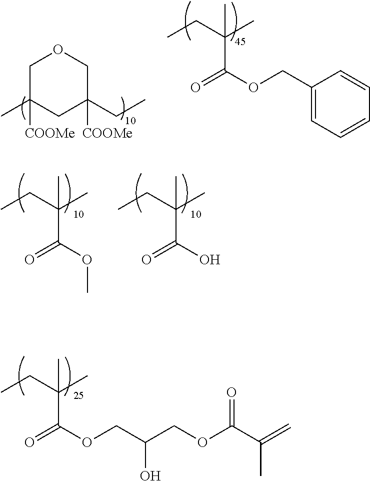

Resin 202: a resin having the following structure (Mw=14000, a numerical value added to a main chain represents a molar ratio of a repeating unit)

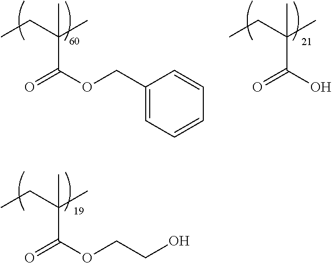

Photoradical polymerization initiator 201: a compound having the following structure

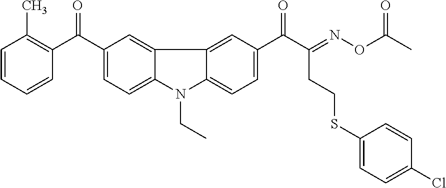 [SMILES: CCn1c2ccc(C(=O)/C(CCSc3ccc(Cl)cc3)=N/OC(C)=O)cc2c2cc(C(=O)c3ccccc3C)ccc21]

Silane coupling agent 201: a compound having the following structure

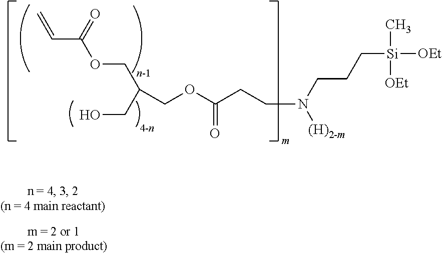

(Pattern Formation)

The curable composition according to Manufacturing Example 102 was applied to a silicon wafer using a spin coating method such that the thickness of the formed film was 1.0 μm, was heated using a hot plate at 100° C. for 2 minutes, and then was heated using a hot plate at 200° C. for 5 minutes. Next, a 2 Tim Bayer pattern (near infrared cut filter) was formed using a dry etching method.

Next, the Red composition was applied to the Bayer pattern of the near infrared cut filter using a spin coating method such that the thickness of the formed film was 1.0 m. Next, the coating film was heated using a hot plate at 100° C. for 2 minutes. Next, using an i-ray stepper exposure device FPA-3000 i5+(manufactured by Canon Corporation) at 1000 mJ/cm², a 2 μm dot pattern was exposed through a mask at 1000 mJ/cm². Next, puddle development was performed at 23° C. for 60 seconds using a tetramethylamnmonium hydroxide (TMAH) 0.3 mass % aqueous solution. Next, the silicon wafer was rinsed by spin showering, was cleaned with pure water, and was heated using a hot plate at 200° C. for 5 minutes. As a result, a cured film of the Red composition was patterned on the Bayer pattern of the near infrared cut filter. Likewise, a cured film of the Green composition and a cured film of the Blue composition were sequentially patterned to form red, blue, and green color patterns.

Next, the curable composition 102 for forming an infrared transmitting filter was applied to the pattern-formed film using a spin coating method such that the thickness of the formed film was 2.0 μm. Next, the coating film was heated using a hot plate at 100° C. for 2 minutes. Next, using an i-ray stepper exposure device FPA-3000 i5+(manufactured by Canon Corporation) at 1000 mJ/cm², a 2 μm Bayer pattern was exposed through a mask at 1000 mJ/cm². Next, puddle development was performed at 23° C. for 60 seconds using a tetramethylammonium hydroxide (TMAH) 0.3 mass % aqueous solution. Next, the silicon wafer was rinsed by spin showering, was cleaned with pure water, and was heated using a hot plate at 200° C. for 5 minutes. As a result, the infrared transmitting filter was patterned on a portion where the Bayer pattern of the near infrared cut filter was not formed. This filter was incorporated into a solid image pickup element using a well-known method

The obtained solid image pickup element was irradiated with light having an emission wavelength of 850 nm emitted from an infrared light emitting diode (infrared LED) light source in a low-illuminance environment (0.001 Lux) to obtain an image. The subject was able to be clearly recognized on the image.

### Test Example 6

<Preparation of Cesium Tungsten Oxide-Containing Composition>

49.84 parts by mass of YMS-01A-2 (cesium tungsten oxide particle dispersion; manufactured by Sumitomo Metal Mining Co., Ltd.), 39.5 parts by mass of the following resin 301 (PGMEA solution having a solid content of 40%), 6.80 parts by mass of KAYARAD DPHA (manufactured by Nippon Kayaku Co., Ltd.), 2.18 parts by mass of IRGACURE 369 (manufactured by BASF SE), and 1.68 parts by mass of PGMEA were mixed and stirred to prepare a cesium tungsten oxide-containing composition.

Resin 301: a resin having the following structure (acid value=70 mgKOH/g, Mw=11000, a numerical value added to a main chain represents a mass ratio of a repeating unit)

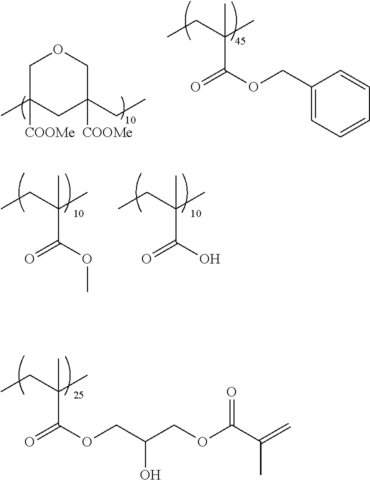

<Preparation of Near Infrared Cut Filter>

The curable composition according to Manufacturing Example 101 was applied to a glass substrate using a spin coating method and then was heated using a hot plate at 100° C. for 2 minutes. As a result, a composition layer was obtained. The obtained composition layer was exposed using an i-ray stepper at an exposure dose of 500 mJ/cm². Next, the exposed composition layer was cured using a hot plate at 220° C. for 5 minutes. As a result, a cured film having a thickness of 1.0 m was obtained. The cesium tungsten oxide-containing composition was applied to the substrate using a spin coating method to a film and then was heated using a hot plate at 100° C. for 2 minutes to obtain a composition layer. The obtained composition layer was exposed using an i-ray stepper at an exposure dose of 500 mJ/cm². Next, the exposed composition layer was cured using a hot plate at 220° C. for 5 minutes. As a result, a cured film having a thickness of 3.0 m was obtained, and a near infrared cut filter was manufactured. The transmittance of the obtained near infrared cut filter in a wavelength range of 800 to 1300 nm was 10% or lower.

### EXPLANATION OF REFERENCES

110: solid image pickup element
111: near infrared cut filter
112: color filter
114: infrared transmitting filter
115: microlens
116: planarizing layer

What is claimed is:

1. A curable composition comprising:
a compound A having a structure in which at least one functional group selected from a group consisting of an acid group having a pKa of 3 or lower and a Clog P value of −1.1 or higher, an anionic group obtained by dissociating one or more hydrogen atoms from the acid group, and a salt of the acid group is bonded to a π-conjugated structure of a colorant skeleton and having a maximum absorption wavelength in a wavelength range of 650 to 1200 nm;
a curable compound; and
a solvent.

2. The curable composition according to claim 1,
wherein the functional group has at least one structure selected from a group consisting of an acid structure selected from a group consisting of an imide acid structure, a methide acid structure, a boronic acid structure, a carboxylic acid structure, and a sulfonic acid structure, an anion obtained by dissociating one or more hydrogen atoms from the acid structure, and a salt of the acid structure.

3. The curable composition according to claim 1,
wherein the functional group includes a partial structure represented by the following Formula (1),
X¹—Y¹—Z¹ (1),
X¹ and Z¹ each independently represent —SO₂—, —CO—, —B(OH)—, or —P(═O)(OH)—,
Y¹ represents —NH—, —N—, or -NM¹-, and
M¹ represents an atom or an atomic group forming a salt.

4. The curable composition according to claim 1,
wherein the functional group is a group represented by the following Formula (10),
-L¹⁰-R⁹—X¹⁰—Y¹⁰—Z¹⁰—R¹⁰ (10),
in Formula (10), L¹⁰ represents a single bond or a divalent linking group,
X¹⁰ and Z¹⁰ each independently represent —SO₂—, —CO—, —B(OH)—, or —P(═O)(OH)—,
Y¹⁰ represents —NH—, —N⁻—, or -NM¹-,
M¹ represents an atom or an atomic group forming a salt,
R⁹ represents a single bond or a hydrocarbon group having 1 or more carbon atoms which may include a substituent, and
R¹⁰ represents a halogen atom, a hydroxyl group, or a hydrocarbon group having 1 or more carbon atoms which may include a substituent.

5. The curable composition according to claim 4,
wherein X¹⁰ represents —CO— and Z¹⁰ represents —SO₂—.

6. The curable composition according to claim 4,
wherein R¹⁰ represents a hydrocarbon group having 1 or more carbon atoms which includes a fluorine atom.

7. The curable composition according to claim 1,
wherein the functional group is a group represented by the following Formula (20) or the following Formula (30), 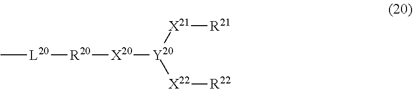
in Formula (20), L²⁰ represents a single bond or a divalent linking group,
X²⁰ to X²² each independently represent —SO₂—, —CO—, —B(OH)—, or —P(═O)(OH)—,
Y²⁰ represents —CH<, —C⁻<, or -CM²<,
M² represents an atom or an atomic group forming a salt,
R²⁰ represents a single bond or a hydrocarbon group having 1 or more carbon atoms which may include a substituent, and
R²¹ and R²² each independently represent a halogen atom, a hydroxyl group, or a hydrocarbon group having 1 or more carbon atoms which may include a substituent, and
-L³⁰-R³⁰—Y³⁰ (30),
in Formula (30), L³⁰ represents a single bond or a divalent linking group,
R³⁰ represents a hydrocarbon group having 1 or more carbon atoms which may include a substituent,
Y³⁰ represents —COOH, —COO⁻, —COOM³, —SO₃H, —SO₃⁻, —SO₃M³, or —B⁻(Rb1)(Rb2)(Rb3),
M³ represents an atom or an atomic group forming a salt, and
Rb1 to Rb3 each independently represent a halogen atom or a hydrocarbon group having 1 or more carbon atoms which may include a substituent.

8. The curable composition according to claim 1,
wherein the colorant skeleton is at least one selected from a group consisting of a pyrrolopyrrole colorant skeleton, a diimonium colorant skeleton, a phthalocyanine colorant skeleton, a naphthalocyanine colorant skeleton, a polymethine colorant skeleton, a pyrromethene colorant skeleton, and a perylene colorant skeleton.

9. The curable composition according to claim 1,
wherein the compound A is a compound represented by Formula (A1), 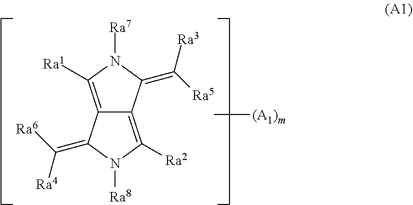
in Formula (A1), Ra¹ and Ra² each independently represent an alkyl group, an aryl group, or a heteroaryl group,
Ra³, Ra⁴, Ra⁵, and Ra⁶ each independently represent a cyano group, an acyl group, an alkoxycarbonyl group, an alkylsulfinyl group, an arylsulfinyl group, or a heteroaryl group,
Ra⁷ and Ra⁸ each independently represent a hydrogen atom, an alkyl group, an aryl group, a heteroaryl group, —BRa⁹Ra¹′, or a metal atom,
Ra⁷ may form a covalent bond or a coordinate bond with Ra¹, Ra³, or Ra⁵,
Ra⁸ may form a covalent bond or a coordinate bond with Ra², Ra⁴, or Ra⁶,
Ra⁹ and Ra¹⁰ each independently represent a hydrogen atom, a halogen atom, an alkyl group, an alkenyl group, an aryl group, a heteroaryl group, an alkoxy group, an aryloxy group, or a heteroaryloxy group,
Ra⁹ and Ra¹⁰ may be bonded to each other to form a ring,
A₁ represents at least one functional group selected from a group consisting of an acid group having a pKa of 3 or lower and a Clog P value of −1.1 or higher, an anionic group obtained by dissociating one or more hydrogen atoms from the acid group, and a salt of the acid group,
m represents an integer of 1 to 10, and
in a case where m represents 2 or more, a plurality of A₁'s may be the same as or different from each other.

10. The curable composition according to claim 1, further comprising:
a colorant other than the compound A.

11. A cured film which is formed using the curable composition according to claim 1.

12. An optical filter comprising:
the cured film according to claim 11.

13. The optical filter according to claim 12,
wherein the optical filter is a near infrared cut filter or an infrared transmitting filter.

14. A solid image pickup element comprising:
the cured film according to claim 11.

15. An image display device comprising:
the cured film according to claim 11.

16. An infrared sensor comprising:
the cured film according to claim 11.

17. A dispersing auxiliary agent comprising:
a compound having a structure in which at least one functional group selected from a group consisting of an acid group having a pKa of 3 or lower and a Clog P value of −1.1 or higher, an anionic group obtained by dissociating one or more hydrogen atoms from the acid group, and a salt of the acid group is bonded to a π-conjugated structure of a colorant skeleton.

18. A dispersion comprising:
a pigment;
the dispersing auxiliary agent according to claim 17;
a dispersant; and
a solvent.

19. A method of manufacturing a dispersion comprising:
dispersing a pigment in the presence of the dispersing auxiliary agent according to claim 17, a dispersant, and a solvent.

20. A compound which is represented by Formula (A2), 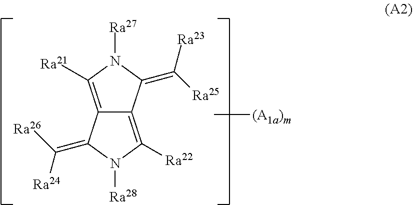
in Formula (A2), Ra²¹ and Ra²² each independently represent an alkyl group, an aryl group, or a heteroaryl group,
Ra²³, Ra²⁴, Ra²⁵, and Ra²⁶ each independently represent a cyano group, an acyl group, an alkoxycarbonyl group, an alkylsulfinyl group, an arylsulfinyl group, or a heteroaryl group,
Ra²⁷ and Ra²⁸ each independently represent —BRa²⁹Ra³⁰,
Ra²⁹ and Ra³⁰ each independently represent a hydrogen atom, a halogen atom, an alkyl group, an alkenyl group, an aryl group, a heteroaryl group, an alkoxy group, an aryloxy group, or a heteroaryloxy group,
Ra²⁹ and Ra³⁰ may be bonded to each other to form a ring,
A₁ₐ represents at least one functional group selected from a group consisting of an acid group having a pKa of 3 or lower and a Clog P value of −1.1 or higher, an anionic group obtained by dissociating one or more hydrogen atoms from the acid group, and a salt of the acid group,
m represents an integer of 1 to 10, and
in a case where m represents 2 or more, a plurality of A₁ₐ's may be the same as or different from each other.
